SEQUENCE LISTING (16 sequences)

SEQ ID NO: 1 - protein, 5 aa, Mus musculus
DYYIT

SEQ ID NO: 2 - protein, 17 aa, Mus musculus
WIYPGSGNTKYNEKFKG

SEQ ID NO: 3 - protein, 8 aa, Mus musculus
YGNYWFAY

SEQ ID NO: 4 - protein, 15 aa, Mus musculus
KASQSVDFDGDSYMN

SEQ ID NO: 5 - protein, 7 aa, Mus musculus
AASNLES

SEQ ID NO: 6 - protein, 9 aa, Mus musculus
QQSNEDPWT

SEQ ID NO: 7 - protein, 117 aa, Mus musculus
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 8 - protein, 111 aa, Mus musculus
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 9 - protein, 447 aa, Artificial Sequence
  cAC10 HC
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 446 aa, Artificial Sequence
  cAC10 HC v2
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 218 aa, Artificial Sequence
  cAC10 LC
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 4 aa, Artificial Sequence
  short peptide
GGGG

SEQ ID NO: 13 - protein, 4 aa, Artificial Sequence
  short peptide
GVKG

SEQ ID NO: 14 - protein, 4 aa, Artificial Sequence
  short peptide
VKGG

SEQ ID NO: 15 - protein, 4 aa, Artificial Sequence
  short peptide
GGFG

SEQ ID NO: 16 - protein, 5 aa, Artificial Sequence
  short peptide
GGFGG

(19) United States
(12) Patent Application Publication
(10) Pub. No.: US 2022/0193069 A1
(43) Pub. Date: Jun. 23, 2022

(54) CAMPTOTHECIN PEPTIDE CONJUGATES

(71) Applicant: Seagen Inc., Bothell, WA (US)

(72) Inventors: Scott JEFFREY, Snohomish, WA (US); Ryan Lyski, Lynnwood, WA (US); Maureen Ryan, Bellevue, WA (US); Julia Cochran, Seattle, WA (US)

(21) Appl. No.: 17/452,516

(22) Filed: Oct. 27, 2021

Related U.S. Application Data
(63) Continuation of application No. 16/376,302, filed on Apr. 5, 2019, now abandoned.
(60) Provisional application No. 62/653,961, filed on Apr. 6, 2018.

(51) Int. Cl. A61K 31/4745; A61K 47/62; A61P 35/00

(52) U.S. Cl. CPC A61K 31/4745; A61P 35/00; A61K 47/62

(57) ABSTRACT

Provided herein are Camptothecin Conjugates, Camptothecin-Linker Compounds, Camptothecin Compounds, intermediates thereof, and method of preparing the same. Also provided herein are methods of treating cancer and autoimmune diseases with the Conjugates described herein.

### CROSS REFERENCE TO RELATED APPLICATIONS

The present application is a continuation of U.S. Ser. No. 16/376,302 filed on Apr. 5, 2019, which claims the priority benefit of U.S. Provisional Patent Application No. 62/653,961, filed Apr. 6, 2018, which are incorporated herein by reference in their entireties for all purposes.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761682003701SEQLIST.TXT, date recorded: Oct. 21, 2021, size: 13,860 bytes).

### BACKGROUND

A great deal of interest has surrounded the use of monoclonal antibodies (mAbs) for the targeted delivery of cytotoxic agents to tumor cells. While a number of different drug classes have been evaluated for delivery via antibodies, only a few drug classes have proved sufficiently active as antibody drug conjugates, while having a suitable toxicity profile, to warrant clinical development. One class receiving interest is the camptothecins.

The design of Antibody Drug Conjugates (ADCs), by attaching a cytotoxic agent to antibody, typically via a linker, involves consideration of a variety of factors, including the presence of a conjugation handle on the drug for attachment to the linker and linker technology for attaching the drug to an antibody in a conditionally stable manner. Certain drug classes thought to be lacking appropriate conjugation handles have been considered unsuitable for use as ADCs. Although it may be possible to modify such a drug to include a conjugation handle, such a modification can negatively interfere with the drug's activity profile.

Linkers comprising esters and carbonates have also typically been used for conjugation of alcohol-containing drugs and result in ADCs having variable stability and drug release profiles. A non-optimal profile can result in reduced ADC potency, insufficient immunologic specificity of the conjugate and increased toxicity due to non-specific release of the drug from the conjugate.

Therefore, a need exists for new linker technologies and conjugates useful for targeted therapy. The present invention addresses those and other needs.

### BRIEF SUMMARY

The invention provides, inter alia, Camptothecin Conjugates, Camptothecin-Linker Compounds and Camptothecin Compounds, methods of preparing and using them, and intermediates useful in the preparation thereof. The Camptothecin Conjugates of the present invention are stable in circulation, yet capable of inflicting cell death once free drug is released from a Conjugate in the vicinity or within tumor cells.

In one principal embodiment, a Camptothecin Conjugate is provided having a formula:

L-(Q-D)ₚ

or a pharmaceutically acceptable form thereof, wherein

### L is a Ligand Unit;

Q is a Linker Unit having a formula selected from the group consisting of:

—Z-A-S*—RL-; —Z-A-L^{P}(S*)—RL-; —Z-A-S*—RL-Y—; and —Z-A-L^{P}(S*)—RL-Y—;
wherein Z is a Stretcher Unit, A is a bond or a Connector Unit; L^{P} is a Parallel Connector Unit; S* is a bond or a Partitioning Agent; RL is a peptide comprising from 2 to 8 amino acids; and Y is a Spacer Unit;
   D is a Drug Unit selected from:

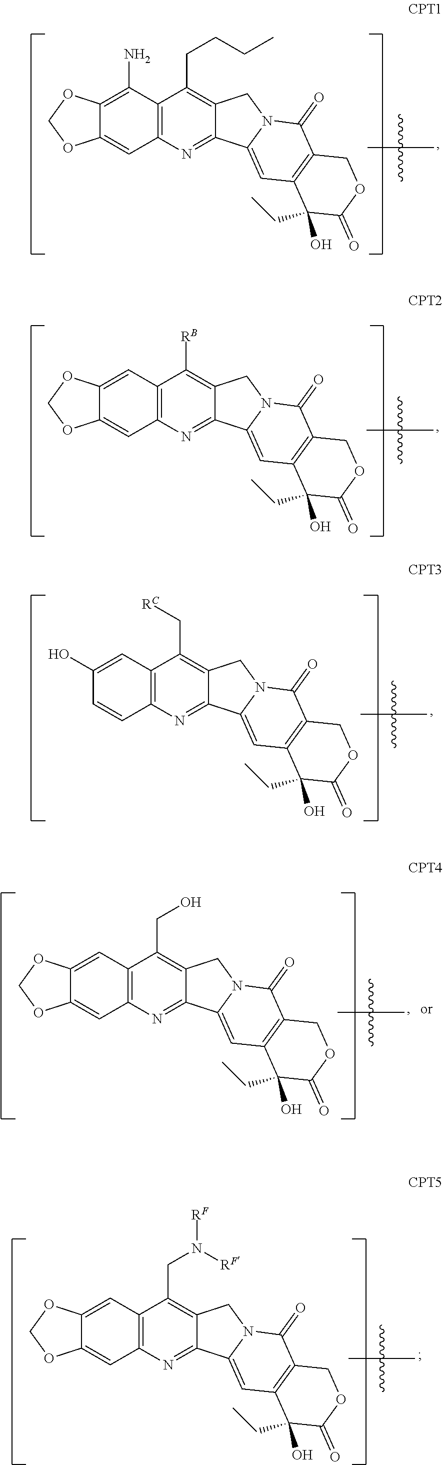

wherein
R^{B} is a member selected from the group consisting of H, C₁-C₈ alkyl, C₁-C₈ haloalkyl, C₃-C₈ cycloalkyl, C₃-C₈cycloalkylC₁-C₄ alkyl, phenyl and phenylC₁-C₄ alkyl;
   R^{C} is a member selected from the group consisting of C₁-C₆ alkyl and C₃-C₆ cycloalkyl;
each R^{F} and R^{F′} is a member independently selected from the group consisting of H, C₁-C₈ alkyl, C₁-C₈ hydroxyalkyl, C₁-C₈ aminoalkyl, C₁-C₄ alkylaminoC₁-C₈ alkyl, (C₁-C₄ hydroxyalkyl)(C₁-C₄ alkyl)aminoC₁-C₈ alkyl, di(C₁-C₄ alkyl)aminoC₁-C₈ alkyl, C₁-C₄ hydroxyalkylC₁-C₈ aminoalkyl, C₂-C₆ heteroalkyl, C₁-C₈ alkylC(O)—, C₁-C₈ hydroxyalkylC(O)—, C₁-C₈ aminoalkylC(O)—, C₃-C₁₀ cycloalkyl, C₃-C₁₀cycloalkylC₁-C₄ alkyl, C₃-C₁₀ heterocycloalkyl, C₃-C₁₀ heterocycloalkylC₁-C₄ alkyl, phenyl, phenylC₁-C₄ alkyl, diphenylC₁-C₄ alkyl, heteroaryl and heteroarylC₁-C₄ alkyl; or R^{F} and R^{F′} are combined with the nitrogen atom to which each is attached to form a 5-, 6- or 7-membered ring having 0 to 3 substituents selected from halogen, C₁-C₄ alkyl, OH, OC₁-C₄ alkyl, NH₂, NHC₁-C₄ alkyl and N(C₁-C₄ alkyl)₂;
and wherein cycloalkyl, heterocycloalkyl, phenyl and heteroaryl portions of R^{B}, R^{C}, R^{F} and R^{F′} are substituted with from 0 to 3 substituents selected from halogen, C₁-C₄ alkyl, OH, OC₁-C₄ alkyl, NH₂, NHC₁-C₄ alkyl and N(C₁-C₄ alkyl)₂; and
   p is from about 1 to about 16;
wherein Q is attached through any one of the hydroxyl or amine groups present on CPT1, CPT2, CPT3, CPT4 or CPT5.

In another principal embodiment, a Camptothecin Conjugate is provided having a formula:

L-(Q-D)ₚ

or a pharmaceutically acceptable salt thereof, wherein

### L is a Ligand Unit;

Q is a Linker Unit having a formula selected from the group consisting of:

—Z-A-S*—RL-; —Z-A-L^{P}(S*)—RL-; —Z-A-S*—RL-Y—; and —Z-A-L^{P}(S*)—RL-Y—;
wherein Z is a Stretcher Unit, A is a bond or a Connector Unit; L^{P} is a Parallel Connector Unit; S* is a bond or a Partitioning Agent; RL is a peptide comprising from 2 to 8 amino acids; and Y is a Spacer Unit;
   D is a Drug Unit selected from the group consisting of:

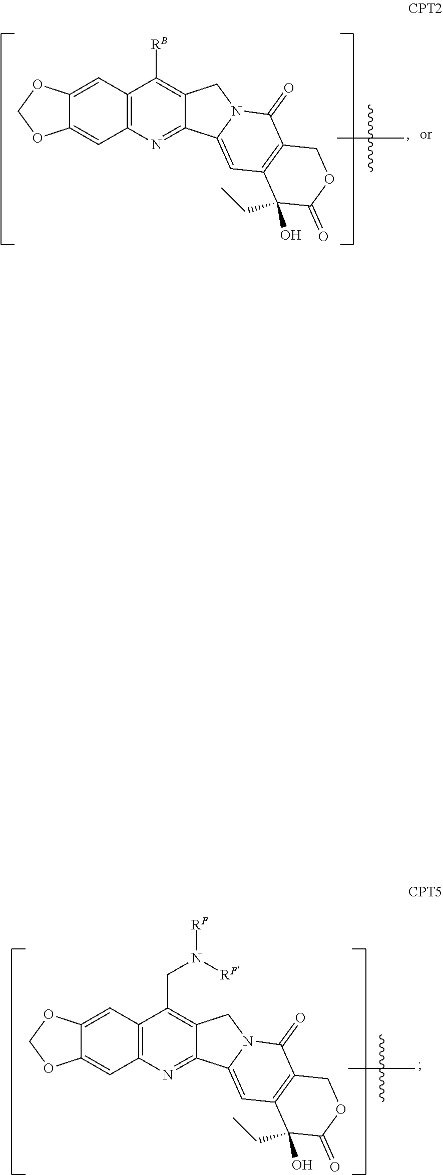

wherein
R^{B} is a member selected from the group consisting of —H, —(C₁-C₄)alkyl-OH, C₁-C₈ alkyl, C₁-C₈ haloalkyl, C₃-C₈ cycloalkyl, C₃-C₈cycloalkylC₁-C₄ alkyl, phenyl and phenylC₁-C₄ alkyl;
each R^{F} and R^{F′} is a member independently selected from the group consisting of H, C₁-C₈ alkyl, C₁-C₈ hydroxyalkyl, C₁-C₈ aminoalkyl, C₁-C₄ alkylaminoC₁-C₈ alkyl, (C₁-C₄ hydroxyalkyl)(C₁-C₄ alkyl)aminoC₁-C₈ alkyl, di(C₁-C₄ alkyl)aminoC₁-C₈ alkyl, C₁-C₄ hydroxyalkylC₁-C₈ aminoalkyl, C₂-C₆ heteroalkyl, C₁-C₈ alkylC(O)—, C₁-C₈ hydroxyalkylC(O)—, C₁-C₈ aminoalkylC(O)—, C₃-C₁₀ cycloalkyl, C₃-C₁₀cycloalkylC₁-C₄ alkyl, C₃-C₁₀ heterocycloalkyl, C₃-C₁₀ heterocycloalkylC₁-C₄ alkyl, phenyl, phenylC₁-C₄ alkyl, diphenylC₁-C₄ alkyl, heteroaryl and heteroarylC₁-C₄ alkyl; or R^{F} and R^{F′} are combined with the nitrogen atom to which each is attached to form a 5-, 6- or 7-membered ring having 0 to 3 substituents selected from halogen, C₁-C₄ alkyl, OH, OC₁-C₄ alkyl, NH₂, NHC₁-C₄ alkyl and N(C₁-C₄ alkyl)₂;
and wherein cycloalkyl, heterocycloalkyl, phenyl and heteroaryl portions of R^{B}, R^{C}, R^{F} and R^{F′} are substituted with from 0 to 3 substituents selected from halogen, C₁-C₄ alkyl, OH, OC₁-C₄ alkyl, NH₂, NHC₁-C₄ alkyl and N(C₁-C₄ alkyl)₂; and
   p is from about 1 to about 16;
wherein Q is attached through any one of the hydroxyl or amine groups present on CPT2 or CPT5.

In yet another principal embodiment, a Camptothecin Conjugate is provided having a formula:

L-(Q-D)ₚ

or a pharmaceutically acceptable salt thereof, wherein

### L is a Ligand Unit;

Q is a Linker Unit having a formula selected from the group consisting of:

—Z-A-S*—RL-; —Z-A-L^{P}(S*)—RL-; —Z-A-S*—RL-Y—; and —Z-A-L^{P}(S*)—RL-Y—;
wherein Z is a Stretcher Unit, A is a bond or a Connector Unit; L^{P} is a Parallel Connector Unit; S* is a bond or a Partitioning Agent; RL is a peptide comprising from 2 to 8 amino acids; and Y is a Spacer Unit;
   D is a Drug Unit having the following structure formula:

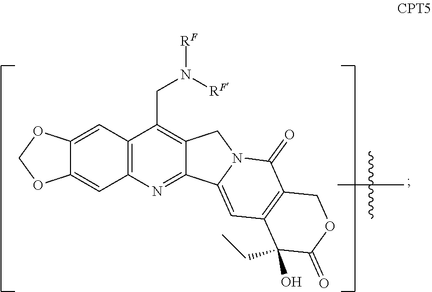

wherein
each R^{F} and R^{F′} is a member independently selected from the group consisting of H, C₁-C₈ alkyl, C₁-C₈ hydroxyalkyl, C₁-C₈ aminoalkyl, C₁-C₄ alkylaminoC₁-C₈ alkyl, (C₁-C₄ hydroxyalkyl)(C₁-C₄ alkyl)aminoC₁-C₈ alkyl, di(C₁-C₄ alkyl)aminoC₁-C₈ alkyl, C₁-C₄ hydroxyalkylC₁-C₈ aminoalkyl, C₂-C₆ heteroalkyl, C₁-C₈ alkylC(O)—, C₁-C₈ hydroxyalkylC(O)—, C₁-C₈ aminoalkylC(O)—, C₃-C₁₀ cycloalkyl, C₃-C₁₀cycloalkylC₁-C₄ alkyl, C₃-C₁₀ heterocycloalkyl, C₃-C₁₀ heterocycloalkylC₁-C₄ alkyl, phenyl, phenylC₁-C₄ alkyl, diphenylC₁-C₄ alkyl, heteroaryl and heteroarylC₁-C₄ alkyl; or R^{F} and R^{F′} are combined with the nitrogen atom to which each is attached to form a 5-, 6- or 7-membered ring having 0 to 3 substituents selected from halogen, C₁-C₄ alkyl, OH, OC₁-C₄ alkyl, NH₂, NHC₁-C₄ alkyl and N(C₁-C₄ alkyl)₂;
and wherein cycloalkyl, heterocycloalkyl, phenyl and heteroaryl portions of R^{B}, R^{C}, R^{F} and R^{F′} are substituted with from 0 to 3 substituents selected from halogen, C₁-C₄ alkyl, OH, OC₁-C₄ alkyl, NH₂, NHC₁-C₄ alkyl and N(C₁-C₄ alkyl)₂; and
   p is from about 1 to about 16;
wherein Q is attached through any one of the hydroxyl or amine groups present on CPT5.

Other principal embodiments as noted above, are Camptothecin-Linker Compounds useful as intermediates for preparing Camptothecin Conjugates, wherein the Camptothecin-Linker Compound is comprised of a Camptothecin (D) and a Linker Unit (Q), wherein the Linker Unit is comprised of a Stretcher Unit precursor (Z′) capable of forming a covalent bond to a targeting ligand that provides for a Ligand Unit, and a Releasable Linker (RL) which is a peptide of from 2 to 8 amino acids.

In another aspect, provided herein are methods of treating cancer comprising administering to a subject in need thereof a Camptothecin Conjugate described herein.

In another aspect, provided herein are methods of treating cancer using Camptothecin-Linker Compounds or Camptothecins described herein.

In another aspect, provided herein are kits comprising a Camptothecin Conjugate described herein.

### BRIEF DESCRIPTION OF THE DRAWINGS

FIGS. 1A and 1B show a mean tumor volume graph for an L540cy subcutaneous mouse xenograft model of Hodgkin lymphoma, comparing activity of peptide-based camptothecin ADCs.

FIG. 2 shows the effect of peptide-based camptothecin ADCs on mean tumor volume for a 786-O renal cell carcinoma subcutaneous mouse xenograft model.

FIGS. 3A-3C show the results of Karpas 299/Karpas299-BVR anaplastic large cell lymphoma bystander subcutaneous xenograft tumor model.

FIGS. 4A-4D show the activity of CD30-directed camptothecin ADCs in DelBVR model.

FIGS. 5A and 5B show the activity of CD30-directed camptothecin ADCs and comparison with brentuximab vedotin in DelBVR model.

FIG. 6 shows the activities CD30-directed camptothecin ADCs in Karpas 299 model using single and repeat dosing.

FIGS. 7A and 7B show the activities CD30-directed camptothecin ADCs in L428 model using single and repeat dosing.

FIG. 8 shows the activities CD30-directed camptothecin ADCs in DEL-15 model using various doses.

FIG. 9 shows the activities CD30-directed camptothecin ADCs in L82 model.

FIG. 10 shows the results of an ADC stability study in mouse plasma.

FIG. 11 shows the pharmacokinetic profile of IgG mAb, and IgG-camptothecin ADCs in Sprague-Dawley rat.

FIG. 12 shows the results of a Kupffer cell ADC uptake assay.

FIG. 13 shows the results of hydrophobic interaction chromatography with unconjugated cAC10 monoclonal antibody and CD30-directed camptothecin ADCs.

FIGS. 14A and 14B show the results of in vitro drug release from CD30-directed camptothecin ADCs in ALCL cell line Karpass 299 and HL cell line L540cy, respectively.

### DETAILED DESCRIPTION

### Definitions

Unless stated otherwise, the following terms and phrases as used herein are intended to have the following meanings. When trade names are used herein, the trade name includes the product formulation, the generic drug, and the active pharmaceutical ingredient(s) of the trade name product, unless otherwise indicated by context.

The term “antibody” as used herein is used in the broadest sense and specifically covers intact monoclonal antibodies, polyclonal antibodies, monospecific antibodies, multispecific antibodies (e.g., bispecific antibodies), and antibody fragments that exhibit the desired biological activity. The native form of an antibody is a tetramer and consists of two identical pairs of immunoglobulin chains, each pair having one light chain and one heavy chain. In each pair, the light and heavy chain variable regions (VL and VH) are together primarily responsible for binding to an antigen. The light chain and heavy chain variable domains consist of a framework region interrupted by three hypervariable regions, also called “complementarity determining regions” or “CDRs.” The constant regions may be recognized by and interact with the immune system. (see, e.g., Janeway et al., 2001, Immunol. Biology, 5th Ed., Garland Publishing, New York). An antibody can be of any type (e.g., IgG, IgE, IgM, IgD, and IgA), class (e.g., IgG1, IgG2, IgG3, IgG4, IgA1 and IgA2) or subclass. The antibody can be derived from any suitable species. In some embodiments, the antibody is of human or murine origin. An antibody can be, for example, human, humanized or chimeric.

The term “monoclonal antibody” as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. The modifier “monoclonal” indicates the character of the antibody as being obtained from a substantially homogeneous population of antibodies, and is not to be construed as requiring production of the antibody by any particular method.

An “intact antibody” is one which comprises an antigen-binding variable region as well as a light chain constant domain (C_{L}) and heavy chain constant domains, C_{H}1, C_{H}2, C_{H}3 and C_{H}4, as appropriate for the antibody class. The constant domains may be native sequence constant domains (e.g., human native sequence constant domains) or amino acid sequence variant thereof.

An “antibody fragment” comprises a portion of an intact antibody, comprising the antigen-binding or variable region thereof. Examples of antibody fragments include Fab, Fab′, F(ab′)₂, and Fv fragments, diabodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragments of any of the above which immunospecifically bind to a target antigen (e.g., a cancer cell antigen, a viral antigen or a microbial antigen).

An “antigen” is an entity to which an antibody specifically binds.

The terms “specific binding” and “specifically binds” mean that the antibody or antibody derivative will bind, in a highly selective manner, with its corresponding epitope of a target antigen and not with the multitude of other antigens. Typically, the antibody or antibody derivative binds with an affinity of at least about 1×10⁻⁷ M, and preferably 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely-related antigen.

The term “inhibit” or “inhibition of” means to reduce by a measurable amount, or to prevent entirely.

The term “therapeutically effective amount” refers to an amount of a conjugate effective to treat a disease or disorder in a mammal. In the case of cancer, the therapeutically effective amount of the conjugate may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may inhibit growth and/or kill existing cancer cells, it may be cytostatic and/or cytotoxic. For cancer therapy, efficacy can, for example, be measured by assessing the time to disease progression (TTP) and/or determining the response rate (RR).

The term “substantial” or “substantially” refers to a majority, i.e. >50% of a population, of a mixture or a sample, preferably more than 50%, 55%, 60%, 65%, 70%, 75%, 80%, 85%, 90%, 91%, 92%, 93%, 94%, 95%, 96%, 97%, 98%, or 99% of a population.

The term “cytotoxic activity” refers to a cell-killing effect of a drug or Camptothecin Conjugate or an intracellular metabolite of a Camptothecin Conjugate. Cytotoxic activity may be expressed as the IC₅₀ value, which is the concentration (molar or mass) per unit volume at which half the cells survive.

The term “cytostatic activity” refers to an anti-proliferative effect of a drug or Camptothecin Conjugate or an intracellular metabolite of a Camptothecin Conjugate.

The term “cytotoxic agent” as used herein refers to a substance that has cytotoxic activity and causes destruction of cells. The term is intended to include chemotherapeutic agents, and toxins such as small molecule toxins or enzymatically active toxins of bacterial, fungal, plant or animal origin, including synthetic analogs and derivatives thereof.

The term “cytostatic agent” as used herein refers to a substance that inhibits a function of cells, including cell growth or multiplication. Cytostatic agents include inhibitors such as protein inhibitors, e.g., enzyme inhibitors. Cytostatic agents have cytostatic activity.

The terms “cancer” and “cancerous” refer to or describe the physiological condition or disorder in mammals that is typically characterized by unregulated cell growth. A “tumor” comprises one or more cancerous cells.

An “autoimmune disease” as used herein refers to a disease or disorder arising from and directed against an individual's own tissues or proteins.

“Patient” as used herein refers to a subject to whom is administered a Camptothecin Conjugate of the present invention. Patient includes, but are not limited to, a human, rat, mouse, guinea pig, non-human primate, pig, goat, cow, horse, dog, cat, bird and fowl. Typically, the patient is a rat, mouse, dog, human or non-human primate, more typically a human.

The terms “treat” or “treatment,” unless otherwise indicated by context, refer to therapeutic treatment and prophylactic wherein the object is to inhibit or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder.

In the context of cancer, the term “treating” includes any or all of: killing tumor cells; inhibiting growth of tumor cells, cancer cells, or of a tumor; inhibiting replication of tumor cells or cancer cells, lessening of overall tumor burden or decreasing the number of cancerous cells, and ameliorating one or more symptoms associated with the disease.

In the context of an autoimmune disease, the term “treating” includes any or all of: inhibiting replication of cells associated with an autoimmune disease state including, but not limited to, cells that produce an autoimmune antibody, lessening the autoimmune-antibody burden and ameliorating one or more symptoms of an autoimmune disease.

The term “pharmaceutically acceptable form” as used herein refers to a form of a disclosed compound including, but is not limited to, pharmaceutically acceptable salts, esters, hydrates, solvates, polymorphs, isomers, prodrugs, and isotopically labeled derivatives thereof. In one embodiment, a “pharmaceutically acceptable form” includes, but is not limited to, pharmaceutically acceptable salts, esters, prodrugs and isotopically labeled derivatives thereof. In some embodiments, a “pharmaceutically acceptable form” includes, but is not limited to, pharmaceutically acceptable isomers and stereoisomers, prodrugs and isotopically labeled derivatives thereof.

In certain embodiments, the pharmaceutically acceptable form is a pharmaceutically acceptable salt. The term “pharmaceutically acceptable salt,” as used herein, refers to pharmaceutically acceptable organic or inorganic salts of a compound (e.g., a Drug, Drug-Linker, or a Camptothecin Conjugate). In some aspects, the compound can contain at least one amino group, and accordingly acid addition salts can be formed with the amino group. Exemplary salts include, but are not limited to, sulfate, trifluoroacetate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

A Linker Unit is a bifunctional moiety that connects a Camptothecin to a Ligand Unit in a Camptothecin Conjugate. The Linker Units of the present invention have several components (e.g., a Stretcher Unit which in some embodiments will have a Basic Unit; a Connector Unit, that can be present or absent; a Parallel Connector Unit, that can also be present or absent; a Peptide Releasable Linking Unit; and a Spacer Unit, that can also be present or absent).

“PEG Unit” as used herein is an organic moiety comprised of repeating ethylene-oxy subunits (PEGs or PEG subunits) and may be polydisperse, monodisperse or discrete (i.e., having discrete number of ethylene-oxy subunits). Polydisperse PEGs are a heterogeneous mixture of sizes and molecular weights whereas monodisperse PEGs are typically purified from heterogeneous mixtures and are therefore provide a single chain length and molecular weight. Preferred PEG Units comprises discrete PEGs, compounds that are synthesized in step-wise fashion and not via a polymerization process. Discrete PEGs provide a single molecule with defined and specified chain length.

The PEG Unit provided herein comprises one or multiple polyethylene glycol chains, each comprised of one or more ethyleneoxy subunits, covalently attached to each other. The polyethylene glycol chains can be linked together, for example, in a linear, branched or star shaped configuration. Typically, at least one of the polyethylene glycol chains prior to incorporation into a Camptothecin Conjugate is derivatized at one end with an alkyl moiety substituted with an electrophilic group for covalent attachment to the carbamate nitrogen of a methylene carbamate unit (i.e., represents an instance of R). Typically, the terminal ethyleneoxy subunit in each polyethylene glycol chains not involved in covalent attachment to the remainder of the Linker Unit is modified with a PEG Capping Unit, typically an optionally substituted alkyl such as —CH₃, CH₂CH₃ or CH₂CH₂CO₂H. A preferred PEG Unit has a single polyethylene glycol chain with 2 to 24 —CH₂CH₂O— subunits covalently attached in series and terminated at one end with a PEG Capping Unit.

Unless otherwise indicated, the term “alkyl” by itself or as part of another term refers to a substituted or unsubstituted straight chain or branched, saturated or unsaturated hydrocarbon having the indicated number of carbon atoms (e.g., “—C₁-C₈ alkyl” or “—C₁-C₁₀” alkyl refer to an alkyl group having from 1 to 8 or 1 to 10 carbon atoms, respectively). When the number of carbon atoms is not indicated, the alkyl group has from 1 to 8 carbon atoms. Representative straight chain “—C₁-C₈ alkyl” groups include, but are not limited to, -methyl, -ethyl, -n-propyl, -n-butyl, -n-pentyl, -n-hexyl, -n-heptyl and -n-octyl; while branched —C₃-C₈ alkyls include, but are not limited to, -isopropyl, -sec-butyl, -isobutyl, -tert-butyl, -isopentyl, and -2-methylbutyl; unsaturated —C₂-C₈ alkyls include, but are not limited to, -vinyl, -allyl, -1-butenyl, -2-butenyl, -isobutylenyl, -1 pentenyl, -2 pentenyl, -3-methyl-1-butenyl, -2 methyl-2-butenyl, -2,3 dimethyl-2-butenyl, -1-hexyl, 2-hexyl, -3-hexyl, -acetylenyl, -propynyl, -1 butynyl, -2 butynyl, -1 pentynyl, -2 pentynyl and -3 methyl 1 butynyl. Sometimes an alkyl group is unsubstituted. An alkyl group can be substituted with one or more groups. In other aspects, an alkyl group will be saturated.

Unless otherwise indicated, “alkylene,” by itself of as part of another term, refers to a substituted or unsubstituted saturated, branched or straight chain or cyclic hydrocarbon radical of the stated number of carbon atoms, typically 1-10 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethylene (—CH₂CH₂—), 1,3-propylene (—CH₂CH₂CH₂—), 1,4-butylene (—CH₂CH₂CH₂CH₂—), and the like. In preferred aspects, an alkylene is a branched or straight chain hydrocarbon (i.e., it is not a cyclic hydrocarbon).

Unless otherwise indicated, “aryl,” by itself or as part of another term, means a substituted or unsubstituted monovalent carbocyclic aromatic hydrocarbon radical of the stated number of carbon atoms, typically 6-20 carbon atoms, derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, biphenyl, and the like. An exemplary aryl group is a phenyl group.

Unless otherwise indicated, an “arylene,” by itself or as part of another term, is an aryl group as defined above which has two covalent bonds (i.e., it is divalent) and can be in the ortho, meta, or para orientations as shown in the following structures, with phenyl as the exemplary group:

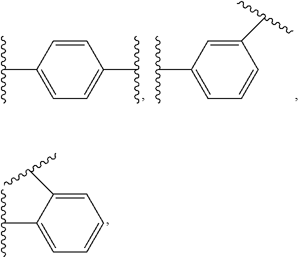

Unless otherwise indicated, a “C₃-C₈ heterocycle,” by itself or as part of another term, refers to a monovalent substituted or unsubstituted aromatic or non-aromatic monocyclic or bicyclic ring system having from 3 to 8 carbon atoms (also referred to as ring members) and one to four heteroatom ring members independently selected from N, O, P or S, and derived by removal of one hydrogen atom from a ring atom of a parent ring system. One or more N, C or S atoms in the heterocycle can be oxidized. The ring that includes the heteroatom can be aromatic or nonaromatic. Heterocycles in which all of the ring atoms are involved in aromaticity are referred to as heteroaryls and otherwise are referred to heterocarbocycles. Unless otherwise noted, the heterocycle is attached to its pendant group at any heteroatom or carbon atom that results in a stable structure. As such a heteroaryl may be bonded through an aromatic carbon of its aromatic ring system, referred to as a C-linked heteroaryl, or through a non-double-bonded N atom (i.e., not ═N—) in its aromatic ring system, which is referred to as an N-linked heteroaryl. Thus, nitrogen-containing heterocycles may be C-linked or N-linked and include pyrrole moieties, such as pyrrol-1-yl (N-linked) and pyrrol-3-yl (C-linked), and imidazole moieties such as imidazol-1-yl and imidazol-3-yl (both N-linked), and imidazol-2-yl, imidazol-4-yl and imidazol-5-yl moieties (all of which are C-linked).

Unless otherwise indicated, a “C₃-C₈ heteroaryl,” is an aromatic C₃-C₈ heterocycle in which the subscript denotes the total number of carbons of the cyclic ring system of the heterocycle or the total number of aromatic carbons of the aromatic ring system of the heteroaryl and does not implicate the size of the ring system or the presence or absence of ring fusion. Representative examples of a C₃-C₈ heterocycle include, but are not limited to, pyrrolidinyl, azetidinyl, piperidinyl, morpholinyl, tetrahydrofuranyl, tetrahydropyranyl, benzofuranyl, benzothiophene, indolyl, benzopyrazolyl, pyrrolyl, thiophenyl (thiophene), furanyl, thiazolyl, imidazolyl, pyrazolyl, pyrimidinyl, pyridinyl, pyrazinyl, pyridazinyl, isothiazolyl, and isoxazolyl. When explicitly given, the size of the ring system of a heterocycle or heteroaryl is indicated by the total number of atoms in the ring. For example, designation as a 5- or 6-membered heteroaryl indicates the total number or aromatic atoms (i.e., 5 or 6) in the heteroaromatic ring system of the heteroaryl, but does not imply the number of aromatic heteroatoms or aromatic carbons in that ring system. Fused heteroaryls are explicitly stated or implied by context as such and are typically indicated by the number of aromatic atoms in each aromatic ring that are fused together to make up the fused heteroaromatic ring system. For example a 5,6-membered heteroaryl is an aromatic 5-membered ring fused to an aromatic 6-membered ring in which one or both of the rings have aromatic heteroatom(s) or where a heteroatom is shared between the two rings.

A heterocycle fused to an aryl or heteroaryl such that the heterocycle remains non-aromatic and is part of a larger structure through attachment with the non-aromatic portion of the fused ring system is an example of an optionally substituted heterocycle in which the heterocycle is substituted by ring fusion with the aryl or heteroaryl. Likewise, an aryl or heteroaryl fused to heterocycle or carbocycle that is part of a larger structure through attachment with the aromatic portion of the fused ring system is an example of an optionally substituted aryl or heterocycle in which the aryl or heterocycle is substituted by ring fusion with the heterocycle or carbocycle.

Unless otherwise indicated, “C₃-C₈ heterocyclo,” by itself or as part of another term, refers to a C₃-C₈ heterocyclic defined above wherein one of the hydrogen atoms of the heterocycle is replaced with a bond (i.e., it is divalent). Unless otherwise indicated, a “C₃-C₈ heteroarylene,” by itself or as part of another term, refers to a C₃-C₈ heteroaryl group defined above wherein one of the heteroaryl group's hydrogen atoms is replaced with a bond (i.e., it is divalent).

Unless otherwise indicated, a “C₃-C₈ carbocycle,” by itself or as part of another term, is a 3-, 4-, 5-, 6-, 7- or 8-membered monovalent, substituted or unsubstituted, saturated or unsaturated non-aromatic monocyclic or bicyclic carbocyclic ring derived by the removal of one hydrogen atom from a ring atom of a parent ring system. Representative —C₃-C₈ carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl.

Unless otherwise indicated, a “C₃-C₈ carbocyclo,” by itself or as part of another term, refers to a C₃-C₈ carbocycle group defined above wherein another of the carbocycle groups' hydrogen atoms is replaced with a bond (i.e., it is divalent).

Unless otherwise indicated, the term “heteroalkyl,” by itself or in combination with another term, means, unless otherwise stated, a stable straight or branched chain hydrocarbon, or combinations thereof, fully saturated or containing from 1 to 3 degrees of unsaturation, consisting of the stated number of carbon atoms and from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group or at the position at which the alkyl group is attached to the remainder of the molecule. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —NH—CH₂—CH₂—NH—C(O)—CH₂—CH₃, —CH₂—CH₂—S(O)₂—CH₃, —CH═CH—O—CH₃, —Si(CH₃)₃, —CH₂—CH═N—O—CH₃, and —CH═CH—N(CH₃)—CH₃. Up to two heteroatoms may be consecutive, such as, for example, —CH₂—NH—OCH₃ and —CH₂—O—Si(CH₃)₃. Typically, a C₁ to C₄ heteroalkyl or heteroalkylene has 1 to 4 carbon atoms and 1 or 2 heteroatoms and a C₁ to C₃ heteroalkyl or heteroalkylene has 1 to 3 carbon atoms and 1 or 2 heteroatoms. In some aspects, a heteroalkyl or heteroalkylene is saturated.

Unless otherwise indicated, the term “heteroalkylene” by itself or in combination with another term means a divalent group derived from heteroalkyl (as discussed above), as exemplified by —CH₂—CH₂—S—CH₂—CH₂— and —CH₂—S—CH₂—CH₂—NH—CH₂—. For heteroalkylene groups, heteroatoms can also occupy either or both of the chain termini. Still further, for alkylene and heteroalkylene linking groups, no orientation of the linking group is implied.

Unless otherwise indicated, “aminoalkyl” by itself or in combination with another term means a heteroalkyl wherein an alkyl moiety as defined herein is substituted with an amino, alkylamino, dialkylamino or cycloalkylamino group. Exemplary non-limiting aminoalkyls are —CH₂NH₂, —CH₂CH₂NH₂, —CH₂CH₂NHCH₃ and —CH₂CH₂N(CH₃)₂ and further includes branched species such as —CH(CH₃)NH₂ and —C(CH₃)CH₂NH₂ in the (R)- or (S)-configuration. Alternatively, an aminoalkyl is an alkyl moiety, group, or substituent as defined herein wherein a sp³ carbon other than the radical carbon has been replaced with an amino or alkylamino moiety wherein its sp³ nitrogen replaces the sp³ carbon of the alkyl provided that at least one sp³ carbon remains. When referring to an aminoalkyl moiety as a substituent to a larger structure or another moiety the aminoalkyl is covalently attached to the structure or moiety through the carbon radical of the alkyl moiety of the aminoalkyl.

Unless otherwise indicated “alkylamino” and “cycloalkylamino” by itself or in combination with another term means an alkyl or cycloalkyl radical, as described herein, wherein the radical carbon of the alkyl or cycloalkyl radical has been replaced with a nitrogen radical, provided that at least one sp³ carbon remains. In those instances where the alkylamino is substituted at its nitrogen with another alkyl moiety the resulting substituted radical is sometimes referred to as a dialkylamino moiety, group or substituent wherein the alkyl moieties substituting nitrogen are independently selected. Exemplary and non-limiting amino, alkylamino and dialkylamino substituents, include those having the structure of —N(R′)₂, wherein R′ in these examples are independently selected from hydrogen or C₁₋₆ alkyl, typically hydrogen or methyl, whereas in cycloalkyl amines, which are included in heterocycloalkyls, both R′ together with the nitrogen to which they are attached define a heterocyclic ring. When both R′ are hydrogen or alkyl, the moiety is sometimes described as a primary amino group and a tertiary amine group, respectively. When one R′ is hydrogen and the other is alkyl, then the moiety is sometimes described as a secondary amino group. Primary and secondary alkylamino moieties are more reactive as nucleophiles towards carbonyl-containing electrophilic centers whereas tertiary amines are more basic.

“Substituted alkyl” and “substituted aryl” mean alkyl and aryl, respectively, in which one or more hydrogen atoms, typically one, are each independently replaced with a substituent. Typical substituents include, but are not limited to a —X, —R′, —OH, —OR′, —SR′, —N(R′)₂, —N(R′)₃, ═NR′, —CX₃, —CN, —NO₂, —NR′C(═O)R′, —C(═O)R′, —C(═O)N(R′)₂, —S(═O)₂R′, —S(═O)₂NR′, —S(═O)R′, —OP(═O)(OR′)₂, —P(═O)(OR′)₂, —PO₃⁼, PO₃H₂, —C(═O)R′, —C(═S)R′, —CO₂R′, —CO₂⁻, —C(═S)OR′, —C(═O)SR′, —C(═S)SR′, —C(═O)N(R′)₂, —C(═S)N(R′)₂, and —C(═NR)N(R′)₂, where each X is independently selected from the group consisting of a halogen: —F, —Cl, —Br, and —I; and wherein each R′ is independently selected from the group consisting of —H, —C₁-C₂₀ alkyl, —C₆-C₂₀ aryl, —C₃-C₁₄ heterocycle, a protecting group, and a prodrug moiety.

More typically substituents are selected from the group consisting of —X, —R′, —OH, —OR′, —SR′, —N(R′)₂, —N(R′)₃, ═NR′, —NR′C(═O)R′, —C(═O)R′, —C(═O)N(R′)₂, —S(═O)₂R′, —S(═O)₂NR′, —S(═O)R′, —C(═O)R′, —C(═S)R′, —C(═O)N(R′)₂, —C(═S)N(R′)₂, and —C(═NR)N(R′)₂, wherein each X is independently selected from the group consisting of —F and —Cl, or are selected from the group consisting of —X, —R′, —OH, —OR′, —N(R′)₂, —N(R′)₃, —NR′C(═O)R′, —C(═O)N(R′)₂, —S(═O)₂R′, —S(═O)₂NR′, —S(═O)R′, —C(═O)R′, —C(═O)N(R′)₂, —C(═NR)N(R′)₂, a protecting group, and a prodrug moiety wherein each X is —F; and wherein each R′ is independently selected from the group consisting of hydrogen, —C₁-C₂₀ alkyl, —C₆-C₂₀ aryl, —C₃-C₁₄ heterocycle, a protecting group, and a prodrug moiety. In some aspects, an alkyl substituent is selected from the group consisting —N(R′)₂, —N(R′)₃ and —C(═NR)N(R′)₂, wherein R′ is selected from the group consisting of hydrogen and —C₁-C₂₀ alkyl. In other aspects, alkyl is substituted with a series of ethyleneoxy moieties to define a PEG Unit. Alkylene, carbocycle, carbocyclo, arylene, heteroalkyl, heteroalkylene, heterocycle, heterocyclo, heteroaryl, and heteroarylene groups as described above may also be similarly substituted.

“Protecting group” as used here means a moiety that prevents or reduces the ability of the atom or functional group to which it is linked from participating in unwanted reactions. Typical protecting groups for atoms or functional groups are given in Greene (1999), “PROTECTIVE GROUPS IN ORGANIC SYNTHESIS, 3^{RD} ED.”, Wiley Interscience. Protecting groups for heteroatoms such as oxygen, sulfur and nitrogen are used in some instances to minimize or avoid unwanted their reactions with electrophilic compounds. In other instances, the protecting group is used to reduce or eliminate the nucleophilicity and/or basicity of the unprotected heteroatom. Non-limiting examples of protected oxygen are given by —OR^{PR}, wherein R^{PR} is a protecting group for hydroxyl, wherein hydroxyl is typically protected as an ester (e.g. acetate, propionate or benzoate). Other protecting groups for hydroxyl avoid interfering with the nucleophilicity of organometallic reagents or other highly basic reagents, where hydroxyl is typically protected as an ether, including alkyl or heterocycloalkyl ethers, (e.g., methyl or tetrahydropyranyl ethers), alkoxymethyl ethers (e.g., methoxymethyl or ethoxymethyl ethers), optionally substituted aryl ethers, and silyl ethers (e.g., trimethylsilyl (TMS), triethylsilyl (TES), tert-butyldiphenylsilyl (TBDPS), tert-butyldimethylsilyl (TBS/TBDMS), triisopropylsilyl (TIPS) and [2-(trimethylsilyl)ethoxy]-methylsilyl (SEM)). Nitrogen protecting groups include those for primary or secondary amines as in —NHR^{PR} or —N(R^{PR})₂—, wherein least one of R^{PR} is a nitrogen atom protecting group or both R^{PR} together comprise a protecting group.

A protecting group is suitable when it is capable of preventing or avoiding unwanted side-reactions or premature loss of the protecting group under reaction conditions required to effect desired chemical transformation elsewhere in the molecule and during purification of the newly formed molecule when desired, and can be removed under conditions that do not adversely affect the structure or stereochemical integrity of that newly formed molecule. By way of example and not limitation, a suitable protecting group may include those previously described for protecting functional groups. A suitable protecting group is sometimes a protecting group used in peptide coupling reactions.

“Aromatic alcohol” by itself or part of a larger structure refers to an aromatic ring system substituted with the hydroxyl functional group —OH. Thus, aromatic alcohol refers to any aryl, heteroaryl, arylene and heteroarylene moiety as described herein having a hydroxyl functional group bonded to an aromatic carbon of its aromatic ring system. The aromatic alcohol may be part of a larger moiety as when its aromatic ring system is a substituent of this moiety, or may be embedded into the larger moiety by ring fusion, and may be optionally substituted with moieties as described herein including one or more other hydroxyl substitutents. A phenolic alcohol is an aromatic alcohol having a phenol group as the aromatic ring.

“Aliphatic alcohol” by itself or part of a larger structure refers to a moiety having a non-aromatic carbon bonded to the hydroxyl functional group —OH. The hydroxy-bearing carbon may be unsubstituted (i.e., methyl alcohol) or may have one, two or three optionally substituted branched or unbranched alkyl substituents to define a primary alcohol, or a secondary or tertiary aliphatic alcohol within a linear or cyclic structure. When part of a larger structure, the alcohol may be a substituent of this structure by bonding through the hydroxy bearing carbon, through a carbon of an alkyl or other moiety as described herein to this hydroxyl-bearing carbon or through a substituent of this alkyl or other moiety. An aliphatic alcohol contemplates a non-aromatic cyclic structure (i.e., carbocycles and heterocarbocycles, optionally substituted) in which a hydroxy functional group is bonded to a non-aromatic carbon of its cyclic ring system.

“Arylalkyl” or “heteroarylalkyl” as used herein means a substituent, moiety or group where an aryl moiety is bonded to an alkyl moiety, i.e., aryl-alkyl-, where alkyl and aryl groups are as described above, e.g., C₆H₅—CH₂— or C₆H₅—CH(CH₃)CH₂—. An arylalkyl or heteroarylalkyl is associated with a larger structure or moiety through a sp³ carbon of its alkyl moiety.

“Electron withdrawing group (EWG)” as used herein means a functional group or electronegative atom that draws electron density away from an atom to which it is bonded either inductively and/or through resonance, whichever is more dominant (i.e., a functional group or atom may be electron withdrawing inductively but may overall be electron donating through resonance), and tends to stabilize anions or electron-rich moieties. The electron withdrawing effect is typically transmitted inductively, albeit in attenuated form, to other atoms attached to the bonded atom that has been made electron deficient by the electron withdrawing group (EWG), thus affecting the electrophilicity of a more remote reactive center. Exemplary electron withdrawing groups include, but are not limited to —C(═O), —CN, —NO₂, —CX₃, —X, —C(═O)OR′, —C(═O)N(R′)₂, —C(═O)R′, —C(═O)X, —S(═O)₂R′, —S(═O)₂OR′, —S(═O)₂NHR′, —S(═O)₂N(R′)₂, —P(═O)(OR′)₂, —P(═O)(CH₃)NHR′, —NO, —N(R′)₃⁺, wherein X is —F, —Br, —Cl, or —I, and R′ in some aspects is, at each occurrence, independently selected from the group consisting of hydrogen and C₁₋₆ alkyl, and certain O-linked moieties as described herein such as acyloxy.

Exemplary EWGs can also include aryl groups (e.g., phenyl) depending on substitution and certain heteroaryl groups (e.g., pyridine). Thus, the term “electron withdrawing groups” also includes aryls or heteroaryls that are further substituted with electron withdrawing groups. Typically, electron withdrawing groups on aryls or heteroaryls are —C(═O), —CN, —NO₂, —CX₃, and —X, wherein X independently selected is halogen, typically —F or —Cl. Depending on their substituents, an alkyl moiety may also be an electron withdrawing group.

“Leaving group ability” relates to the ability of an alcohol-, thiol-, amine- or amide-containing compound corresponding to a Camptothecin in a Camptothecin Conjugate to be released from the Conjugate as a free drug subsequent to activation of a self-immolative event within the Conjugate. That release can be variable without the benefit of a methylene carbamate unit to which its Camptothecin is attached (i.e., when the Camptothecin is directly attached to a self-immolative moiety and does not have an intervening methylene carbamate unit). Good leaving groups are usually weak bases and the more acidic the functional group that is expelled from such conjugates the weaker the conjugate base is. Thus, the leaving group ability of an alcohol-, thiol-, amine- or amide-containing free drug from a Camptothecin will be related to the pKa of the drug's functional group that is expelled from a conjugate in cases where methylene carbamate unit (i.e., one in which a Camptothecin is directly attached to a self-immolative moiety) is not used. Thus, a lower pKa for that functional group will increase its leaving group ability. Although other factors may contribute to release of free drug from conjugates not having the benefit of a methylene carbamate unit, generally a drug having a functional group with a lower pKa value will typically be a better leaving group that a drug attached via a functional group with a higher pKa value. Another consideration is that, a functional group having too low of a pKa value may result in an unacceptable activity profile due to premature loss of the Camptothecin via spontaneous hydrolysis. For conjugates employing a methylene carbamate unit, a common functional group (i.e., a carbamic acid) having a pKa value that allows for efficient release of free drug, without suffering unacceptable loss of Camptothecin, is produced upon self-immolation.

“Succinimide moiety” as used herein refers to an organic moiety comprised of a succinimide ring system, which is present in one type of Stretcher Unit (Z) that is typically further comprised of an alkylene-containing moiety bonded to the imide nitrogen of that ring system. A succinimide moiety typically results from Michael addition of a sulfhydryl group of a Ligand Unit to the maleimide ring system of a Stretcher Unit precursor (Z′). A succinimide moiety is therefore comprised of a thio-substituted succinimide ring system and when present in a Camptothecin Conjugate has its imide nitrogen substituted with the remainder of the Linker Unit of the Camptothecin Conjugate and is optionally substituted with substituent(s) that were present on the maleimide ring system of Z′.

“Acid-amide moiety” as used herein refers to succinic acid having an amide substituent that results from the thio-substituted succinimide ring system of a succinimide moiety having undergone breakage of one of its carbonyl-nitrogen bonds by hydrolysis. Hydrolysis resulting in a succinic acid-amide moiety provides a Linker Unit less likely to suffer premature loss of the Ligand Unit to which it is bonded through elimination of the antibody-thio substituent. Hydrolysis of the succinimide ring system of the thio-substituted succinimide moiety is expected to provide regiochemical isomers of acid-amide moieties that are due to differences in reactivity of the two carbonyl carbons of the succinimide ring system attributable at least in part to any substituent present in the maleimide ring system of the Stretcher Unit precursor and to the thio substituent introduced by the targeting ligand.

The term “Prodrug” as used herein refers to a less biologically active or inactive compound which is transformed within the body into a more biologically active compound via a chemical or biological process (i.e., a chemical reaction or an enzymatic biotransformation). Typically, a biologically active compound is rendered less biologically active (i.e., is converted to a prodrug) by chemically modifying the compound with a prodrug moiety. In some aspects the prodrug is a Type II prodrug, which are bioactivated outside cells, e.g., in digestive fluids, or in the body's circulation system, e.g., in blood. Exemplary prodrugs are esters and 3-D-glucopyranosides.

In many instances, the assembly of the conjugates, linkers and components described herein will refer to reactive groups. A “reactive group” or RG is a group that contains a reactive site (RS) that is capable of forming a bond with either the components of the Linker unit (i.e., A, W, Y) or the Camptothecin D. RS is the reactive site within a Reactive Group (RG). Reactive groups include sulfhydryl groups to form disulfide bonds or thioether bonds, aldehyde, ketone, or hydrazine groups to form hydrazone bonds, carboxylic or amino groups to form peptide bonds, carboxylic or hydroxy groups to form ester bonds, sulfonic acids to form sulfonamide bonds, alcohols to form carbamate bonds, and amines to form sulfonamide bonds or carbamate bonds. The following table is illustrative of Reactive Groups, Reactive Sites, and exemplary functional groups that can form after reaction of the reactive site. The table is not limiting. One of skill in the art will appreciate that the noted R′ and R″ portions in the table are effectively any organic moiety (e.g., an alkyl group, aryl group, heteroaryl group, or substituted alkyl, aryl, or heteroaryl, group) which is compatible with the bond formation provided in converting RG to one of the Exemplary Functional Groups. It will also be appreciated that, as applied to the embodiments of the present invention, R′ may represent one or more components of the self-stabilizing linker or optional secondary linker, as the case may be, and R″ may represent one or more components of the optional secondary linker, Camptothecin, stabilizing unit, or detection unit, as the case may be.

| | | Exemplary Functional |
|---|---|---|
| RG | RS | Groups |
| 1) R′—SH | —S— | R′—S—R″ |
| | | R′—S—S—R″ |
| 2) R′—C(═O)OH | —C(═O)— | R′—C(═O)NH—R″ |
| 3) R′—C(═O)ONHS | —C(═O)— | R′—C(═O)NH—R″ |
| 4) R′S(═O)₂—OH | —S(═O)₂— | R′S(═O)₂NH—R″ |
| 5) R′—CH₂—X | —CH₂— | R′—CH₂—S—R″ |
| (X is Br, I, Cl) | | |
| 6) R′—NH₂ | —N— | R′—NHC(═O)R″ |

Isotopically-labeled compounds are also within the scope of the present disclosure. As used herein, an “isotopically-labeled compound” or “isotope derivative” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art. Benefits may also be obtained from replacement of normally abundant ¹²C with ¹³C. (See, WO 2007/005643, WO 2007/005644, WO 2007/016361, and WO 2007/016431.)

For example, deuterium (²H) can be incorporated into a compound disclosed herein for the purpose in order to manipulate the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate in rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}/k_{D}=2-7 are typical. If this rate difference is successfully applied to a compound disclosed herein that is susceptible to oxidation, the profile of this compound in vivo can be drastically modified and result in improved pharmacokinetic properties.

When discovering and developing therapeutic agents, the person skilled in the art is able to optimize pharmacokinetic parameters while retaining desirable in vitro properties. It is reasonable to assume that many compounds with poor pharmacokinetic profiles are susceptible to oxidative metabolism. In vitro liver microsomal assays currently available provide valuable information on the course of oxidative metabolism of this type, which in turn permits the rational design of deuterated compounds of those disclosed herein with improved stability through resistance to such oxidative metabolism. Significant improvements in the pharmacokinetic profiles of compounds disclosed herein are thereby obtained, and can be expressed quantitatively in terms of increases in the in vivo half-life (t/2), concentration at maximum therapeutic effect (Cₘₐₓ), area under the dose response curve (AUC), and F; and in terms of reduced clearance, dose and materials costs.

The following is intended to illustrate the above: a compound which has multiple potential sites of attack for oxidative metabolism, for example benzylic hydrogen atoms and hydrogen atoms bonded to a nitrogen atom, is prepared as a series of analogues in which various combinations of hydrogen atoms are replaced by deuterium atoms, so that some, most or all of these hydrogen atoms have been replaced by deuterium atoms. Half-life determinations enable favorable and accurate determination of the extent of the extent to which the improvement in resistance to oxidative metabolism has improved. In this way, it is determined that the half-life of the parent compound can be extended by up to 100% as the result of deuterium-hydrogen exchange of this type.

Deuterium-hydrogen exchange in a compound disclosed herein can also be used to achieve a favorable modification of the metabolite spectrum of the starting compound in order to diminish or eliminate undesired toxic metabolites. For example, if a toxic metabolite arises through oxidative carbon-hydrogen (C—H) bond cleavage, it can reasonably be assumed that the deuterated analogue will greatly diminish or eliminate production of the unwanted metabolite, even if the particular oxidation is not a rate-determining step. Further information on the state of the art with respect to deuterium-hydrogen exchange may be found, for example in Hanzlik et al., J. Org. Chem. 55, 3992-3997, 1990, Reider et al., J. Org. Chem. 52, 3326-3334, 1987, Foster, Adv. Drug Res. 14, 1-40, 1985, Gillette et al, Biochemistry 33(10) 2927-2937, 1994, and Jarman et al. Carcinogenesis 16(4), 683-688, 1993.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

### EMBODIMENTS

A number of embodiments of the invention are described below, which are not meant to limit the invention in any way, and are followed by a more detailed discussion of the components that make up the conjugates. One of skill in the art will understand that each of the conjugates identified and any of the selected embodiments thereof is meant to include the full scope of each component and linker.

### Camptothecin Conjugates

In one aspect, provided herein are camptothecin conjugates having a formula:

L-(Q-D)ₚ

or a pharmaceutically acceptable form thereof, wherein

### L is a Ligand Unit;

Q is a Linker Unit having a formula selected from the group consisting of:

—Z-A-S*—RL-; —Z-A-L^{P}(S*)—RL-; —Z-A-S*—RL-Y—; and —Z-A-L^{P}(S*)—RL-Y—;
wherein Z is a Stretcher Unit, A is a bond or a Connector Unit; L^{P} is a Parallel Connector Unit; S* is a bond or a Partitioning Agent; RL is a peptide comprising from 2 to 8 amino acids; and Y is a Spacer Unit;
   D is a Drug Unit selected from:

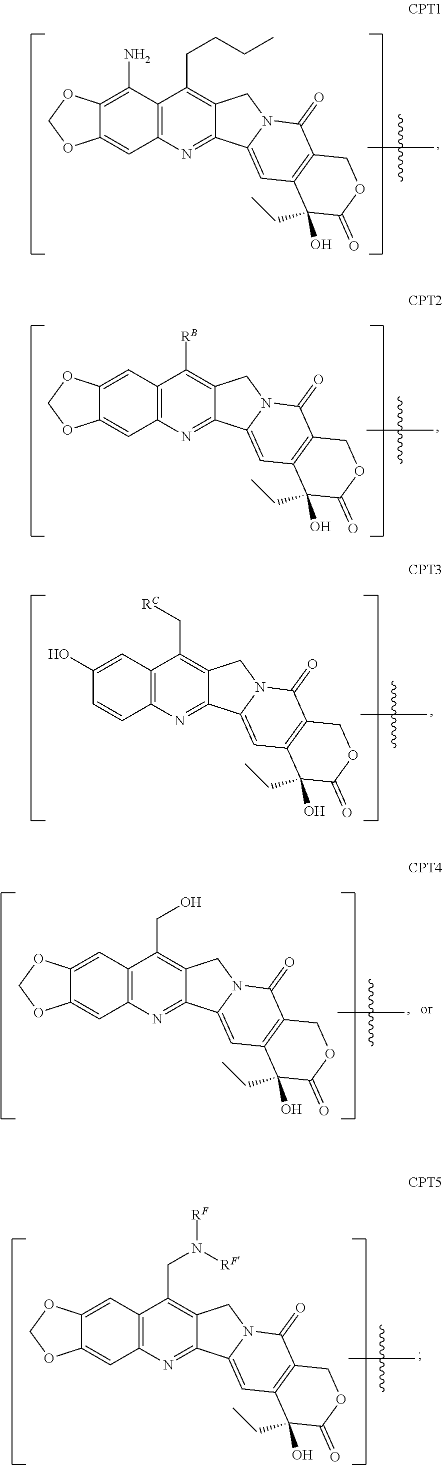

wherein
R^{B} is a member selected from the group consisting of H, C₁-C₈ alkyl, C₁-C₈ haloalkyl, C₃-C₈ cycloalkyl, C₃-C₈cycloalkylC₁-C₄ alkyl, phenyl and phenylC₁-C₄ alkyl;
R^{C} is a member selected from the group consisting of C₁-C₆ alkyl and C₃-C₆ cycloalkyl;
each R^{F} and R^{F′} is a member independently selected from the group consisting of H, C₁-C₈ alkyl, C₁-C₈ hydroxyalkyl, C₁-C₈ aminoalkyl, C₁-C₄ alkylaminoC₁-C₈ alkyl, (C₁-C₄ hydroxyalkyl)(C₁-C₄ alkyl)aminoC₁-C₈ alkyl, di(C₁-C₄ alkyl)aminoC₁-C₈ alkyl, C₁-C₄ hydroxyalkylC₁-C₈ aminoalkyl, C₂-C₆ heteroalkyl, C₁-C₈ alkylC(O)—, C₁-C₈ hydroxyalkylC(O)—, C₁-C₈ aminoalkylC(O)—, C₃-C₁₀ cycloalkyl, C₃-C₁₀cycloalkylC₁-C₄ alkyl, C₃-C₁₀ heterocycloalkyl, C₃-C₁₀ heterocycloalkylC₁-C₄ alkyl, phenyl, phenylC₁-C₄ alkyl, diphenylC₁-C₄ alkyl, heteroaryl and heteroarylC₁-C₄ alkyl; or R^{F} and R^{F′} are combined with the nitrogen atom to which each is attached to form a 5-, 6- or 7-membered ring having 0 to 3 substituents selected from halogen, C₁-C₄ alkyl, OH, OC₁-C₄ alkyl, NH₂, NHC₁-C₄ alkyl and N(C₁-C₄ alkyl)₂; and wherein cycloalkyl, heterocycloalkyl, phenyl and heteroaryl portions of R^{B}, R^{C}, R^{F} and R^{F′} are substituted with from 0 to 3 substituents selected from halogen, C₁-C₄ alkyl, OH, OC₁-C₄ alkyl, NH₂, NHC₁-C₄ alkyl and N(C₁-C₄ alkyl)₂; and
   p is from about 1 to about 16;
wherein Q is attached through any one of the hydroxyl or amine groups present on CPT1, CPT2, CPT3, CPT4 or CPT5.

In another aspect, provided herein are camptothecin conjugates having a formula:

L-(Q-D)ₚ

or a pharmaceutically acceptable form thereof, wherein

### L is a Ligand Unit;

Q is a Linker Unit having a formula selected from the group consisting of:

—Z-A-S*—RL-; —Z-A-L^{P}(S*)—RL-; —Z-A-S*—RL-Y—; and —Z-A-L^{P}(S*)—RL-Y—;
wherein Z is a Stretcher Unit, A is a bond or a Connector Unit; L^{P} is a Parallel Connector Unit; S* is a bond or a Partitioning Agent; RL is a peptide comprising from 2 to 8 amino acids; and Y is a Spacer Unit;
   D is a Drug Unit selected from:

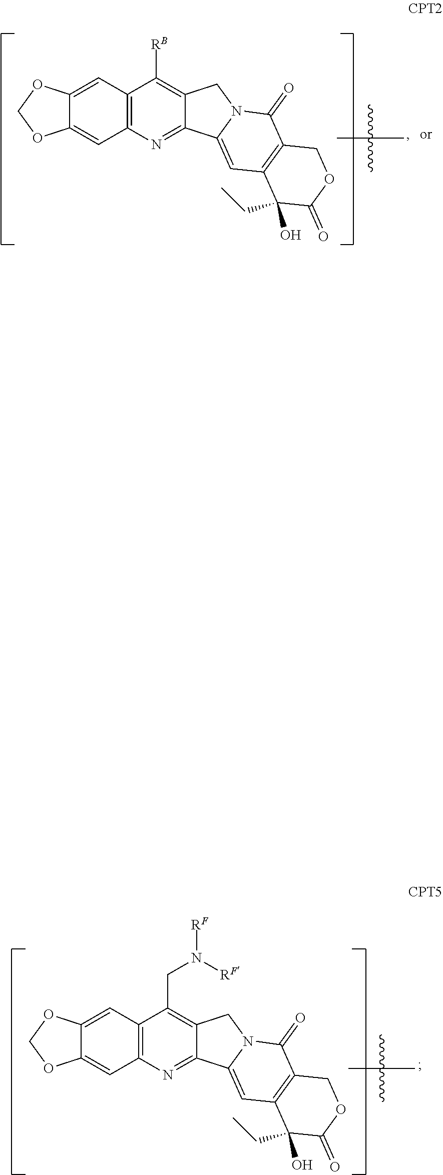

wherein
R^{B} is a member selected from the group consisting of —H, —(C₁-C₄)alkyl-OH, C₁-C₈ alkyl, C₁-C₈ haloalkyl, C₃-C₈ cycloalkyl, C₃-C₈cycloalkylC₁-C₄ alkyl, phenyl and phenylC₁-C₄ alkyl;
each R^{F} and R^{F′} is a member independently selected from the group consisting of H, C₁-C₈ alkyl, C₁-C₈ hydroxyalkyl, C₁-C₈ aminoalkyl, C₁-C₄ alkylaminoC₁-C₈ alkyl, (C₁-C₄ hydroxyalkyl)(C₁-C₄ alkyl)aminoC₁-C₈ alkyl, di(C₁-C₄ alkyl)aminoC₁-C₈ alkyl, C₁-C₄ hydroxyalkylC₁-C₈ aminoalkyl, C₂-C₆ heteroalkyl, C₁-C₈ alkylC(O)—, C₁-C₈ hydroxyalkylC(O)—, C₁-C₈ aminoalkylC(O)—, C₃-C₁₀ cycloalkyl, C₃-C₁₀cycloalkylC₁-C₄ alkyl, C₃-C₁₀ heterocycloalkyl, C₃-C₁₀ heterocycloalkylC₁-C₄ alkyl, phenyl, phenylC₁-C₄ alkyl, diphenylC₁-C₄ alkyl, heteroaryl and heteroarylC₁-C₄ alkyl; or R^{F} and R^{F′} are combined with the nitrogen atom to which each is attached to form a 5-, 6- or 7-membered ring having 0 to 3 substituents selected from halogen, C₁-C₄ alkyl, OH, OC₁-C₄ alkyl, NH₂, NHC₁-C₄ alkyl and N(C₁-C₄ alkyl)₂;
and wherein cycloalkyl, heterocycloalkyl, phenyl and heteroaryl portions of R^{B}, R^{F} and R^{F′} are substituted with from 0 to 3 substituents selected from halogen, C₁-C₄ alkyl, OH, OC₁-C₄ alkyl, NH₂, NHC₁-C₄ alkyl and N(C₁-C₄ alkyl)₂; and
   p is from about 1 to about 16;
wherein Q is attached through any one of the hydroxyl or amine groups present on CPT2 or CPT5.

In yet another aspect, provided herein are camptothecin conjugates having a formula:

L-(Q-D)ₚ

or a pharmaceutically acceptable form thereof, wherein

### L is a Ligand Unit;

Q is a Linker Unit having a formula selected from the group consisting of:

—Z-A-S*—RL-; —Z-A-L^{P}(S*)—RL-; —Z-A-S*—RL-Y—; and —Z-A-L^{P}(S*)—RL-Y—;
wherein Z is a Stretcher Unit, A is a bond or a Connector Unit; L^{P} is a Parallel Connector Unit; S* is a bond or a Partitioning Agent; RL is a peptide comprising from 2 to 8 amino acids; and Y is a Spacer Unit;
   D is a Drug Unit having the following structure formula:

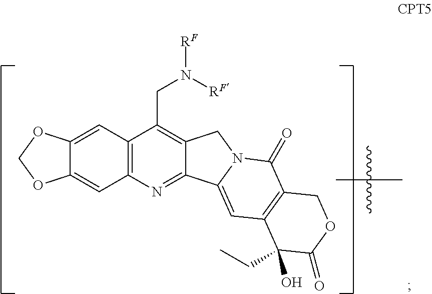

wherein
each R^{F} and R^{F′} is a member independently selected from the group consisting of H, C₁-C₈ alkyl, C₁-C₈ hydroxyalkyl, C₁-C₈ aminoalkyl, C₁-C₄ alkylaminoC₁-C₈ alkyl, (C₁-C₄ hydroxyalkyl)(C₁-C₄ alkyl)aminoC₁-C₈ alkyl, di(C₁-C₄ alkyl)aminoC₁-C₈ alkyl, C₁-C₄ hydroxyalkylC₁-C₈ aminoalkyl, C₂-C₆ heteroalkyl, C₁-C₈ alkylC(O)—, C₁-C₈ hydroxyalkylC(O)—, C₁-C₈ aminoalkylC(O)—, C₃-C₁₀ cycloalkyl, C₃-C₁₀cycloalkylC₁-C₄ alkyl, C₃-C₁₀ heterocycloalkyl, C₃-C₁₀ heterocycloalkylC₁-C₄ alkyl, phenyl, phenylC₁-C₄ alkyl, diphenylC₁-C₄ alkyl, heteroaryl and heteroarylC₁-C₄ alkyl; or R^{F} and R^{F′} are combined with the nitrogen atom to which each is attached to form a 5-, 6- or 7-membered ring having 0 to 3 substituents selected from halogen, C₁-C₄ alkyl, OH, OC₁-C₄ alkyl, NH₂, NHC₁-C₄ alkyl and N(C₁-C₄ alkyl)₂;
and wherein cycloalkyl, heterocycloalkyl, phenyl and heteroaryl portions of R^{F} and R^{F′} are substituted with from 0 to 3 substituents selected from halogen, C₁-C₄ alkyl, OH, OC₁-C₄ alkyl, NH₂, NHC₁-C₄ alkyl and N(C₁-C₄ alkyl)₂; and
   p is from about 1 to about 16;
wherein Q is attached through any one of the hydroxyl or amine groups present on CPT5.

In one group of embodiments, D has formula CPT5.

In one group of embodiments, D has formula CPT2.

In one group of embodiments, D has formula CPT3.

In one group of embodiments, D has formula CPT4.

In one group of embodiments, D has formula CPT1.

In some embodiments, the pharmaceutically acceptable form is a pharmaceutically acceptable salt.

In one group of embodiments, Q has a formula selected from the group consisting of:

—Z-A-S*—RL- and —Z-A-S*—RL-Y—.

In another group of embodiments, Q has a formula selected from the group consisting of: —Z-A-L^{P}(S*)—RL- and —Z-A-L^{P}(S*)—RL-Y—.

In one group of embodiments, the Camptothecin Conjugates comprise a Drug Unit having formula CPT1, and are represented by a formula selected from:

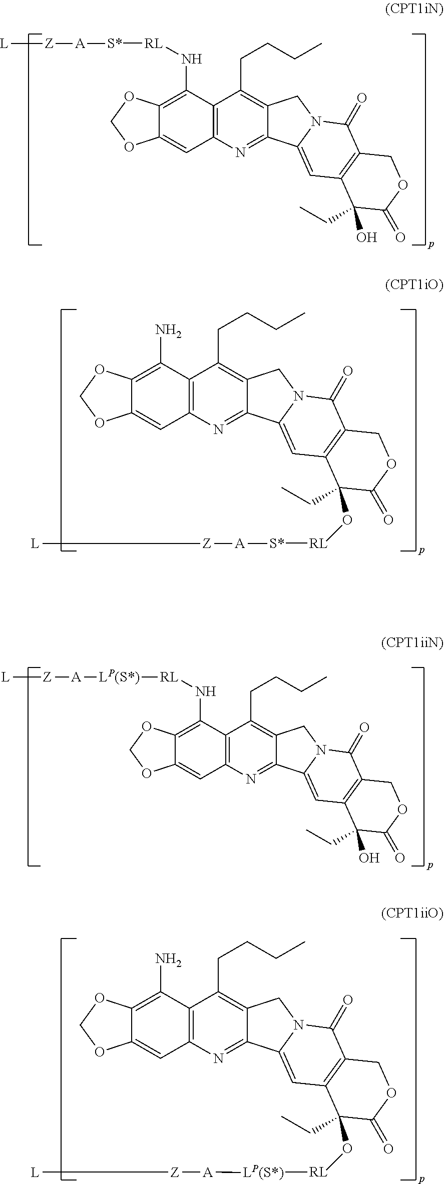

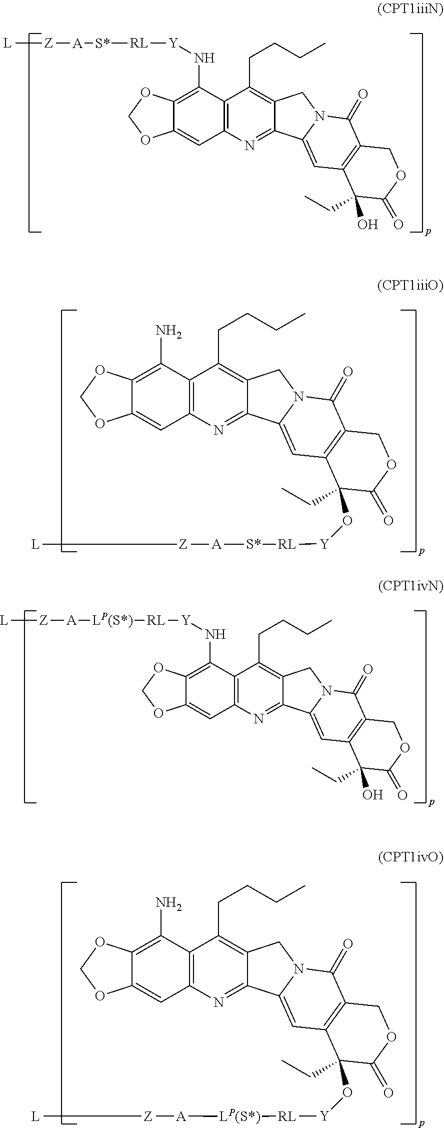

wherein the groups L, Z, A, S*, L^{P}, RL and Y have the meanings provided above and in the any of the embodiments specifically recited herein.

In another group of embodiments, the Camptothecin Conjugates comprise a Drug Unit having formula CPT2, and are represented by a formula selected from:

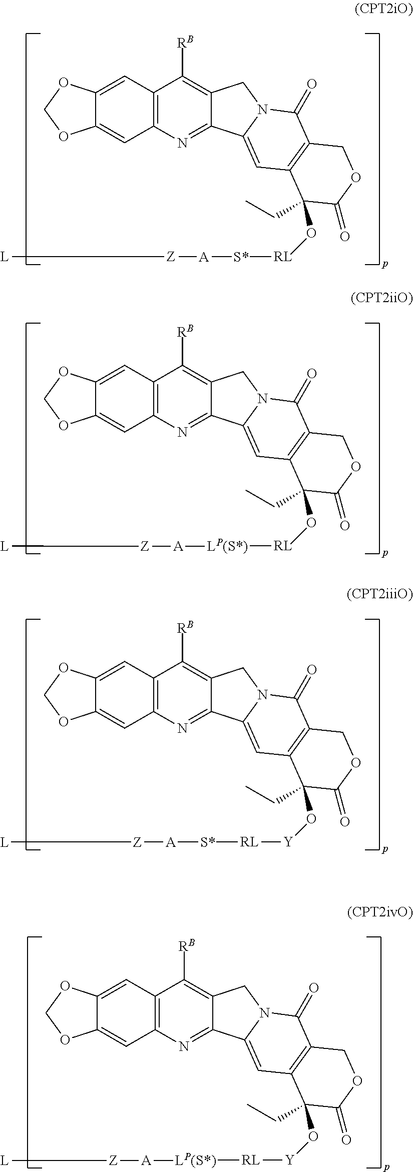

wherein the groups L, Z, A, S*, L^{P}, RL and Y have the meanings provided above and in the any of the embodiments specifically recited herein.

In one group of embodiments, R^{B} is a member selected from the group consisting of H, C₁-C₈ alkyl, and C₁-C₈ haloalkyl.

In one group of embodiments, R^{B} is a member selected from the group consisting of C₃-C₈ cycloalkyl, C₃-C₈cycloalkylC₁-C₄ alkyl, phenyl and phenylC₁-C₄ alkyl, and wherein the cycloalkyl and phenyl portions of R^{B} are substituted with from 0 to 3 substituents selected from halogen, C₁-C₄ alkyl, OH, OC₁-C₄ alkyl, NH₂, NHC₁-C₄ alkyl and N(C₁-C₄ alkyl)₂.

In another group of embodiments, R^{B} is H, methyl, ethyl, propyl, isopropyl, butyl, isobutyl, sec-butyl, tert-butyl, pentyl, isopentyl, 1-ethylpropyl, or hexyl. In other embodiments, R^{B} is chloromethyl or bromomethyl. In other embodiments, R^{B} is phenyl or halo-substituted phenyl. In other embodiments, R^{B} is phenyl or fluorophenyl.

In another group of embodiments, the Camptothecin Conjugates comprise a Drug Unit having formula CPT3, and are represented by a formula selected from:

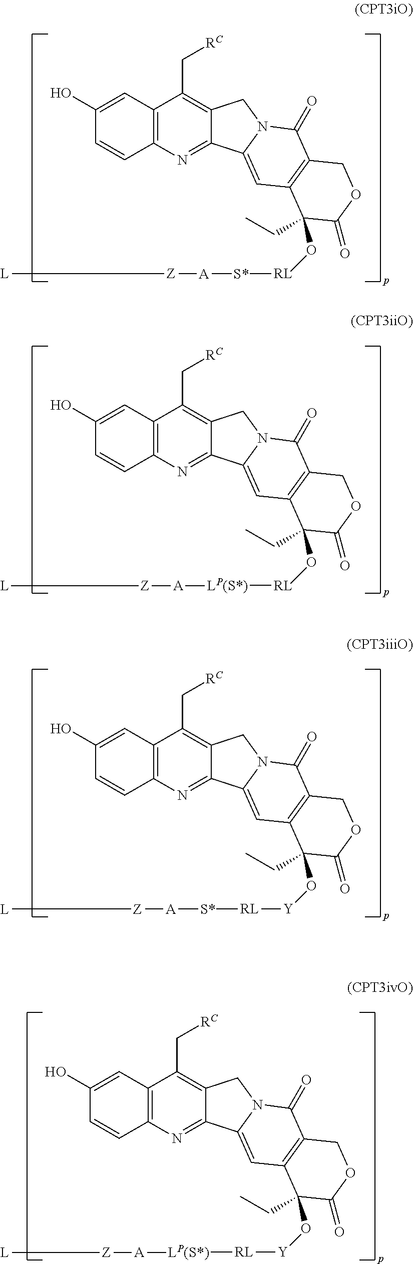

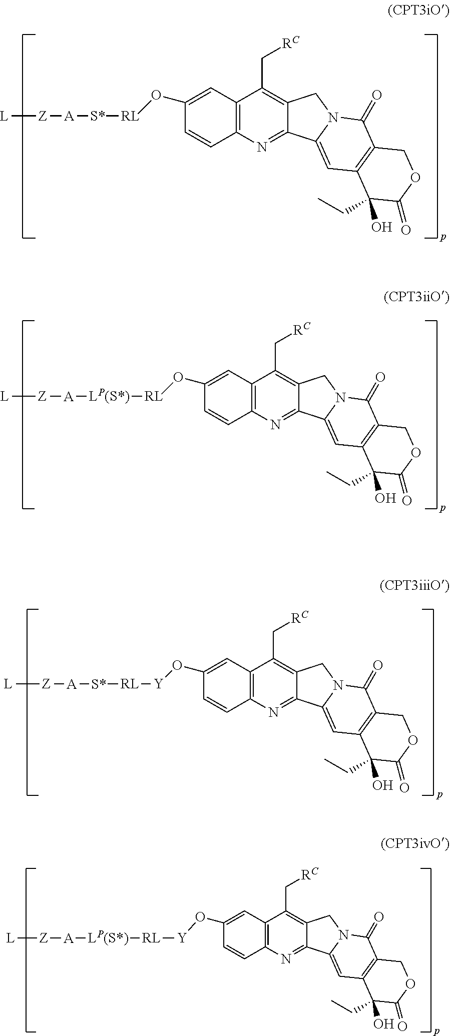

wherein the groups L, Z, A, S*, L^{P}, RL and Y have the meanings provided above and in the any of the embodiments specifically recited herein.

In one group of embodiments, R^{C} is C₁-C₆ alkyl. In some embodiments, R^{C} is methyl.

In one group of embodiments, R^{C} is C₃-C₆ cycloalkyl.

In another group of embodiments, the Camptothecin Conjugates comprise a Drug Unit having formula CPT4, and are represented by a formula selected from:

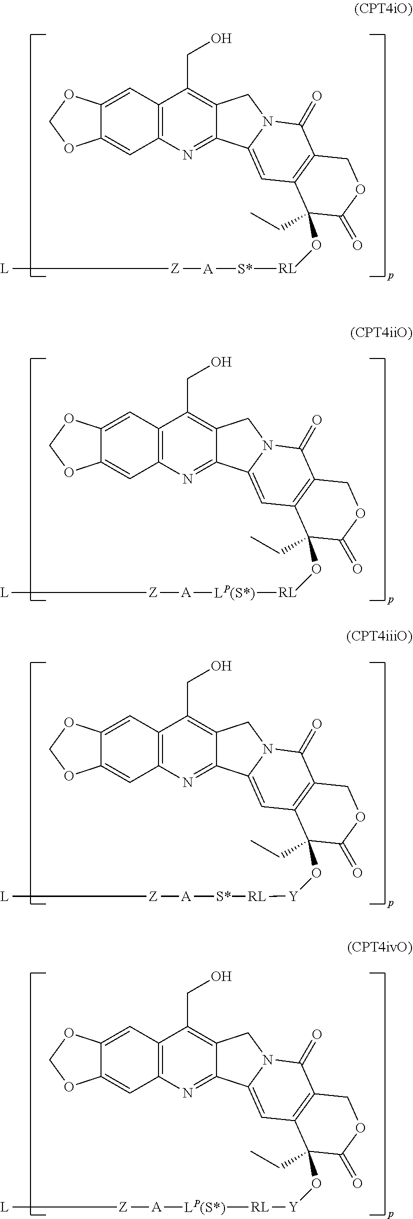

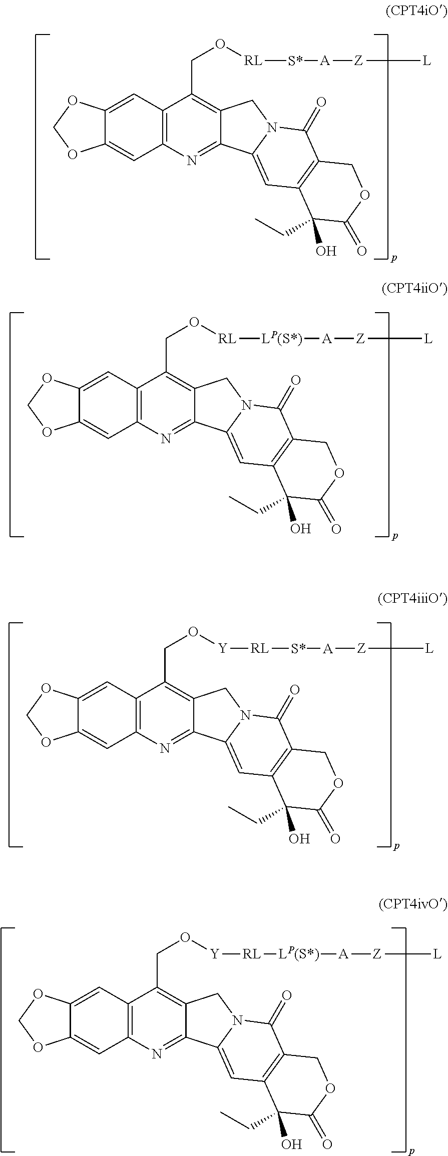

wherein the groups L, Z, A, S*, L^{P}, RL and Y have the meanings provided above and in the any of the embodiments specifically recited herein.

In another group of embodiments, the Camptothecin Conjugates comprise a Drug Unit having formula CPT5, and are represented by a formula selected from:

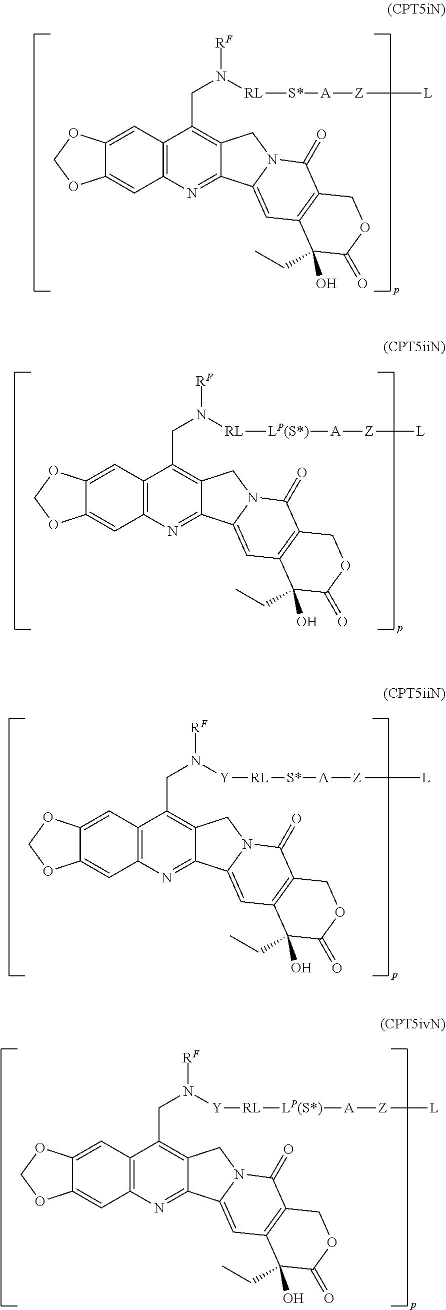

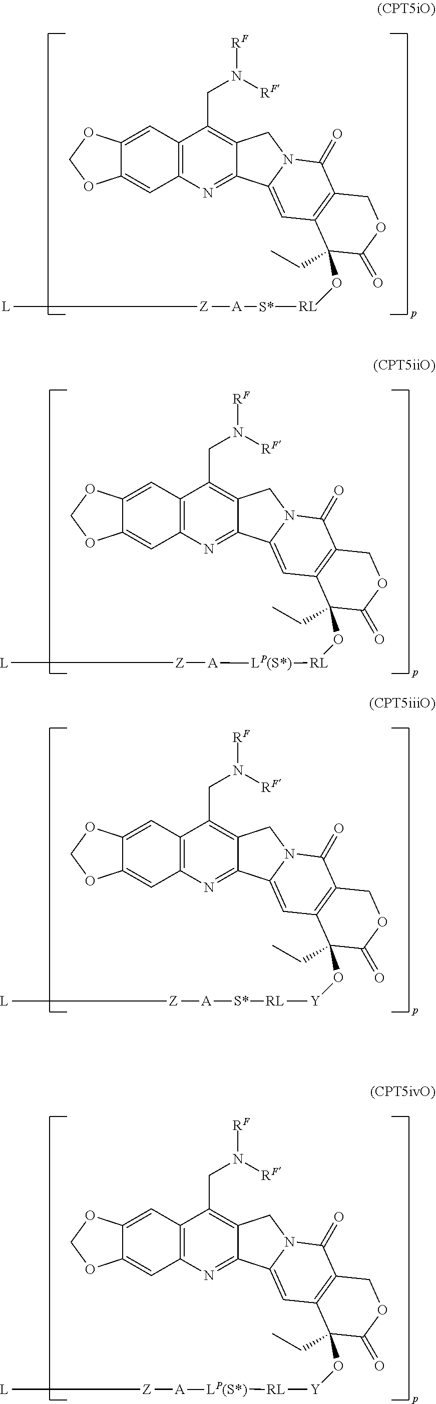

wherein the groups L, Z, A, S*, L^{P}, RL and Y have the meanings provided above and in the any of the embodiments specifically recited herein.

In one group of embodiments, both R^{F} and R^{F′} are H.

In one group of embodiments, at least one of R^{F} and R^{F′} is a member independently selected from the group consisting of C₁-C₈ alkyl, C₁-C₈ hydroxyalkyl, C₁-C₈ aminoalkyl, C₁-C₄ alkylaminoC₁-C₈ alkyl, (C₁-C₄ hydroxyalkyl)(C₁-C₄ alkyl)aminoC₁-C₈ alkyl, di(C₁-C₄ alkyl)aminoC₁-C₈ alkyl, C₁-C₄ hydroxyalkylC₁-C₈ aminoalkyl, C₂-C₆ heteroalkyl, C₁-C₈ alkylC(O)—C₁-C₈ hydroxyalkylC(O)—, and C₁-C₈ aminoalkylC(O)—.

In one group of embodiments, each R^{F} and R^{F′} is a member independently selected from the group consisting of C₁-C₈ alkyl, C₁-C₈ hydroxyalkyl, C₁-C₈ aminoalkyl, C₁-C₄ alkylaminoC₁-C₈ alkyl, (C₁-C₄ hydroxyalkyl)(C₁-C₄ alkyl)aminoC₁-C₈ alkyl, di(C₁-C₄ alkyl)aminoC₁-C₈ alkyl, C₁-C₄ hydroxyalkylC₁-C₈ aminoalkyl, C₂-C₆ heteroalkyl, C₁-C₈ alkylC(O)—, C₁-C₈ hydroxyalkylC(O)—, and C₁-C₈ aminoalkylC(O)—.

In one group of embodiments, at least one of R^{F} and R^{F′} is a member independently selected from the group consisting of C₃-C₁₀ cycloalkyl, C₃-C₁₀cycloalkylC₁-C₄ alkyl, C₃-C₁₀ heterocycloalkyl, C₃-C₁₀ heterocycloalkylC₁-C₄ alkyl, phenyl, phenylC₁-C₄ alkyl, diphenylC₁-C₄ alkyl, heteroaryl and heteroarylC₁-C₄ alkyl, and wherein cycloalkyl, heterocycloalkyl, phenyl and heteroaryl portions of R^{F} and R^{F′} are substituted with from 0 to 3 substituents selected from halogen, C₁-C₄ alkyl, OH, OC₁-C₄ alkyl, NH₂, NHC₁-C₄ alkyl and N(C₁-C₄ alkyl)₂.

In one group of embodiments, each R^{F} and R^{F′} is a member independently selected from the group consisting of H, C₃-C₁₀ cycloalkyl, C₃-C₁₀cycloalkylC₁-C₄ alkyl, C₃-C₁₀ heterocycloalkyl, C₃-C₁₀ heterocycloalkylC₁-C₄ alkyl, phenyl, phenylC₁-C₄ alkyl, diphenylC₁-C₄ alkyl, heteroaryl and heteroarylC₁-C₄ alkyl, and wherein cycloalkyl, heterocycloalkyl, phenyl and heteroaryl portions of R^{F} and R^{F′} are substituted with from 0 to 3 substituents selected from halogen, C₁-C₄ alkyl, OH, OC₁-C₄ alkyl, NH₂, NHC₁-C₄ alkyl and N(C₁-C₄ alkyl)₂.

In some embodiments, R^{F} is H and R^{F′} is C₁₋₈ alkyl.

In one group of embodiments, R^{F} and R^{F′} are combined with the nitrogen atom to which each is attached to form a 5-, 6- or 7-membered ring having 0 to 3 substituents selected from halogen, C₁-C₄ alkyl, OH, OC₁-C₄ alkyl, NH₂, NHC₁-C₄ alkyl and N(C₁-C₄ alkyl)₂.

In some embodiments, the Camptothecin Conjugates have Formula (IC):

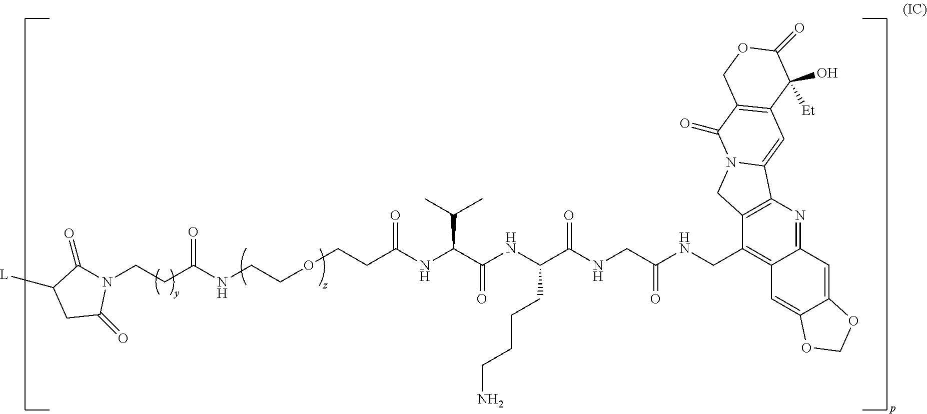

or a pharmaceutically acceptable salt thereof;

wherein

y is 1, 2, 3, or 4, or is 1 or 4; and

z is an integer from 2 to 12, or is 2, 4, 8, or 12;

and p is 1-16.

In some aspect of these embodiments, p is 2, 3, 4, 5, 6, 7, 8, 9, or 10. In some aspect, p is 2, 4 or 8.

In some embodiments, the Camptothecin Conjugates have formula:

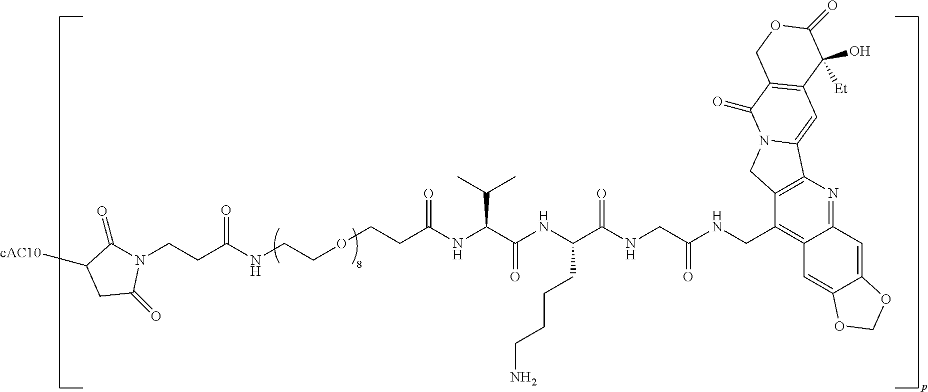

or a pharmaceutically acceptable salt thereof;

wherein p is 2, 4, or 8, preferably p is 8.

In some embodiments, the Camptothecin Conjugates have formula:

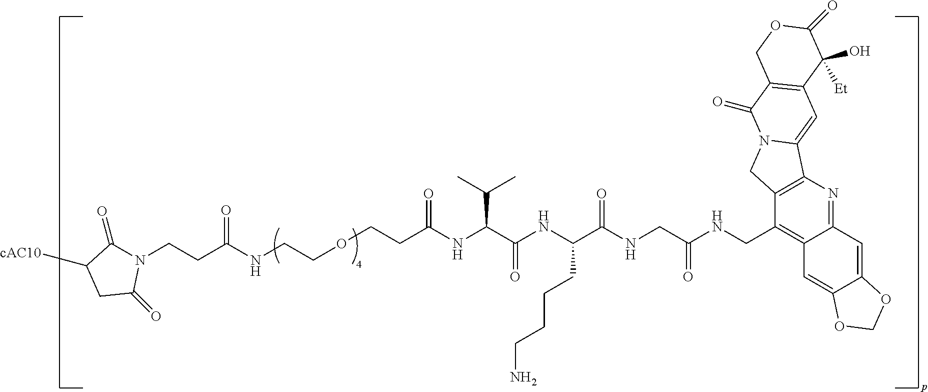

or a pharmaceutically acceptable salt thereof;

wherein p is 2, 4, or 8, preferably p is 8.

In some aspect of these embodiments, p is 8.

### Camptothecin-Linker Compounds

In some aspects, when preparing the Camptothecin Conjugates, it will be desirable to synthesize the full drug-linker combination, or the drug in combination with a portion of the linker, prior to conjugation to a targeting ligand. In such embodiments, Camptothecin-Linker Compounds as described herein, are intermediate compounds. In these embodiments, the Stretcher Unit in a Camptothecin-Linker Compound is not yet covalently attached to the Ligand Unit and therefore has a functional group for conjugation to a targeting ligand (i.e., is a Stretcher Unit precursor, Z′). In one aspect, a Camptothecin-Linker Compound comprises a Camptothecin (shown herein as formulae CPT1, CPT2, CPT3, CPT4 and CPT5), and a Linker Unit (Q) comprising a Peptide Releasable Linker (RL) through which the Ligand Unit is connected to the Camptothecin. Thus, the Linker Unit comprises, in addition to RL (which is a Peptide Linker), a Stretcher Unit precursor (Z′) comprising a functional group for conjugation to a Ligand Unit and capable of (directly or indirectly) connecting the RL to the Ligand Unit. A Parallel Connector Unit (L^{P}) can be present in some embodiments when it is desired to add a Partitioning Agent (S*) as a side chain appendage. In some embodiments, a Connector Unit (A) is present when it is desirable to add more distance between the Stretcher Unit and RL.

In one aspect, a Camptothecin-Linker Compound is comprised of a Camptothecin having formula CPT1, CPT2, CPT3, CPT4 or CPT5, and a Linker Unit (Q), wherein Q comprises a Peptide Releasable Linker, directly attached to a Stretcher Unit precursor (Z′) or indirectly to Z′ through attachment to intervening component(s) of the Camptothecin-Linker Compound's Linker Unit (i.e., A, S* and/or L^{P}(S*)), wherein Z′ is comprised of a functional group capable of forming a covalent bond to a targeting ligand.

In the context of the Camptothecin Conjugates and/or the Camptothecin-Linker Compounds—the assembly is best described in terms of its component groups. While some procedures are also described herein, the order of assembly and the general conditions to prepare the Conjugates and Compounds will be well understood by one of skill in the art.

### Component Groups

### Ligand Units:

In some embodiments of the invention, a Ligand Unit is present. The Ligand Unit (L-) is a targeting agent that specifically binds to a target moiety. In one group of embodiments, the Ligand Unit specifically and selectively binds to a cell component (a Cell Binding Agent) or to other target molecules of interest. The Ligand Unit acts to target and present the camptothecin (CPT1, CPT2, CPT3, CPT4 or CPT5) or a drug component containing camptothecin to the particular target cell population with which the Ligand Unit interacts due to the presence of its targeted component or molecule and allows for subsequent release of free drug within (i.e., intracellularly) or within the vicinity of the target cells (i.e., extracellularly). Ligand Units, L, include, but are not limited to, proteins, polypeptides and peptides. Suitable Ligand Units include, for example, antibodies, e.g., full-length antibodies and antigen binding fragments thereof, interferons, lymphokines, hormones, growth factors and colony-stimulating factors, vitamins, nutrient-transport molecules (such as, but not limited to, transferrin), or any other cell binding molecule or substance. In some embodiments, the Ligand Unit (L) is an antibody or a non-antibody protein targeting agent.

In one group of embodiments a Ligand Unit is bonded to Q (a Linker Unit) which comprises a Peptide Releasable Linker. As noted above, still other linking components can be present in the conjugates described herein to serve the purpose of providing additional space between the Camptothecin drug compound and the Ligand Unit (e.g., a Stretcher Unit and optionally a Connector Unit, A), or providing attributes to the composition to increases solubility (e.g., a Partitioning Agent, S*). In some of those embodiments, the Ligand Unit is bonded to Z of the Linker Unit via a heteroatom of the Ligand Unit. Heteroatoms that may be present on a Ligand Unit for that bonding include sulfur (in one embodiment, from a sulfhydryl group of a targeting ligand), oxygen (in one embodiment, from a carboxyl or hydroxyl group of a targeting ligand) and nitrogen, optionally substituted (in one embodiment, from a primary or secondary amine functional group of a targeting ligand or in another embodiment from an optionally substituted amide nitrogen). Those heteroatoms can be present on the targeting ligand in the ligand's natural state, for example in a naturally-occurring antibody, or can be introduced into the targeting ligand via chemical modification or biological engineering.

In one embodiment, a Ligand Unit has a sulfhydryl functional group so that the Ligand Unit is bonded to the Linker Unit via the sulfur atom of the sulfhydryl functional group.

In another embodiment, a Ligand Unit has one or more lysine residues that are capable of reacting with activated esters (such esters include, but are not limited to, N-hydroxysuccimide, pentafluorophenyl, and p-nitrophenyl esters) of a Stretcher Unit precursor of a Camptothecin-Linker Compound intermediate and thus provides an amide bond consisting of the nitrogen atom of the Ligand Unit and the C═O group of the Linker Unit's Stretcher Unit.

In yet another aspect, a Ligand Unit has one or more lysine residues capable of chemical modification to introduce one or more sulfhydryl groups. In those embodiments, the Ligand Unit is covalently attached to the Linker Unit via the sulfhydryl functional group's sulfur atom. The reagents that can be used to modify lysines in that manner include, but are not limited to, N-succinimidyl S-acetylthioacetate (SATA) and 2-Iminothiolane hydrochloride (Traut's Reagent).

In another embodiment, a Ligand Unit has one or more carbohydrate groups capable of modification to provide one or more sulfhydryl functional groups. The chemically modified Ligand Unit in a Camptothecin Conjugate is bonded to a Linker Unit component (e.g., a Stretcher Unit) via the sulfur atom of the sulfhydryl functional group.

In yet another embodiment, the Ligand Unit has one or more carbohydrate groups that can be oxidized to provide an aldehyde (—CHO) functional group (see, e.g., Laguzza, et al., 1989, J. Med. Chem. 32(3):548-55). In these embodiments, the corresponding aldehyde interacts with a reactive site on a Stretcher Unit precursor to form a bond between the Stretcher Unit and the Ligand Unit. Reactive sites on a Stretcher Unit precursor that capable of interacting with a reactive carbonyl-containing functional group on a targeting Ligand Unit include, but are not limited to, hydrazine and hydroxylamine. Other protocols for the modification of proteins for the attachment of Linker Units (Q) or related species are described in Coligan et al., Current Protocols in Protein Science, vol. 2, John Wiley & Sons (2002) (incorporated herein by reference).

In some aspects, a Ligand Unit is capable of forming a bond by interacting with a reactive functional group on a Stretcher Unit precursor (Z′) to form a covalent bond between the Stretcher Unit (Z) and the Ligand Unit corresponding to the targeting ligand. The functional group of Z′ having that capability for interacting with a targeting ligand will depend on the nature of the Ligand Unit. In some embodiments, the reactive group is a maleimide that is present on a Stretcher Unit prior to its attachment to form a Ligand Unit (i.e., a maleimide moiety of a Stretcher Unit precursor). Covalent attachment of a Ligand Unit to a Stretcher Unit is accomplished through a sulfhydryl functional group of a Ligand Unit interacting with the maleimide functional group of Z′ to form a thio-substituted succinimide. The sulfhydryl functional group can be present on the Ligand Unit in the Ligand Unit's natural state, for example, in a naturally-occurring residue, or can be introduced into the Ligand Unit via chemical modification or by biological engineering.

In still another embodiment, the Ligand Unit is an antibody and the sulfhydryl group is generated by reduction of an interchain disulfide of the antibody. Accordingly, in some embodiments, the Linker Unit is conjugated to a cysteine residue from reduced interchain disulfide(s).

In yet another embodiment, the Ligand Unit is an antibody and the sulfhydryl functional group is chemically introduced into the antibody, for example, by introduction of a cysteine residue. Accordingly, in some embodiments, the Linker Unit (with or without an attached Camptothecin) is conjugated to a Ligand Unit through an introduced cysteine residue of a Ligand Unit.

It has been observed for bioconjugates that the site of drug conjugation can affect a number of parameters including ease of conjugation, drug-linker stability, effects on biophysical properties of the resulting bioconjugates, and in-vitro cytotoxicity. With respect to drug-linker stability, the site of conjugation of a drug-linker moiety to a Ligand Unit can affect the ability of the conjugated drug-linker moiety to undergo an elimination reaction, in some instances, to cause premature release of free drug. Sites for conjugation on a targeting ligand include, for example, a reduced interchain disulfide as well as selected cysteine residues at engineered sites. In some embodiments conjugation methods to form Camptothecin Conjugates as described herein use thiol residues at genetically engineered sites that are less susceptible to the elimination reaction (e.g., positions 239 according to the EU index as set forth in Kabat) in comparison to conjugation methods that use thiol residues from a reduced disulfide bond. In other embodiments conjugation methods to form Camptothecin Conjugates as described herein use thiol residues at sites that are more susceptible to the elimination reaction (e.g. resulting from interchain disulfide reduction).

In some embodiments, a Camptothecin Conjugate comprises a non-immunoreactive protein, polypeptide, or peptide, as its Ligand Unit. Accordingly, in some embodiments, the Ligand Unit is a non-immunoreactive protein, polypeptide, or peptide. Examples include, but are not limited to, transferrin, epidermal growth factors (“EGF”), bombesin, gastrin, gastrin-releasing peptide, platelet-derived growth factor, IL-2, IL-6, transforming growth factors (“TGF”), such as TGF-α and TGF-β, vaccinia growth factor (“VGF”), insulin and insulin-like growth factors I and II, somatostatin, lectins and apoprotein from low density lipoprotein.

Particularly preferred Ligand Units are from antibodies. In fact, in any of the embodiments described herein, the Ligand Unit can be from an antibody. Useful polyclonal antibodies are heterogeneous populations of antibody molecules derived from the sera of immunized animals. Useful monoclonal antibodies are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest can be prepared by using any technique known in the art, which provides for the production of antibody molecules by continuous cell lines in culture.

Useful monoclonal antibodies include, but are not limited to, human monoclonal antibodies, humanized monoclonal antibodies, or chimeric human-mouse (or other species) monoclonal antibodies. The antibodies include full-length antibodies and antigen binding fragments thereof. Human monoclonal antibodies can be made by any of numerous techniques known in the art (e.g., Teng et al., 1983, Proc. Natl. Acad. Sci. USA. 80:7308-7312; Kozbor et al., 1983, Immunology Today 4:72-79; and Olsson et al., 1982, Meth. Enzymol. 92:3-16).

The antibody can be a functionally active fragment, derivative or analog of an antibody that immunospecifically binds to target cells (e.g., cancer cell antigens, viral antigens, or microbial antigens) or other antibodies bound to tumor cells or matrix. In this regard, “functionally active” means that the fragment, derivative or analog is able to immunospecifically binds to target cells. To determine which CDR sequences bind the antigen, synthetic peptides containing the CDR sequences can be used in binding assays with the antigen by any binding assay method known in the art (e.g., the BIA core assay) (See, e.g., Kabat et al., 1991, Sequences of Proteins of Immunological Interest, Fifth Edition, National Institute of Health, Bethesda, Md.; Kabat E et al., 1980, J. Immunology 125(3):961-969).

Other useful antibodies include fragments of antibodies such as, but not limited to, F(ab′)₂ fragments, Fab fragments, Fvs, single chain antibodies, diabodies, triabodies, tetrabodies, scFv, scFv-FV, or any other molecule with the same specificity as the antibody.

Additionally, recombinant antibodies, such as chimeric and humanized monoclonal antibodies, comprising both human and non-human portions, which can be made using standard recombinant DNA techniques, are useful antibodies. A chimeric antibody is a molecule in which different portions are derived from different animal species, such as for example, those having a variable region derived from a murine monoclonal and human immunoglobulin constant regions. (See, e.g., U.S. Pat. Nos. 4,816,567; and 4,816,397, which are incorporated herein by reference in their entirety.) Humanized antibodies are antibody molecules from non-human species having one or more complementarity determining regions (CDRs) from the non-human species and a framework region from a human immunoglobulin molecule. (See, e.g., U.S. Pat. No. 5,585,089, which is incorporated herein by reference in its entirety.) Such chimeric and humanized monoclonal antibodies can be produced by recombinant DNA techniques known in the art, for example using methods described in International Publication No. WO 87/02671; European Patent Publication No. 0 184 187; European Patent Publication No. 0 171 496; European Patent Publication No. 0 173 494; International Publication No. WO 86/01533; U.S. Pat. No. 4,816,567; European Patent Publication No. 012 023; Berter et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Cancer. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, BioTechniques 4:214; U.S. Pat. No. 5,225,539; Jones et al., 1986, Nature 321:552-525; Verhoeyan et al., 1988, Science 239:1534; and Beidler et al., 1988, J. Immunol. 141:4053-4060; each of which is incorporated herein by reference in its entirety.

Completely human antibodies in some instances (e.g., when immunogenicity to a non-human or chimeric antibody may occur) are more desirable and can be produced using transgenic mice that are incapable of expressing endogenous immunoglobulin heavy and light chains genes, but which can express human heavy and light chain genes.

Antibodies include analogs and derivatives that are either modified, i.e., by the covalent attachment of any type of molecule as long as such covalent attachment permits the antibody to retain its antigen binding immunospecificity. For example, but not by way of limitation, derivatives and analogs of the antibodies include those that have been further modified, e.g., by glycosylation, acetylation, PEGylation, phosphorylation, amidation, derivatization by known protecting/blocking groups, proteolytic cleavage, linkage to a cellular antibody unit or other protein, etc. Any of numerous chemical modifications can be carried out by known techniques including, but not limited to, specific chemical cleavage, acetylation, formylation, metabolic synthesis in the presence of tunicamycin, etc. Additionally, the analog or derivative can contain one or more unnatural amino acids.

Antibodies can have modifications (e.g., substitutions, deletions or additions) in amino acid residues that interact with Fc receptors. In particular, antibodies can have modifications in amino acid residues identified as involved in the interaction between the anti-Fc domain and the FcRn receptor (see, e.g., International Publication No. WO 97/34631, which is incorporated herein by reference in its entirety).

Antibodies immunospecific for a cancer cell antigen can be obtained commercially or produced by any method known to one of skill in the art such as, recombinant expression techniques. The nucleotide sequence encoding antibodies immunospecific for a cancer cell antigen can be obtained, e.g., from the GenBank database or a database like it, the literature publications, or by routine cloning and sequencing.

In a specific embodiment, a known antibody for the treatment of cancer can be used.

In another specific embodiment, antibodies for the treatment of an autoimmune disease are used in accordance with the compositions and methods of the invention.

In certain embodiments, useful antibodies can bind to a receptor or a receptor complex expressed on an activated lymphocyte. The receptor or receptor complex can comprise an immunoglobulin gene superfamily member, a TNF receptor superfamily member, an integrin, a cytokine receptor, a chemokine receptor, a major histocompatibility protein, a lectin, or a complement control protein.

In some aspects, the antibody that is incorporated into a Camptothecin Conjugate will specifically bind to CD19, CD30, CD33, CD70 or LIV-1.

In some aspects, the antibody that is incorporated into a Camptothecin Conjugate specifically binds to CD30. In another aspects, the antibody that is incorporated into a Camptothecin Conjugate is a cAC10 anti-CD30 antibody, which is described in International Patent Publication No. WO 02/43661. In some embodiments, the anti-CD30 antibody comprises CDR-H1, CDR-H2, CDR-H3, CDR-L1, CDR-L2, and CDR-L3 comprising the amino acid sequences of SEQ ID NOs: 1, 2, 3, 4, 5, and 6, respectively. In some embodiments, the anti-CD30 antibody comprises a heavy chain variable region comprising an amino acid sequence that is at least 95%, at least 96%, at least 97%, at last 98%, at least 99%, or 100% identical to the amino acid sequence of SEQ ID NO: 7 and a light chain variable region comprising an amino acid sequence that is at least 95% at least 96%, at least 97%, at last 98%, at least 99%, or 100% identical to the amino acid sequence of SEQ ID NO: 8. In some embodiments, the anti-CD30 antibody comprises a heavy chain comprising the amino acid sequence of SEQ ID NO: 9 or SEQ ID NO: 10 and a light chain comprising the amino acid sequence of SEQ ID NO: 11.

In some aspects, the antibody that is incorporated into a Camptothecin Conjugate specifically binds to CD70. In another aspects, the antibody that is incorporated into a Camptothecin Conjugate is a h1F6 anti-CD70 antibody, which is described in International Patent Publication No. WO 2006/113909. In some aspects, the antibody that is incorporated into a Camptothecin Conjugate specifically binds to CD48. In another aspects, the antibody that is incorporated into a Camptothecin Conjugate is a hMEM102 anti-CD48 antibody, which is described in International Patent Publication No. WO 2016/149535. In some aspects, the antibody that is incorporated into a Camptothecin Conjugate specifically binds to NTB-A. In another aspects, the antibody that is incorporated into a Camptothecin Conjugate is a h20F3 anti-NTB-A antibody, which is described in International Patent Publication No. WO 2017/004330.

### Camptothecins:

The Camptothecins utilized in the various aspects and embodiments described herein are represented by the formulae:

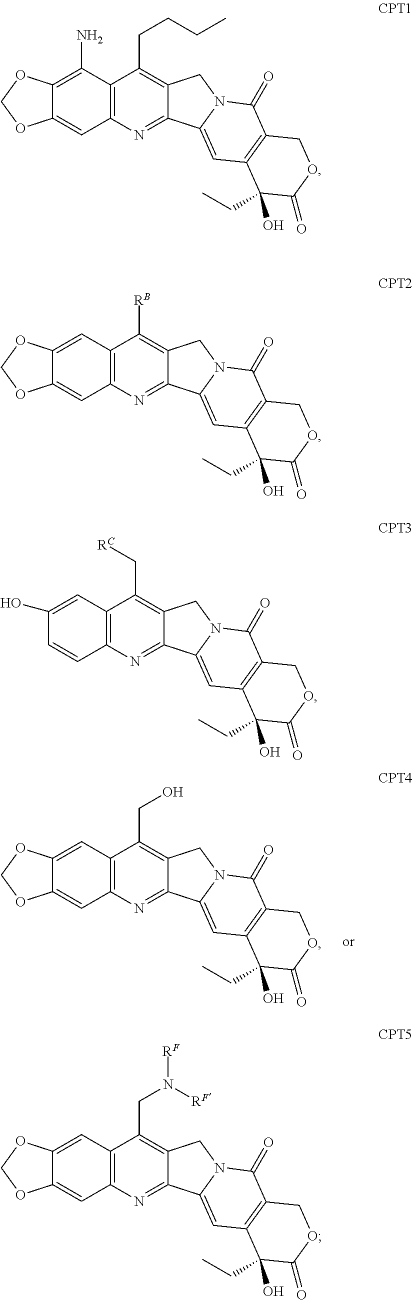

as described herein.

In a specific embodiment, the Camptothecins is of formula:

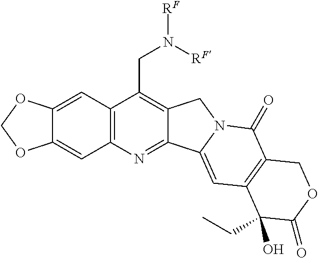

wherein each R^{F} and R^{F′} is independently H, glycyl, hydroxyacetyl, ethyl, or 2-(2-(2-aminoethoxy)ethoxy)ethyl, or wherein R^{F} and R^{F′} are combined with the nitrogen atom to which each is attached to form a 5-, 6-, or 7-membered heterocycloalkyl ring. In some aspects, R^{F} and R^{F′} are combined with the nitrogen atom to which each is attached to form a 6-membered ring. In some aspects, the 6-membered ring is a morpholinyl or piperazinyl group. In some aspects, R^{F′} is H and R^{F} is glycyl, hydroxyacetyl, ethyl, or 2-(2-(2-aminoethoxy)ethoxy)ethyl. In some aspects, R^{F′} is H and R^{F} comprises an aliphatic group. R^{F′} is H and R^{F} comprises an aryl group. In some aspects, R^{F′} is H and R^{F} comprises an amide group. In some aspects, R^{F′} is H and R^{F} comprises an ethylene oxide group.

In a specific embodiment, the Camptothecins is of formula:

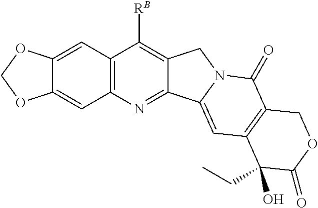

or a pharmaceutically acceptable salt thereof,
wherein R^{B} is —H, —(C₁-C₄)alkyl-OH, —(C₁-C₄)alkyl-O—(C₁-C₄)alkyl-NH₂, —C₁-C₈ alkyl, C₁-C₈ haloalkyl, C₃-C₈ cycloalkyl, C₃-C₈cycloalkylC₁-C₄ alkyl, phenyl or phenylC₁-C₄ alkyl. In some aspects, R^{B} comprises a C₁-C₈ alkyl. In some aspects, R^{B} comprises a cyclopropyl, pentyl, hexyl, tert-butyl, or cyclopentyl group.

Still other Camptothecins are useful in the context of the Conjugates and Compounds described herein. Effectively, the Camptothecin will have a five- or six-ring fused framework analogous to those structures provided as formulae CPT1, CPT2, CPT3, CPT4 and CPT5, but may have additional groups including, but not limited to a hydroxyl, thiol, amine or amide functional group whose oxygen, sulfur or optionally substituted nitrogen heteroatom is capable of incorporation into a linker, and is capable of being released from the conjugate as a free drug. In some aspects, that functional group provides the only site on a drug available for attachment to the Linker Unit (Q). The resulting drug-linker moiety is one that can release active free drug from a Camptothecin Conjugate having that moiety at the site targeted by its Ligand Unit in order to exert a cytotoxic, cytostatic or immunosuppressive effect.

“Free drug” refers to drug, as it exists once released from the drug-linker moiety. In some embodiments, the free drug includes a fragment of the Peptide Releasable Linker (RL) or Spacer Unit (Y) group. In some embodiments, the free drug that includes a fragment of the Peptide Releasable Linker group is biologically active. Free drug that includes a fragment of the Peptide Releasable Linker or Spacer Unit (Y) are released from the remainder of the drug-linker moiety via cleavage of the releasable linker or released via the cleavage of a bond in the Spacer Unit (Y) group and are active after release. In some embodiments, the free drug differs from the conjugated drug in that the functional group of the drug for attachment to the self-immolative assembly unit is no longer associated with components of the Camptothecin Conjugate (other than a previously shared heteroatom). For example, the free hydroxyl functional group of an alcohol-containing drug can be represented as D-O*H, whereas in the conjugated form the oxygen heteroatom designated by O* is incorporated into the methylene carbamate unit of a self-immolative unit. Upon activation of the self-immolative moiety and release of free drug, the covalent bond to O* is replaced by a hydrogen atom so that the oxygen heteroatom designated by O* is present on the free drug as —O—H.

In some embodiments, the Camptothecins are biologically active. In some embodiments, such Camptothecins are useful in a method of inhibiting topoisomerase, killing tumor cells, inhibiting growth of tumor cells, cancer cells, or of a tumor, inhibiting replication of tumor cells or cancer cells, lessening of overall tumor burden or decreasing the number of cancerous cells, or ameliorating one or more symptoms associated with a cancer or autoimmune disease. Such methods comprise, for example, contacting cancer cells with a Camptothecin compound.

### Linker Units (Q)

As noted above, is some embodiments, the linking group Q has a formula selected from the group consisting of:

—Z-A-S*—RL-

—Z-A-L^{P}(S*)—RL-

—Z-A-S*—RL-Y—; and

—Z-A-L^{P}(S*)—RL-Y—,

wherein Z is a Stretcher Unit, A is a Connector Unit; L^{P} is a Parallel Connector Unit; S* is a Partitioning Agent; RL is a Peptide Releasable Linker; and Y is a Spacer Unit.

In one group of embodiments, Q has a formula selected from the group consisting of:

—Z-A-S*—RL-; and —Z-A-S*—RL-Y—;

wherein Z is a Stretcher Unit, A is a bond or a Connector Unit; S* is a Partitioning Agent; and Y is a Spacer Unit.

### Stretcher Unit (Z) or (Z′):

A Stretcher Unit (Z) is a component of a Camptothecin Conjugate or a Camptothecin-Linker Compound or other Intermediate that acts to connect the Ligand Unit to the remainder of the conjugate. In that regard a Stretcher Unit, prior to attachment to a Ligand Unit (i.e. a Stretcher Unit precursor, Z′), has a functional group that can form a bond with a functional group of a targeting ligand.

In some aspects, a Stretcher Unit precursor (Z′) has an electrophilic group that is capable of interacting with a reactive nucleophilic group present on a Ligand Unit (e.g., an antibody) to provide a covalent bond between a Ligand Unit and the Stretcher Unit of a Linker Unit. Nucleophilic groups on an antibody having that capability include but are not limited to, sulfhydryl, hydroxyl and amino functional groups. The heteroatom of the nucleophilic group of an antibody is reactive to an electrophilic group on a Stretcher Unit precursor and provides a covalent bond between the Ligand Unit and Stretcher Unit of a Linker Unit or Drug-Linker moiety. Useful electrophilic groups for that purpose include, but are not limited to, maleimide, haloacetamide groups, and NHS esters. The electrophilic group provides a convenient site for antibody attachment to form a Camptothecin Conjugate or Ligand Unit-Linker intermediate.

In another embodiment, a Stretcher Unit precursor has a reactive site which has a nucleophilic group that is reactive to an electrophilic group present on a Ligand Unit (e.g., an antibody). Useful electrophilic groups on an antibody for that purpose include, but are not limited to, aldehyde and ketone carbonyl groups. The heteroatom of a nucleophilic group of a Stretcher Unit precursor can react with an electrophilic group on an antibody and form a covalent bond to the antibody. Useful nucleophilic groups on a Stretcher Unit precursor for that purpose include, but are not limited to, hydrazide, hydroxylamine, amino, hydrazine, thiosemicarbazone, hydrazine carboxylate, and arylhydrazide. The electrophilic group on an antibody provides a convenient site for antibody attachment to form a Camptothecin Conjugate or Ligand Unit-Linker intermediate.

In some embodiments, a sulfur atom of a Ligand Unit is bound to a succinimide ring system of a Stretcher Unit formed by reaction of a thiol functional group of a targeting ligand with a maleimide moiety of the corresponding Stretcher Unit precursor. In other embodiments, a thiol functional group of a Ligand Unit reacts with an alpha haloacetamide moiety to provide a sulfur-bonded Stretcher Unit by nucleophilic displacement of its halogen substituent.

Representative Stretcher Units of those embodiments include those within the square brackets of Formulas Za and Zb (where the Ligand Unit L is shown for reference):

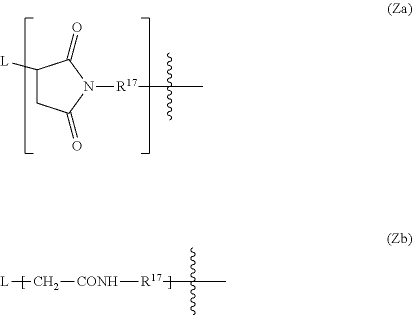

wherein the wavy line indicates attachment to the Parallel Connector Unit (L^{P}) or Connector Unit (A) if L^{P} is absent, or a Partitioning Agent (S*), if L^{P} is absent, and R¹⁷ is —C₁-C₁₀ alkylene-C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—.

In some aspects, the R¹⁷ group of formula Za is optionally substituted by a Basic Unit (BU) such as an aminoalkyl moiety, e.g. —(CH₂)ₓNH₂, —(CH₂)ₓNHR^{a}, and —(CH₂)ₓNR^{a}₂, wherein x is an integer of from 1-4 and each Ra is independently selected from the group consisting of C₁₋₆ alkyl and C₁₋₆ haloalkyl, or two Ra groups are combined with the nitrogen to which they are attached to form an azetidinyl, pyrrolidinyl or piperidinyl group.

An illustrative Stretcher Unit is that of Formula Za or Zb wherein R¹⁷ is —C₁-C₁₀ alkylene-C(═O)—, —C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, or —(C₃-C₈ heterocyclo)-C₁-C₁₀ alkylene-C(═O)—.

Another illustrative Stretcher Unit is that of formula Za wherein R¹⁷ is —C₁-C₅ alkylene-C(═O)—, wherein the alkylene is optionally substituted by a Basic Unit (BU) such as an optionally substituted aminoalkyl, e.g., —(CH₂)ₓNH₂, —(CH₂)ₓNHR^{a}, and —(CH₂)ₓN(R^{a})₂, wherein x is an integer of from 1-4 and each Ra is independently selected from the group consisting of C₁₋₆ alkyl and C₁₋₆ haloalkyl, or two Ra groups are combined with the nitrogen to which they are attached to form an azetidinyl, pyrrolidinyl or piperidinyl group. During synthesis, the basic amino functional group of the Basic Unit can be protected by a protecting group.

Exemplary embodiments of Stretcher Units bonded to a Ligand Unit are as follows:

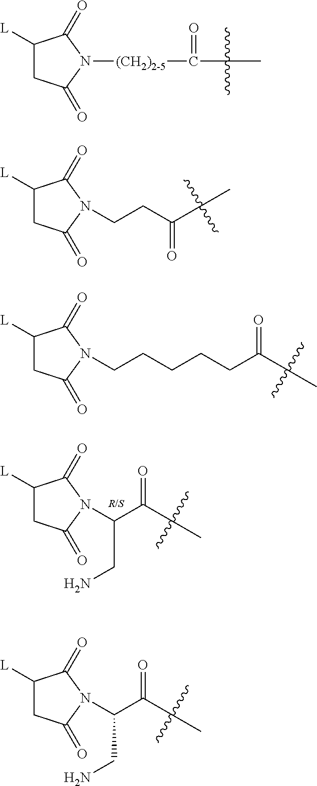

wherein the wavy line adjacent the carbonyl indicates attachment to L^{P}, A, or S* in the formulae above depending on the presence or absence of A and/or L^{P}.

In some preferred embodiments a Stretcher unit (Z) is comprised of a succinimide moiety, that when bonded to L is represented by the structure of formula Za′:

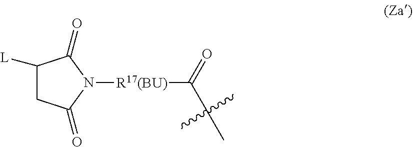

wherein the wavy line adjacent the carbonyl indicates attachment to L^{P}, A, or S* in the formulae above depending on the presence or absence of A and/or L^{P}; R¹⁷ is —C₁-C₅ alkylene-, wherein the alkylene is substituted by a Basic Unit (BU), wherein BU is —(CH₂)ₓNH₂, —(CH₂)ₓNHR^{a}, or —(CH₂)ₓN(R^{a})₂, wherein x is an integer of from 1-4 and each Ra is independently selected from the group consisting of C₁₋₆ alkyl and C₁₋₆ haloalkyl, or both Ra together with the nitrogen to which they are attached define an azetidinyl, pyrrolidinyl or piperidinyl group.

It will be understood that a Ligand Unit-substituted succinimide may exist in hydrolyzed form(s). Those forms are exemplified below for hydrolysis of Za′ bonded to L, wherein the structures representing the regioisomers from that hydrolysis are formula Zb′ and Zc′. Accordingly, in other preferred embodiments a Stretcher unit (Z) is comprised of an acid-amide moiety that when bonded to L is represented by the following:

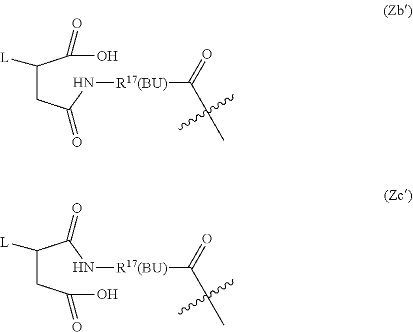

the wavy line adjacent to the carbonyl bonded to R¹⁷ is as defined for Za′, depending on the presence or absence of A and/or L^{P}; and R¹⁷ is —C₁-C₅ alkylene-, wherein the alkylene is substituted by a Basic Unit (BU), wherein BU is —(CH₂)ₓNH₂, —(CH₂)ₓNHR^{a}, or —(CH₂)ₓN(R^{a})₂, wherein x is an integer of from 1-4 and each Ra is independently selected from the group consisting of C₁₋₆ alkyl and C₁₋₆ haloalkyl, or both Ra together with the nitrogen to which they are attached define an azetidinyl, pyrrolidinyl or piperidinyl group.

In some embodiments a Stretcher unit (Z) is comprised of an acid-amide moiety that when bonded to L is represented by the structure of formula Zd′ or Ze′:

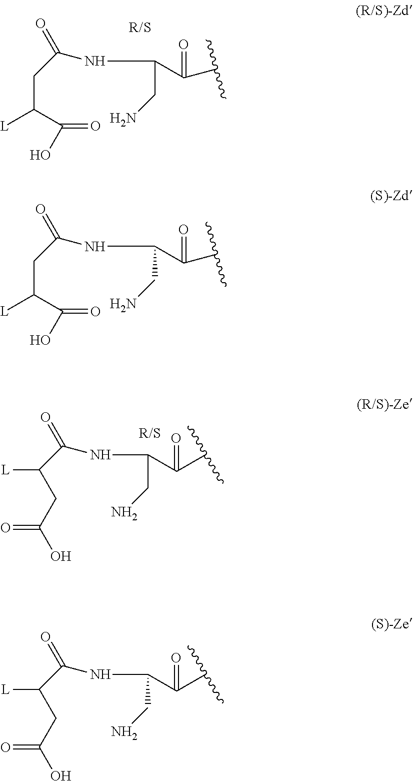

wherein the wavy line adjacent to the carbonyl is as defined for Za′.

In preferred embodiments a Stretcher unit (Z) is comprised of a succinimide moiety that when bonded to L is represented by the structure of

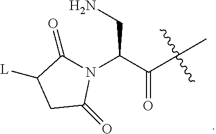

which is generated from a maleimido-amino-propionyl (mDPR) analog (a 3-amino-2-(2,5-dioxo-2,5-dihydro-1H-pyrrol-1-yl)propanoic acid derivative), or is comprised of an acid-amide moiety that when bonded to L is represented by the structure of:

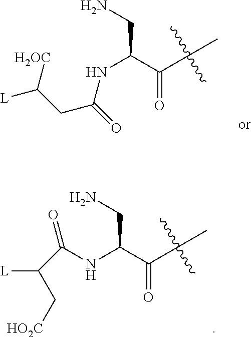

Illustrative Stretcher Units bonded to a Ligand Unit (L) and a Connector Unit (A) have the following structures, which are comprised of the structure from Za, Za′, Zb′ or Zc′, wherein —R¹⁷— or —R¹⁷(BU)— is —CH₂—, —CH₂CH₂— or —CH(CH₂NH₂)—:

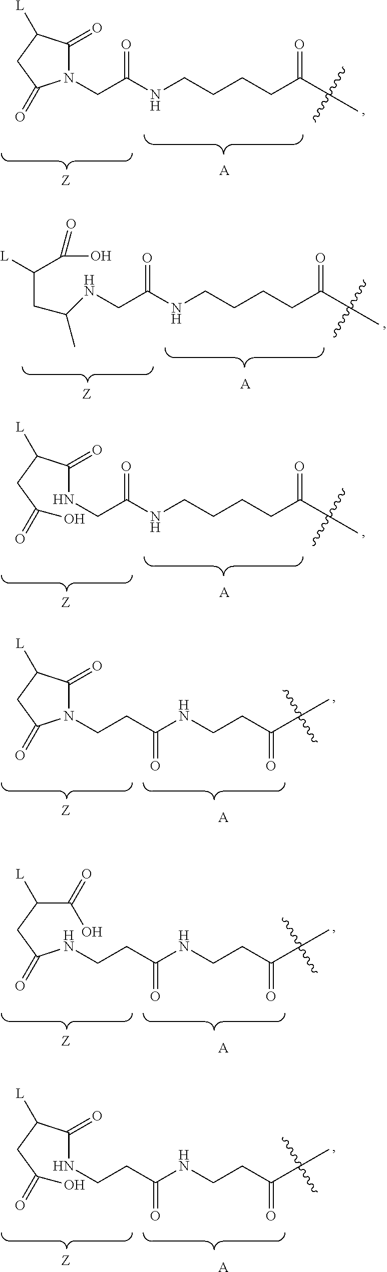

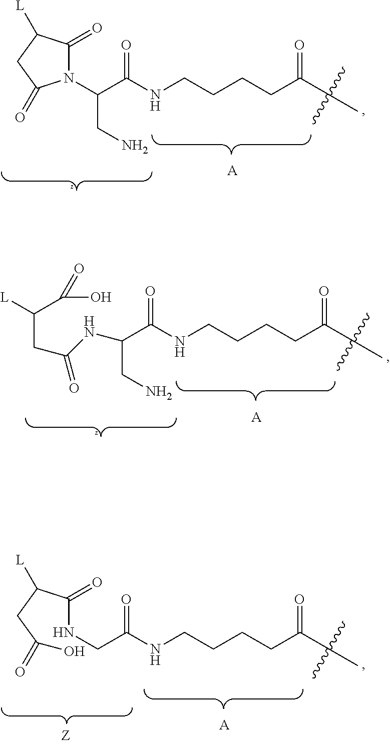

wherein the wavy line adjacent to the carbonyl is as defined for Za′.

In one group of embodiments, Z-A- comprises a maleimido-alkanoic acid component or an mDPR component. See, for example, see WO 2013/173337. In one group of embodiments, Z-A-is a maleimidopropionyl component.

Other Stretcher Units bonded to a Ligand Unit (L) and a Connector Unit (A) have the structures above wherein A in the above Z-A structures is replaced by a Parallel Connector Unit having the structure of

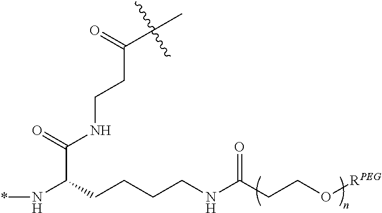

wherein n ranges from 8 to 24; R^{PEG} is a PEG Unit capping group, preferably —CH₃ or —CH₂CH₂CO₂H, the asterisk (*) indicates covalent attachment to a Stretcher Unit corresponding in structure to formula Za, Za′, Zb′ or Zc′ and the wavy line indicates covalent attachment to the Releasable Linker (RL).

Illustrative Stretcher Units prior to conjugation to the Ligand Unit (i.e., Stretcher Unit precursors) are comprised of a maleimide moiety and are represented by structures including that of formula Z′a:

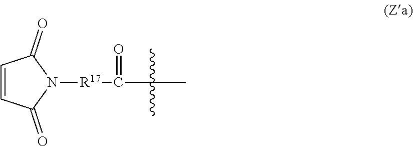

wherein the wavy line adjacent to the carbonyl is as defined for Za′; and R¹⁷ is —(CH₂)₁₋₅—, optionally substituted with a Basic Unit such as an optionally substituted aminoalkyl, e.g., —(CH₂)ₓNH₂, —(CH₂)ₓNHR^{a}, and —(CH₂)ₓN(R^{a})₂, wherein x is an integer of from 1-4 and each Ra is independently selected from the group consisting of C₁₋₆ alkyl and C₁₋₆ haloalkyl, or two Ra groups are combined with the nitrogen to which they are attached to form an azetidinyl, pyrrolidinyl or piperidinyl group.

In some preferred embodiments of formula Z′a, a Stretcher Unit precursor (Z′) is represented by one of the following structures:

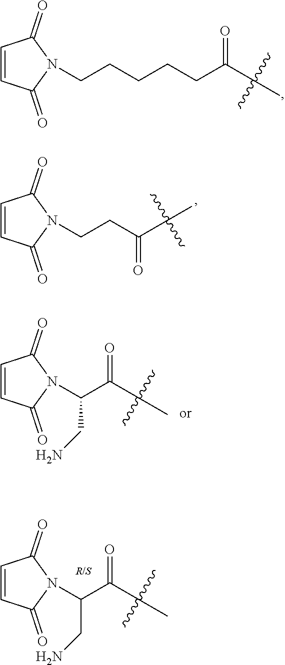

wherein the wavy line adjacent to the carbonyl is as defined for Za′.

In other preferred embodiments a Stretcher Unit precursor (Z′) is comprised of a maleimide moiety and is represented by the structure of formula Za′:

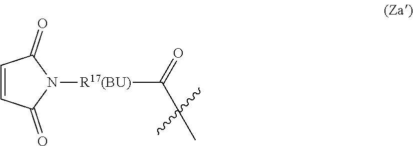

wherein the wavy line adjacent to the carbonyl bonded to R¹⁷ is as defined for Za′; and R¹⁷ is —C₁-C₅ alkylene-, wherein the alkylene is substituted by a Basic Unit (BU), wherein BU is —(CH₂)ₓNH₂, —(CH₂)ₓNHR^{a}, or —(CH₂)ₓN(R^{a})₂, wherein x is an integer of from 1-4 and each Ra is independently selected from the group consisting of C₁₋₆ alkyl and C₁₋₆ haloalkyl, or both Ra together with the nitrogen to which they are attached define an azetidinyl, pyrrolidinyl or piperidinyl group.

In more preferred embodiments the Stretcher unit precursor (Z′) is comprised of a maleimide moiety and is represented by the structure of:

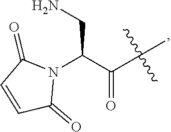

wherein the wavy line adjacent to the carbonyl is as defined for Za′.

In Stretcher Units having a BU moiety, it will be understood that the amino functional group of that moiety may be protected by an amino protecting group during synthesis, e.g., an acid labile protecting group (e.g., BOC).

Illustrative Stretcher Unit precursors covalently attached to a Connector Unit which are comprised of the structure from Za or Za′ wherein —R¹⁷— or —R¹⁷(BU)— is —CH₂—, —CH₂CH₂— or —CH(CH₂NH₂)— have the following structures:

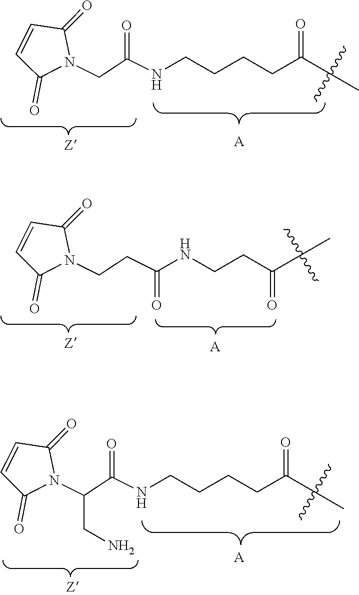

wherein the wavy line adjacent to the carbonyl is as defined for Za′.

Other Stretcher Unit precursors bonded a Connector Unit (A) have the structures above wherein A in the above Z′-A structures is replaced by a Parallel Connector Unit and Partitioning Agent (-L^{P}(S*)—) having the structure of

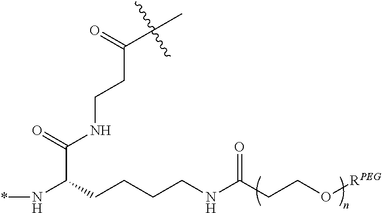

wherein n ranges from 8 to 24; R^{PEG} is a PEG Unit capping group, preferably —CH₃ or —CH₂CH₂CO₂H, the asterisk (*) indicates covalent attachment to the Stretcher Unit precursor corresponding in structure to formula Za or Za′ and the wavy line indicates covalent attachment to RL. In instances such as those shown here, the shown PEG group is meant to be exemplary of a variety of Partitioning Agents including PEG groups of different lengths and other Partitioning Agents that can be directly attached or modified for attachment to the Parallel Connector Unit.

In another embodiment, the Stretcher Unit is attached to the Ligand Unit via a disulfide bond between a sulfur atom of the Ligand Unit and a sulfur atom of the Stretcher unit. A representative Stretcher Unit of this embodiment is depicted within the square brackets of Formula Zb:

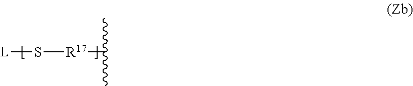

wherein the wavy line indicates attachment to the Parallel Connector Unit (L^{P}) or Connector Unit (A) if L^{P} is absent or a Partitioning Agent (S*), if A and L^{P} are absent and R¹⁷ is —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—.

In yet another embodiment, the reactive group of a Stretcher Unit precursor contains a reactive site that can form a bond with a primary or secondary amino group of a Ligand Unit. Examples of these reactive sites include, but are not limited to, activated esters such as succinimide esters, 4-nitrophenyl esters, pentafluorophenyl esters, tetrafluorophenyl esters, anhydrides, acid chlorides, sulfonyl chlorides, isocyanates and isothiocyanates. Representative Stretcher Units of this embodiment are depicted within the square brackets of Formulas Zci, Zcii and Zciii:

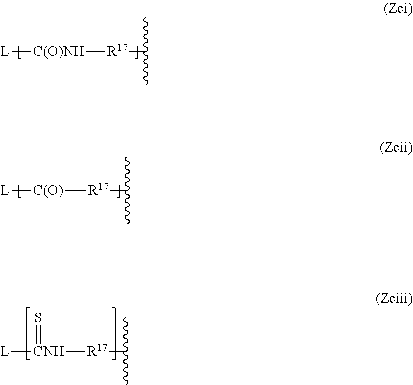

wherein the wavy line indicates attachment to the Parallel Connector Unit (L^{P}) or Connector Unit (A) if L^{P} is absent or a Partitioning Agent (S*), if A and L^{P} are absent and R¹⁷ is —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—.

In yet another aspect, the reactive group of the Stretcher Unit precursor contains a reactive nucleophile that is capable of reacting with an electrophile present on, or introduced to, a Ligand Unit. For example, a carbohydrate moiety on a targeting ligand can be mildly oxidized using a reagent such as sodium periodate and the resulting electrophilic functional group (—CHO) of the oxidized carbohydrate can be condensed with a Stretcher Unit precursor that contains a reactive nucleophile such as a hydrazide, an oxime, a primary or secondary amine, a hydrazine, a thiosemicarbazone, a hydrazine carboxylate, or an arylhydrazide such as those described by Kaneko, T. et al. (1991) Bioconjugate Chem. 2:133-41. Representative Stretcher Units of this embodiment are depicted within the square brackets of Formulas Zdi, Zdii, and Zdiii:

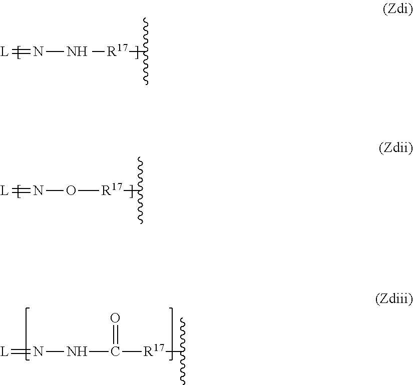

wherein the wavy line indicates attachment to the Parallel Connector Unit (L^{P}) or Connector Unit (A), or a Partitioning Agent (S*), if A and L^{P} are absent and R¹⁷ is —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—.

In some aspects of the prevent invention the Stretcher Unit has a mass of no more than about 1000 daltons, no more than about 500 daltons, no more than about 200 daltons, from about 30, 50 or 100 daltons to about 1000 daltons, from about 30, 50 or 100 daltons to about 500 daltons, or from about 30, 50 or 100 daltons to about 200 daltons.

### Connector Unit (A)

A Connector Unit (A) serves to bind the Stretcher Unit (Z) to the Partitioning Agent (S*) or Parallel Connector Unit/Partitioning Agent combination (-L^{P}(S*)—). In some embodiments, the Connector Unit (A) is a bond that directly links the components. In some embodiments, a Connector Unit (A) is included in a Camptothecin Conjugate or Camptothecin-Linker Compound to add additional distance between the Stretcher Unit (Z) or precursor thereof (Z′) and the Peptide Releasable Linker (RL). In some aspects, the extra distance will aid with activation within RL. Accordingly, the Connector Unit (A), when present, extends the framework of the Linker Unit. In that regard, a Connector Unit (A) is covalently bonded with the Stretcher Unit (or its precursor) at one terminus and is covalently bonded to the optional Parallel Connector Unit (L^{P}) or the Partitioning Agent (S*) at its other terminus.

The skilled artisan will appreciate that the Connector Unit can be any group that serves to provide for attachment of the Partitioning Agent/Peptide Releasable Linker portion (—S*—RL-) or the Parallel Connector Unit/Partitioning Agent/Peptide Releasable Linker portion (-L^{P}(S*)—RL-) to the remainder of the Linker Unit (Q). The Connector Unit can be, for example, comprised of one or more (e.g., 1-10, preferably, 1, 2, 3, or 4) natural or non-natural amino acid, amino alcohol, amino aldehyde, diamino residues. In some aspects, the Connector Unit is a single natural or non-natural amino acid, amino alcohol, amino aldehyde, or diamino residue. An exemplary amino acid capable of acting as Connector units is β-alanine.

In some aspects, the Connector Unit has the formula denoted below:

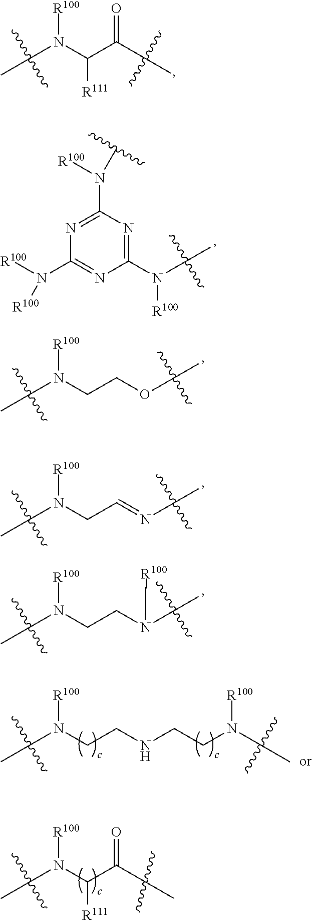

wherein the wavy lines indicate attachment of the Connector Unit within the Camptothecin Conjugate or Camptothecin Linker Compound; and wherein R¹¹¹ is independently selected from the group consisting of hydrogen, p-hydroxybenzyl, methyl, isopropyl, isobutyl, sec-butyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-,

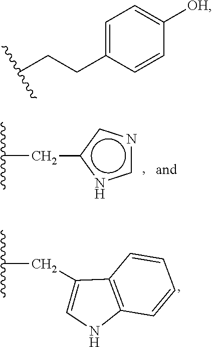

and each R¹⁰⁰ is independently selected from hydrogen or —C₁-C₃ alkyl, preferably hydrogen or CH₃; and the subscript c is an independently selected integer from 1 to 10, preferably 1 to 3.

A representative Connector Unit having a carbonyl group for attachment to the Partitioning Agent (S*) or to -L^{P}(S*)— is as follows:

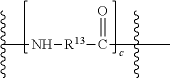

wherein in each instance R¹³ is independently selected from the group consisting of —C₁-C₆ alkylene-, —C₃-C₈carbocyclo-, -arylene-, —C₁-C₁₀ heteroalkylene-, —C₃-C₈heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈ heterocyclo)-, and —(C₃-C₈ heterocyclo)-C₁-C₁₀ alkylene-, and the subscript c is an integer ranging from 1 to 4. In some embodiments R¹³ is —C₁-C₆ alkylene and c is 1.

Another representative Connector Unit having a carbonyl group for attachment to Partitioning Agent (S*) or to -L^{P}(S*)— is as follows:

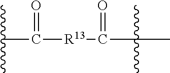

wherein R¹³ is —C₁-C₆ alkylene-, —C₃-C₈carbocyclo-, -arylene-, —C₁-C₁₀ heteroalkylene-, —C₃-C₈heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈ heterocyclo)-, or —(C₃-C₈ heterocyclo)-C₁-C₁₀ alkylene-. In some embodiments R¹³ is —C₁-C₆ alkylene.

A representative Connector Unit having a NH moiety that attaches to Partitioning Agent (S*) or to -L^{P}(S*)— is as follows:

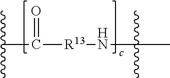

wherein in each instance, R¹³ is independently selected from the group consisting of —C₁-C₆ alkylene-, —C₃-C₈carbocyclo-, -arylene-, —C₁-C₁₀ heteroalkylene-, —C₃-C₈heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈ heterocyclo)-, and —(C₃-C₈ heterocyclo)-C₁-C₁₀ alkylene-, and the subscript c is from 1 to 14. In some embodiments R¹³ is —C₁-C₆ alkylene and the subscript c is 1.

Another representative Connector Unit having a NH moiety that attaches to Partitioning Agent (S*) or to -L^{P}(S*)— is as follows:

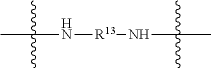

wherein R¹³ is —C₁-C₆ alkylene-, —C₃-C₈carbocyclo-, -arylene-, —C₁-C₁₀ heteroalkylene-, —C₃-C₈heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈ heterocyclo)-, —(C₃-C₈ heterocyclo)-C₁-C₁₀ alkylene-, —C(═O)C₁-C₆ alkylene- or —C₁-C₆ alkylene-C(═O)—C₁-C₆ alkylene.

Selected embodiments of Connector Units include those having the following structure

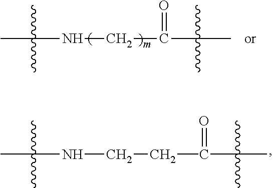

wherein the wavy line adjacent to the nitrogen indicates covalent attachment a Stretcher Unit (Z) (or its precursor Z′), and the wavy line adjacent to the carbonyl indicates covalent attachment to Partitioning Agent (S*) or to -L^{P}(S*)—; and m is an integer ranging from 1 to 6, preferably 2 to 6, more preferably 2 to 4.

### Peptide Releasable Linker (RL):

In some embodiments, the Peptide Releasable Linker (RL) will comprise two or more contiguous or non-contiguous sequences of amino acids (e.g., so that RL has 2 to no more than 12 amino acids). The Peptide Releasable Linker can comprise or consist of, for example, a dipeptide, tripeptide, tetrapeptide, pentapeptide, hexapeptide, heptapeptide, octapeptide, nonapeptide, decapeptide, undecapeptide or dodecapeptide unit. In some aspects, in the presence of an enzyme (e.g., a tumor-associated protease), an amide linkage between the amino acids is cleaved, which ultimately leads to release of free drug.

Each amino acid can be natural or unnatural and/or a D- or L-isomer provided that RL comprises a cleavable bond that, when cleaved, initiates release of the Camptothecin. In some embodiments, the Peptide Releasable Linker will comprise only natural amino acids. In some aspects, the Peptide Releasable Linker will have from 2 to no more than 12 amino acids in contiguous sequence.

In some embodiments, each amino acid is independently selected from the group consisting of alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, selenocysteine, ornithine, penicillamine, β-alanine, aminoalkanoic acid, aminoalkynoic acid, aminoalkanedioic acid, aminobenzoic acid, amino-heterocyclo-alkanoic acid, heterocyclo-carboxylic acid, citrulline, statine, diaminoalkanoic acid, and derivatives thereof. In some embodiments, each amino acid is independently selected from the group consisting of alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, and selenocysteine. In some embodiments, each amino acid is independently selected from the group consisting of alanine, arginine, aspartic acid, asparagine, histidine, glycine, glutamic acid, glutamine, phenylalanine, lysine, leucine, serine, tyrosine, threonine, isoleucine, proline, tryptophan, and valine. In some embodiments, each amino acid is selected from the proteinogenic or the non-proteinogenic amino acids.

In another embodiment, each amino acid is independently selected from the group consisting of the following L-(natural) amino acids: alanine, arginine, aspartic acid, asparagine, histidine, glycine, glutamic acid, glutamine, phenylalanine, lysine, leucine, serine, tyrosine, threonine, isoleucine, tryptophan and valine.

In another embodiment, each amino acid is independently selected from the group consisting of the following D-isomers of these natural amino acids: alanine, arginine, aspartic acid, asparagine, histidine, glycine, glutamic acid, glutamine, phenylalanine, lysine, leucine, serine, tyrosine, threonine, isoleucine, tryptophan and valine.

In certain embodiments, the Peptide Releasable Linker is comprised only of natural amino acids. In other embodiments, the Peptide Releasable Linker is comprised only of non-natural amino acids. In some embodiments, the Peptide Releasable Linker is comprised of a natural amino acid attached to a non-natural amino acid. In some embodiments, Peptide Releasable Linker is comprised of a natural amino acid attached to a D-isomer of a natural amino acid.

In another embodiment, each amino acid is independently selected from the group consisting of β-alanine, N-methylglycine, glycine, lysine, valine and phenylalanine.

Exemplary Peptide Releasable Linkers include dipeptides or tripeptides with-Val-Lys-Gly-, -Val-Cit-, -Phe-Lys- or -Val-Ala-.

Useful Peptide Releasable Linkers can be designed and optimized in their selectivity for enzymatic cleavage by a particular enzyme, for example, a tumor-associated protease. In some embodiments, cleavage of a linkage is catalyzed by cathepsin B, C or D, or a plasmin protease.

In some embodiments, the Peptide Releasable Linker (RL) will be represented by -(-AA-)₂₋₁₂-, or (-AA-AA-)₁₋₆ wherein AA is at each occurrence independently selected from natural or non-natural amino acids. In one aspect, AA is at each occurrence independently selected from natural amino acids. In another aspect, RL is a tripeptide having the formula: AA₁-AA₂-AA₃, wherein AA₁, AA₂ and AA₃ are each independently an amino acid and wherein AA₁ attaches to —NH— and AA₃ attaches to S*. In yet another aspect, AA₃ is gly or β-ala.

In some embodiments, the Peptide Releasable Linker has the formula denoted below in the square brackets, the subscript w is an integer ranging from 2 to 12, or w is 2, 3, or 4, or w is 3:

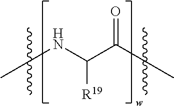

wherein R¹⁹ is, in each instance, independently selected from the group consisting of hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

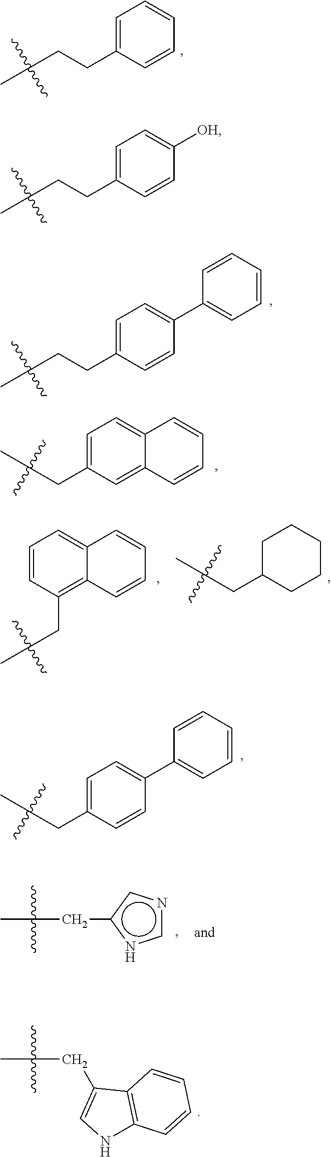

In some aspects, each R¹⁹ is independently hydrogen, methyl, isopropyl, isobutyl, sec-butyl, —(CH₂)₃NH₂, or —(CH₂)₄NH₂. In some aspects, each R¹⁹ is independently hydrogen, isopropyl, or —(CH₂)₄NH₂.

Illustrative Peptide Releasable Linkers are represented by formulae (Pa), (Pb) and (Pc)

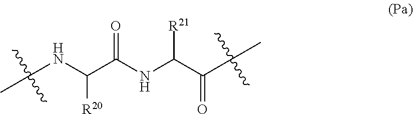

wherein R²⁰ and R²¹ are as follows:

| R²⁰ | R²¹ |
|---|---|
| benzyl | (CH₂)₄NH₂; |
| methyl | (CH₂)₄NH₂; |
| isopropyl | (CH₂)₄NH₂; |
| isopropyl | (CH₂)₃NHCONH₂; |
| benzyl | (CH₂)₃NHCONH₂; |
| isobutyl | (CH₂)₃NHCONH₂; |
| sec-butyl | (CH₂)₃NHCONH₂; |
| 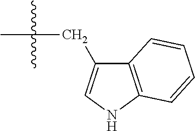 [SMILES: *Cc1c[nH]c2ccccc12] | (CH₂)₃NHCONH₂; |
| benzyl | methyl; and |
| benzyl | (CH₂)₃NHC(═NH)NH₂; |

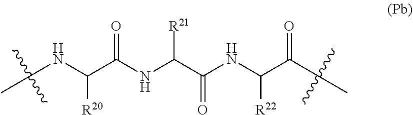

wherein R²⁰, R²¹ and R²² are as follows:

| R²⁰ | R²¹ | R²² |
|---|---|---|
| benzyl | benzyl | —(CH₂)₄NH₂ |
| isopropyl | benzyl | —(CH₂)₄NH₂ |
| H | Benzyl | —(CH₂)₄NH₂ |
| isopropyl | —(CH₂)₄NH₂ | —H |

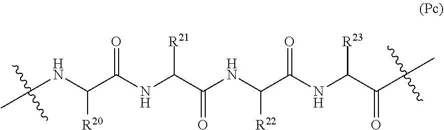

wherein R²⁰, R²¹, R²² and R²³ are as follows:

| R²⁰ | R²¹ | R²² | R²³ |
|---|---|---|---|
| H methyl | benzyl isobutyl | isobutyl methyl | H; and isobutyl. |

In some embodiments, RL comprises a peptide selected from the group consisting of gly-gly, gly-gly-gly, gly-gly-gly-gly, val-gly-gly, val-cit-gly, val-gln-gly, val-glu-gly, phe-lys-gly, leu-lys-gly, gly-val-lys-gly, val-lys-gly-gly, val-lys-gly, val-lys-ala, val-lys-leu, leu-leu-gly, gly-gly-phe-gly, gly-gly-phe-gly-gly, val-gly, and val-lys-β-ala.

In other embodiments, RL comprises a peptide selected from the group consisting of gly-gly-gly, gly-gly-gly-gly, val-gly-gly, val-cit-gly, val-gln-gly, val-glu-gly, phe-lys-gly, leu-lys-gly, gly-val-lys-gly, val-lys-gly-gly, val-lys-gly, val-lys-ala, val-lys-leu, leu-leu-gly, gly-gly-phe-gly, and val-lys-β-ala.

In still other embodiments, RL comprises a peptide selected from the group consisting of gly-gly-gly, val-gly-gly, val-cit-gly, val-gln-gly, val-glu-gly, phe-lys-gly, leu-lys-gly, val-lys-gly, val-lys-ala, val-lys-leu, leu-leu-gly and val-lys-β-ala.

In yet other embodiments, RL comprises a peptide selected from the group consisting of gly-gly-gly-gly, gly-val-lys-gly, val-lys-gly-gly, and gly-gly-phe-gly.

In other embodiments, RL is a peptide selected from the group consisting of val-gln-gly, val-glu-gly, phe-lys-gly, leu-lys-gly, val-lys-gly, val-lys-ala, val-lys-leu, leu-leu-gly and val-lys-β-ala.

In still other embodiments, RL is val-lys-gly.

In still other embodiments, RL is val-lys-β-ala.

### Partitioning Agent (S*):

The Camptothecin Conjugates described herein can also include a Partitioning Agent (S*). The Partitioning Agent portions are useful, for example, to mask the hydrophobicity of particular Camptothecins or other Linking Unit components.

Representative Partitioning Agents include polyethylene glycol (PEG) units, cyclodextrin units, polyamides, hydrophilic peptides, polysaccharides and dendrimers.

When the polyethylene glycol (PEG) units, cyclodextrin units, polyamides, hydrophilic peptides, polysaccharides or dendrimers are included in Q, the groups may be present as an ‘in line’ component or as a side chain or branched component. For those embodiments in which a branched version is present, the Linker Units will typically include a lysine residue (or Parallel Connector Unit, L^{P}) that provides simple functional conjugation of, for example, the PEG Unit, to the remainder of the Linking Unit.

### Polyethylene Glycol (PEG) Unit

Polydisperse PEGs, monodisperse PEGs and discrete PEGs can be used as part of the Partitioning Agents in the Compounds of the present invention. Polydisperse PEGs are a heterogeneous mixture of sizes and molecular weights whereas monodisperse PEGs are typically purified from heterogeneous mixtures and are therefore provide a single chain length and molecular weight. Preferred PEGs are discrete PEGs, compounds that are synthesized in step-wise fashion and not via a polymerization process. Discrete PEGs provide a single molecule with defined and specified chain length.

The PEGs provided herein comprises one or multiple polyethylene glycol chains. A polyethylene glycol chain is composed of at least two ethylene oxide (CH₂CH₂O) subunits. The polyethylene glycol chains can be linked together, for example, in a linear, branched or star shaped configuration. Typically, at least one of the PEG chains is derivatized at one end for covalent attachment to an appropriate site on a component of the Linker Unit (e.g. L^{P}) or can be used as an in-line (e.g., bifunctional) linking group within to covalently join two of the Linker Unit components (e.g., Z-A-S*—RL-, Z-A-S*—RL-Y—). Exemplary attachments within the Linker Unit are by means of non-conditionally cleavable linkages or via conditionally cleavable linkages. Exemplary attachments are via amide linkage, ether linkages, ester linkages, hydrazone linkages, oxime linkages, disulfide linkages, peptide linkages or triazole linkages. In some aspects, attachment within the Linker Unit is by means of a non-conditionally cleavable linkage. In some aspects, attachment within the Linker Unit is not via an ester linkage, hydrazone linkage, oxime linkage, or disulfide linkage. In some aspects, attachment within the Linker Unit is not via a hydrazone linkage.

A conditionally cleavable linkage refers to a linkage that is not substantially sensitive to cleavage while circulating in the plasma but is sensitive to cleavage in an intracellular or intratumoral environment. A non-conditionally cleavable linkage is one that is not substantially sensitive to cleavage in any biological environment. Chemical hydrolysis of a hydrazone, reduction of a disulfide, and enzymatic cleavage of a peptide bond or glycosidic linkage are examples of conditionally cleavable linkages.

In some embodiments, the PEG Unit will be directly attached to a Parallel Connector Unit B. The other terminus (or termini) of the PEG Unit can be free and untethered and may take the form of a methoxy, carboxylic acid, alcohol or other suitable functional group. The methoxy, carboxylic acid, alcohol or other suitable functional group acts as a cap for the terminal PEG subunit of the PEG Unit. By untethered, it is meant that the PEG Unit will not be attached at that untethered site to a Camptothecin, to an antibody, or to another linking component. The skilled artisan will understand that the PEG Unit in addition to comprising repeating ethylene glycol subunits may also contain non-PEG material (e.g., to facilitate coupling of multiple PEG chains to each other). Non-PEG material refers to the atoms in the PEG Unit that are not part of the repeating —CH₂CH₂O— subunits. In some embodiments provided herein, the PEG Unit comprises two monomeric PEG chains attached to each other via non-PEG elements. In other embodiments provided herein, the PEG Unit comprises two linear PEG chains attached to a central core or Parallel Connector Unit (i.e., the PEG Unit itself is branched).

There are a number of PEG attachment methods available to those skilled in the art, [see, e.g., Goodson, et al. (1990) Bio/Technology 8:343 (PEGylation of interleukin-2 at its glycosylation site after site-directed mutagenesis); EP 0 401 384 (coupling PEG to G-CSF); Malik, et al., (1992) Exp. Hematol. 20:1028-1035 (PEGylation of GM-CSF using tresyl chloride); ACT Pub. No. WO 90/12874 (PEGylation of erythropoietin containing a recombinantly introduced cysteine residue using a cysteine-specific mPEG derivative); U.S. Pat. No. 5,757,078 (PEGylation of EPO peptides); U.S. Pat. No. 5,672,662 (Poly(ethylene glycol) and related polymers monosubstituted with propionic or butanoic acids and functional derivatives thereof for biotechnical applications); U.S. Pat. No. 6,077,939 (PEGylation of an N-terminal .alpha.-carbon of a peptide); Veronese et al., (1985) Appl. Biochem. Bioechnol 11:141-142 (PEGylation of an N-terminal α-carbon of a peptide with PEG-nitrophenylcarbonate (“PEG-NPC”) or PEG-trichlorophenylcarbonate); and Veronese (2001) Biomaterials 22:405-417 (Review article on peptide and protein PEGylation)].

For example, PEG may be covalently bound to amino acid residues via a reactive group. Reactive groups are those to which an activated PEG molecule may be bound (e.g., a free amino or carboxyl group). For example, N-terminal amino acid residues and lysine (K) residues have a free amino group; and C-terminal amino acid residues have a free carboxyl group. Thiol groups (e.g., as found on cysteine residues) are also useful as a reactive group for attaching PEG. In addition, enzyme-assisted methods for introducing activated groups (e.g., hydrazide, aldehyde, and aromatic-amino groups) specifically at the C-terminus of a polypeptide have been described (see Schwarz, et al. (1990) Methods Enzymol. 184:160; Rose, et al. (1991) Bioconjugate Chem. 2:154; and Gaertner, et al. (1994) J. Biol. Chem. 269:7224].

In some embodiments, PEG molecules may be attached to amino groups using methoxylated PEG (“mPEG”) having different reactive moieties. Non-limiting examples of such reactive moieties include succinimidyl succinate (SS), succinimidyl carbonate (SC), mPEG-imidate, para-nitrophenylcarbonate (NPC), succinimidyl propionate (SPA), and cyanuric chloride. Non-limiting examples of such mPEGs include mPEG-succinimidyl succinate (mPEG-SS), mPEG₂-succinimidyl succinate (mPEG₂-SS); mPEG-succinimidyl carbonate (mPEG-SC), mPEG₂-succinimidyl carbonate (mPEG₂-SC); mPEG-imidate, mPEG-para-nitrophenylcarbonate (mPEG-NPC), mPEG-imidate; mPEG₂-para-nitrophenylcarbonate (mPEG₂-NPC); mPEG-succinimidyl propionate (mPEG-SPA); mPEG₂-succinimidyl propionate (mPEG, -SPA); mPEG-N-hydroxy-succinimide (mPEG-NHS); mPEG₂-N-hydroxy-succinimide (mPEG₂-NHS); mPEG-cyanuric chloride; mPEG₂-cyanuric chloride; mPEG₂-Lysinol-NPC, and mPEG₂-Lys-NHS.

Generally, at least one of the PEG chains that make up the PEG Unit is functionalized so that it is capable of covalent attachment to other Linker Unit components.

Functionalization includes, for example, via an amine, thiol, NHS ester, maleimide, alkyne, azide, carbonyl, or another functional group. In some embodiments, the PEG Unit further comprises non-PEG material (i.e., material not comprised of —CH₂CH₂O—) that provides coupling to other Linker Unit components or to facilitate coupling of two or more PEG chains.

The presence of the PEG Unit (or other Partitioning Agent) in the Linker Unit can have two potential impacts upon the pharmacokinetics of the resulting Camptothecin Conjugate. The desired impact is a decrease in clearance (and consequent increase in exposure) that arises from the reduction in non-specific interactions induced by the exposed hydrophobic elements of the Camptothecin Conjugate or to the Camptothecin itself. The second impact is undesired and is a decrease in volume and rate of distribution that sometimes arises from the increase in the molecular weight of the Camptothecin Conjugate. Increasing the number of PEG subunits increases the hydrodynamic radius of a conjugate, typically resulting in decreased diffusivity. In turn, decreased diffusivity typically diminishes the ability of the Camptothecin Conjugate to penetrate into a tumor (Schmidt and Wittrup, Mol Cancer Ther 2009; 8:2861-2871). Because of these two competing pharmacokinetic effects, it is desirable to use a PEG that is sufficiently large to decrease the Camptothecin Conjugate clearance thus increasing plasma exposure, but not so large as to greatly diminish its diffusivity, to an extent that it interferes with the ability of the Camptothecin Conjugate to reach the intended target cell population. See the examples (e.g., examples 1, 18, and 21 of US2016/0310612), which is incorporated by reference herein, for methodology for selecting an optimal PEG size for a particular drug-linker.

In one group of embodiments, the PEG Unit comprises one or more linear PEG chains each having at least 2 subunits, at least 3 subunits, at least 4 subunits, at least 5 subunits, at least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, at least 20 subunits, at least 21 subunits, at least 22 subunits, at least 23 subunits, or at least 24 subunits. In preferred embodiments, the PEG Unit comprises a combined total of at least 4 subunits, at least 6 subunits, at least 8 subunits, at least 10 subunits, or at least 12 subunits. In some such embodiments, the PEG Unit comprises no more than a combined total of about 72 subunits, preferably no more than a combined total of about 36 subunits.

In another group of embodiments, the PEG Unit comprises a combined total of from 4 to 72, 4 to 60, 4 to 48, 4 to 36 or 4 to 24 subunits, from 5 to 72, 5 to 60, 5 to 48, 5 to 36 or 5 to 24 subunits, from 6 to 72, 6 to 60, 6 to 48, 6 to 36 or from 6 to 24 subunits, from 7 to 72, 7 to 60, 7 to 48, 7 to 36 or 7 to 24 subunits, from 8 to 72, 8 to 60, 8 to 48, 8 to 36 or 8 to 24 subunits, from 9 to 72, 9 to 60, 9 to 48, 9 to 36 or 9 to 24 subunits, from 10 to 72, 10 to 60, 10 to 48, 10 to 36 or 10 to 24 subunits, from 11 to 72, 11 to 60, 11 to 48, 11 to 36 or 11 to 24 subunits, from 12 to 72, 12 to 60, 12 to 48, 12 to 36 or 12 to 24 subunits, from 13 to 72, 13 to 60, 13 to 48, 13 to 36 or 13 to 24 subunits, from 14 to 72, 14 to 60, 14 to 48, 14 to 36 or 14 to 24 subunits, from 15 to 72, 15 to 60, 15 to 48, 15 to 36 or 15 to 24 subunits, from 16 to 72, 16 to 60, 16 to 48, 16 to 36 or 16 to 24 subunits, from 17 to 72, 17 to 60, 17 to 48, 17 to 36 or 17 to 24 subunits, from 18 to 72, 18 to 60, 18 to 48, 18 to 36 or 18 to 24 subunits, from 19 to 72, 19 to 60, 19 to 48, 19 to 36 or 19 to 24 subunits, from 20 to 72, 20 to 60, 20 to 48, 20 to 36 or 20 to 24 subunits, from 21 to 72, 21 to 60, 21 to 48, 21 to 36 or 21 to 24 subunits, from 22 to 72, 22 to 60, 22 to 48, 22 to 36 or 22 to 24 subunits, from 23 to 72, 23 to 60, 23 to 48, 23 to 36 or 23 to 24 subunits, or from 24 to 72, 24 to 60, 24 to 48, 24 to 36 or 24 subunits.

In some embodiments, the Partitioning Agent S* is a linear PEG Unit comprising from 2 to 20, or from 2 to 12, or from 4 to 12, or 4, 8, or 12 —CH₂CH₂O— subunits. In some embodiments, the linear PEG Unit is connected at one end of the PEG Unit to the RL Unit and at the other end of the PEG Unit to the Stretcher/Connector Units (Z-A-). In some embodiments, the PEG Unit is connected to the RL Unit via a —CH₂CH₂C(O)— group that forms an amide bond with the RL Unit (e.g., —(CH₂CH₂O)ₙ—CH₂CH₂C(O)—RL) and to the Stretcher Unit/Connector Unit (Z-A-) via an —NH— group (e.g., Z-A-NH—(CH₂CH₂O)ₙ—) that forms an amide bond with the Z-A- portion.

Illustrative embodiments for PEG Units that are connected to the RL and Stretcher/Connector Units (Z-A-) are shown below:

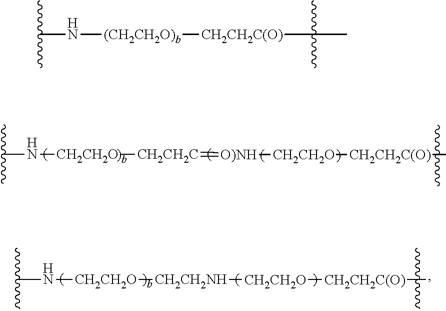

and in a particular embodiment, the PEG Unit is:

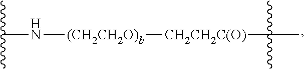

wherein the wavy line on the left indicates the site of attachment to Z-A-, the wavy line on the right indicates the site of attachment to RL, and each b is independently selected from 2 to 72, 4 to 72, 6 to 72, 8 to 72, 10 to 72, 12 to 72, 2 to 24, 4 to 24, 6 to 24, or 8 to 24, 2 to 12, 4 to 12, 6 to 12, and 8 to 12. In some embodiments, subscript b is 2, 4, 8, 12, or 24. In some embodiments, subscript b is 2. In some embodiments, subscript b is 4. In some embodiments, subscript b is 8. In some embodiments, subscript b is 12.

In some embodiments, the linear PEG Unit that is connected to the Parallel Connector Unit at one end and comprises a terminal cap at the other end. In some embodiments, the PEG Unit is connected to the Parallel Connector Unit via a carbonyl group that forms an amide bond with the Parallel Connector Unit lysine residue amino group (e.g., —(OCH₂CH₂)ₙ—C(O)-L^{P}-) and includes a PEG Unit terminal cap group selected from the group consisting of C₁₋₄alkyl and C₁₋₄alkyl-CO₂H. In some embodiments, the Partitioning Agent S* is a linear PEG Unit comprising 4, 8, or 12 —CH₂CH₂O— subunits and a terminal methyl cap.

Illustrative linear PEG Units that can be used in any of the embodiments provided herein are as follows:

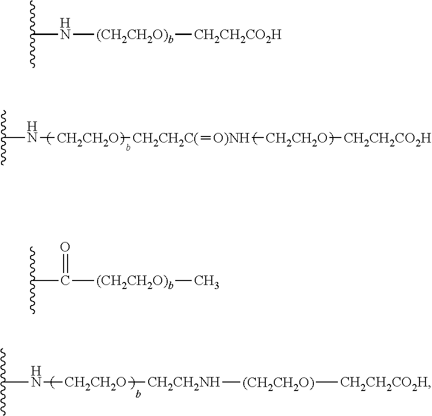

and in a particular embodiment, the PEG Unit is:

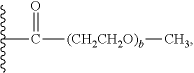

wherein the wavy line indicates site of attachment to the Parallel Connector Unit (L^{P}), and each n is independently selected from 4 to 72, 6 to 72, 8 to 72, 10 to 72, 12 to 72, 6 to 24, or 8 to 24. In some embodiments, subscript b is about 4, about 8, about 12, or about 24.

As used to herein, terms “PEG2”, “PEG4”, “PEG8”, and “PEG12” refers to specific embodiments of PEG Unit which comprises the number of PEG subunits (i.e., the number of subscription “b”). For example, “PEG2” refers to embodiments of PEG Unit that comprises 2 PEG subunits, “PEG4” refers to embodiments of PEG Unit that comprises 4 PEG subunits, “PEG8” refers to embodiments of PEG Unit that comprises 8 PEG subunits, and “PEG12” refers to embodiments of PEG Unit that comprises 12 PEG subunits.camptothecin-liner compounds

As described herein, the number of PEG subunits is selected such that it improves clearance of the resultant Camptothecin Conjugate but does not significantly impact the ability of the Conjugate to penetrate into the tumor. In embodiments, the number of PEG subunits to be selected for use will preferably have from 2 subunits to about 24 subunits, from 4 subunits to about 24 subunits, more preferably about 4 subunits to about 12 subunits.

In preferred embodiments of the present disclosure the PEG Unit is from about 300 daltons to about 5 kilodaltons; from about 300 daltons, to about 4 kilodaltons; from about 300 daltons, to about 3 kilodaltons; from about 300 daltons, to about 2 kilodaltons; or from about 300 daltons, to about 1 kilodalton. In some such aspects, the PEG Unit has at least 6 subunits or at least 8, 10 or 12 subunits. In some such aspects, the PEG Unit has at least 6 subunits or at least 8, 10 or 12 subunits but no more than 72 subunits, preferably no more than 36 subunits.

It will be appreciated that when referring to PEG subunits, and depending on context, the number of subunits can represent an average number, e.g., when referring to a population of Camptothecin Conjugates or Camptothecin-Linker Compounds, and using polydisperse PEGs.

### Parallel Connector Unit (L^{P}):

In some embodiments, the Camptothecin Conjugates and Camptothecin Linker Compounds will comprise a Parallel Connector Unit to provide a point of attachment to a Partitioning Agent (shown in the Linker Units as -L^{P}(S*)—). As a general embodiment, the PEG Unit can be attached to a Parallel Connector Unit such as lysine as shown below wherein the wavy line and asterisks indicate covalent linkage within the Linker Unit of a Camptothecin Conjugate or Camptothecin Linker Compound:

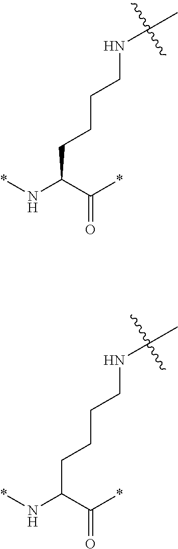 [SMILES: *NCCCC[C@H](N*)C(*)=O ; *NCCCCC(N*)C(*)=O]

In some embodiments, the Parallel Connector Unit (L^{P}) and Partitioning Agent (S*) (together, -L^{P}(S*)—) have the structure of

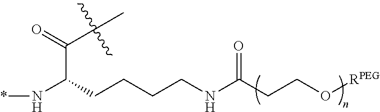

wherein n ranges from 8 to 24; R^{PEG} is a PEG Unit capping group, preferably —CH₃ or —CH₂CH₂CO₂H, the asterisk (*) indicates covalent attachment to a Connector Unit A corresponding in formula Za, Za′, Zb′ or Zc′ and the wavy line indicates covalent attachment to the Releasable Linker (RL). In some embodiments, the structure is attached to a Connector Unit A in formula Za or Za′. In some embodiments, n is 2, 4, 8, or 12. In instances such as those shown here, the shown PEG group is meant to be exemplary of a variety of Partitioning Agents including PEG groups of different lengths and other Partitioning Agents that can be directly attached or modified for attachment to the Parallel Connector Unit.

### Spacer (Y):

In some embodiments, the Camptothecin Conjugates provided herein will have a Spacer (Y) between the Releasable Linker (RL) and the Camptothecin. The Spacer can be a functional group to facilitate attachment of RL to the Camptothecin, or it can provide additional structural components to further facilitate release of the Camptothecin from the remainder of the Conjugate (e.g., a self-immolative para-aminobenzyl (PAB) component).

Still other Spacer Units are represented by the formulae:

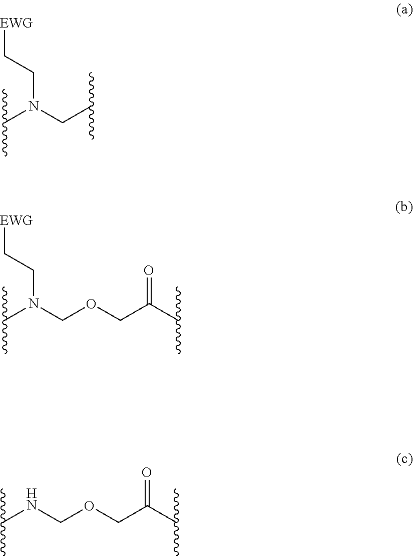

wherein in each instance EWG represents an electron-withdrawing group. In some embodiments, EWG is selected from the group consisting of —CN, —NO₂, —CX₃, —X′, C(═O)OR′, —C(═O)N(R′)₂, —C(═O)R′, —C(═O)X, —S(═O)₂R′, —S(═O)₂OR′, —S(═O)₂NHR′, —S(═O)₂N(R′)₂, —P(═O)(OR′)₂, —P(═O)(CH₃)NHR′, —NO, —N(R′)₃⁺, wherein X is —F, —Br, —Cl, or —I, and R′ is independently selected from the group consisting of hydrogen and C₁₋₆ alkyl.

In still other embodiments, Spacer Units are represented by the formulae:

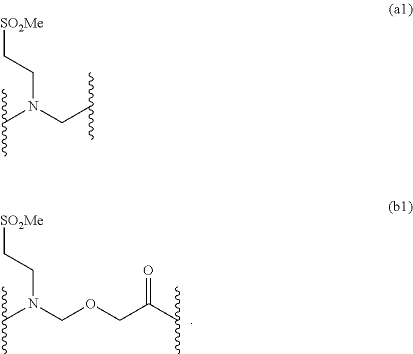 [SMILES: *CN(*)CCS(C)(=O)=O ; *C(=O)COCN(*)CCS(C)(=O)=O]

In still other embodiments, Spacer Units are represented by the formulae:

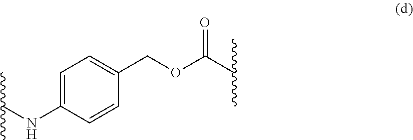 [SMILES: *Nc1ccc(COC(*)=O)cc1]

The Subscript “p”

In one aspect of the invention, the subscript p represents the number of Drug Linker moieties on a Ligand Unit of an individual Camptothecin Conjugate and is an integer preferably ranging from 1 to 16, 1 to 12, 1 to 10, or 1 to 8. Individual Camptothecin Conjugates can be also be referred to as a Camptothecin Conjugate compound. In any of the embodiments herein, there can be 1, 2, 3, 4, 5, 6, 7, 8, 9, 10, 11, 12, 13, 14, 15, or 16 Drug Linker moieties conjugated to a Ligand Unit of an individual Camptothecin Conjugate. In another aspect of the invention, one group of embodiments describes a population of individual Camptothecin Conjugates substantially identical except for the number of Camptothecin Linker Compound moieties bound to each Ligand Unit (i.e., a Camptothecin Conjugate composition) so that p represents the average number of Camptothecin Linker Compound moieties bound to the Ligand Units of the Camptothecin Conjugate composition. In that group of embodiments, p is a number ranging from 1 to about 16, 1 to about 12, 1 to about 10, or 1 to about 8, from 2 to about 16, 2 to about 12, 2 to about 10, or 2 to about 8. In some aspects, p is about 2. In some aspects, p is about 4. In some aspects, p is about 8. In some aspects, p is about 16. In some aspects, p is 2. In some aspects, p is 4. In some aspects, p is 8. In some aspects, p is 16. In some aspects, the p value refers to the average drug loading as well as the drug loading of the predominate ADC in the composition.

In some aspects, conjugation will be via the interchain disulfides and there will from 1 to about 8 Camptothecin Linker Compound (Q-D) molecules conjugated to a ligand molecule. In some aspects, conjugation will be via an introduced cysteine residue as well as interchain disulfides and there will be from 1 to 10 or 1 to 12 or 1 to 14 or 1 to 16 Camptothecin Linker Compound molecules conjugated to a ligand molecule. In some aspects, conjugation will be via an introduced cysteine residue and there will be 2 or 4 Camptothecin Linker Compound molecules conjugated to a ligand molecule.

### Partially Released Free Drug

In some embodiments are compounds where the RL unit in the conjugate has been cleaved, leaving the drug moiety with one amino acid residue bound thereto. In some embodiments, the partially release Free Drug (Drug-Amino Acid Conjugate) is a compound of Formula (IV):

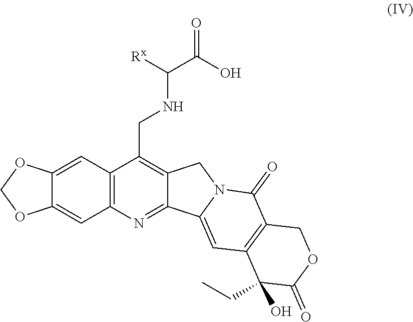

or a stereoisomer or mixture of stereoisomers thereof, or a pharmaceutically acceptable salt thereof, wherein R^{x} is an amino acid sidechain as described herein. In some embodiments, R^{x} is H, methyl, isopropyl, benzyl, or —(CH₂)₄—NH₂. In some embodiments, R^{x} is H or methyl. In some embodiments, R^{x} is H. In some embodiments, R^{x} is methyl.

In some embodiments, the compound of Formula (IV) is a biologically active compound. In some embodiments, such compounds are useful in a method of inhibiting topoisomerase, killing tumor cells, inhibiting growth of tumor cells, cancer cells, or of a tumor, inhibiting replication of tumor cells or cancer cells, lessening of overall tumor burden or decreasing the number of cancerous cells, or ameliorating one or more symptoms associated with a cancer or autoimmune disease. Such methods comprise, for example, contacting a cancer cell with a compound of Formula (IV).

### Camptothecin Conjugate Mixtures and Compositions

The present invention provides Camptothecin Conjugate mixtures and pharmaceutical compositions comprising any of the Camptothecin Conjugates described herein. The mixtures and pharmaceutical compositions comprise a plurality of conjugates. In some aspects, each of the conjugates in the mixture or composition is identical or substantially identical, however, the distribution of drug-linkers on the ligands in the mixture or compositions may vary as well as the drug loading. For example, the conjugation technology used to conjugate drug-linkers to antibodies as the targeting ligand can result in a composition or mixture that is heterogeneous with respect to the distribution of Camptothecin Linker Compounds on the antibody (Ligand Unit) within the mixture and/or composition. In some aspects, the loading of Camptothecin Linker Compounds on each of the antibody molecules in a mixture or composition of such molecules is an integer that ranges from 1 to 14.

In those aspects, when referring to the composition as a whole the loading of drug-linkers is a number ranging from 1 to about 14. Within the composition or mixture, there may also be a small percentage of unconjugated antibodies. The average number of drug-linkers per Ligand Unit in the mixture or composition (i.e., average drug-load) is an important attribute as it determines the maximum amount of drug that can be delivered to the target cell. The average drug load can be 1, 2 or about 2, 3 or about 3, 4 or about 4, 5 or about 5, 6 or about 6, 7 or about 7, 8 or about 8, 9 or about 9, 10 or about 10, 11 or about 11, 12 or about 12, 13 or about 13, 14 or about 14, 15 or about 15, 16 or about 16.

In some aspects, the mixtures and pharmaceutical compositions comprise a plurality (i.e., population) of conjugates, however, the conjugates are identical or substantially identical and are substantially homogenous with respect to the distribution of drug-linkers on the ligand molecules within the mixture and/or composition and with respect to loading of drug-linkers on the ligand molecules within the mixture and/or composition. In some such aspects, the loading of drug-linkers on an antibody Ligand Unit is 2 or 4. Within the composition or mixture, there may also be a small percentage of unconjugated antibodies. The average drug load in such embodiments is about 2 or about 4. Typically, such compositions and mixtures result from the use of site-specific conjugation techniques and conjugation is due to an introduced cysteine residue.

The average number of Camptothecins or Camptothecin-Linker Compounds per Ligand Unit in a preparation from a conjugation reaction may be characterized by conventional means such as mass spectrometry, ELISA assay, HPLC (e.g., HIC). The quantitative distribution of Camptothecin Conjugates in terms of p may also be determined. In some instances, separation, purification, and characterization of homogeneous Camptothecin Conjugates may be achieved by means such as reverse phase HPLC or electrophoresis.

In some aspects, the compositions are pharmaceutical compositions comprising the Camptothecin Conjugates described herein and a pharmaceutically acceptable carrier. In some aspects, the pharmaceutical composition is in liquid form. In some aspects, the pharmaceutical composition is a solid. In some aspects, the pharmaceutical composition is a lyophilized powder.

The compositions, including pharmaceutical compositions, can be provided in purified form. As used herein, “purified” means that when isolated, the isolate contains at least 95%, and in another aspect at least 98%, of Conjugate by weight of the isolate.

### Methods of Use

### Treatment of Cancer

The Camptothecin Conjugates described herein are useful for inhibiting the multiplication of a tumor cell or cancer cell, causing apoptosis in a tumor or cancer cell, or for treating cancer in a patient. Accordingly, provide herein are methods of treating cancer in a subject in need thereof, the method includes administering to the subject one or more Captothecin Conjugates described herein.

The Camptothecin Conjugates can be used accordingly in a variety of settings for the treatment of cancers. The Camptothecin Conjugates can be used to deliver a drug to a tumor cell or cancer cell. Without being bound by theory, in one embodiment, the Ligand Unit of a Camptothecin Conjugate binds to or associates with a cancer-cell or a tumor-cell-associated antigen, and the Camptothecin Conjugate can be taken up (internalized) inside the tumor cell or cancer cell through receptor-mediated endocytosis or other internalization mechanism. The antigen can be attached to a tumor cell or cancer cell or can be an extracellular matrix protein associated with the tumor cell or cancer cell. Once inside the cell, the drug is released via peptide cleavage within the cell. In an alternative embodiment, the free drug is released from the Camptothecin Conjugate outside the tumor cell or cancer cell, and the free drug subsequently penetrates the cell.

In one embodiment, the Ligand Unit binds to the tumor cell or cancer cell.

In another embodiment, the Ligand Unit binds to a tumor cell or cancer cell antigen which is on the surface of the tumor cell or cancer cell.

In another embodiment, the Ligand Unit binds to a tumor cell or cancer cell antigen which is an extracellular matrix protein associated with the tumor cell or cancer cell.

The specificity of the Ligand Unit for a particular tumor cell or cancer cell can be important for determining the tumors or cancers that are most effectively treated. For example, Camptothecin Conjugates that target a cancer cell antigen present in hematopoietic cancers can be useful treating hematologic malignancies (e.g., anti-CD30, anti-CD70, anti-CD19, anti-CD33 binding Ligand Unit (e.g., antibody) can be useful for treating hematologic malignancies). Camptothecin Conjugates that target a cancer cell antigen present on solid tumors can be useful treating such solid tumors.

Cancers that can be treated with a Camptothecin Conjugate include, but are not limited to, hematopoietic cancers such as, for example, lymphomas (Hodgkin Lymphoma and Non-Hodgkin Lymphomas) and leukemias and solid tumors. Examples of hematopoietic cancers include, follicular lymphoma, anaplastic large cell lymphoma, mantle cell lymphoma, acute myeloblastic leukemia, chronic myelocytic leukemia, chronic lymphocytic leukemia, diffuse large B cell lymphoma, and multiple myeloma. Examples of solid tumors include fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma.

In preferred embodiments, the cancers treated are any one of the above-listed lymphomas and leukemias.

### Multi-Modality Therapy for Cancer

Cancers, including, but not limited to, a tumor, metastasis, or other disease or disorder characterized by uncontrolled cell growth, can be treated or inhibited by administration of a Camptothecin Conjugate.

In other embodiments, methods for treating cancer are provided, including administering to a patient in need thereof an effective amount of a Camptothecin Conjugate and a chemotherapeutic agent. In one embodiment, the chemotherapeutic agent is that with which treatment of the cancer has not been found to be refractory. In another embodiment, the chemotherapeutic agent is that with which the treatment of cancer has been found to be refractory. The Camptothecin Conjugates can be administered to a patient that has also undergone surgery as treatment for the cancer.

In some embodiments, the patient also receives an additional treatment, such as radiation therapy. In a specific embodiment, the Camptothecin Conjugate is administered concurrently with the chemotherapeutic agent or with radiation therapy. In another specific embodiment, the chemotherapeutic agent or radiation therapy is administered prior or subsequent to administration of a Camptothecin Conjugate.

A chemotherapeutic agent can be administered over a series of sessions. Any one or a combination of the chemotherapeutic agents, such a standard of care chemotherapeutic agent(s), can be administered.

Additionally, methods of treatment of cancer with a Camptothecin Conjugate are provided as an alternative to chemotherapy or radiation therapy where the chemotherapy or the radiation therapy has proven or can prove too toxic, e.g., results in unacceptable or unbearable side effects, for the subject being treated. The patient being treated can, optionally, be treated with another cancer treatment such as surgery, radiation therapy or chemotherapy, depending on which treatment is found to be acceptable or bearable.

### Treatment of Autoimmune Diseases

The Camptothecin Conjugates are useful for killing or inhibiting the unwanted replication of cells that produces an autoimmune disease or for treating an autoimmune disease.

The Camptothecin Conjugates can be used accordingly in a variety of settings for the treatment of an autoimmune disease in a patient. The Camptothecin Conjugates can be used to deliver a drug to a target cell. Without being bound by theory, in one embodiment, the Camptothecin Conjugate associates with an antigen on the surface of a pro-inflammatory or inappropriately-stimulated immune cell, and the Camptothecin Conjugate is then taken up inside the targeted cell through receptor-mediated endocytosis. Once inside the cell, the Linker unit is cleaved, resulting in release of the Camptothecin. The released Camptothecin is then free to migrate in the cytosol and induce cytotoxic or cytostatic activities. In an alternative embodiment, the Drug is cleaved from the Camptothecin Conjugate outside the target cell, and the Camptothecin subsequently penetrates the cell.

In one embodiment, the Ligand Unit binds to an autoimmune antigen. In one aspect, the antigen is on the surface of a cell involved in an autoimmune condition.

In one embodiment, the Ligand Unit binds to activated lymphocytes that are associated with the autoimmune disease state.

In a further embodiment, the Camptothecin Conjugate kills or inhibits the multiplication of cells that produce an autoimmune antibody associated with a particular autoimmune disease.

Particular types of autoimmune diseases that can be treated with the Camptothecin Conjugates include, but are not limited to, Th2 lymphocyte related disorders (e.g., atopic dermatitis, atopic asthma, rhinoconjunctivitis, allergic rhinitis, Omenn's syndrome, systemic sclerosis, and graft versus host disease); Th1 lymphocyte-related disorders (e.g., rheumatoid arthritis, multiple sclerosis, psoriasis, Sjorgren's syndrome, Hashimoto's thyroiditis, Grave's disease, primary biliary cirrhosis, Wegener's granulomatosis, and tuberculosis); and activated B lymphocyte-related disorders (e.g., systemic lupus erythematosus, Goodpasture's syndrome, rheumatoid arthritis, and type I diabetes).

### Multi-Drug Therapy of Autoimmune Diseases

Methods for treating an autoimmune disease are also disclosed including administering to a patient in need thereof an effective amount of a Camptothecin Conjugate and another therapeutic agent known for the treatment of an autoimmune disease.

### Compositions and Methods of Administration

The present invention provides pharmaceutical compositions comprising the Camptothecin Conjugates described herein and a pharmaceutically acceptable carrier. The Camptothecin Conjugates can be in any form that allows the compound to be administered to a patient for treatment of a disorder associated with expression of the antigen to which the Ligand Unit binds. For example, the conjugates can be in the form of a liquid or solid. The preferred route of administration is parenteral. Parenteral administration includes subcutaneous injections, intravenous, intramuscular, intrasternal injection or infusion techniques. In one aspect, the compositions are administered parenterally. In one aspect, the conjugates are administered intravenously. Administration can be by any convenient route, for example by infusion or bolus injection

Pharmaceutical compositions can be formulated to allow a compound to be bioavailable upon administration of the composition to a patient. Compositions can take the form of one or more dosage units.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of animal (e.g., human), the particular form of the compound, the manner of administration, and the composition employed.

The composition can be, for example, in the form of a liquid. The liquid can be useful for delivery by injection. In a composition for administration by injection, one or more of a surfactant, preservative, wetting agent, dispersing agent, suspending agent, buffer, stabilizer and isotonic agent can also be included.

The liquid compositions, whether they are solutions, suspensions or other like form, can also include one or more of the following: sterile diluents such as water for injection, saline solution, preferably physiological saline, Ringer's solution, isotonic sodium chloride, fixed oils such as synthetic mono or digylcerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as amino acids, acetates, citrates or phosphates; detergents, such as nonionic surfactants, polyols; and agents for the adjustment of tonicity such as sodium chloride or dextrose. A parenteral composition can be enclosed in ampoule, a disposable syringe or a multiple-dose vial made of glass, plastic or other material. Physiological saline is an exemplary adjuvant. An injectable composition is preferably sterile.

The amount of the conjugate that is effective in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances.

The compositions comprise an effective amount of a compound such that a suitable dosage will be obtained. Typically, this amount is at least about 0.01% of a compound by weight of the composition.

For intravenous administration, the composition can comprise from about 0.01 to about 100 mg of a Camptothecin Conjugate per kg of the animal's body weight. In one aspect, the composition can include from about 1 to about 100 mg of a Camptothecin Conjugate per kg of the animal's body weight. In another aspect, the amount administered will be in the range from about 0.1 to about 25 mg/kg of body weight of a compound. Depending on the drug used, the dosage can be even lower, for example, 1.0 μg/kg to 5.0 mg/kg, 4.0 mg/kg, 3.0 mg/kg, 2.0 mg/kg or 1.0 mg/kg, or 1.0 μg/kg to 500.0 μg/kg of the subject's body weight.

Generally, the dosage of a conjugate administered to a patient is typically about 0.01 mg/kg to about 100 mg/kg of the subject's body weight or from 1.0 μg/kg to 5.0 mg/kg of the subject's body weight. In some embodiments, the dosage administered to a patient is between about 0.01 mg/kg to about 15 mg/kg of the subject's body weight. In some embodiments, the dosage administered to a patient is between about 0.1 mg/kg and about 15 mg/kg of the subject's body weight. In some embodiments, the dosage administered to a patient is between about 0.1 mg/kg and about 20 mg/kg of the subject's body weight. In some embodiments, the dosage administered is between about 0.1 mg/kg to about 5 mg/kg or about 0.1 mg/kg to about 10 mg/kg of the subject's body weight. In some embodiments, the dosage administered is between about 1 mg/kg to about 15 mg/kg of the subject's body weight. In some embodiments, the dosage administered is between about 1 mg/kg to about 10 mg/kg of the subject's body weight. In some embodiments, the dosage administered is between about 0.1 to 4 mg/kg, even more preferably 0.1 to 3.2 mg/kg, or even more preferably 0.1 to 2.7 mg/kg of the subject's body weight over a treatment cycle.

The term “carrier” refers to a diluent, adjuvant or excipient, with which a compound is administered. Such pharmaceutical carriers can be liquids, such as water and oils, including those of petroleum, animal, vegetable or synthetic origin, such as peanut oil, soybean oil, mineral oil, sesame oil. The carriers can be saline, gum acacia, gelatin, starch paste, talc, keratin, colloidal silica, urea. In addition, auxiliary, stabilizing, thickening, lubricating and coloring agents can be used. In one embodiment, when administered to a patient, the compound or compositions and pharmaceutically acceptable carriers are sterile.

Water is an exemplary carrier when the compounds are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol. The present compositions, if desired, can also contain minor amounts of wetting or emulsifying agents, or pH buffering agents.

In an embodiment, the conjugates are formulated in accordance with routine procedures as a pharmaceutical composition adapted for intravenous administration to animals, particularly human beings. Typically, the carriers or vehicles for intravenous administration are sterile isotonic aqueous buffer solutions. Where necessary, the compositions can also include a solubilizing agent. Compositions for intravenous administration can optionally comprise a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachets indicating the quantity of active agent. Where a conjugate is to be administered by infusion, it can be dispensed, for example, with an infusion bottle containing sterile pharmaceutical grade water or saline. Where the conjugate is administered by injection, an ampoule of sterile water for injection or saline can be provided so that the ingredients can be mixed prior to administration.

The pharmaceutical compositions are generally formulated as sterile, substantially isotonic and in full compliance with all Good Manufacturing Practice (GMP) regulations of the U.S. Food and Drug Administration.

### Methods of Preparing Camptothecin Conjugates

The Camptothecin Conjugates described herein can be prepared in either a serial construction of antibodies, linkers, and drug units, or in a convergent fashion by assembling portions followed by a completed assembly step.

In one group of embodiments, Camptothecin-Linker Compounds as provided herein, are combined with a suitable Ligand Unit to facilitate covalent attachment of the Camptothecin-Linker Compounds to the Ligand Unit. In some embodiments, the Ligand Unit is an antibody that has at least 2, at least 4, at least 6 or 8 thiols available for attachment of the Linker Compounds as a result of reducing interchain disulfide linkages. In some embodiments, the Camptothecin-Linker Compounds are attached to the Ligand Unit through an introduced cysteine moiety on the antibody.

### Kits for Therapeutic Use

In some aspects, kits for use in cancer treatment and the treatment of autoimmune diseases are provided. Such kits can include a pharmaceutical composition that comprises a Camptothecin Conjugate described herein.

In some embodiments, the kit can include instructions for use in any of the therapeutic methods described herein. The included instructions can provide a description of administration of the pharmaceutical compositions to a subject to achieve the intended activity, e.g., treatment of a disease or condition such as cancer, in a subject. In some embodiments, the instructions relating to the use of the pharmaceutical compositions described herein can include information as to dosage, dosing schedule, and route of administration for the intended treatment. The containers can be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

In some embodiments, the kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. In some embodiments, a kit can have a sterile access port (for example, the container can be an intravenous solution bag or a vial having a stopper pierceable by a hypodermic injection needle).

In some embodiments, the kits provided herein include an additional therapeutic agent useful in treating a cancer of autoimmune disease as described herein.

### EXEMPLARY EMBODIMENTS

Embodiment 1: A Camptothecin Conjugate having a formula:

L-(Q-D)ₚ

or a pharmaceutically acceptable salt thereof, wherein

### L is a Ligand Unit;

Q is a Linker Unit having a formula selected from the group consisting of:

—Z-A-S*—RL-; —Z-A-L^{P}(S*)—RL-; —Z-A-S*—RL-Y—; and —Z-A-L^{P}(S*)—RL-Y—;
wherein Z is a Stretcher Unit, A is a bond or a Connector Unit; L^{P} is a Parallel Connector Unit; S* is a Partitioning Agent; RL is a peptide comprising from 2 to 8 amino acids; and Y is a Spacer Unit;
   D is a Drug Unit selected from the group consisting of:

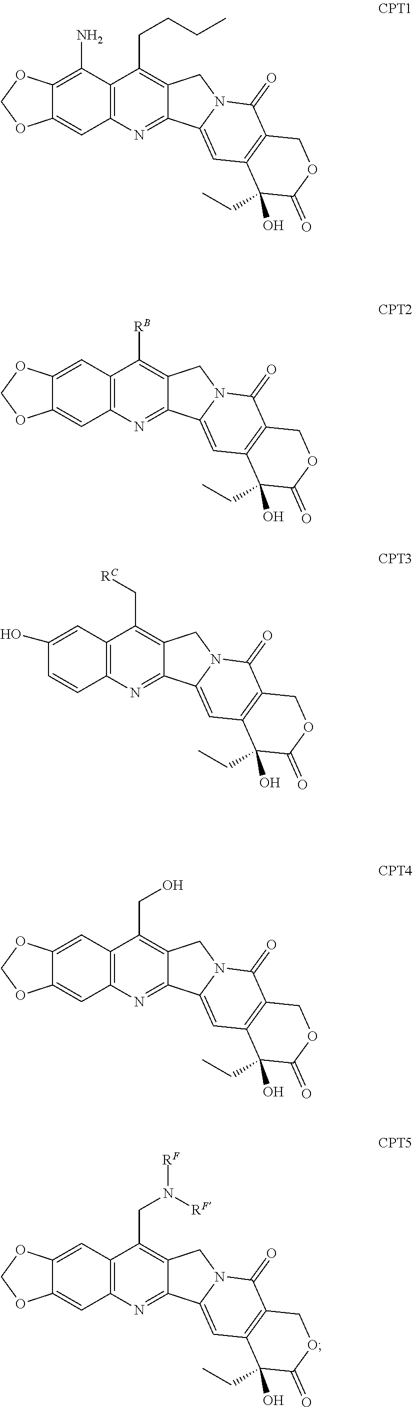

wherein
R^{B} is a member selected from the group consisting of H, C₁-C₈ alkyl, C₁-C₈ haloalkyl, C₃-C₈ cycloalkyl, C₃-C₈cycloalkylC₁-C₄ alkyl, phenyl and phenylC₁-C₄ alkyl;
R^{C} is a member selected from the group consisting of C₁-C₆ alkyl and C₃-C₆ cycloalkyl;
each R^{F} and R^{F′} is a member independently selected from the group consisting of H, C₁-C₈ alkyl, C₁-C₈ hydroxyalkyl, C₁-C₈ aminoalkyl, C₁-C₄ alkylaminoC₁-C₈ alkyl, (C₁-C₄ hydroxyalkyl)(C₁-C₄ alkyl)aminoC₁-C₈ alkyl, di(C₁-C₄ alkyl)aminoC₁-C₈ alkyl, C₁-C₄ hydroxyalkylC₁-C₈ aminoalkyl, C₁-C₈ alkylC(O)—, C₁-C₈ hydroxyalkylC(O)—, C₁-C₈ aminoalkylC(O)—, C₃-C₁₀ cycloalkyl, C₃-C₁₀cycloalkylC₁-C₄ alkyl, C₃-C₁₀ heterocycloalkyl, C₃-C₁₀ heterocycloalkylC₁-C₄ alkyl, phenyl, phenylC₁-C₄ alkyl, diphenylC₁-C₄ alkyl, heteroaryl and heteroarylC₁-C₄ alkyl; or R^{F} and R^{F′} are combined with the nitrogen atom to which each is attached to form a 5-, 6- or 7-membered ring having 0 to 3 substituents selected from halogen, C₁-C₄ alkyl, OH, OC₁-C₄ alkyl, NH₂, NHC₁-C₄ alkyl and N(C₁-C₄ alkyl)₂;
and wherein cycloalkyl, heterocycloalkyl, phenyl and heteroaryl portions of R^{B}, R^{C}, R^{F} and R^{F′} are substituted with from 0 to 3 substituents selected from halogen, C₁-C₄ alkyl, OH, OC₁-C₄ alkyl, NH₂, NHC₁-C₄ alkyl and N(C₁-C₄ alkyl)₂;
   the subscript p is an integer of from 1 to 16; and
wherein Q is attached through any of the hydroxyl and amine groups present on CPT1, CPT2, CPT3, CPT4 or CPT5.

Embodiment 2: A Camptothecin Conjugate of Embodiment 1, wherein D has formula CPT5. Embodiment 3: A Camptothecin Conjugate of Embodiment 1, wherein D has formula CPT2. Embodiment 4: A Camptothecin Conjugate of Embodiment 1, wherein D has formula CPT3. Embodiment 5: A Camptothecin Conjugate of Embodiment 1, wherein D has formula CPT4. Embodiment 6: A Camptothecin Conjugate of Embodiment 1, wherein D has formula CPT1. Embodiment 7: A Camptothecin Conjugate of Embodiment 1, wherein L is an antibody. Embodiment 8: A Camptothecin Conjugate of Embodiment 1 or 3, wherein R^{B} is a member selected from the group consisting of H, C₁-C₈ alkyl, and C₁-C₈ haloalkyl. Embodiment 9: A Camptothecin Conjugate of Embodiment 1 or 3, wherein R^{B} is a member selected from the group consisting of C₃-C₈ cycloalkyl, C₃-C₈cycloalkylC₁-C₄ alkyl, phenyl and phenylC₁-C₄ alkyl, and wherein the cycloalkyl and phenyl portions of R^{B} are substituted with from 0 to 3 substituents selected from halogen, C₁-C₄ alkyl, OH, OC₁-C₄ alkyl, NH₂, NHC₁-C₄ alkyl and N(C₁-C₄ alkyl)₂. Embodiment 10: A Camptothecin Conjugate of Embodiment 1 or 4, wherein R^{C} is C₁-C₆ alkyl. Embodiment 11: A Camptothecin Conjugate of Embodiment 1 or 4, wherein R^{C} is C₃-C₆ cycloalkyl. Embodiment 12: A Camptothecin Conjugate of Embodiment 1 or 2, wherein both R^{F} and R^{F′} are H. Embodiment 13: A Camptothecin Conjugate of Embodiment 1 or 2, wherein at least one of R^{F} and R^{F′} is a member independently selected from the group consisting of C₁-C₈ alkyl, C₁-C₈ hydroxyalkyl, C₁-C₈ aminoalkyl, C₁-C₄ alkylaminoC₁-C₈ alkyl, (C₁-C₄ hydroxyalkyl)(C₁-C₄ alkyl)aminoC₁-C₈ alkyl, di(C₁-C₄ alkyl)aminoC₁-C₈ alkyl, C₁-C₄ hydroxyalkylC₁-C₈ aminoalkyl, C₁-C₈ alkylC(O)—, C₁-C₈ hydroxyalkylC(O)—, and C₁-C₈ aminoalkylC(O)—. Embodiment 14: A Camptothecin Conjugate of Embodiment 1 or 2, wherein each R^{F} and R^{F′} is a member independently selected from the group consisting of C₁-C₈ alkyl, C₁-C₈ hydroxyalkyl, C₁-C₈ aminoalkyl, C₁-C₄ alkylaminoC₁-C₈ alkyl, (C₁-C₄ hydroxyalkyl)(C₁-C₄ alkyl)aminoC₁-C₈ alkyl, di(C₁-C₄ alkyl)aminoC₁-C₈ alkyl, C₁-C₄ hydroxyalkylC₁-C₈ aminoalkyl, C₁-C₈ alkylC(O)—, C₁-C₈ hydroxyalkylC(O)—, and C₁-C₈ aminoalkylC(O)—. Embodiment 15: A Camptothecin Conjugate of Embodiment 1 or 2, wherein at least one of R^{F} and R^{F′} is a member independently selected from the group consisting of C₃-C₁₀ cycloalkyl, C₃-C₁₀cycloalkylC₁-C₄ alkyl, C₃-C₁₀ heterocycloalkyl, C₃-C₁₀ heterocycloalkylC₁-C₄ alkyl, phenyl, phenylC₁-C₄ alkyl, diphenylC₁-C₄ alkyl, heteroaryl and heteroarylC₁-C₄ alkyl, and wherein cycloalkyl, heterocycloalkyl, phenyl and heteroaryl portions of R^{F} and R^{F′} are substituted with from 0 to 3 substituents selected from halogen, C₁-C₄ alkyl, OH, OC₁-C₄ alkyl, NH₂, NHC₁-C₄ alkyl and N(C₁-C₄ alkyl)₂. Embodiment 16: A Camptothecin Conjugate of Embodiment 1 or 2, wherein each R^{F} and R^{F′} is a member independently selected from the group consisting of C₃-C₁₀ cycloalkyl, C₃-C₁₀cycloalkylC₁-C₄ alkyl, C₃-C₁₀ heterocycloalkyl, C₃-C₁₀ heterocycloalkylC₁-C₄ alkyl, phenyl, phenylC₁-C₄ alkyl, diphenylC₁-C₄ alkyl, heteroaryl and heteroarylC₁-C₄ alkyl, and wherein cycloalkyl, heterocycloalkyl, phenyl and heteroaryl portions of R^{F} and R^{F′} are substituted with from 0 to 3 substituents selected from halogen, C₁-C₄ alkyl, OH, OC₁-C₄ alkyl, NH₂, NHC₁-C₄ alkyl and N(C₁-C₄ alkyl)₂. Embodiment 17: A Camptothecin Conjugate of Embodiment 1 or 2, wherein R^{F} and R^{F′} are combined with the nitrogen atom to which each is attached to form a 5-, 6- or 7-membered ring having 0 to 3 substituents selected from halogen, C₁-C₄ alkyl, OH, OC₁-C₄ alkyl, NH₂, NHC₁-C₄ alkyl and N(C₁-C₄ alkyl)₂. Embodiment 18: A Camptothecin Conjugate of Embodiment 1, wherein Q is a Linker Unit having a formula selected from the group consisting of:

—Z-A-S*—RL-; and —Z-A-S*—RL-Y—;

wherein Z is a Stretcher Unit, A is a bond or a Connector Unit; S* is a Partitioning Agent; and Y is a Spacer Unit. Embodiment 19: A Camptothecin Conjugate of Embodiment 18, wherein Z-A- comprises a maleimido-alkanoic acid component or an mDPR component. Embodiment 20: A Camptothecin Conjugate of Embodiment 18, wherein RL is a dipeptide. Embodiment 21: A Camptothecin Conjugate of Embodiment 1, wherein RL is a tripeptide. Embodiment 22: A Camptothecin Conjugate of Embodiment 18, wherein RL is a tetrapeptide. Embodiment 23: A Camptothecin Conjugate of Embodiment 18, wherein RL is a pentapeptide. Embodiment 24: A Camptothecin Conjugate of any one of Embodiments 18 to 23, wherein RL comprises amino acids selected from the group consisting of β-alanine, N-methylglycine, glycine, lysine, valine and phenylalanine. Embodiment 25: A Camptothecin Conjugate of Embodiment 1, wherein Y is present and comprises:

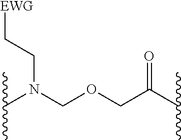

wherein EWG is an electron-withdrawing group. Embodiment 26: A Camptothecin Conjugate of Embodiment 1, wherein Y is present and comprises:

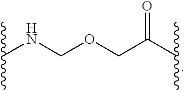 [SMILES: *NCOCC(*)=O]

Embodiment 27: A Camptothecin Conjugate of Embodiment 1, wherein Y is present and comprises:

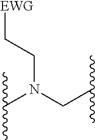

wherein EWG is an electron-withdrawing group. Embodiment 28: A Camptothecin Conjugate of Embodiment 25 or 27, wherein EWG is a member selected from the group consisting of —CN, —NO₂, —CX₃, —X′, C(═O)OR′, —C(═O)N(R′)₂, —C(═O)R′, —C(═O)X, —S(═O)₂R′, —S(═O)₂OR′, —S(═O)₂NHR′, —S(═O)₂N(R′)₂, —P(═O)(OR′)₂, —P(═O)(CH₃)NHR′, —NO, —N(R′)₃⁺, wherein X is —F, —Br, —Cl, or —I, and R′ is independently selected from the group consisting of hydrogen and C₁₋₆ alkyl. Embodiment 29: A Camptothecin Conjugate of any one of Embodiments 1 to 27, wherein RL is a peptide selected from the group consisting of gly-gly, gly-gly-gly, gly-gly-gly-gly, val-gly-gly, val-cit-gly, val-gln-gly, val-glu-gly, phe-lys-gly, leu-lys-gly, gly-val-lys-gly, val-lys-gly-gly, val-lys-gly, val-lys-ala, val-lys-leu, leu-leu-gly, gly-gly-phe-gly, gly-gly-phe-gly-gly, val-gly, and val-lys-β-ala. Embodiment 30: A Camptothecin Conjugate of any one of Embodiments 1 to 27, wherein RL is a peptide selected from the group consisting of gly-gly, gly-gly-gly, gly-gly-gly-gly, val-gly-gly, val-cit-gly, val-gln-gly, val-glu-gly, phe-lys-gly, leu-lys-gly, gly-val-lys-gly, val-lys-gly-gly, val-lys-gly, val-lys-ala, val-lys-leu, leu-leu-gly, gly-gly-phe-gly, gly-gly-phe-gly-gly, val-gly, and val-lys-β-ala; Y is a PEG Unit; and Z—X is a maleimido-alkanoic acid component, or a mDPR component. Embodiment 31: A Camptothecin Conjugate of any one of Embodiments 1 to 27, wherein S* is a PEG Unit; and Z-A- is a maleimidopropionyl component or a mDPR component. Embodiment 32: A Camptothecin Conjugate of Embodiment 31, wherein Z-A- is a maleimidopropionyl component. Embodiment 33: A Camptothecin Conjugate of Embodiment 31, wherein Q has the formula:

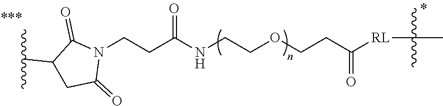

wherein n is an integer from 2 to 20; RL is a di-, tri-, tetra- or pentapeptide; the wavy line marked with a single * indicates the site of attachment to D, or to a Spacer Unit (Y); and the wavy line marked with *** indicates the point of attachment to a sulfur atom of L. Embodiment 34: A Camptothecin Conjugate of Embodiment 33, wherein n is an integer of from 4 to 10. Embodiment 35: A Camptothecin Conjugate of any one of Embodiments 1 to 34, wherein L is an antibody that specifically binds to an antigen selected from the group consisting of CD19, CD30, CD33, CD70 and LIV-1. Embodiment 36: A Camptothecin Conjugate of Embodiment 1, wherein the Conjugate is of the formula:

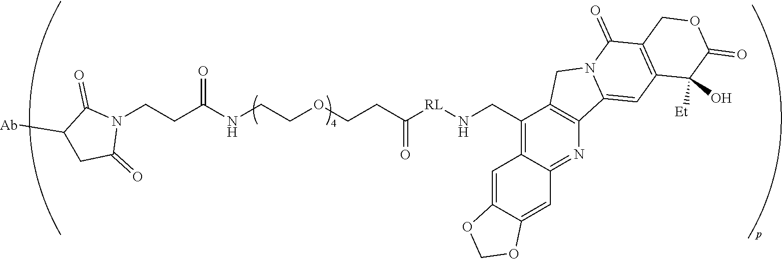

wherein Ab is an antibody specific for an antigen selected from the group consisting of CD19, CD30, CD33, CD70 and LIV-1, RL is a peptide selected from the group consisting of gly-gly-gly-gly, val-lys-β-ala, val-gln-gly, val-lys-ala, phe-lys-gly, val-lys-gly-gly, gly-gly, val-lys-gly, val-gly-gly, leu-leu-gly, leu-lys-gly, val-glu-gly, gly-gly-gly, val-asp-gly, val-lys, val-gly and gly-val-lys-gly; and p is an integer of from 1 to 16. Embodiment 37: A Camptothecin Conjugate of Embodiment 36, wherein RL is selected from the group consisting of val-lys-β-ala, val-gln-gly, val-lys-ala, phe-lys-gly, val-lys-gly, val-gly-gly, leu-leu-gly, leu-lys-gly, val-glu-gly, gly-gly-gly and val-asp-gly. Embodiment 38: A Camptothecin Conjugate of Embodiment 36, wherein RL is selected from the group consisting of val-lys-β-ala, val-gln-gly, val-lys-ala, phe-lys-gly, val-lys-gly, val-gly-gly, leu-lys-gly, val-glu-gly and val-asp-gly. Embodiment 39: A Camptothecin Conjugate of Embodiment 36, wherein RL is val-lys-gly.

Embodiment 40: A Camptothecin-Linker Compound having a formula selected from the group consisting of:

Z′-A S*—RL-D;

Z′-A-L^{P}(S*)—RL-D;

Z′-A-S*—RL-Y-D; and

Z′-A-L^{P}(S*)—RL-Y-D;

wherein Z′ is a Stretcher Unit; A is a bond or a Connecter Unit; L^{P} is a Parallel Connector Unit; S* is a Partitioning Agent; RL is a peptide comprising from 2 to 8 amino acids; Y is a Spacer Unit; and D is a Drug Unit selected from the group consisting of

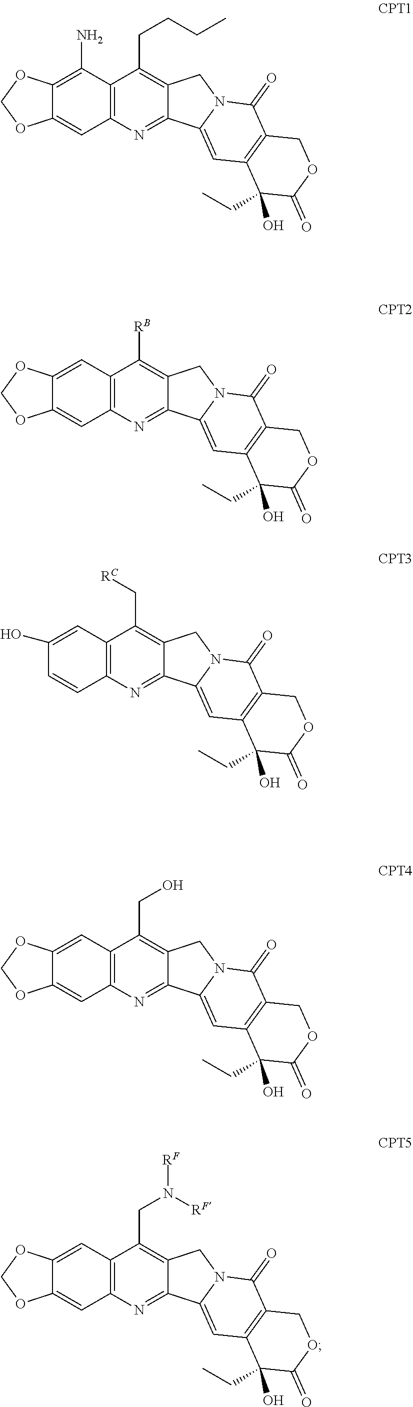

wherein
R^{B} is a member Selected from the group consisting of H, C₁-C₈ alkyl, C₁-C₈ haloalkyl, C₃-C₈ cycloalkyl, C₃-C₈cycloalkylC₁-C₄ alkyl, phenyl and phenylC₁-C₄ alkyl;
R^{C} is a member selected from the group consisting of C₁-C₆ alkyl and C₃-C₆ cycloalkyl; each R^{F} and R^{F′} is a member independently selected from the group consisting of H, C₁-C₈ alkyl, C₁-C₈ hydroxyalkyl, C₁-C₈ aminoalkyl, C₁-C₄ alkylaminoC₁-C₈ alkyl, (C₁-C₄ hydroxyalkyl)(C₁-C₄ alkyl)aminoC₁-C₈ alkyl, di(C₁-C₄ alkyl)aminoC₁-C₈ alkyl, C₁-C₄ hydroxyalkylC₁-C₈ aminoalkyl, C₁-C₈ alkylC(O)—, C₁-C₈ hydroxyalkylC(O)—, C₁-C₈ aminoalkylC(O)—, C₃-C₁₀ cycloalkyl, C₃-C₁₀cycloalkylC₁-C₄ alkyl, C₃-C₁₀ heterocycloalkyl, C₃-C₁₀ heterocycloalkylC₁-C₄ alkyl, phenyl, phenylC₁-C₄ alkyl, diphenylC₁-C₄ alkyl, heteroaryl and heteroarylC₁-C₄ alkyl; or R^{F} and R^{F′} are combined with the nitrogen atom to which each is attached to form a 5-, 6- or 7-membered ring having 0 to 3 substituents selected from halogen, C₁-C₄ alkyl, OH, OC₁-C₄ alkyl, NH₂, NHC₁-C₄ alkyl and N(C₁-C₄ alkyl)₂;
and wherein cycloalkyl, heterocycloalkyl, phenyl and heteroaryl portions of R^{B}, R_{C}, R^{F} and R^{F′} are substituted with from 0 to 3 substituents selected from halogen, C₁-C₄ alkyl, OH, OC₁-C₄ alkyl, NH₂, NHC₁-C₄ alkyl and N(C₁-C₄ alkyl)₂;
the subscript p is an integer of from 1 to 16; and
wherein Q is attached through any of the hydroxyl and amine groups present on CPT1, CPT2, CPT3, CPT4 or CPT5.

Embodiment 41: A Camptothecin-Linker Compound of Embodiment 40, having formula (i) or (iii). Embodiment 42: A Camptothecin-Linker Compound of Embodiment 40, having formula (ii) or (iv). Embodiment 43: A Camptothecin-Linker Compound of Embodiment 40, having formula (i). Embodiment 44: A Camptothecin-Linker Compound of Embodiment 40, having formula (iii). Embodiment 45: A Camptothecin-Linker Compound of any one of Embodiment 40 to 44, wherein D is CPT5. Embodiment 46: A Camptothecin-Linker Compound of any one of Embodiments 40 to 44, wherein R^{B} is a member selected from the group consisting of H, C₁-C₈ alkyl, and C₁-C₈ haloalkyl. Embodiment 47: A Camptothecin-Linker Compound of any one of Embodiments 40 to 44, wherein R^{B} is a member selected from the group consisting of C₃-C₈ cycloalkyl, C₃-C₈cycloalkylC₁-C₄ alkyl, phenyl and phenylC₁-C₄ alkyl, and wherein the cycloalkyl and phenyl portions of R^{B} are substituted with from 0 to 3 substituents selected from halogen, C₁-C₄ alkyl, OH, OC₁-C₄ alkyl, NH₂, NHC₁-C₄ alkyl and N(C₁-C₄ alkyl)₂. Embodiment 48: A Camptothecin-Linker Compound of any one of Embodiments 40 to 44, wherein R^{C} is C₁-C₆ alkyl. Embodiment 49: A Camptothecin-Linker Compound of any one of Embodiments 40 to 44, wherein R^{C} is C₃-C₆ cycloalkyl. Embodiment 50: A Camptothecin-Linker Compound of any one of Embodiments 40 to 44, wherein both R^{F} and R^{F′} are H. Embodiment 51: A Camptothecin-Linker Compound of any one of Embodiments 40 to 44, wherein at least one of R^{F} and R^{F′} is a member independently selected from the group consisting of C₁-C₈ alkyl, C₁-C₈ hydroxyalkyl, C₁-C₈ aminoalkyl, C₁-C₄ alkylaminoC₁-C₈ alkyl, (C₁-C₄ hydroxyalkyl)(C₁-C₄ alkyl)aminoC₁-C₈ alkyl, di(C₁-C₄ alkyl)aminoC₁-C₈ alkyl, C₁-C₄ hydroxyalkylC₁-C₈ aminoalkyl, C₁-C₈ alkylC(O)—, C₁-C₈ hydroxyalkylC(O)—, and C₁-C₈ aminoalkylC(O)—. Embodiment 52: A Camptothecin-Linker Compound of any one of Embodiments 40 to 44, wherein each R^{F} and R^{F′} is a member independently selected from the group consisting of H, C₁-C₈ alkyl, C₁-C₈ hydroxyalkyl, C₁-C₈ aminoalkyl, C₁-C₄ alkylaminoC₁-C₈ alkyl, (C₁-C₄ hydroxyalkyl)(C₁-C₄ alkyl)aminoC₁-C₈ alkyl, di(C₁-C₄ alkyl)aminoC₁-C₈ alkyl, C₁-C₄ hydroxyalkylC₁-C₈ aminoalkyl, C₁-C₈ alkylC(O)—, C₁-C₈ hydroxyalkylC(O)—, and C₁-C₈ aminoalkylC(O)—. Embodiment 53: A Camptothecin-Linker Compound of any one of Embodiments 40 to 44, wherein at least one of R^{F} and R^{F′} is a member independently selected from the group consisting of C₃-C₁₀ cycloalkyl, C₃-C₁₀cycloalkylC₁-C₄ alkyl, C₃-C₁₀ heterocycloalkyl, C₃-C₁₀ heterocycloalkylC₁-C₄ alkyl, phenyl, phenylC₁-C₄ alkyl, diphenylC₁-C₄ alkyl, heteroaryl and heteroarylC₁-C₄ alkyl, and wherein cycloalkyl, heterocycloalkyl, phenyl and heteroaryl portions of R^{F} and R^{F′} are substituted with from 0 to 3 substituents selected from halogen, C₁-C₄ alkyl, OH, OC₁-C₄ alkyl, NH₂, NHC₁-C₄ alkyl and N(C₁-C₄ alkyl)₂. Embodiment 54: A Camptothecin-Linker Compound of any one of Embodiments 40 to 44, wherein each R^{F} and R^{F′} is a member independently selected from the group consisting of H, C₃-C₁₀ cycloalkyl, C₃-C₁₀cycloalkylC₁-C₄ alkyl, C₃-C₁₀ heterocycloalkyl, C₃-C₁₀ heterocycloalkylC₁-C₄ alkyl, phenyl, phenylC₁-C₄ alkyl, diphenylC₁-C₄ alkyl, heteroaryl and heteroarylC₁-C₄ alkyl, and wherein cycloalkyl, heterocycloalkyl, phenyl and heteroaryl portions of R^{F} and R^{F′} are substituted with from 0 to 3 substituents selected from halogen, C₁-C₄ alkyl, OH, OC₁-C₄ alkyl, NH₂, NHC₁-C₄ alkyl and N(C₁-C₄ alkyl)₂. Embodiment 55: A Camptothecin-Linker Compound of any one of Embodiments 40 to 44, wherein R^{F} and R^{F′} are combined with the nitrogen atom to which each is attached to form a 5-, 6- or 7-membered ring having 0 to 3 substituents selected from halogen, C₁-C₄ alkyl, OH, OC₁-C₄ alkyl, NH₂, NHC₁-C₄ alkyl and N(C₁-C₄ alkyl)₂. Embodiment 56: A Camptothecin-Linker Compound of any one of Embodiments 40 to 55, wherein Z′-A- is maleimidopropionyl, mDPR, maleimidocaproyl or maleimidopropionyl-β-Alanyl. Embodiment 57: A Camptothecin-Linker Compound of Embodiment 56, wherein Z′-A- is maleimidopropionyl. Embodiment 58: A Camptothecin-Linker Compound of Embodiment 56, wherein Z′-A- is mDPR. Embodiment 59: A Camptothecin-Linker Compound of Embodiment 40, wherein S* is a PEG group. Embodiment 60: A Camptothecin-Linker Compound of Embodiment 40, wherein RL comprises a peptide selected from the group consisting of gly-gly, gly-gly-gly, gly-gly-gly-gly, val-gly-gly, val-cit-gly, val-gln-gly, val-glu-gly, phe-lys-gly, leu-lys-gly, gly-val-lys-gly, val-lys-gly-gly, val-lys-gly, val-lys-ala, val-lys-leu, leu-leu-gly, gly-gly-phe-gly, gly-gly-phe-gly-gly, val-gly, and val-lys-β-ala. Embodiment 62: A Camptothecin-Linker Compound of Embodiment 40, wherein RL comprises a peptide selected from the group consisting of gly-gly, gly-gly-gly, gly-gly-gly-gly, val-gly-gly, val-cit-gly, val-gln-gly, val-glu-gly, phe-lys-gly, leu-lys-gly, gly-val-lys-gly, val-lys-gly-gly, val-lys-gly, val-lys-ala, val-lys-leu, leu-leu-gly, gly-gly-phe-gly, gly-gly-phe-gly-gly, val-gly, and val-lys-β-ala; Z′-A- is maleimidopropionyl, mDPR or maleimidopropionyl-β-Alanyl; and S* is a PEG group. Embodiment 62: A Camptothecin-Linker Compound of Embodiment 40, selected from the group consisting of:

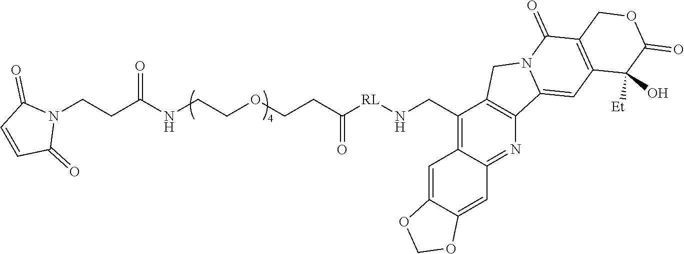

wherein RL is a peptide selected from the group consisting of gly-gly-gly-gly, val-lys-β-ala, val-gln-gly, val-lys-ala, phe-lys-gly, val-lys-gly-gly, gly-gly, val-lys-gly, val-gly-gly, leu-leu-gly, leu-lys-gly, val-glu-gly, gly-gly-gly, val-asp-gly, val-lys, val-gly and gly-val-lys-gly. Embodiment 63: A Camptothecin-Linker Compound of Embodiment 62, wherein RL is selected from the group consisting of val-lys-β-ala, val-gln-gly, val-lys-ala, phe-lys-gly, val-lys-gly, val-gly-gly, leu-leu-gly, leu-lys-gly, val-glu-gly, gly-gly-gly and val-asp-gly. Embodiment 64: A Camptothecin-Linker Compound of Embodiment 62, wherein RL is selected from the group consisting of val-lys-β-ala, val-gln-gly, val-lys-ala, phe-lys-gly, val-lys-gly, val-gly-gly, leu-lys-gly, val-glu-gly and val-asp-gly. Embodiment 65: A Camptothecin-Linker Compound of Embodiment 62, wherein RL is val-lys-gly.

Embodiment 66: A Camptothecin compound having the formula:

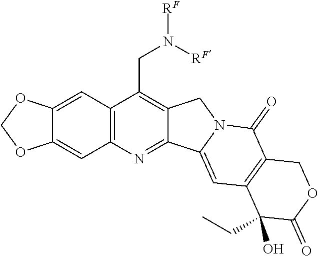

wherein each R^{F} and R^{F′} is independently a member selected from the group consisting of H, glycyl, hydroxyacetyl, ethyl, and 2-(2-(2-aminoethoxy)ethoxy)ethyl, or wherein R^{F} and R^{F′} are combined with the nitrogen atom to which each is attached to form a morpholino. Embodiment 67: The Camptothecin compound of Embodiment 66, wherein R^{F} is H and R^{F′} is glycyl, hydroxyacetyl, ethyl, 2-(2-(2-aminoethoxy)ethoxy)ethyl. Embodiment 68: The Camptothecin compound of Embodiment 66, wherein R^{F} and R^{F′} are combined with the nitrogen atom to which each is attached to form a morpholino.

Embodiment 69: A Camptothecin compound having the formula:

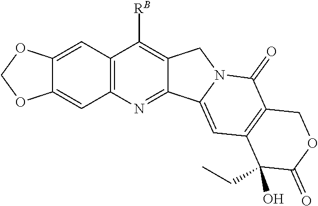

wherein R^{B} is a member selected from the group consisting of cyclopropyl, pentyl, hexyl, tert-butyl and cyclopentyl.

Embodiment 70: A method of treating cancer in a subject in need thereof, said method comprising administering to the subject a Camptothecin Conjugate of any one of Embodiments 1 to 39. Embodiment 71: The method of Embodiment 70, wherein said cancer is selected from the group consisting of lymphomas, leukemias, and solid tumors. Embodiment 72: The method of Embodiment 70, wherein said cancer is a lymphoma or a leukemia. Embodiment 73: The method of any one of Embodiments 70 to 73, further comprising an additional therapeutic agent. Embodiment 74: The method of Embodiment 73, wherein said additional therapeutic agent is one or more chemotherapeutic agents or radiation therapy.

Embodiment 75: A method of treating an autoimmune disease in a subject in need thereof, said method comprising administering the subject a Camptothecin Conjugate of any one of Embodiments 1 to 39. Embodiment 76: The method of Embodiment 75, wherein said autoimmune disease is selected from the group consisting of Th2 lymphocyte related disorders, Th1 lymphocyte-related disorders, and activated B lymphocyte-related disorders.

Embodiment 77: A method of preparing a Camptothecin Conjugate of any one of Embodiments 1 to 39, said method comprising reacting an antibody with a Camptothecin-Linker Compound of any one of Embodiments 40 to 65.

Embodiment 78: A kit comprising a Camptothecin Conjugate of any one of Embodiments 1 to 39. Embodiment 79: The kit of Embodiment 77, further comprising an additional therapeutic agent.

### EXAMPLES

### Experimental Procedures

### Abbreviations for Synthesis

Abbreviations for Synthesis

| | |
|---|---|
| AcOH | acetic acid |
| Boc | tert-butyloxycarbonyl protecting group |
| DCM | dichloromethane |
| DIPEA | N, N-diisopropylethylamine |
| DMA | N,N-dimethyacetamide |
| DMF | N,N-dimethylformamide |
| EtOAc | ethyl acetate |
| EtOH | ethanol |
| Fmoc | 9-fluorenylmethyl carbamates |
| HATU | 1-[bis(dimethylamino)methylene]-1H-1,2,3-triazolo[4,5- |
| | b]pyridinium 3-oxid hexafluorophosphate |
| Hex | hexanes |
| HPLC | high performance liquid chromatography |
| MeCN | acetonitrile |
| MeOH | methanol |
| Mal | 3-maleimido |
| MP | 3-maleimidopropionyl |
| MC | 3-maleimidocaproyl |
| mDPR | maleimido-amino-propionyl |
| MS | mass spectrometry |
| OSu | N-hydroxysuccinimide |
| PPTS | pyridinium para-toluene sulfonic acid |
| Prep | Preparative |
| TFA | triflouroacetic acid |
| TSTU | N,N,N′,N′-tetramethyl-O-(N-succinimidyl)uronium |
| | tetrafluoroborate |
| UPLC | Ultra Performance Liquid Chromatography |

### Materials and Methods

The following materials and methods are applicable to the synthetic procedures described in this section unless indicated otherwise. All commercially available anhydrous solvents were used without further purification. Starting materials, reagents and solvents were purchased from commercial suppliers (SigmaAldrich and Fischer). Products were purified by flash column chromatography utilizing a Biotage Isolera One flash purification system (Charlotte, N.C.). UPLC-MS was performed on a Waters single quad detector mass spectrometer interfaced to a Waters Acquity UPLC system equipped with a Waters Acuity UPLC BEH C18 2.1×50 mm, 1.7 μm, reversed-phase column. The eluent consisted of the solvents acetonitrile with 0.1% formic acid and 0.1% aqueous formic acid. The general method was a gradient of 3-60% acetonitrile over 1.7 min, then a linear gradient from 60-95% to 2.0 min, followed by isocratic of 95% acetonitrile to 2.5 min, then a equilibration of the column to 3% from 2.8 to 3.0 min with a flow rate of 0.5 mL/min. 2=0.4 mL/min), equipped with an Acquity UPLC BEH C18 2.1×50 mm, 1.7 μm reverse phase column. Preparative HPLC was carried out on a Waters 2454 Binary Gradient Module solvent delivery system configured with a Wasters 2998 PDA detector. Products were purified with the appropriate diameter of column of a Phenomenex Max-RP 4 μm Synergi 80 Å 250 mm reverse phase column eluting with 0.05% trifluoroacetic acid in water and 0.05% trifluoroacetic acid in acetonitrile.

### Camptothecin Compound Preparations

The Camptothecin Compounds provided in the following Examples can be used in preparing Camptothecin-Linker Compounds as well as Camptothecin Conjugates as described herein.

### Example 1

### TBS Protection of SN-38:

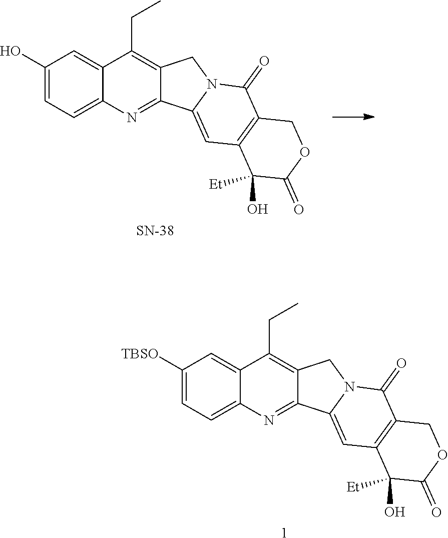 [SMILES: CCc1c2c(nc3ccc(O)cc13)-c1cc3c(c(=O)n1C2)COC(=O)[C@]3(O)CC ; "SN-38" ; CCc1c2c(nc3ccc(O[Si](C)(C)C(C)(C)C)cc13)-c1cc3c(c(=O)n1C2)COC(=O)[C@]3(O)CC ; "1"]

7-Ethyl-10-hydroxy-camptothecin (SN-38) (160.0 mg, 0.4077 mmmol) purchased from MedChemExpress was suspended in anhydrous DCM (2 mL). DIPEA (0.22 mL, 1.3 mmol) was added followed by TBSCl (154 mg, 1.02 mmol). The reaction was stirred for 30 minutes until SN-38 becomes soluble and complete conversion was observed by UPLC-MS. The reaction was quenched with MeOH, filtered through plug of silica, and concentrated in vacuo. The colorless oil obtained was triturated with Hex. The product precipitated out of solution. The precipitate was collected by filtration and rinsed with Hex to afford TBS-SN-38 (1) as an off-white solid (200 mg, 0.395 mmol, 97%). Rt=1.86 min Hydrophobic Method UPLC. MS (m/z) [M+H]⁺ calc. for C₂₈H₃₅N₂O₅Si 507.23, found 506.96.

### Example 2

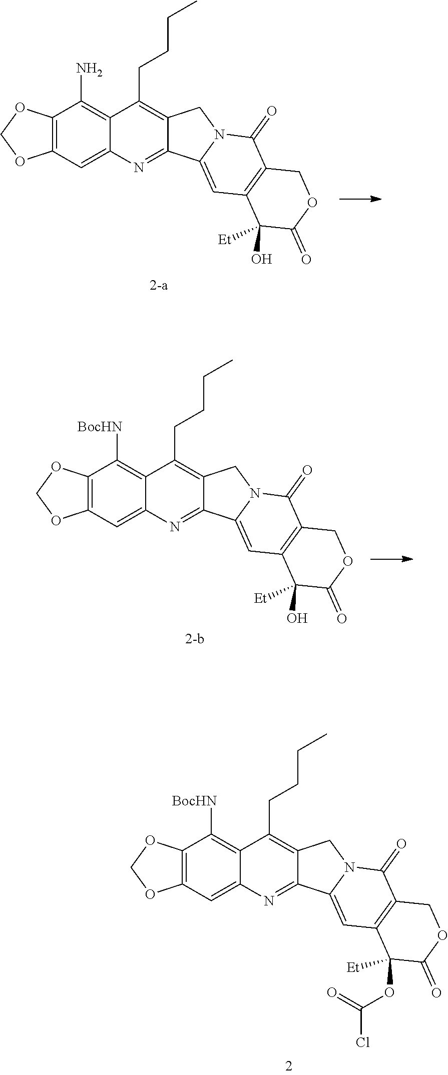 [SMILES: CCCCc1c2c(nc3cc4c(c(N)c13)OCO4)-c1cc3c(c(=O)n1C2)COC(=O)[C@]3(O)CC ; CCCCc1c2c(nc3cc4c(c(NC(=O)OC(C)(C)C)c13)OCO4)-c1cc3c(c(=O)n1C2)COC(=O)[C@]3(O)CC ; CCCCc1c2c(nc3cc4c(c(NC(=O)OC(C)(C)C)c13)OCO4)-c1cc3c(c(=O)n1C2)COC(=O)[C@@]3(CC)OC(=O)Cl]

Compound 2-a was synthesized according to the procedure described by Burke, P. J., Jeffrey, S. C. et al. in Bioconjugate Chem. 2009, 20, 1242-1250. Compound 2-a (50 mg, 0.108 mmol) dissolved in DCM (1 mL). DMAP (13 mg, 0.11 mmol) was added to the reaction followed by Boc₂O (24 mg, 0.11 mmol). The reaction was stirred for 5 minutes at which time complete conversion to the desired product was observed. The protected product was purified by column chromatography 10G Biotage Ultra 0-5% MeOH in DCM. Fractions containing the desired product were concentrated in vacuo to afford compound 2-b as a yellow solid (49 mg, 0.087 mmol, 80%). Rt=2.24 min General Method UPLC. MS (m/z) [M+H]⁺ calc. for C₃₀H₃₄N₃O₈ 564.23, found 564.10.

Compound 2-b (49 mg, 0.087 mmol) was dissolved in anhydrous DCM (2 mL). DMAP (37 mg, 0.304 mmol) was added and the reaction was cooled to 0° C. Triphosgene (12 mg, 0.039 mmol) dissolved 10 mg/mL in DCM was added dropwise to the reaction over 15 minutes. A 2 uL aliquot was quenched into 98 uL MeOH diluent and injected onto the UPLC-MS. Complete conversion to the MeOH adduct was observed by UPLC-MS. The reaction mixture (compound 2) can be used directly in coupling steps with suitable linkers. Rt=2.09 min General Method UPLC. MS (m/z) [M+H]⁺ calc. for C₃₂H₃₆N₃O₁₀ 622.24, found 622.02.

### Example 3

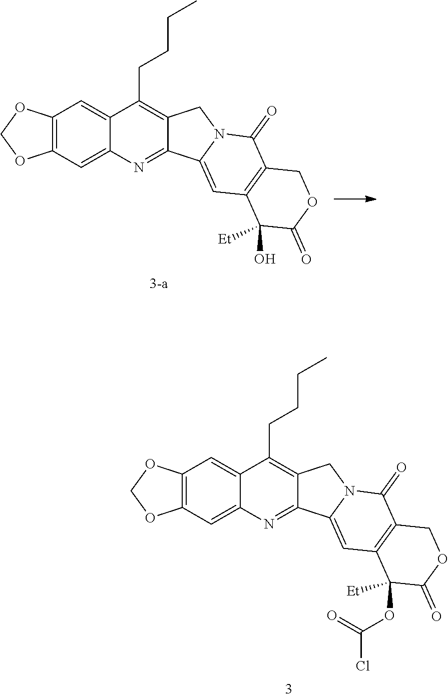 [SMILES: CCCCc1c2c(nc3cc4c(cc13)OCO4)-c1cc3c(c(=O)n1C2)COC(=O)[C@]3(O)CC ; CCCCc1c2c(nc3cc4c(cc13)OCO4)-c1cc3c(c(=O)n1C2)COC(=O)[C@@]3(CC)OC(=O)Cl]

Compound 3-a (150 mg, 0.334 mmol) was dissolved in anhydrous DCM (2 mL). DMAP (143 mg, 1.17 mmol) was added. Triphosgene (45 mg, 0.15 mmol) dissolved in anhydrous DCM (50 mg/mL) was added dropwise over 5 minutes. The reaction was stirred for 30 minutes at room temperature. A 2 uL aliquot of the reaction mixture was quenched in 98 uL MeOH diluent. Nearly complete conversion to MeOH carbonate observed indicating chloroformate formation. The product 3 can be used without further purification in coupling steps with suitable linkers. Rt=1.55 min General Method UPLC. MS (m/z) [M+H]⁺ calc. for C₂₇H₂₇N₂O₈ 507.18, found 507.06.

### Example 4

### Preparation of 7-Methylamino Derivative-Methylenedioxy Camptothecin (Referred to Herein as 7-MAD-MDCPT or Compound 4)

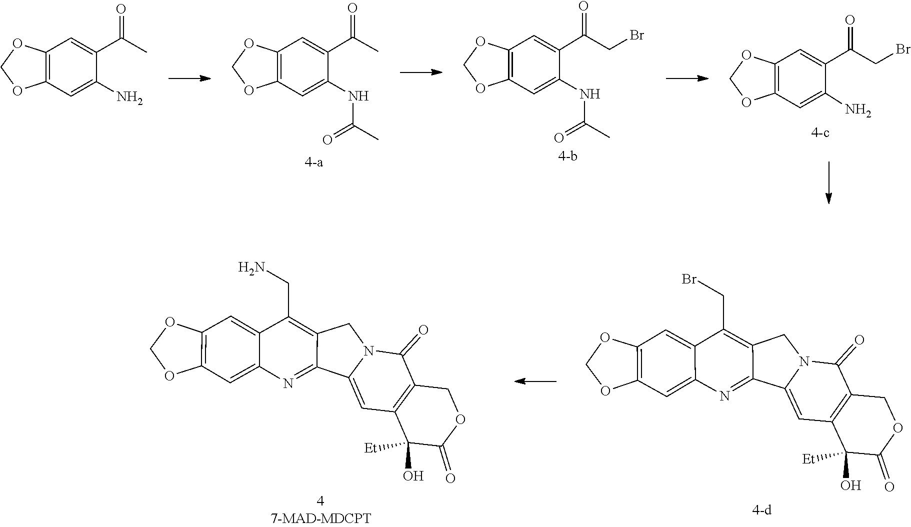 [SMILES: CC(=O)c1cc2c(cc1N)OCO2 ; CC(=O)Nc1cc2c(cc1C(C)=O)OCO2 ; CC(=O)Nc1cc2c(cc1C(=O)CBr)OCO2 ; Nc1cc2c(cc1C(=O)CBr)OCO2 ; CC[C@@]1(O)C(=O)OCc2c1cc1n(c2=O)Cc2c-1nc1cc3c(cc1c2CN)OCO3 ; CC[C@@]1(O)C(=O)OCc2c1cc1n(c2=O)Cc2c-1nc1cc3c(cc1c2CBr)OCO3]

6-Amino-3,4-(methylenedioxy)acetophenone (5.00 g, 27.9 mmol) was dissolved in DCM (100 mL). The reaction was cooled to 0° C. and DIPEA (7.29 mL, 41.9 mmol) was added followed by slow addition of acetyl chloride (2.49 mL, 34.9 mL). The reaction was allowed to warm to room temperature and stirred for 30 minutes. Complete conversion was observed by UPLC-MS. The reaction was quenched with MeOH (5 mL), and the reaction was concentrated in vacuo to afford compound 4-a as a white solid used in the next step without further purification. Rt=1.37 min General Method UPLC. MS (m/z) [M+H]⁺ calc. for C₁₁H₁₂NO₄ 222.08, found 222.11.

Compound 4-a (27.9 mmol) from previous step was dissolved in AcOH (100 mL). HBr 33% w/w in AcOH (9.78 mL, 55.8 mmoL) was added slowly. Bromine (1.44 mL, 27.9 mmol) was added dropwise over 15 minutes. The reaction was stirred for 30 minutes at which time conversion to desired product was observed. The reaction was poured over ice water and the precipitate was collected by filtration and washed with water. The filtrate was dried to afford a yellow powder which was a mixture of the desired product compound 4-b with starting material and dibrominated product impurities which was used in the next step without further purification (7.2 g, 24 mmol, 86%). Rt=1.58 min General Method UPLC. MS (m/z) [M+H]⁺ calc. for C₁₁H₁₁BrNO₄ 299.99, found 299.90.

Compound 4-b (7.2 g, 24 mmol) was dissolved in EtOH (100 mL). Concentrated HBr (5 mL) was added and the reaction was heated to reflux for 60 minutes. Nearly complete conversion to the deprotected product was observed. The reaction was concentrated in vacuo, diluted with DCM (200 mL) and H₂O (200) mL. The aqueous phase was extracted with DCM (3×200 mL), the collected organic phases were dried with MgSO₄, filtered and concentrated in vacuo. The crude product was purified by column chromatography 0-10% MeOH in DCM. Fractions containing the desired product with minor impurity were concentrated to afford compound 4-c as a yellow powder (4.05 g, 15.7 mmol, 65%). Rt=1.57 min General Method UPLC. MS (m/z) [M+H]⁺ calc. for C₉H₉BrNO₃ 257.98, found 257.71.

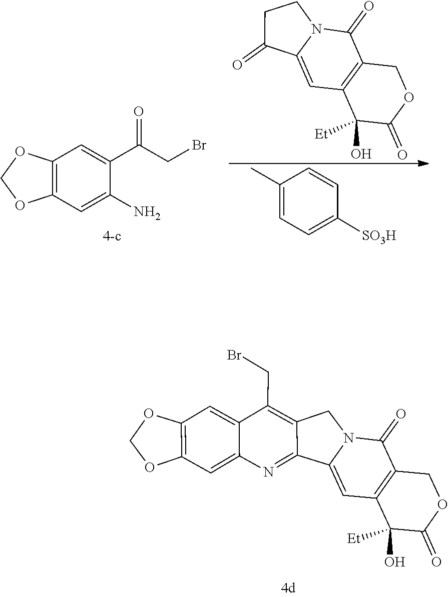

Compound 4-c (1.00 g, 3.87 mmol), p-TSA (667 mg, 3.87 mmol), and 4-Ethyl-4-hydroxy-7,8-dihydro-1H-pyrano[3,4-f]indolizine-3,6,10(4H)-trione (1.02 g, 3.87 mmol, obtained from Avra Laboratories Pvt. Ltd.) were charged in a flask. DCM (5 mL) was added to homogenize the solids, and then evaporated under nitrogen. The neat solids were then heated to 120° C. under high vacuum (1 mbar) for 60 minutes. Reaction was cooled to room temperature, the crude product precipitated with H₂O, filtered and washed with H₂O. The precipitate was purified by column chromatography 0-10% MeOH in DCM. Fractions containing the desired product were concentrated in vacuo to afford compound 4-d as a brown solid (989 mg, 2.04 mmol, 53%). Rt=1.57 min General Method UPLC. MS (m/z) [M+H]⁺ calc. for C₉H₉BrNO₃ 257.98, found 257.71. Rt=1.62 min General Method UPLC. MS (m/z) [M+H]⁺ calc. for C₂₂H₁₇BrN₂O₆ 485.03, found 484.95.

Compound 4-d (188 mg, 0.387 mmol) was dissolved in EtOH (5 mL). Hexamethylenetetramine (163 mg, 1.16 mmol) was added and the reaction as stirred at reflux for 90 minutes. The reaction was cooled and aq. conc. HCl (0.1 mL) was added. The reaction was concentrated and purified by prep-HPLC. Fractions containing the desired product were lyophilized to afford Compound 4 as an off white solid (109 mg, 0.259 mmol, 67%).

The following compounds can be prepared from 7-MAD-MDCPT (Compound 4) or from Compound 4-d, using conventional methods:

TABLE I

| | | Parent | Calc'd | Observed | |
|---|---|---|---|---|---|
| Compound | | Exact | MS (m/z) | MS | |
| No. | Structure | Mass | [M + H]⁺ | (m/z) | RT |
| 4a | 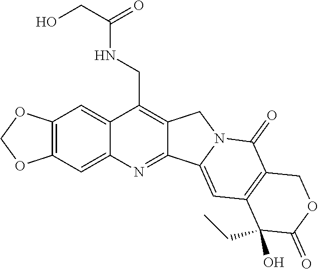 [SMILES: CC[C@@]1(O)C(=O)OCc2c1cc1n(c2=O)Cc2c-1nc1cc3c(cc1c2CNC(=O)CO)OCO3] | 479.13 | 480.14 | 480.08 | 1.20 |
| 4b | 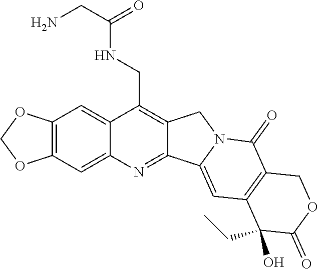 [SMILES: CC[C@@]1(O)C(=O)OCc2c1cc1n(c2=O)Cc2c-1nc1cc3c(cc1c2CNC(=O)CN)OCO3] | 478.15 | 479.16 | 479.11 | 1.05 |
| 4c | 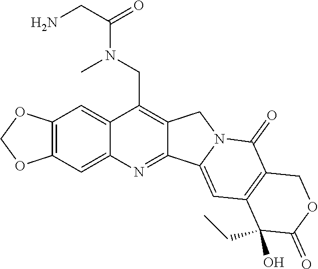 [SMILES: CC[C@@]1(O)C(=O)OCc2c1cc1n(c2=O)Cc2c-1nc1cc3c(cc1c2CN(C)C(=O)CN)OCO3] | 492.16 | 493.17 | 493.00 | 1.4 |
| 4d | 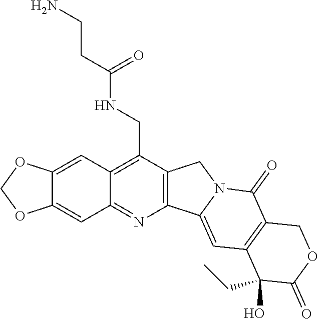 [SMILES: CC[C@@]1(O)C(=O)OCc2c1cc1n(c2=O)Cc2c-1nc1cc3c(cc1c2CNC(=O)CCN)OCO3] | 492.16 | 493.17 | 493.20 | 0.99 |
| 4e | 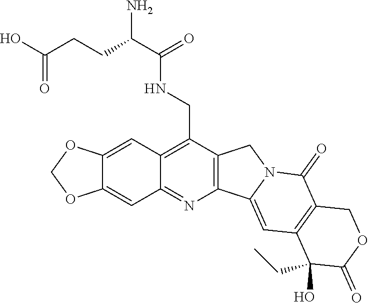 [SMILES: CC[C@@]1(O)C(=O)OCc2c1cc1n(c2=O)Cc2c-1nc1cc3c(cc1c2CNC(=O)[C@@H](N)CCC(=O)O)OCO3] | 550.17 | 551.18 | 551.20 | 0.94 |

### Example 5

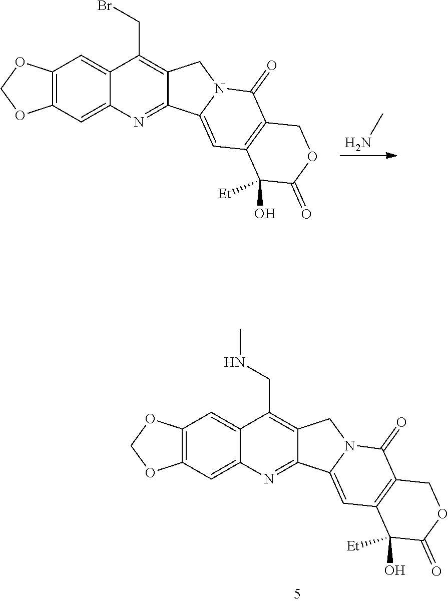

Substrate (4-d from Example 4, 10.0 mg, 20.6 μmol) was dissolved in anhydrous DMF (0.25 mL). Methylamine (2M in THF, 0.031 mL, 62 μmol) was added. The reaction was stirred for 30 minutes, then quenched with AcOH (20 μL). The reaction was purified by prep-HPLC. Fractions containing the desired product (5) were lyophilized to afford a yellow solid (3.27 mg, 7.51 μmol, 36%). Rt=1.57 min General Method UPLC. MS (m/z) [M+H]⁺ calc. for C₉H₉BrNO₃ 257.98, found 257.71. Rt=0.93 min General Method UPLC. MS (m/z) [M+H]⁺ calc. for C₂₃H₂₂N₃O₆ 436.15, found 435.78.

Examples 5a-5aa were made following the general procedures outlined for Compound 5.

TABLE II

| | | | Calc'd MS | Obsv'd | |
|---|---|---|---|---|---|
| Compound | | Parent Exact | (m/z) | MS | |
| No. | Structure | Mass | [M + H] | (m/z) | RT |
| 5a | 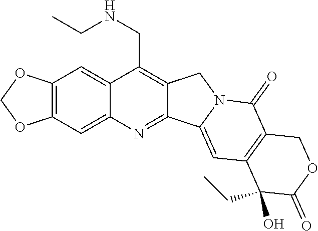 [SMILES: CCNCc1c2c(nc3cc4c(cc13)OCO4)-c1cc3c(c(=O)n1C2)COC(=O)[C@]3(O)CC] | 449.158685 | 450.17 | 450.14 | 1.19 |
| 5b | 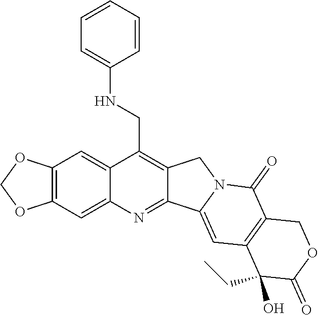 [SMILES: CC[C@@]1(O)C(=O)OCc2c1cc1n(c2=O)Cc2c-1nc1cc3c(cc1c2CNc1ccccc1)OCO3] | 497.158685 | 498.17 | 498.05 | 1.22 |
| 5c | 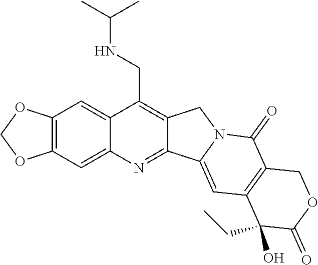 [SMILES: CC[C@@]1(O)C(=O)OCc2c1cc1n(c2=O)Cc2c-1nc1cc3c(cc1c2CNC(C)C)OCO3] | 463.174336 | 464.18 | 464.00 | 0.98 |
| 5d | 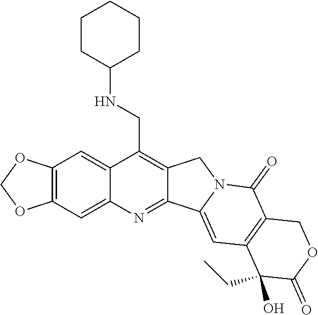 [SMILES: CC[C@@]1(O)C(=O)OCc2c1cc1n(c2=O)Cc2c-1nc1cc3c(cc1c2CNC1CCCCC1)OCO3] | 503.205636 | 504.22 | 504.16 | 1.16 |
| 5e | 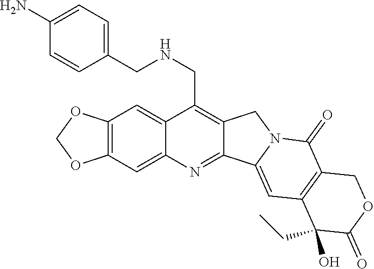 [SMILES: CC[C@@]1(O)C(=O)OCc2c1cc1n(c2=O)Cc2c-1nc1cc3c(cc1c2CNCc1ccc(N)cc1)OCO3] | 526.185235 | 527.20 | 526.08 | 1.11 |
| 5f | 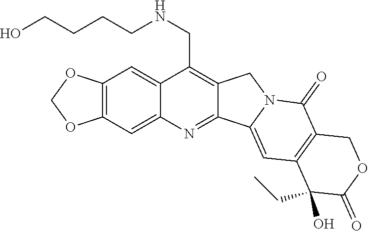 [SMILES: CC[C@@]1(O)C(=O)OCc2c1cc1n(c2=O)Cc2c-1nc1cc3c(cc1c2CNCCCCO)OCO3] | 493.1849 | 494.19 | 493.88 | 1.03 |
| 5g | 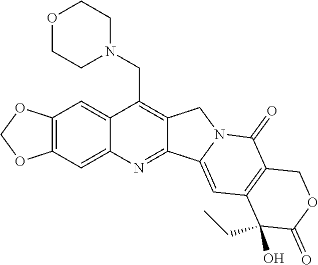 [SMILES: CC[C@@]1(O)C(=O)OCc2c1cc1n(c2=O)Cc2c-1nc1cc3c(cc1c2CN1CCOCC1)OCO3] | 491.16925 | 492.18 | 491.74 | 1.19 |
| 5h | 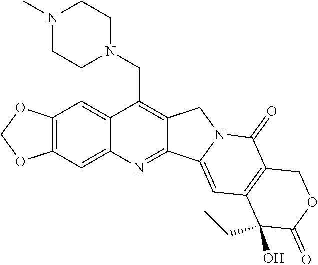 [SMILES: CC[C@@]1(O)C(=O)OCc2c1cc1n(c2=O)Cc2c-1nc1cc3c(cc1c2CN1CCN(C)CC1)OCO3] | 504.200885 | 505.21 | 504.93 | 1.10 |
| 5i | 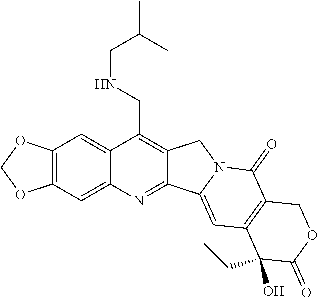 [SMILES: CC[C@@]1(O)C(=O)OCc2c1cc1n(c2=O)Cc2c-1nc1cc3c(cc1c2CNCC(C)C)OCO3] | 477.189986 | 478.20 | 478.26 | 1.30 |
| 5j | 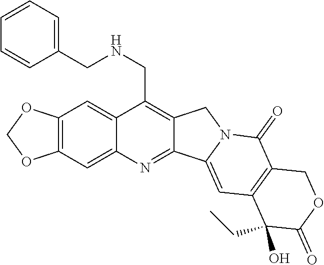 [SMILES: CC[C@@]1(O)C(=O)OCc2c1cc1n(c2=O)Cc2c-1nc1cc3c(cc1c2CNCc1ccccc1)OCO3] | 511.174336 | 512.18 | 512.21 | 1.20 |
| 5k | 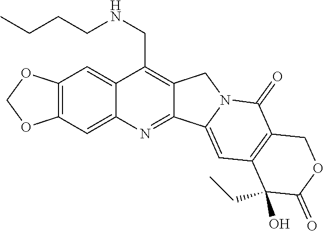 [SMILES: CCCCNCc1c2c(nc3cc4c(cc13)OCO4)-c1cc3c(c(=O)n1C2)COC(=O)[C@]3(O)CC] | 477.189986 | 478.20 | 477.68 | 1.13 |
| 5l | 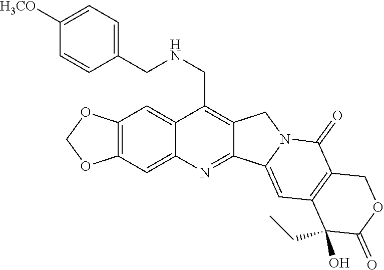 [SMILES: CC[C@@]1(O)C(=O)OCc2c1cc1n(c2=O)Cc2c-1nc1cc3c(cc1c2CNCc1ccc(OC)cc1)OCO3] | 541.1849 | 542.19 | 542.37 | 1.30 |
| 5m | 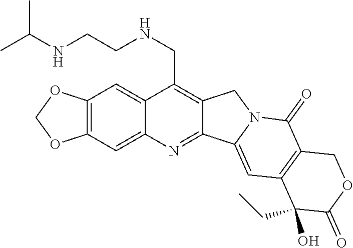 [SMILES: CC[C@@]1(O)C(=O)OCc2c1cc1n(c2=O)Cc2c-1nc1cc3c(cc1c2CNCCNC(C)C)OCO3] | 506.216535 | 507.23 | 507.94 | 0.76 |
| 5n | 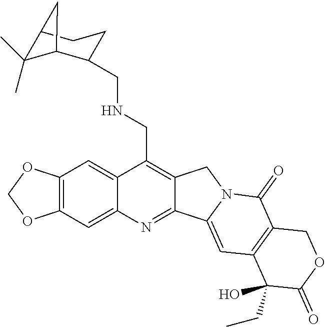 [SMILES: CC[C@@]1(O)C(=O)OCc2c1cc1n(c2=O)Cc2c-1nc1cc3c(cc1c2CNCC1CCC2CC1C2(C)C)OCO3] | 557.252586 | 558.26 | 557.89 | 1.51 |
| 5o | 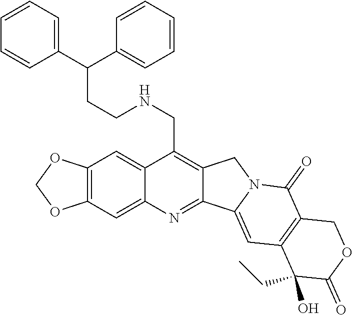 [SMILES: CC[C@@]1(O)C(=O)OCc2c1cc1n(c2=O)Cc2c-1nc1cc3c(cc1c2CNCCC(c1ccccc1)c1ccccc1)OCO3] | 615.236936 | 616.25 | 615.60 | 1.56 |
| 5p | 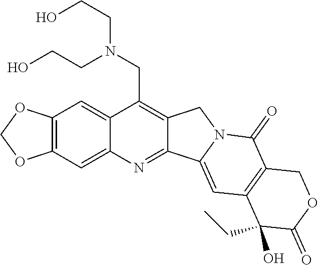 [SMILES: CC[C@@]1(O)C(=O)OCc2c1cc1n(c2=O)Cc2c-1nc1cc3c(cc1c2CN(CCO)CCO)OCO3] | 509.179815 | 510.19 | 509.69 | 1.09 |
| 5q | 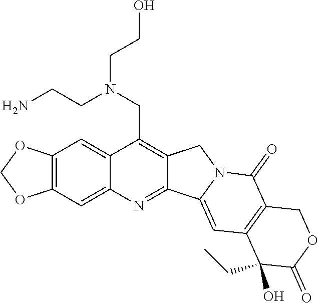 [SMILES: CC[C@@]1(O)C(=O)OCc2c1cc1n(c2=O)Cc2c-1nc1cc3c(cc1c2CN(CCN)CCO)OCO3] | 508.195799 | 509.21 | 508.91 | 1.11 |
| 5r | 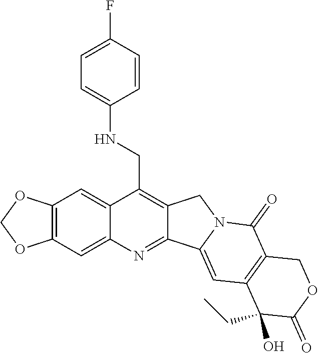 [SMILES: CC[C@@]1(O)C(=O)OCc2c1cc1n(c2=O)Cc2c-1nc1cc3c(cc1c2CNc1ccc(F)cc1)OCO3] | 515.149264 | 516.16 | 515.09 | 1.33 |
| 5s | 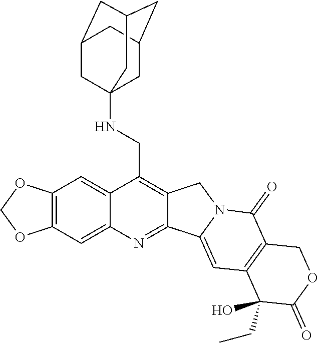 [SMILES: CC[C@@]1(O)C(=O)OCc2c1cc1n(c2=O)Cc2c-1nc1cc3c(cc1c2CNC12CC4CC(CC(C4)C1)C2)OCO3] | 555.236936 | 556.25 | 555.85 | 1.49 |
| 5t | 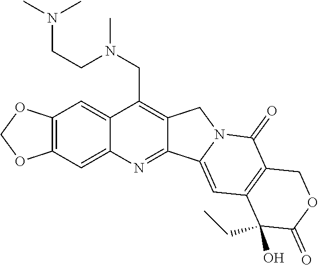 [SMILES: CC[C@@]1(O)C(=O)OCc2c1cc1n(c2=O)Cc2c-1nc1cc3c(cc1c2CN(C)CCN(C)C)OCO3] | 506.216535 | 507.23 | 506.58 | 1.17 |
| 5u | 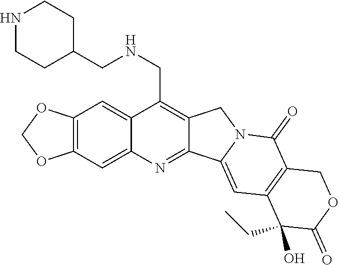 [SMILES: CC[C@@]1(O)C(=O)OCc2c1cc1n(c2=O)Cc2c-1nc1cc3c(cc1c2CNCC1CCNCC1)OCO3] | 518.216535 | 519.23 | 519.09 | 1.00 |
| 5w | 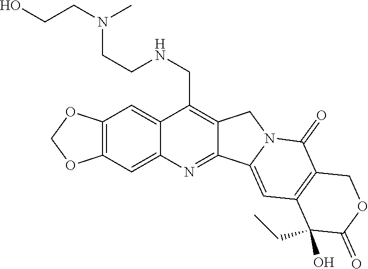 [SMILES: CC[C@@]1(O)C(=O)OCc2c1cc1n(c2=O)Cc2c-1nc1cc3c(cc1c2CNCCN(C)CCO)OCO3] | 522.211449 | 523.22 | 522.68 | 1.04 |
| 5x | 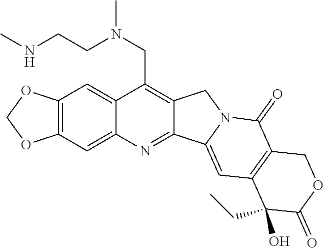 [SMILES: CC[C@@]1(O)C(=O)OCc2c1cc1n(c2=O)Cc2c-1nc1cc3c(cc1c2CN(C)CCNC)OCO3] | 492.200885 | 493.21 | 492.71 | 1.07 |
| 5y | 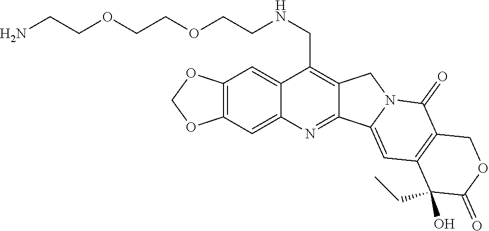 [SMILES: CC[C@@]1(O)C(=O)OCc2c1cc1n(c2=O)Cc2c-1nc1cc3c(cc1c2CNCCOCCOCCN)OCO3] | 552.222014 | 553.23 | 553.14 | 1.08 |
| 5z | 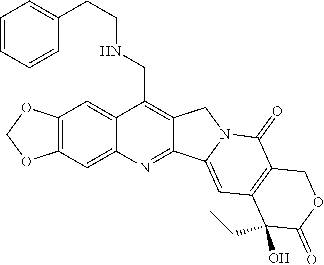 [SMILES: CC[C@@]1(O)C(=O)OCc2c1cc1n(c2=O)Cc2c-1nc1cc3c(cc1c2CNCCc1ccccc1)OCO3] | 525.189986 | 526.20 | 525.59 | 1.31 |
| 5aa | 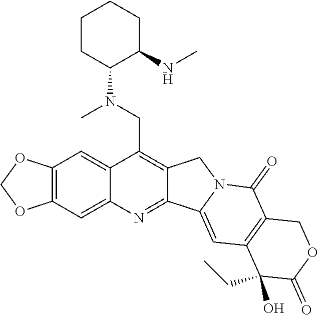 [SMILES: CC[C@@]1(O)C(=O)OCc2c1cc1n(c2=O)Cc2c-1nc1cc3c(cc1c2CN(C)[C@@H]1CCCC[C@H]1NC)OCO3] | 546.247835 | 547.26 | 546.64 | 1.26 |

### Example 6

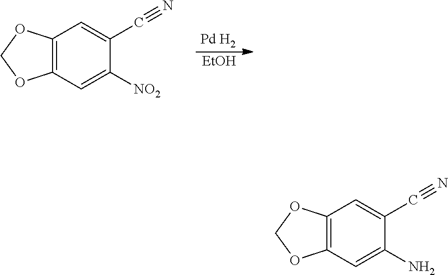 [SMILES: "EtOH" ; N#Cc1cc2c(cc1[N+](=O)[O-])OCO2 ; "Pd H2" ; N#Cc1cc2c(cc1N)OCO2]

6-nitro-1,3-benzodioxole-5-carbonitrile (2.00 g, 10.4 mmol) was dissolved in EtOH (50 mL). Reaction was placed under Nitrogen atmosphere. Pd/C (2.22 g, 10% w/w, 2.08 mmol) added to the reaction. Reaction placed under hydrogen atmosphere. The reaction was stirred for 2 hours. The reaction was filtered through a bed of Celite, and rinsed with MeOH. The eluent was concentrated in vacuo and purified by flash chromatography 0-10% DCM in MeOH. Fractions containing the desired product were concentrated to afford a red solid (1.46 g, 9.00 mmol, 87%). Rt=1.14 min General Method UPLC. MS (m/z) [M+H]⁺ calc. for C₈H₇N₂O₂ 163.05, found 162.37.

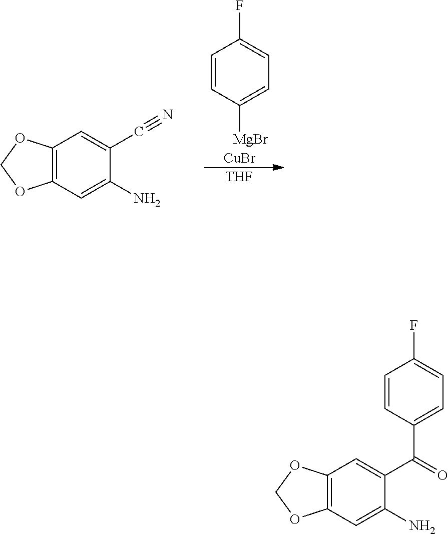

6-amino-1,3-benzodioxole-5-carbonitrile (50 mg, 0.31 mmol) was placed under nitrogen atmosphere and dissolved in anhydrous THF (1 mL). CuBr (1.5 mg, 0.010 mmol) was added followed by 4-fluorophenylmagnesium bromide 1M in THF (1.23 mL). The reaction was heated to 60° C. for 30 minutes, and then cooled to room temperature. A solution of 15% H₂SO₄ was added to the reaction slowly, and stirred for 30 minutes. The reaction was poured into sat. NaHCO₃ (50 mL), and extracted with EtOAc (3×50 mL). The organic was dried with MgSO₄, filtered and concentrated in vacuo. The crude product was purified by column chromatography 10G Biotage Ultra 0-10% EtOAc in Hex. Fractions containing the desired product were concentrated in vacuo to afford a red solid (46.2 mg, 0.178 mmol, 58%). Rt=1.81 min General Method UPLC. MS (m/z) [M+H]⁺ calc. for C₁₄H₁₁FNO₃ 260.07, found 259.46.

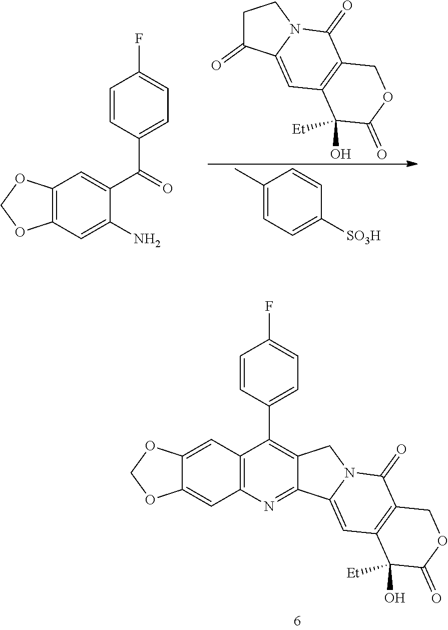

Substrate (46.2 mg, 0.178 mmol), p-TSA (30.7 mg, 0.178 mmol), and 4-Ethyl-4-hydroxy-7,8-dihydro-1H-pyrano[3,4-f]indolizine-3,6,10(4H)-trione (46.9 mg, 0.178 mmol) were charged in a scintillation vial. DCM (1 mL) was added to homogenate the solids. The solvent was concentrated under nitrogen. The neat solids were the heated to 120° C. under high vacuum (1 mbar) for 60 minutes. The reaction was reconstituted in DCM (50 mL), washed with H₂O, the organic phase wash dried with MgSO₄, filtered and concentrated in vacuo. The crude product was purified by column chromatography 10G Biotage Ultra 0-10% MeOH in DCM. Fractions containing the desired product (6) were concentrated in vacuo to afford a red solid (32.9 mg, 0.0676 mmol, 38%). Rt=1.81 min General Method UPLC. MS (m/z) [M+H]⁺ calc. for C₂₇H₂₀FN₂O₆ 487.13, found 487.19.

Examples 6a-6o were synthesized using a similar procedure as above for Compound 6.

TABLE III

| | | | Calc'd | | |
|---|---|---|---|---|---|
| Compound | | Parent Exact | MS (m/z) | Observed | |
| No. | Structure | Mass | [M + H]⁺ | MS (m/z) | RT |
| 6a | 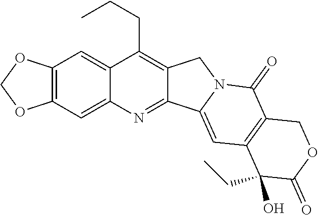 [SMILES: CCCc1c2c(nc3cc4c(cc13)OCO4)-c1cc3c(c(=O)n1C2)COC(=O)[C@]3(O)CC] | 434.147786 | 435.16 | 434.81 | 1.62 |
| 6b | 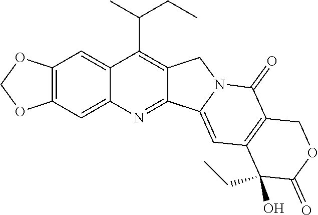 [SMILES: CCC(C)c1c2c(nc3cc4c(cc13)OCO4)-c1cc3c(c(=O)n1C2)COC(=O)[C@]3(O)CC] | 448.163437 | 449.17 | 448.78 | 1.71 |
| 6c | 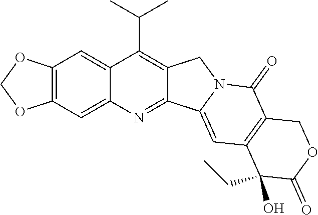 [SMILES: CC[C@@]1(O)C(=O)OCc2c1cc1n(c2=O)Cc2c-1nc1cc3c(cc1c2C(C)C)OCO3] | 434.147786 | 435.16 | 434.81 | 1.59 |
| 6d | 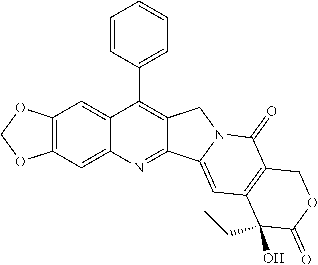 [SMILES: CC[C@@]1(O)C(=O)OCc2c1cc1n(c2=O)Cc2c-1nc1cc3c(cc1c2-c1ccccc1)OCO3] | 468.132136 | 469.14 | 469.15 | 1.77 |
| 6e | 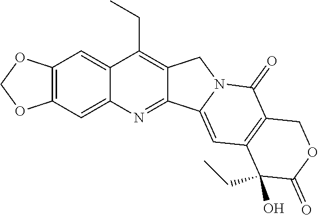 [SMILES: CCc1c2c(nc3cc4c(cc13)OCO4)-c1cc3c(c(=O)n1C2)COC(=O)[C@]3(O)CC] | 420.132136 | 421.14 | 420.85 | 1.48 |
| 6f | 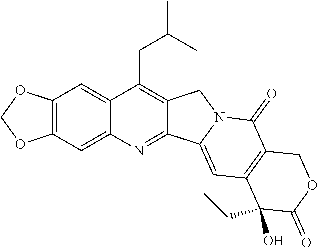 [SMILES: CC[C@@]1(O)C(=O)OCc2c1cc1n(c2=O)Cc2c-1nc1cc3c(cc1c2CC(C)C)OCO3] | 448.163437 | 449.17 | 448.78 | 1.76 |
| 6g | 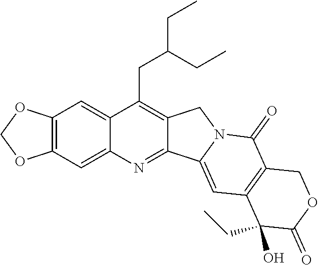 [SMILES: CCC(CC)Cc1c2c(nc3cc4c(cc13)OCO4)-c1cc3c(c(=O)n1C2)COC(=O)[C@]3(O)CC] | 476.194737 | 477.20 | 476.81 | 2.00 |
| 6h | 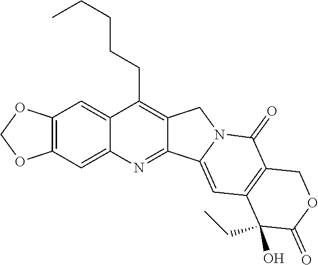 [SMILES: CCCCCc1c2c(nc3cc4c(cc13)OCO4)-c1cc3c(c(=O)n1C2)COC(=O)[C@]3(O)CC] | 462.179087 | 463.19 | 462.94 | 1.93 |
| 6i | 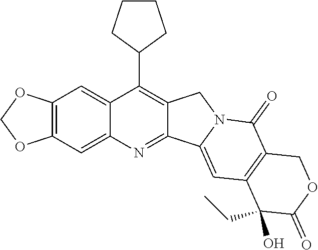 [SMILES: CC[C@@]1(O)C(=O)OCc2c1cc1n(c2=O)Cc2c-1nc1cc3c(cc1c2C1CCCC1)OCO3] | 460.163437 | 461.17 | 460.80 | 1.79 |
| 6j | 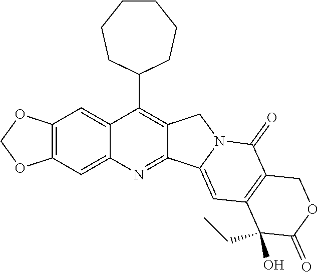 [SMILES: CC[C@@]1(O)C(=O)OCc2c1cc1n(c2=O)Cc2c-1nc1cc3c(cc1c2C1CCCCCC1)OCO3] | 488.194737 | 489.20 | 489.12 | 2.03 |
| 6k | 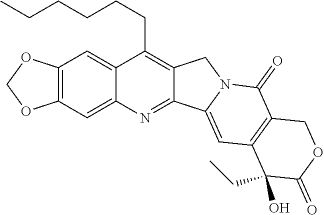 [SMILES: CCCCCCc1c2c(nc3cc4c(cc13)OCO4)-c1cc3c(c(=O)n1C2)COC(=O)[C@]3(O)CC] | 476.194737 | 477.20 | 478.07 | 2.06 |
| 6l | 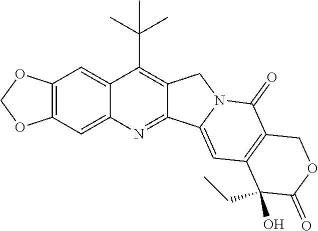 [SMILES: CC[C@@]1(O)C(=O)OCc2c1cc1n(c2=O)Cc2c-1nc1cc3c(cc1c2C(C)(C)C)OCO3] | 448.163437 | 449.17 | 448.87 | 1.69 |
| 6m | 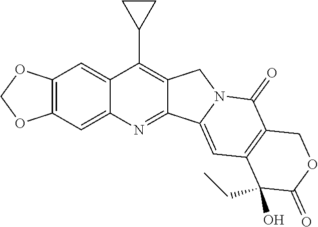 [SMILES: CC[C@@]1(O)C(=O)OCc2c1cc1n(c2=O)Cc2c-1nc1cc3c(cc1c2C1CC1)OCO3] | 432.132136 | 433.14 | 433.16 | 1.56 |
| 6n | 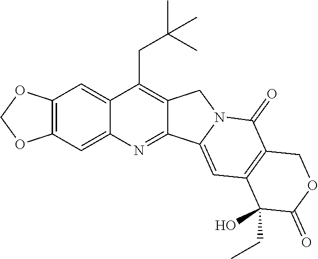 [SMILES: CC[C@@]1(O)C(=O)OCc2c1cc1n(c2=O)Cc2c-1nc1cc3c(cc1c2CC(C)(C)C)OCO3] | 462.179087 | 463.19 | 463.04 | 1.83 |
| 6o | 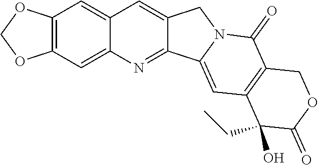 [SMILES: CC[C@@]1(O)C(=O)OCc2c1cc1n(c2=O)Cc2cc3cc4c(cc3nc2-1)OCO4] | 392.100836 | 393.11 | 393.01 | 1.31 |
| 6p | 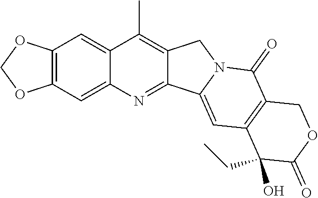 [SMILES: CC[C@@]1(O)C(=O)OCc2c1cc1n(c2=O)Cc2c-1nc1cc3c(cc1c2C)OCO3] | | | | |

### Example 7

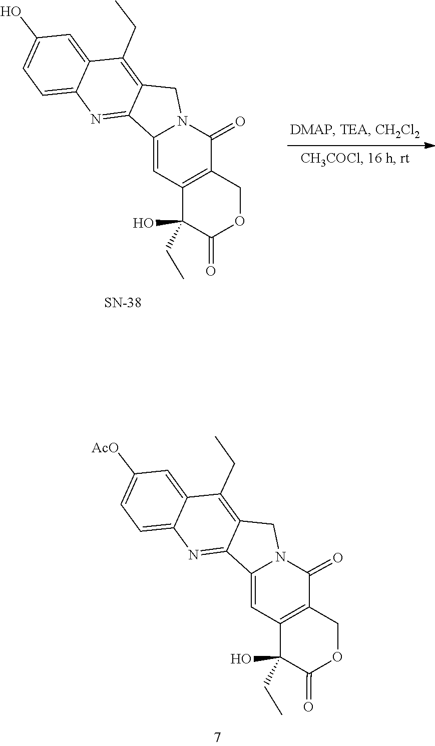 [SMILES: CCc1c2c(nc3ccc(O)cc13)-c1cc3c(c(=O)n1C2)COC(=O)[C@]3(O)CC ; "SN-38" ; CCc1c2c(nc3ccc(OC(C)=O)cc13)-c1cc3c(c(=O)n1C2)COC(=O)[C@]3(O)CC]

7-Ethyl-10-hydroxy-camptothecin (SN-38) (76.0 mg, 0.19 mmol) was dissolved in dichloromethane, followed by addition of triethylamine (128 μL, 0.92 mmol) and DMAP (2.60 mg, 0.02 mmol). Mixture was cooled to 0° C. in an ice bath, followed by dropwise addition of acetyl chloride (15.9 μL, 0.22 mmol). The reaction mixture was stirred at room temperature for 16 h. The reaction was diluted with dichloromethane, washed with saturated NH₄C₁, water, and brine. The organic phase was then dried over MgSO₄, filtered, concentrated and purified over silica via Biotage flash column chromatography (CH₂Cl₂/MeOH 0-15%) to yield acetylated SN-38 (7). MS (m/z) calculated 435.15 (M+H)⁺, found 435.07.

### Example 8

Compounds in Example 8 was prepared according to published procedures, and general methods.

TABLE IV

| | | Calc'd | | |
|---|---|---|---|---|
| Compound | | MS (m/z) | Observed | |
| No. | Structure | [M + H]⁺ | MS (m/z) | RT |
| 8a | 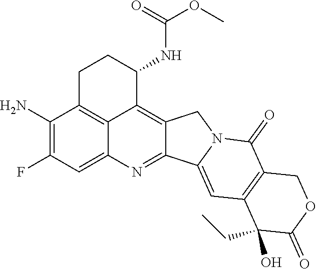 [SMILES: CC[C@@]1(O)C(=O)OCc2c1cc1n(c2=O)Cc2c-1nc1cc(F)c(N)c3c1c2[C@@H](NC(=O)OC)CC3] | 494.17 | 494.05 | 1.23 |
| 8b | 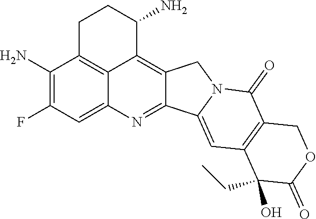 [SMILES: CC[C@@]1(O)C(=O)OCc2c1cc1n(c2=O)Cc2c-1nc1cc(F)c(N)c3c1c2[C@@H](N)CC3] | | | |
| 8c | 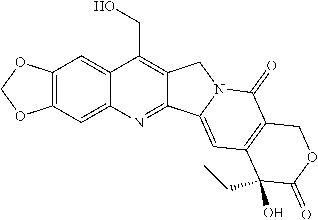 [SMILES: CC[C@@]1(O)C(=O)OCc2c1cc1n(c2=O)Cc2c-1nc1cc3c(cc1c2CO)OCO3] | 423.12 | 423.04 | 1.29 |
| 8d | 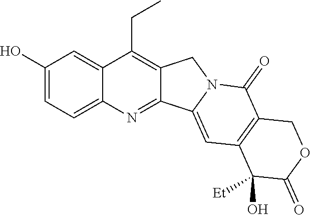 [SMILES: CCc1c2c(nc3ccc(O)cc13)-c1cc3c(c(=O)n1C2)COC(=O)[C@]3(O)CC] | | | |

### Camptothecin Linker Preparations

### Example 1-1

### Preparation of MC-Gly-Gly-Phe-Gly-aminomethoxyacetyl-7-MAD-MDCPT

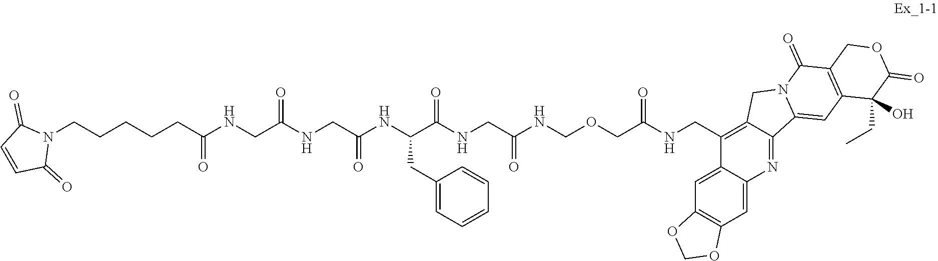 [SMILES: CC[C@@]1(O)C(=O)OCc2c1cc1n(c2=O)Cc2c-1nc1cc3c(cc1c2CNC(=O)COCNC(=O)CNC(=O)[C@H](Cc1ccccc1)NC(=O)CNC(=O)CNC(=O)CCCCCN1C(=O)C=CC1=O)OCO3]

MC-GGFG-Hemiaminal-Glycolic acid Synthesis

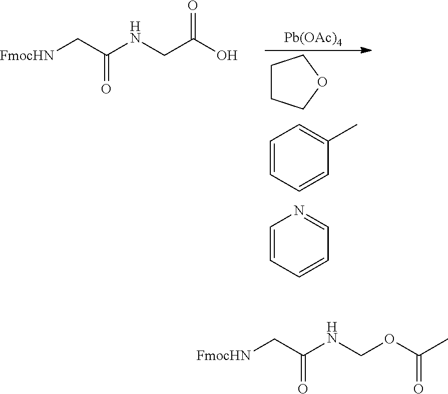

Based on the published procedure (WO 2015/155998 PCT/JP2015/002020) Fmoc-Gly-Gly-OH (4.70 g, 13.3 mmol) was partially dissolved in THF (120 mL), toluene (40 mL), and pyridine (2 mL). Lead tetraacetate (7.35 g, 16.6 mmol) was added to solution. The solution became orange. The reaction was heated to reflux. The solution turned colorless with a white precipitate after 1 hour. The reaction was stirred for a total of 3 hours then filtered through a bed of celite, rinsed with EtOAc, and concentrated in vacuo. The crude residue was purified by column chromatography 100G KP-Sil 10-100% EtOAc in Hex. Fractions containing the desired product were concentrated in vacuo to afford a colorless solid (3.39 g, 9.19 mmol, 69%). Rt=1.85 min General Method UPLC. Only able to observed iminium due to fragmentation of hemiaminal by MS (m/z) [M+H]⁺ calc. for C₁₈H₁₇N₂O₃ 309.12, found 309.13.

### PPTS Substitution:

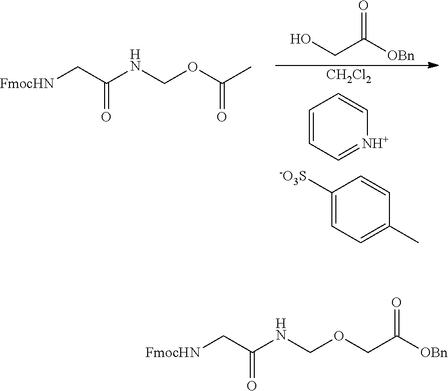

Substrate (3.39 g, 9.19 mmol) was dissolved in anhydrous DCM (50 mL). Benzyl glycoate (13.05 mL, 91.94 mmol) was added followed by PPTS (231 mg, 0.919 mmol) and the reaction was refluxed overnight. Nearly complete conversion observed by UPLC-MS. The reaction mixture was diluted with EtOAC (200 mL), washed with water (3×200 mL), dried MgSO4, filtered and concentrated in vacuo. The crude residue was purified by column chromatography 10-100% EtOAc in Hex. Fraction containing the desired product were concentrated to afford a white powder (4.30 g, 9.06 mmol, 99%). Rt=2.18 min General Method UPLC. MS (m/z) [M+Na]⁺ calc. for C₂₇H₂₆N₂NaO₆ 497.17, found 497.06.

### Fmoc Deprotection:

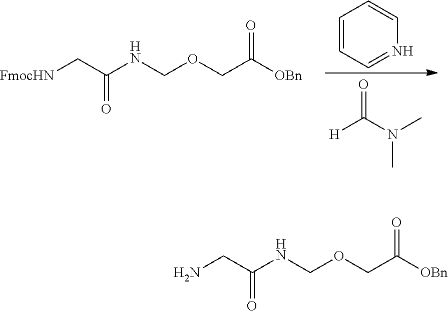

Substrate (1.00 g, 2.11 mmol) was dissolved in 20% piperidine in DMF and stirred for 20 minutes. The reaction was concentrated in vacuo and used in next step without further purification.

### Fmoc-Phe-Osu Coupling:

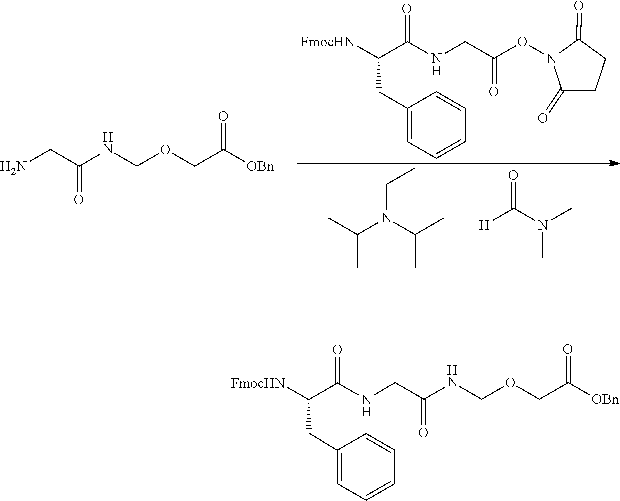

Crude product (2.11 mmol) from previous step was dissolved in DMF (2 mL). DIPEA (0.73 mL, 4.2 mmol) was added followed by Fmoc-Phe-OSu (1.71 g, 3.16 mmol). The reaction was stirred for 30 minutes at room temperature then concentrated in vacuo and purified by column chromatography 100G KP-Sil, 10-100% EtOAc in Hex. Fractions containing the desired product were concentrated to afford a white solid (910 mg, 1.46 mmol, 70%). Rt=2.28 min General Method UPLC. MS (m/z) [M+Na]⁺ calc. for C₃₆H₃₅N₃NaO₇ 644.24, found 644.04.

### Fmoc Deprotection:

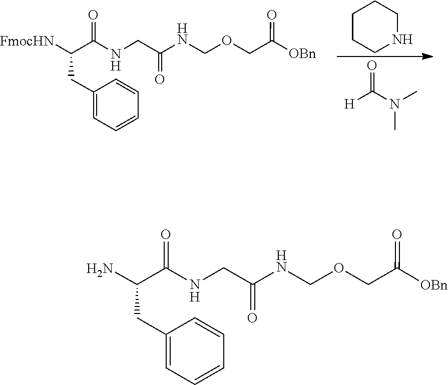

Substrate (910 mg, 1.46 mmol) was dissolved in 20% piperidine in DMF and stirred for 20 minutes. The reaction was concentrated in vacuo and used in next step without further purification.

### Fmoc Dipeptide Coupling:

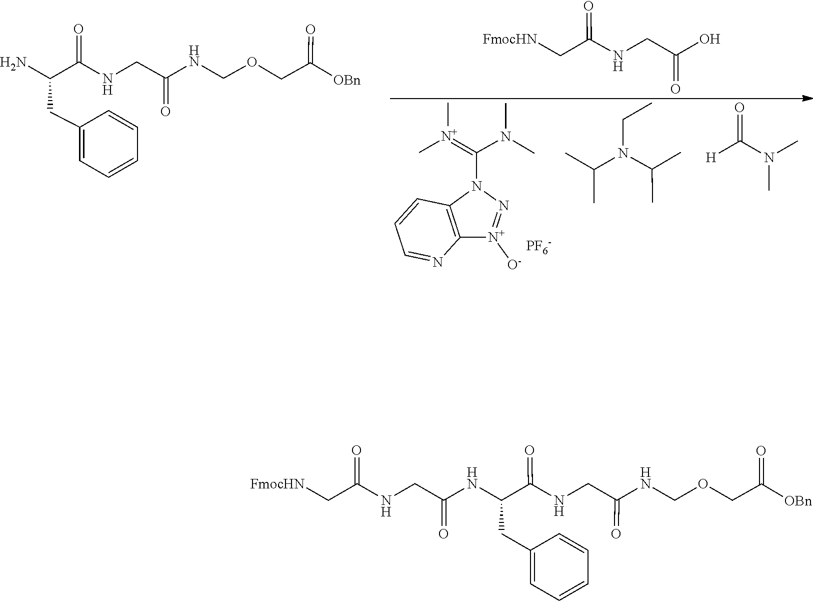

Crude product (1.46 mmol) from previous step was dissolved in anhydrous DMF (2 mL). DIPEA (1.00 mL, 5.76 mmol) and Fmoc-Gly-Gly-OH (1.07 g, 3.02 mmol) were added to the reaction followed by HATU (1.09 g, 2.88 mmol). The reaction was stirred for 30 minutes. The reaction was quenched with AcOH and purified by Prep-HPLC 50 mm 10-95% MeCN in H₂O 0.05% TFA. Fractions containing the desired product were concentrated in vacuo to afford a white solid (650 mg, 0.88 mmol, 61%). Rt=2.13 min General Method UPLC. MS (m/z) [M+Na]⁺ calc. for C₄₀H₄₁N₅NaO₉ 758.28, found 758.13.

### Pd Catalyzed Benzyl Ester Deprotection:

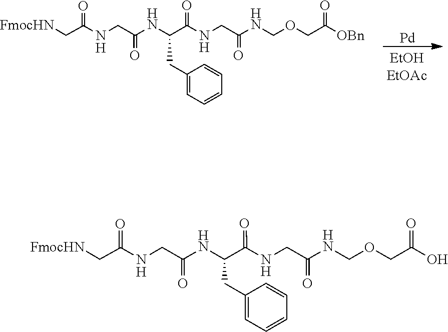

Substrate (650 mg, 0.88 mmol) was suspended in 2:1 EtOH:EtOAc (12 mL) and placed under nitrogen atmosphere. Pd/C (10% w/w, 132 mg, 0.124 mmol) was added to solution. Hydrogen was bubbled through reaction (1 atm) for 1 hours. Reaction was filtered through celite, rinsed MeOH, and concentrated in vacuo. Used in next step without further purification.

### Fmoc Deprotection:

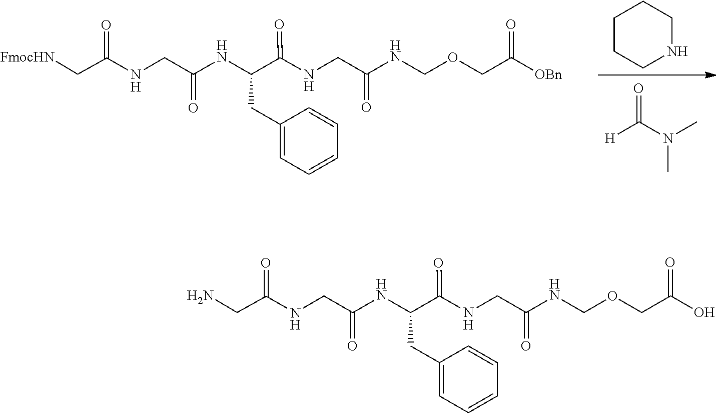

Crude solid (0.88 mmol) from previous step was dissolved in DMF (8 mL). Piperidine (2 mL) added. Stirred for 10 minutes. The reaction was concentrated in vacuo to afford a white solid. Used in next step without further purification.

### MC-OSu Coupling:

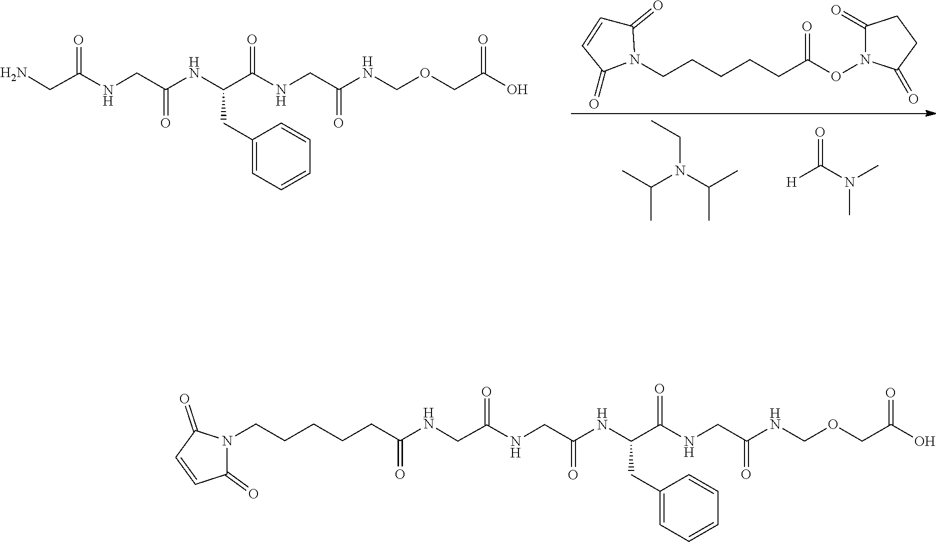

Crude product (0.88 mmol) from previous step was dissolved in DMF (10 mL). DIPEA (1 mL) was added followed by MC-OSu (407 mg, 1.32 mmol). The reaction was stirred for 10 minutes. Complete conversion was observed by UPLC-MS. AcOH (1 mL) was added to quench the reaction. Purified by Prep-HPLC 50 mm 10-95% MeCN in H₂O 0.05% TFA. Fractions containing the desired product were lyophilized to afford a white solid (453 mg, 0.735 mmol, 83%). Rt=1.21 min General Method UPLC. MS (m/z) [M+H]⁺ calc. for C₂₈H₃N₆O₁₀ 617.26, found 617.07.

MC-GGFG-Glycolic Linker Coupling with 7-MAD-MDCPT:

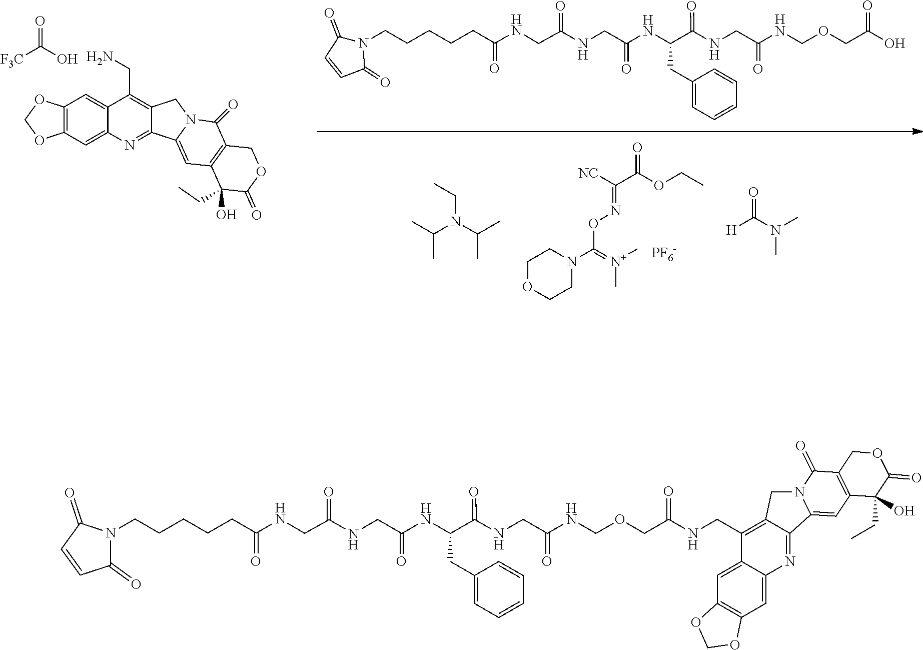

MC-GGFG-Glycolic Acid (46 mg, 0.075 mmol) was dissolved in DMF (0.5 mL). DIPEA (26 μL, 0.149 mmol) was added followed by COMU (32 mg, 0.075 mmol). The reaction was stirred for 30 minutes at room temperature. Activated acid solution was added directly to 7-MAD-MDCPT drug solid (from Example 4). Complete conversion was observed by UPLC-MS after 5 minutes. The reaction was quenched with AcOH, purified by Prep-HPLC 10-95% MeCN in H₂O 0.05% TFA. Fractions containing the desired product were lyophilized to afford the desired product as a white solid (8.00 mg, 7.84 μmol, 21%). Rt=1.93 min General Method UPLC. MS (m/z) [M+H]⁺ calc. for C₅₀H₅₄N₉O₁₅ 1020.37, found 1020.09.

### Example 2-1

### Preparation of MC-Val-Cit-PABA-7-MAD-MDCPT

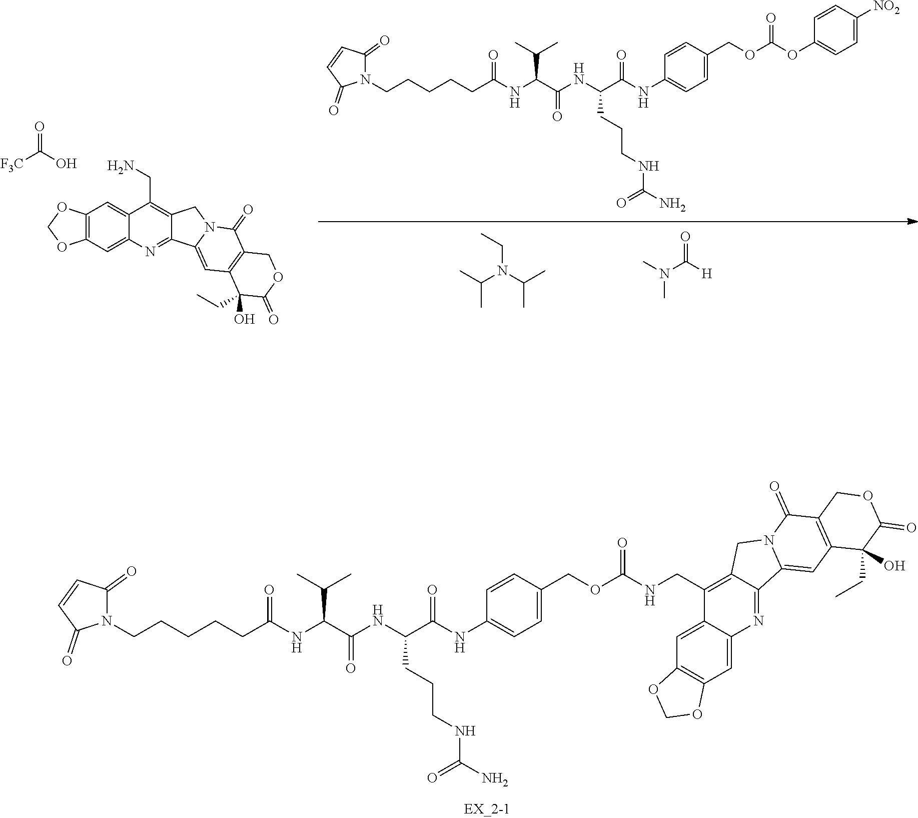

7-MAD-MDCPT TFA salt (20.0 mg, 0.0374 mmol) and MC-Val-Cit-PABA-PNP (82.7 mg, 0.112 mmol) were dissolved in anhydrous DMF (0.5 mL). DIPEA (26 μL, 0.149 mmol) was added. Complete conversion was observed by UPLC-MS after 10 minutes. The reaction was quenched with AcOH, and purified by prep-HPLC 21 mm 10-95% MeCN in H₂O 0.05% TFA. Fractions containing the desired product were lyophilized to afford a white powder (2.4 mg, 2.4 μmol, 6%). Rt=1.59 min General Method UPLC. MS (m/z) [M+H]⁺ calc. for C₅₁H₅₈N₉O₁₄ 1020.41, found 1020.09.

### Example 3-1

### Preparation of MP-PEG4-Val-Lys-7-MAD-MDCPT

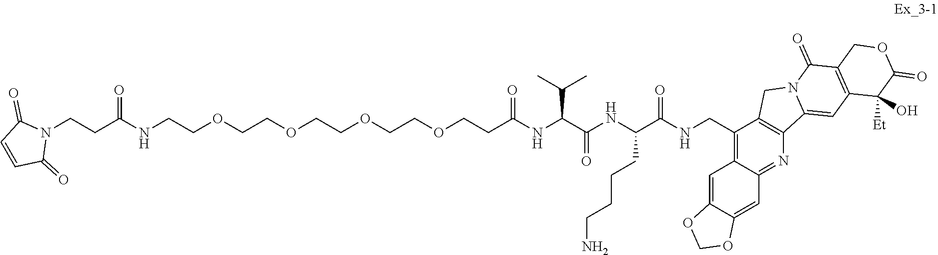 [SMILES: CC[C@@]1(O)C(=O)OCc2c1cc1n(c2=O)Cc2c-1nc1cc3c(cc1c2CNC(=O)[C@H](CCCCN)NC(=O)[C@@H](NC(=O)CCOCCOCCOCCOCCNC(=O)CCN1C(=O)C=CC1=O)C(C)C)OCO3]

### Solid Phase Peptide Synthesis of MP-PEG4-VK(Boc)-OH

2-chlorotrityl resin (1.6 mmol/g, 2 grams) was added to reaction vessel, and washed with DMF 2 times. The resin was swelled in 20 mL DMF for 10 minutes, and then drained. Fmoc-Lys(Boc)-OH (937 mg, 2 mmol) and DIPEA (0.7 mL, 4 mmol) dissolved in 10 mL DMF was added to the resin and shake for 30 minutes at room temperature. MeOH (5 mL) was added to the resin and shaken for 5 min, then drained, and washed with DMF 5 times. The substitution was assumed to be 1 mmol/g. The resin washed with DCM 3 times, washed with MeOH 3 times, then dried under high vacuum overnight. The prepared Fmoc-Lys(Boc)-2-chlorotrityl resin (1 gram) was added to a reaction vessel. The resin washed with DMF 3 times and swelled in 10 mL DMF for 10 minutes, then drained. The Fmoc was deprotected using the general deprotected procedure. Using the general coupling procedure Fmoc-Val-OH was coupling to the resin, followed by the general deprotection procedure. MP-PEG4-OH was coupled using the general coupling procedure. The resin was then washed with DCM 3 times, followed by MeOH 3 times, and placed under high vacuum overnight. The peptide was cleaved off resin by stirring the resin in a solution of 1 mL Acetic Acid, 2 mL hexaflouroisopropanol, and 7 mL DCM for 1 hour. Resin was then filtered and rinsed with DCM 3 times, and then the solution was concentrated in vacuo. The white powder was dissolved in 2:1 DMA:H₂O (3 mL) and purified by preparative HPLC using a 30×250 mm Phenomenex Max-RP 4 μm Synergi 80 Å reverse phase column using a 5-60-95% gradient elution of MeCN (0.05% TFA) in aqueous 0.05% TFA described below. Fractions containing the desired product were lyophilized to afford a white powder (343 mg, 0.461 mmol, 46%). Rt=1.50 min General Method UPLC. MS (m/z) [M+H]⁺ calc. for C₃₄H₅₇N₅O₁₃ 744.16, found 744.40.

### 5-60-95% Gradient Elution

| Time (min) | Flow (mL/min) | % MeCN |
|---|---|---|
| Initial | 8 | 5 |
| 3 | 8 | 5 |
| 5 | 15 | 5 |
| 48 | 15 | 60 |
| 50 | 15 | 95 |
| 55 | 15 | 95 |
| 56 | 15 | 5 |
| 60 | 15 | 5 |

Coupling MP-PEG4-VK(Boc)-OH with 7-MAD-MDCPT and Boc Deprotection

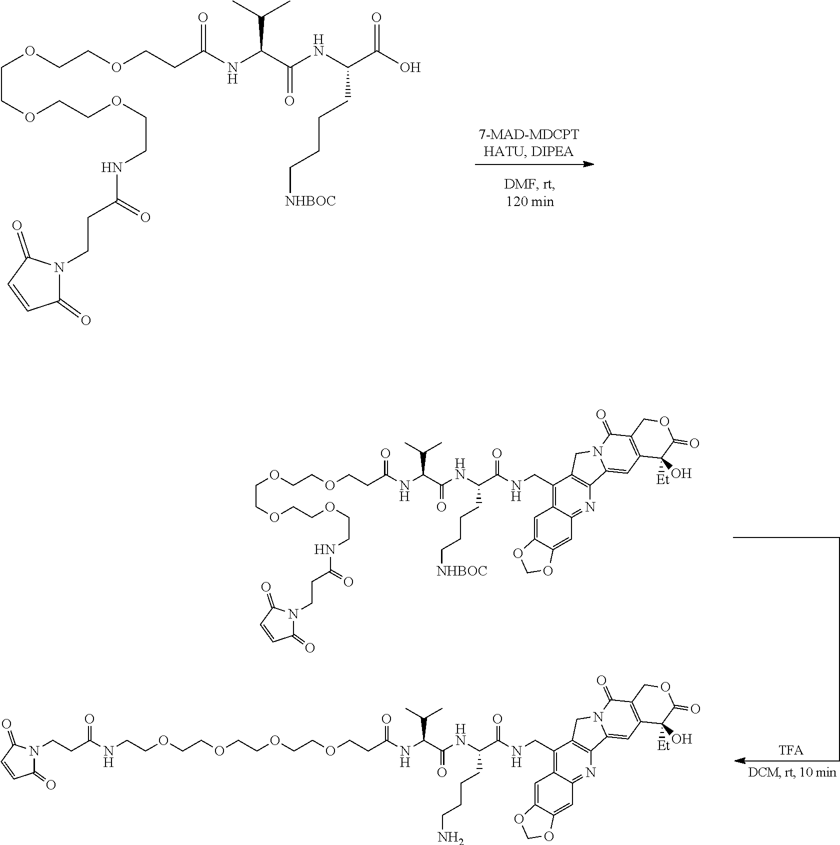

MP-PEG4-VK(Boc)-OH (30 mg, 0.040 mmol) was dissolved in anhydrous DMF (0.5 mL) and DIPEA (50 μL, 0.28 mmol). HATU (15.3 mg, 0.0403 mmol) was added to the solution. Reaction was stirred at room temperature for 30 minutes. The activated acid solution was added directly to the 7-MAD-MDCPT solid (17 mg, 0.04 mmol). The reaction as monitor for completion by UPLC-MS. Complete conversion was observed after 120 minutes. The reaction was acidified with AcOH (50 μL, 0.87 mmol), and purified by purified by preparative HPLC using a 21×250 mm Phenomenex Max-RP 4 μm Synergi 80 Å reverse phase column using a 5-60-95% gradient elution of MeCN (0.05% TFA) in aqueous 0.05% TFA described previously. Fractions containing the desired product were lyophilized to afford a yellow powder (5 mg, 0.0044 mmol, 11%). Rt=1.70 min General Method UPLC. MS (m/z) [M+H]⁺ calc. for C₅₇H₇₅N₇O₁₈ 1146.52, found 1147.19.

MP-PEG4-VK(Boc)-7-MAD-MDCPT was dissolved in 20% TFA in DCM. Reaction was monitored for completion by UPLC-MS. Complete conversion after 10 minutes. The reaction was concentrated in vacuo, reconstituted in 10% AcOH in 2:1 DMA:H₂O, and purified by preparative HPLC using a 21×250 mm Phenomenex Max-RP 4 μm Synergi 80 Å reverse phase column using a 5-60-95% gradient elution of MeCN (0.05% TFA) in aqueous 0.05% TFA described previously. Fractions with absorbance at 385 nm were collected. The fractions containing the desired product were lyophilized to afford compound 3-1 as yellow powder (2.5 mg, 0.0023 mmol, 55%). Rt=1.12 min General Method UPLC. MS (m/z) [M+H]⁺ calc. for C₅₂H₆₇N₇O₁₆ 1046.47, found 1047.26.

### Example 4-1

### Preparation of MP-PEG4-Val-Lys-Gly-7-MAD-MDCPT

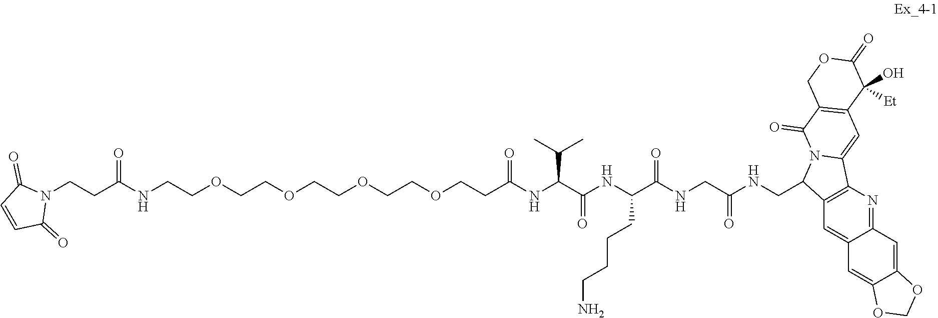 [SMILES: CC[C@@]1(O)C(=O)OCc2c1cc1n(c2=O)C(CNC(=O)CNC(=O)[C@H](CCCCN)NC(=O)[C@@H](NC(=O)CCOCCOCCOCCOCCNC(=O)CCN2C(=O)C=CC2=O)C(C)C)c2cc3cc4c(cc3nc2-1)OCO4]

### Solid Phase Peptide Synthesis of MP-PEG4-VK(Boc)G-OH

Unprotected glycine pre-loaded 1.1 mmol/g on 2-chlorotryityl resin was purchased from BAChem. Resin (1 gram) was added to reaction vessel. Resin washed with DMF 4 times and drained completely. Resin swelled by shaking in DMF for 30 minutes, and drained. Using the general coupling procedure Fmoc-Lys(Boc)-OH was coupled to the resin. The Fmoc was deprotected using the general deprotection procedure. Using the general coupling procedure Fmoc-Val-OH was coupled to the resin, followed by the general deprotection procedure. MP-PEG4-OH was coupled using the general coupling procedure. The resin was then washed with DCM 3 times, followed by MeOH 3 times, and placed under high vacuum overnight. The peptide was cleaved from the resin by stirring the resin in a solution of 1 mL Acetic Acid, 2 mL hexaflouroisopropanol, and 7 mL DCM for 1 hour. Resin was then filtered and rinsed with DCM 3 times, and then the solution was concentrated in vacuo. The white powder was dissolved in 2:1 DMA:H2O (3 mL) and purified by preparative HPLC using a 30×250 mm Phenomenex Max-RP 4 μm Synergi 80 Å reverse phase column using a 5-60-95% gradient elution of MeCN (0.05% TFA) in aqueous 0.05% TFA described below. Fractions containing the desired product were lyophilized to afford a white powder (354 mg, 0.442 mmol, 40%). Rt=1.39 min General Method UPLC. MS (m/z) [M+H]⁺ calc. for C₃₆H₅₉N₆O₁₄ 801.42, found 801.02.

### 5-60-95% Gradient Elution

| Time (min) | Flow (mL/min) | % MeCN |
|---|---|---|
| Initial | 8 | 5 |
| 3 | 8 | 5 |
| 5 | 15 | 5 |
| 48 | 15 | 60 |
| 50 | 15 | 95 |
| 55 | 15 | 95 |
| 56 | 15 | 5 |
| 60 | 15 | 5 |

### General Fmoc Deprotection Procedure

A solution of 20% piperidine in DMF (10 mL) was added to the resin, shaken for 1 minute, and drained. Another 10 mL of 20% piperidine in DMF was added to the resin, shaken for 30 minutes, and drained. The resin washed with DMF 4 times and drained completely.

### General Coupling Procedure

A solution was prepared in DMF (10 mL) of Fmoc Amino Acid (3 mmol), HATU (3 mmol), DIPEA (6 mmol). The solution was added to the resin, and shaken for 60 minutes. The reaction vessel was drained and washed with DMF 4 times.

### Synthesis of MP-PEG4-VK(Boc)G-OSu

MP-PEG4-VKG-OH (90.0 mg, 0.112 mmol) was dissolved in anhydrous DMF (0.3 mL) and DIPEA (0.05 mL, 0.302 mmol) was added. TSTU (67.6 mg, 0.224 mmol) was added to the reaction vessel, and conversion to the N-hydroxysuccinimide (OSu) activated ester was monitored by UPLC-MS. Complete conversion was observed after 5 minutes. The reaction was acidified with AcOH (0.05 mL, 0.874 mmol). The reaction was purified by Biotage flash chromatography using 10G Ultra silica gel column with a gradient elution of 0-10% MeOH in DCM. Fractions containing the desired product were concentrated in vacuo to afford a white solid which was the desired product MP-PEG4-VK(Boc)G-OSu (91.2 mg, 0.102 mmol, 90%). Rt=1.48 General Method UPLC. MS (m/z) [M+H]⁺ calc. for C₄₀H₆₂N₇O₁₆ 898.44, found 898.33.

Coupling MP-PEG4-VK(Boc)G-OSu with 7-MAD-MDCPT

A solution of 7-MAD-MDCPT (24 mg, 0.057 mmol) dissolved in anhydrous DMF (0.48 mL) was added directly to the reaction vessel with the MP-PEG4-VK(Boc)G-OSu (50 mg, 0.056 mmol). DIPEA (0.05 mL, 0.303 mmol) was added to the reaction vessel. The clear yellow solution turned opaque upon the addition of base. The reaction was monitored for completion by UPLC-MS. Complete conversion to the desired coupled product was observed after 5 minutes. The reaction was acidified with AcOH (0.05 mL, 0.87 mmol) and purified by filtration through silica gel column with a gradient elution of 0-10% MeOH in DCM. The eluent was concentrated in vacuo to afford a yellow solid which was the desired product MP-PEG4-VKG-7-MAD-MDCPT (32 mg, 0.027 mmol, 48%). Rt=1.59 min General Method UPLC. MS (m/z) [M+H]⁺ calc. for C₅₈H₇₇N₉O₁₉ 1204.54, found 1204.25.

### Boc Deprotection of MP-PEG4-VK(Boc)G-7-MAD-MDCPT

MP-PEG4-VK(Boc)-G-7-MAD-MDCPT was dissolved in 20% TFA in DCM. Reaction was monitored for completion by UPLC-MS. Complete conversion was observed after 10 minutes. The reaction was concentrated in vacuo, reconstituted in 10% AcOH in 2:1 DMA:H₂O, and purified by preparative HPLC using a 21×250 mm Phenomenex Max-RP 4 μm Synergi 80 Å reverse phase column using a 5-60-95% gradient elution of MeCN (0.05% TFA) in aqueous 0.05% TFA described previously. Fractions with absorbance at 385 nm were collected. The fractions containing the desired product were lyophilized to afford Compound Ex_4-1 as yellow powder (33 mg, 0.030 mmol, 80%). Rt=1.12 min General Method UPLC. MS (m/z) [M+H]⁺ calc. for C₅₃H₆₉N₉O₁₇ 1104.49, found 1104.70.

### Example 4-2

### Preparation of MP-PEG2-Val-Lys-Gly-7-MAD-MDCPT

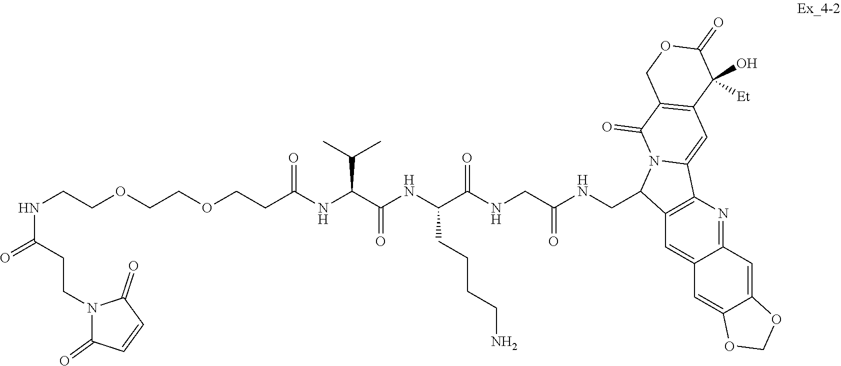 [SMILES: CC[C@@]1(O)C(=O)OCc2c1cc1n(c2=O)C(CNC(=O)CNC(=O)[C@H](CCCCN)NC(=O)[C@@H](NC(=O)CCOCCOCCNC(=O)CCN2C(=O)C=CC2=O)C(C)C)c2cc3cc4c(cc3nc2-1)OCO4]

Compound Ex_4-2 was synthesized using the general procedure described in Example 4-1, by replacing PEG4 with PEG2.

### Example 4-3

### Preparation of MP-PEG8-Val-Lys-Gly-7-MAD-MDCPT

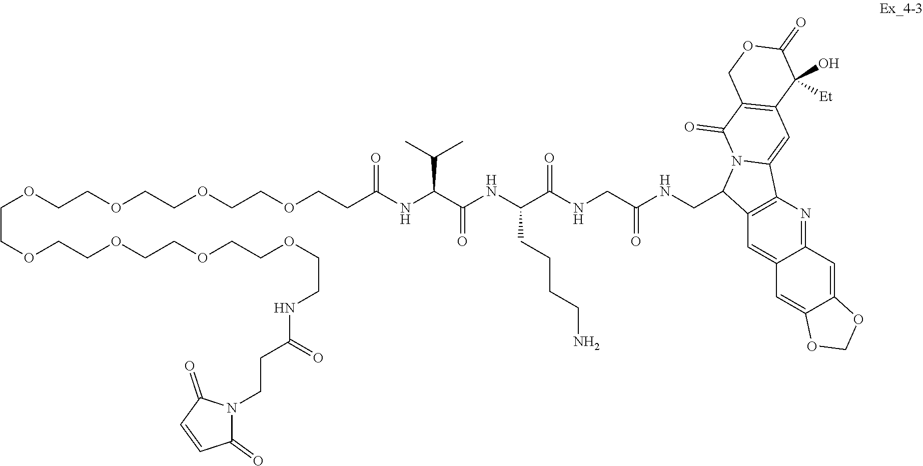 [SMILES: CC[C@@]1(O)C(=O)OCc2c1cc1n(c2=O)C(CNC(=O)CNC(=O)[C@H](CCCCN)NC(=O)[C@@H](NC(=O)CCOCCOCCOCCOCCOCCOCCOCCOCCNC(=O)CCN2C(=O)C=CC2=O)C(C)C)c2cc3cc4c(cc3nc2-1)OCO4]

Compound Ex_4-3 was synthesized using the general procedure described in Example 4-1, by replacing PEG4 with PEG8.

### Example 4-4

### Preparation of MP-PEG12-Val-Lys-Gly-7-MAD-MDCPT

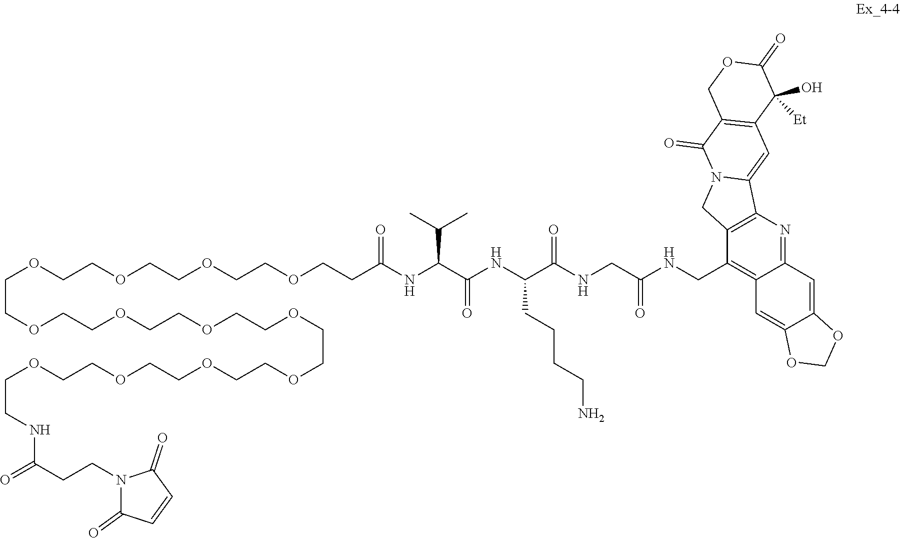 [SMILES: CC[C@@]1(O)C(=O)OCc2c1cc1n(c2=O)Cc2c-1nc1cc3c(cc1c2CNC(=O)CNC(=O)[C@H](CCCCN)NC(=O)[C@@H](NC(=O)CCOCCOCCOCCOCCOCCOCCOCCOCCOCCOCCOCCOCCNC(=O)CCN1C(=O)C=CC1=O)C(C)C)OCO3]

Compound Ex_4-4 was synthesized using the general procedure described in Example 4-1, by replacing PEG4 with PEG12.

The following table summarizes the characterization data for Compounds Ex_4-2, Ex_4-3 and Ex_4-4.

TABLE V

| | Parent Exact | Calc'd MS | Observed | |
|---|---|---|---|---|
| Compound No. | Mass | (m/z) [M + H]⁺ | MS (m/z) | RT |
| Ex_4-2 | 1015.428712 | 1016.44 | 1016.29 | 1.14 |
| Ex_4-3 | 1279.586001 | 1280.60 | 1280.54 | 1.20 |
| Ex_4-4 | 1455.69086 | 1456.70 | 1456.71 | 1.24 |

### Example 4-5

Preparation of MP-Lys[(C(O)(CH₂CH₂O)₁₂—CH₃)]-Val-Lys-Gly-7-MAD-MDCPT

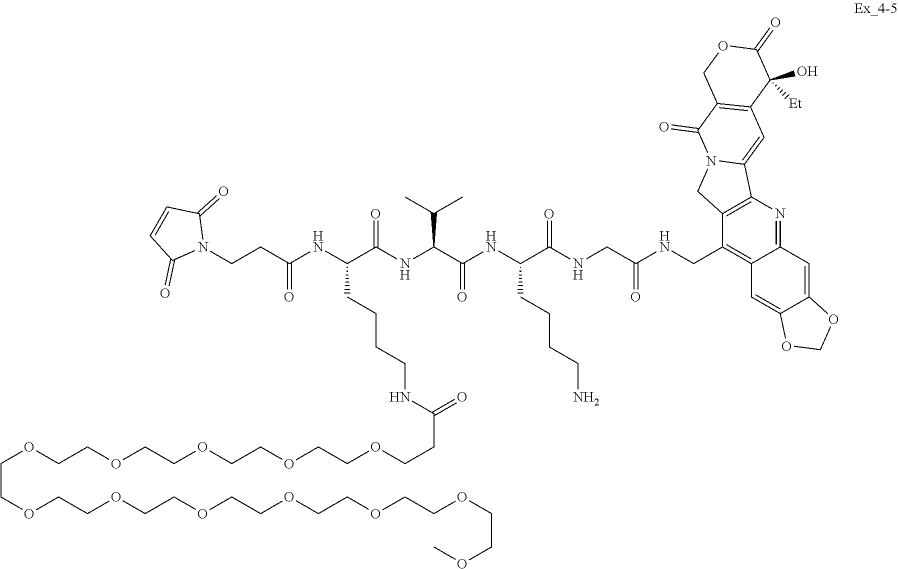 [SMILES: CC[C@@]1(O)C(=O)OCc2c1cc1n(c2=O)Cc2c-1nc1cc3c(cc1c2CNC(=O)CNC(=O)[C@H](CCCCN)NC(=O)[C@@H](NC(=O)[C@H](CCCCNC(=O)CCOCCOCCOCCOCCOCCOCCOCCOCCOCCOCCOCCOC)NC(=O)CCN1C(=O)C=CC1=O)C(C)C)OCO3]

Solid Phase Peptide Synthesis of MP-Lys[(C(O)(CH₂CH₂O)₁₂—CH₃)]-Val-Lys(Boc)-Gly-OH:

Unprotected glycine pre-loaded 0.87 mmol/g on 2-chlorotryityl resin was purchased from Iris Biotech. Resin (0.287 gram, 0.25 mmol) was added to reaction vessel. Resin was washed with DMF 3 times and drained completely. Resin swelled by shaking in DMF for 30 minutes, and drained. Using the general coupling procedure Fmoc-Lys(Boc)-OH was coupled to the resin. The Fmoc was deprotected using the general deprotection procedure. Using the general coupling procedure Fmoc-Val-OH was coupling to the resin, followed by the general deprotection procedure. Fmoc-Lys(PEG12)-OSu (WO 2015057699) was coupled using the general coupling procedure without addition of HATU. The Fmoc was deprotected using the general deprotection procedure. 3-(Maleimido)propionic acid N-hydroxysuccinimide ester was coupled using the general coupling procedure without the addition of HATU. The resin was then washed with DCM 3 times, followed by Et₂O 3 times, and placed under high vacuum overnight. The peptide was cleaved off the resin by stirring the resin in a solution of 1 mL Acetic Acid, 2 mL trifluoroethanol, and 7 mL DCM for 1 hour. Resin was then filtered and rinsed with DCM 3 times, and then the solution was concentrated in vacuo. The crude material was dissolved in DMSO (2 mL) and purified by preparative HPLC using a 21×250 mm Phenomenex Max-RP 4 μm Synergi 80 Å reverse phase column using a 5-60-95% gradient elution of MeCN (0.1% Formic Acid) in aqueous 0.1% Formic acid. Fractions containing the desired product were concentrated to afford a viscous oil. The oil was dissolved in MeCN (2 mL) and precipitated with Et₂O. The product was collected by filtration to afford a colorless amorphous solid (170.7 mg, 0.136 mmol, 55%). Rt=1.32 min General Method UPLC. MS (m/z) [M+H]⁺ calc. for C₅₇H₁₀₂N₇O₂₃ 1252.70, found 1252.79.

### General Fmoc Deprotection Procedure

A solution of 20% piperidine in DMF (5 mL) was added to the resin, shaken for 1 minute, and drained. Another 5 mL of 20% piperidine in DMF was added to the resin, shaken for 30 minutes, and drained. The resin washed with DMF 4 times and drained completely.

### General Coupling Procedure

A solution was prepared in DMF (5 mL) of Fmoc Amino Acid (0.75 mmol), HATU (0.75 mmol), DIPEA (1.5 mmol). The solution was added to the resin, and shaken for 60 minutes. The reaction vessel was drained and washed with DMF 4 times.

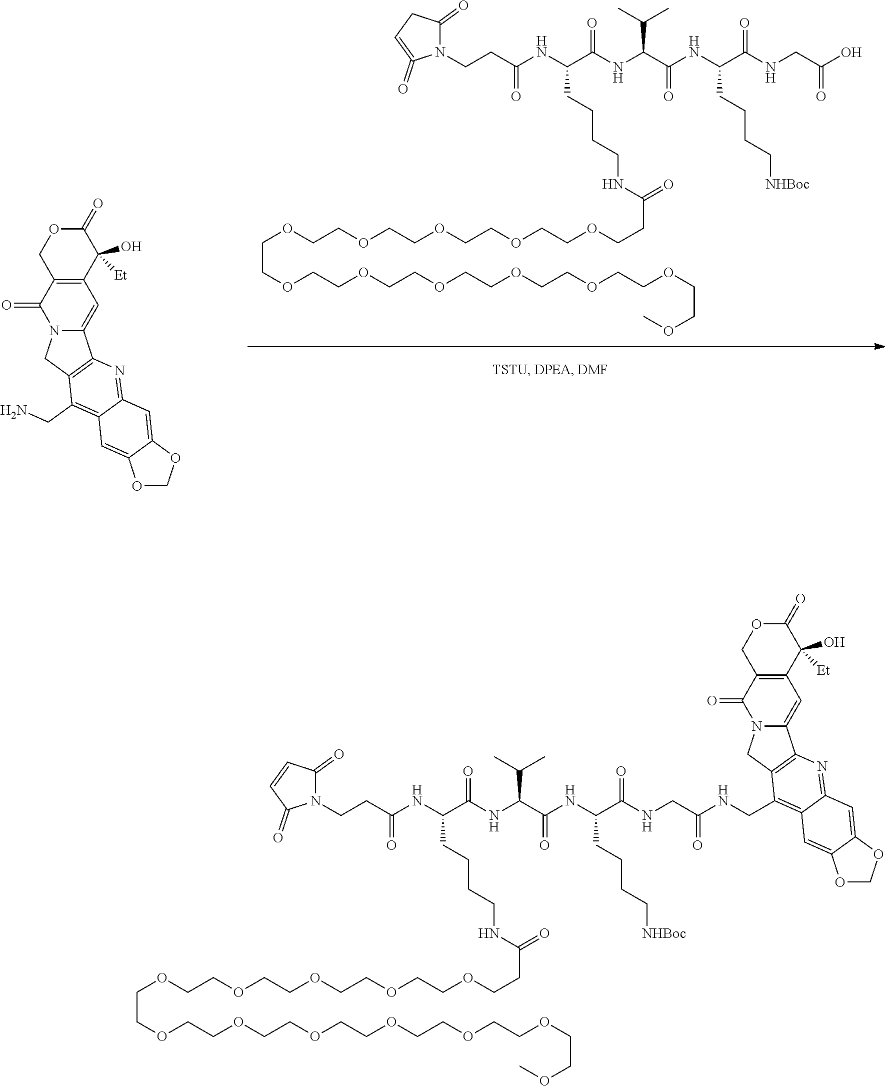

MP-Lys[(C(O)(CH₂CH₂O)₁₂—CH₃)]-Val-Lys(Boc)-Gly-OH (59.4 mg, 0.0475 mmol) was dissolved in anhydrous DMF (0.1 mL). DIPEA (12.4 μL, 0.0712 mmol) was added followed by TSTU (14.3 mg, 0.0475 mmol). The reaction was stirred for 10 minutes to allow for complete activation of the acid to the NHS ester. 7-MAD-MDCPT (10.0 mg, 0.02373 mmol, 100 mg/mL in DMF) was added to the reaction. Complete conversion was observed after 5 minutes. The reaction was quenched with AcOH (25 μL) and purified by prep-HPLC 5-60-95% MeCN in H2O 0.1% TFA. Fractions containing the desired product were concentrated in vacuo to afford a yellow solid (12.3 mg, 0.00740 mmol, 31%). Rt=1.56 min General Method UPLC. MS (m/z) [M+H]⁺ calc. for C₇₉H₁₁₈N₁₀O₂₈ 1655.82, found 1655.89.

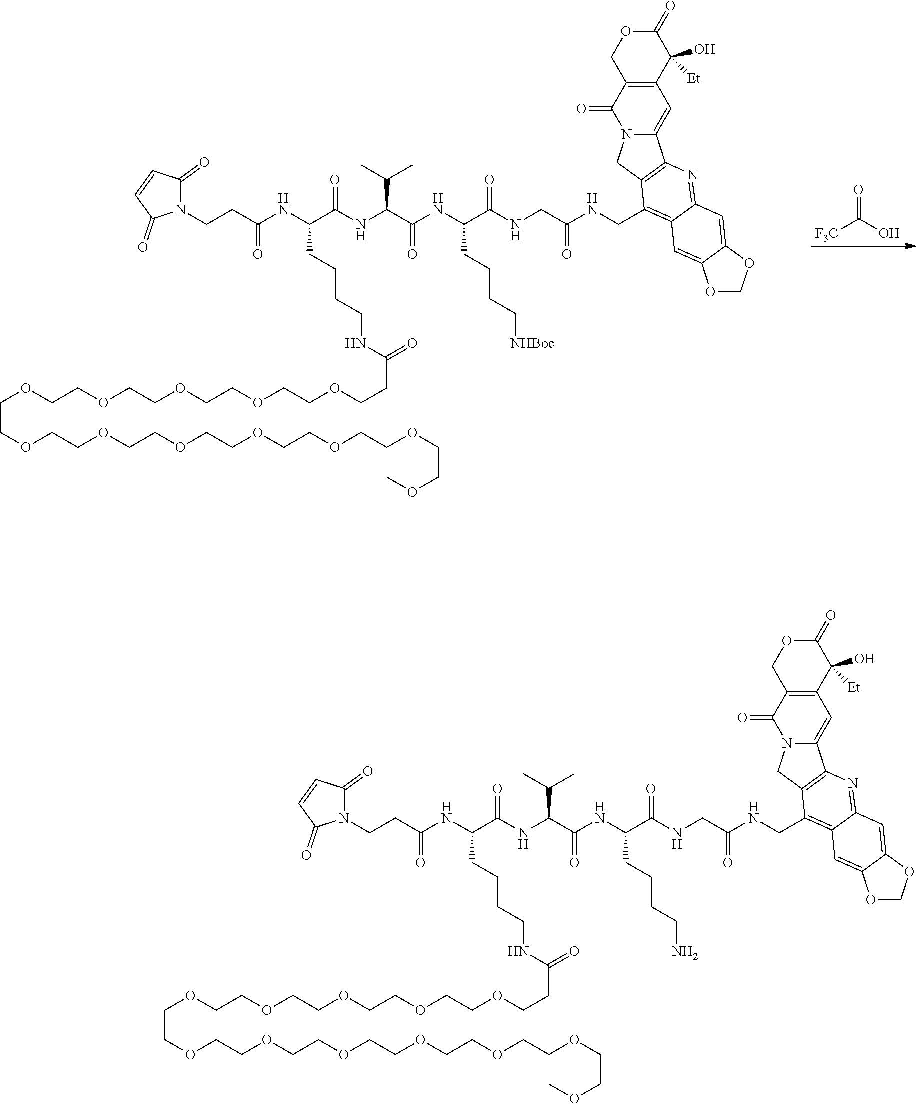 [SMILES: CC[C@@]1(O)C(=O)OCc2c1cc1n(c2=O)Cc2c-1nc1cc3c(cc1c2CNC(=O)CNC(=O)[C@H](CCCCNC(=O)OC(C)(C)C)NC(=O)[C@@H](NC(=O)[C@H](CCCCNC(=O)CCOCCOCCOCCOCCOCCOCCOCCOCCOCCOCCOCCOC)NC(=O)CCN1C(=O)C=CC1=O)C(C)C)OCO3 ; O=C(O)C(F)(F)F ; CC[C@@]1(O)C(=O)OCc2c1cc1n(c2=O)Cc2c-1nc1cc3c(cc1c2CNC(=O)CNC(=O)[C@H](CCCCN)NC(=O)[C@@H](NC(=O)[C@H](CCCCNC(=O)CCOCCOCCOCCOCCOCCOCCOCCOCCOCCOCCOCCOC)NC(=O)CCN1C(=O)C=CC1=O)C(C)C)OCO3]

Compound was dissolved in 20% TFA in DCM. The reaction was monitored for completion by UPLC-MS. Complete conversion was observed after 10 minutes. The reaction was concentrated in vacuo, reconstituted in 40% MeCN in H2O 0.05% TFA and lyophilized to afford compound Ex_4-5 a yellow powder assumed to be the TFA salt (12.99 mg, 0.00778 mmol, 105.16%). Rt=1.27 min General Method UPLC. MS (m/z) [M+H]⁺ calc. for C₇₄H₁₁₁N₁₀O₂₆ 1555.77, found 1555.86.

### Example 5-1

### Preparation of MP-PEG4-Gly-Gly-7-MAD-MDCPT

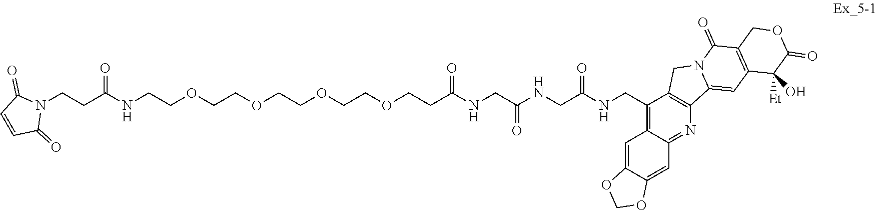 [SMILES: CC[C@@]1(O)C(=O)OCc2c1cc1n(c2=O)Cc2c-1nc1cc3c(cc1c2CNC(=O)CNC(=O)CNC(=O)CCOCCOCCOCCOCCNC(=O)CCN1C(=O)C=CC1=O)OCO3]

The peptide MP-PEG4-Gly-Gly-OH was synthesized by solid phase peptide synthesis using the following general procedure.

### General Procedure for Swelling:

Unprotected amino acid resin (200 mg) pre-loaded 1.1 mmol/g on 2-chlorotryityl resin was purchased from BAChem. Resin was added to reaction vessel. The resin was washed with DMF (4×2 mL) and drained completely. The resin was swelled by shaking in DMF (2 mL) for 30 minutes, and drained.

### General Fmoc Deprotection Procedure:

A solution of 20% piperidine in DMF (2 mL) was added to the resin, shaken for 1 minute, and drained. Another 2 mL of 20% piperidine in DMF was added to the resin, shaken for 30 minutes, and drained. The resin washed with DMF (4×2 mL) and drained completely.

### General Coupling Procedure:

A solution was prepared in DMF (2 mL) of Fmoc Amino Acid (0.6 mmol), HATU (0.6 mmol), DIPEA (0.6 mmol). The solution was added to the resin, and shaken for 60 minutes. The reaction vessel was drained and washed with DMF (4×2 mL) and drained completely.

### General Cleavage Procedure:

The peptide was cleaved off resin by stirring the resin in a solution of 1:2:7 AcOH:hexaflouroisopropanol:DCM (5 mL) for 1 hour. The resin was then filtered and rinsed with DCM (3×10 mL), and then the solution was concentrated in vacuo and purified by preparative HPLC using a 21×250 mm Phenomenex Max-RP 4 μm Synergi 80 Å reverse phase column using a 5-60-95% gradient elution of MeCN (0.05% TFA) in aqueous 0.05% TFA. Fractions containing the desired product were lyophilized to afford a white powder.

Using the general procedure for solid phase peptide synthesis the peptide MP-PEG4-Gly-Gly-OH was synthesized to afford a white powder (45 mg, 0.085 mmol, 42%). Rt=0.83 min General Method UPLC. MS (m/z) [M+H]⁺ calc. for C₂₂H₃₅N₄O₁₁ 531.23, found 530.82.

### TSTU Coupling Procedure:

The peptide (45 mg, 0.085 mmol) was dissolved in 0.2 mL DMF. TSTU (28 mg, 0.093 mmol, 1.1 eq) was added. DIPEA (1.2 eq) was added and the reaction was stirred 30 minutes. The reaction was quenched AcOH. Purified by FCC 0-10% MeOH in DCM. Fractions containing the desired product were concentrated in vacuo to afford a white powder (10 mg, 0.016 mmol, 19%). Rt=0.96 min General Method UPLC. MS (m/z) [M+H]⁺ calc. for C₂₆H₃₈N₅O₁₃ 628.25, found 627.94.

7-MAD-MDCPT (1.1 eq) 20 mg/mL in DMF was added to NHS ester peptide directly. DIPEA was added (18 μL, 0.10 mmol, 1.2 eq) and stirred for 30 minutes. The reaction was quenched with AcOH and purified by prep-HPLC. Fractions containing the desired product were lyophilized to afford a white powder. Rt=1.25 min General Method UPLC. MS (m/z) [M+H]⁺ calc. for C₄₄H₅₂N₇O₁₆ 934.35, found 934.52.

### General Deprotection Procedure:

Peptide based drug linkers with acid labile protecting groups were dissolved in 20% TFA in DCM (2 mL) and stirred for 30 minutes. The reaction was concentrated in vacuo.

Compounds 5-1a to 5-1s were synthesized using the general procedure reported for Example 5-1. The drug moiety in each example is of formula CPT5, linked via an N-linkage at the aminomethyl nitrogen as shown for Example 5-1.

TABLE VI

| Compound | | | | | Camptothecin |
|---|---|---|---|---|---|
| No. | Z′-A | S* or L^{P}(S*) | RL | Y | (N link) |
| Ex_5-1a | Mal-CH₂CH₂C(O)— | —NH(CH₂CH₂O)₄—CH₂CH₂C(O)— | Gly-Gly-Gly | — | 7-MAD-MDCPT |
| Ex_5-1b | Mal-CH₂CH₂C(O)— | —NH(CH₂CH₂O)₄—CH₂CH₂C(O)— | Val-Gly-Gly | — | 7-MAD-MDCPT |
| Ex_5-1c | Mal-CH₂CH₂C(O)— | —NH(CH₂CH₂O)₄—CH₂CH₂C(O)— | Val-Glu-Gly | — | 7-MAD-MDCPT |
| Ex_5-1d | Mal-CH₂CH₂C(O)— | —NH(CH₂CH₂O)₄—CH₂CH₂C(O)— | Val-Gln-Gly | — | 7-MAD-MDCPT |
| Ex_5-1e | Mal-CH₂CH₂C(O)— | —NH(CH₂CH₂O)₄—CH₂CH₂C(O)— | Leu-Lys-Gly | — | 7-MAD-MDCPT |
| Ex_5-1f | Mal-CH₂CH₂C(O)— | —NH(CH₂CH₂O)₄—CH₂CH₂C(O)— | Gly-Val-Lys-Gly | — | 7-MAD-MDCPT |
| Ex_5-1g | Mal-CH₂CH₂C(O)— | —NH(CH₂CH₂O)₄—CH₂CH₂C(O)— | Val-Lys-Gly-Gly | — | 7-MAD-MDCPT |
| Ex_5-1h | Mal-CH₂CH₂C(O)— | —NH(CH₂CH₂O)₄—CH₂CH₂C(O)— | Phe-Lys-Gly | — | 7-MAD-MDCPT |
| Ex_5-1i | Mal-(CH₂)₅C(O)— | — | Val-Lys-Gly | — | 7-MAD-MDCPT |
| Ex_5-1j | Mal-CH₂CH₂C(O)— | —NH(CH₂CH₂O)₄—CH₂CH₂C(O)— | Leu-Leu-Gly | — | 7-MAD-MDCPT |
| Ex_5-1k | Mal-(CH₂)₅C(O)— | — | Gly-Gly-Phe-Gly | — | 7-MAD-MDCPT |
| Ex_5-1l | Mal-(CH₂)₅C(O)— | — | Gly-Gly-Phe-Gly-Gly | — | 7-MAD-MDCPT |
| Ex_5-1m | Mal-CH₂CH₂C(O)— | —NH(CH₂CH₂O)₄—CH₂CH₂C(O)— | Val-Lys-Ala | — | 7-MAD-MDCPT |
| Ex_5-1n | Mal-CH₂CH₂C(O)— | —NH(CH₂CH₂O)₄—CH₂CH₂C(O)— | (Gly)₄ | — | 7-MAD-MDCPT |
| Ex_5-1o | Mal-CH₂CH₂C(O)— | —NH(CH₂CH₂O)₄—CH₂CH₂C(O)— | Val-Cit-Gly | — | 7-MAD-MDCPT |
| Ex_5-1p | Mal-CH₂CH₂C(O)— | —NH(CH₂CH₂O)₄—CH₂CH₂C(O)— | Val-Gly | — | 7-MAD-MDCPT |
| Ex_5-1q | Mal-CH₂CH₂C(O)— | —NH(CH₂CH₂O)₄—CH₂CH₂C(O)— | Val-Lys-β-Ala | — | 7-MAD-MDCPT |
| Ex_5-1r | Mal-CH₂CH₂C(O)— | —NH(CH₂CH₂O)₄—CH₂CH₂C(O)— | Val-Lys-Glu | — | 7-MAD-MDCPT |
| Ex_5-1s | Mal-CH₂CH₂C(O)— | —NH(CH₂CH₂O)₄—CH₂CH₂C(O)— | Val-Glu-Glu | — | 7-MAD-MDCPT |

### Characterization Data:

| Compound | Parent Exact | Calc'd MS | Observed | |
|---|---|---|---|---|
| No. | Mass | (m/z) [M + H]⁺ | MS (m/z) | RT |
| Ex_5-1a | 990.360692 | 991.37 | 991.55 | 1.23 |
| Ex_5-1b | 1032.407643 | 1033.42 | 1033.55 | 1.33 |
| Ex_5-1c | 1104.428772 | 1105.44 | 1105.53 | 1.34 |
| Ex_5-1d | 1103.444756 | 1104.45 | 1104.66 | 1.31 |
| Ex_5-1e | 1117.496792 | 1118.51 | 1118.63 | 1.21 |
| Ex_5-1f | 1160.502606 | 1161.51 | 1161.31 | 1.13 |
| Ex_5-1g | 1160.502606 | 1161.51 | 1161.70 | 1.11 |
| Ex_5-1h | 1151.481142 | 1152.49 | 1152.68 | 1.22 |
| Ex_5-1i | 898.386119 | 899.40 | 899.31 | 1.23 |
| Ex_5-1j | 1102.485893 | 1103.50 | 1103.40 | 1.60 |
| Ex_5-1k | 932.334084 | 933.34 | 933.16 | 1.51 |
| Ex_5-1l | 989.355547 | 990.37 | 990.58 | 1.46 |
| Ex_5-1m | 1117.496792 | 1118.51 | 1118.63 | 1.18 |
| Ex_5-1n | 1047.382156 | 1048.39 | 1048.49 | 1.23 |
| Ex_5-1o | 1132.471305 | 1133.48 | 1133.57 | 1.30 |
| Ex_5-1p | 975.386179 | 976.40 | 976.61 | 1.37 |
| Ex_5-1q | 1117.496792 | 1118.51 | 1118.63 | 0.99 |
| Ex_5-1r | 1175.502271 | 1176.51 | 1176.41 | 1.11 |
| Ex_5-1s | 1176.449901 | 1177.46 | 1177.03 | 1.24 |

### Example 6-1

Preparation of MC-Gly-Gly-Phe-Gly-7-NHCH₂OCH₂-MDCPT

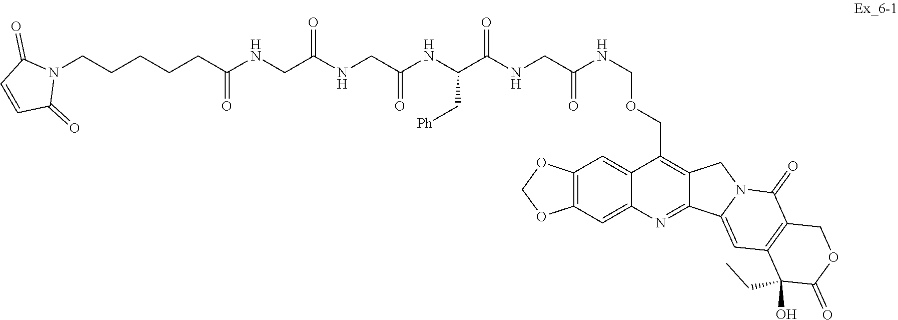 [SMILES: CC[C@@]1(O)C(=O)OCc2c1cc1n(c2=O)Cc2c-1nc1cc3c(cc1c2COCNC(=O)CNC(=O)[C@H](Cc1ccccc1)NC(=O)CNC(=O)CNC(=O)CCCCCN1C(=O)C=CC1=O)OCO3]

### Solid Phase Peptide Synthesis of MC-Gly-Gly-Phe-OH

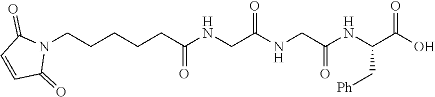 [SMILES: O=C(CCCCCN1C(=O)C=CC1=O)NCC(=O)NCC(=O)N[C@@H](Cc1ccccc1)C(=O)O]

Unprotected phenylalanine pre-loaded 1.1 mmol/g on 2-chlorotryityl resin was purchased from BAChem. Resin (1 gram) was added to reaction vessel. Resin washed with DMF 4 times and drained completely. Resin was swelled by shaking in DMF for 30 minutes, and drained. Using the general coupling procedure Fmoc-Gly-OH was coupled to the resin. The Fmoc was deprotected using the general deprotection procedure. Using the general coupling procedure Fmoc-Gly-OH was coupled to the resin, followed by the general deprotection procedure. MC-OH was coupled using the general coupling procedure. The resin was then washed with DCM 3 times, followed by MeOH 3 times, and placed under high vacuum overnight. The peptide was cleaved off resin by stirring the resin in a solution of 1 mL Acetic Acid, 2 mL hexaflouroisopropanol, and 7 mL DCM for 1 hour. The resin was then filtered and rinsed with DCM 3 times, and the solution was concentrated in vacuo. The white solid was purified by preparative HPLC using a 30×250 mm Phenomenex Max-RP 4 μm Synergi 80 Å reverse phase column using a 5-60-95% gradient elution of MeCN (0.05% TFA) in aqueous 0.05% TFA. Fractions containing the desired product were lyophilized to afford a white powder (207 mg, 0.438 mmol, 44%). Rt=1.28 min General Method UPLC. MS (m/z) [M+H]⁺ calc. for C₂₃H₂₉N₄O₇ 473.20, found 473.00.

Preparation of FmocGly-7-NHCH₂OCH₂-MDCPT

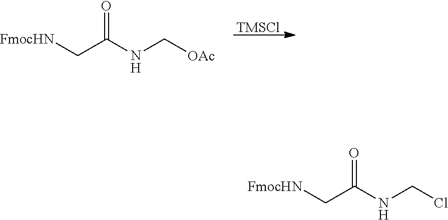

Substrate (52 mg, 0.014 mmol) was dissolved in DCM (1 mL). TMSCl (0.25 mL) was added. The reaction mixture was stirred for 20 minutes then concentrated in vacuo. The crude product was used immediately in the next step.

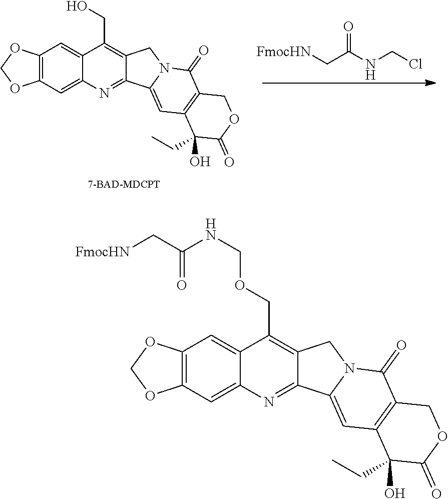

The activated linker from the previous step was dissolved in anhydrous DCM (1 mL) and added directly to 7-BAD-MDCPT (20.0 mg, 0.0474 mmol) solid. The reaction vessel was sealed at stirred at 60° C. for 24 hours. The reaction was quenched with MeOH and concentrated in vacuo. The crude product was purified by column chromatography 10G Biotage Ultra 0-10% MeOH in DCM. Fractions containing the desired product and free drug impurity were concentrated in vacuo to afford a yellow solid (25 mg, 50% purity 0.017 mmol, 36%). Rt=1.77 min General Method UPLC. MS (m/z) [M+H]⁺ calc. for C₄₀H₃₅N₄O₁₀ 731.24, found 731.07.

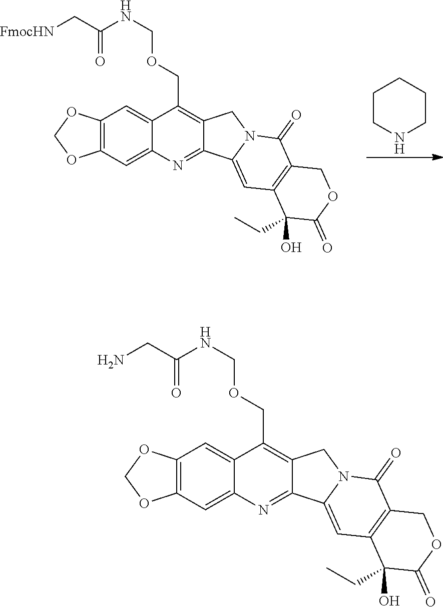

Substrate (0.017 mmol) was dissolved in 20% piperidine in DMF (1 mL). The reaction was stirred for 5 minutes then concentrated in vacuo. The reaction was purified by Prep-HPLC 21 mm 10-95% MeCN in H₂O 0.05% TFA. Fractions containing the desired product were lyophilized to afford a yellow solid (5.2 mg, 0.010 mmol, 60%). Rt=1.02 min General Method UPLC. MS (m/z) [M+H]⁺ calc. for C₂₅H₂₄N₄O₈ 509.17, found 509.00.

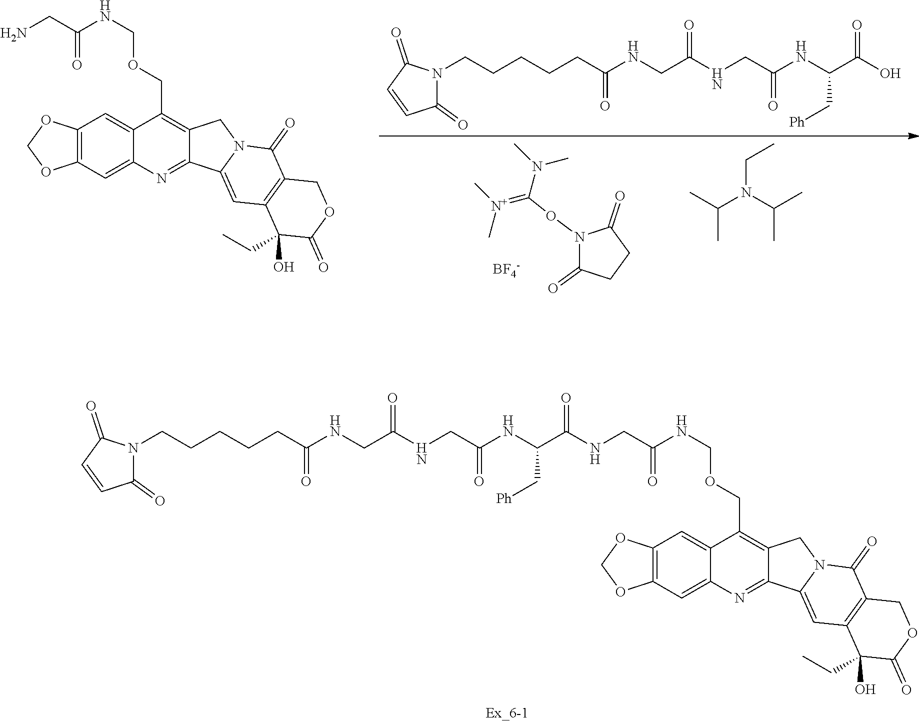

MC-GGFG-OH (14.5 mg, 0.0307 mmol) was dissolved in DMF (0.5 mL). DIPEA (9 μL, 0.05 mmol) was added followed by TSTU (9.3 mg, 0.031 mol). The reaction was stirred for 5 minutes. Complete conversion to the NHS ester product observed by UPLC-MS. The activated NHS ester solution was added directly to Drug-Gly solid. Complete conversion observed by UPLC-MS after 5 minutes. The reaction was quenched with AcOH and purified by Prep-HPLC. Fractions containing the desired product were lyophilized to afford a yellow powder (3.30 mg, 3.43 μmol, 34%). Rt=1.53 min General Method UPLC. MS (m/z) [M+H]⁺ calc. for C₄₈H₅₁N₈O₁₄ 963.35, found 963.14.

### Example 7-1

### Preparation of MP-PEG4-Val-Lys-PABA-7-MAD-MDCPT

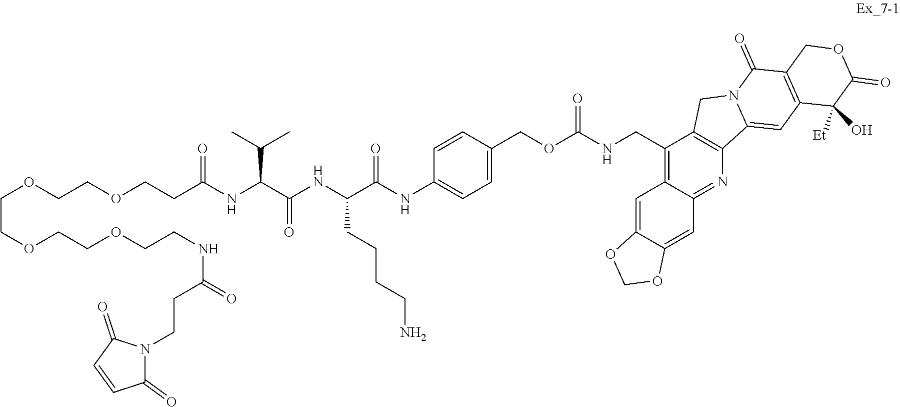 [SMILES: CC[C@@]1(O)C(=O)OCc2c1cc1n(c2=O)Cc2c-1nc1cc3c(cc1c2CNC(=O)OCc1ccc(NC(=O)[C@H](CCCCN)NC(=O)[C@@H](NC(=O)CCOCCOCCOCCOCCNC(=O)CCN2C(=O)C=CC2=O)C(C)C)cc1)OCO3]

### EEDQ Coupling Fmoc-Lys(Boc)-PABA

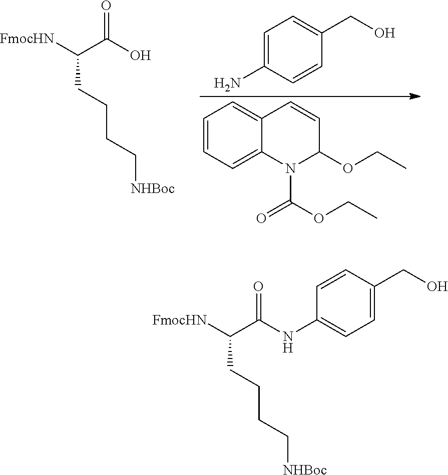

Fmoc-Lys(Boc)-OH (500 mg, 1.07 mmol) suspended in 1 mL DCM and stirred. EEDQ (528 mg, 2.13 mmol) added followed by PABA (263 mg, 2.13 mmol). Reactants became soluble after 1 minute and then precipitated out of mixture after 10 minutes. Complete conversion was observed by UPLC-MS. Precipitate filtered, and washed with DCM (3×50 mL). Desired product was obtained as a white solid (612 mg, 1.07 mmol, quantitative). Used in next step without further purification. Rt=2.08 min General Method UPLC. MS (m/z) [M+H]⁺ calc. for C₃₃H₄₀N₃O₆ 574.29, found 574.28.

### Deprotection

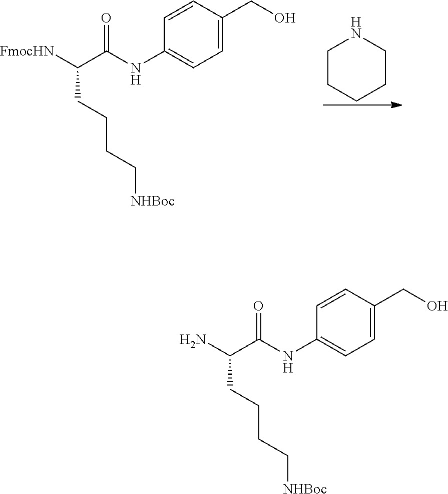

Substrate (612 mg, 1.07 mmol) dissolved in 5 mL of a 20% piperidine in DMF solution. The reaction was stirred for 10 minutes at room temperature. Complete conversion was observed by UPLC-MS. The reaction was concentrated in vacuo, and used in the next step without further purification. Rt=0.80 min General Method UPLC. MS (m/z) [M+H]⁺ calc. for C₁₈H₃₀N₃O₄ 352.22, found 351.69.

### Fmoc-Val-OSu Coupling

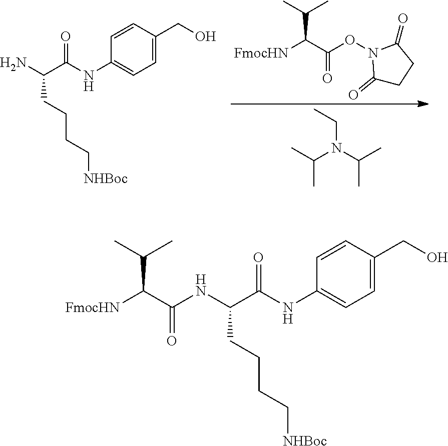

Crude substrate (1.07 mmol) from previous step was dissolved in DMF (2 mL). Fmoc-Val-OSu (581 mg, 1.33 mmol) added followed by DIPEA (0.37 mL, 2.13 mmol) and stirred for 30 minutes. Complete conversion was observed by UPLC-MS. The reaction was quenched with AcOH, concentrated in vacuo, and purified by FCC 100G KP-Sil 0-10% MeOH in DCM. Fractions containing the desired product were concentrated in vacuo to afford a white solid (716 mg, 1.06 mmol, 99%). Rt=2.12 min General Method UPLC. MS (m/z) [M+H]⁺ calc. for C₃₈H₄₉N₄O₇ 673.36, found 673.31.

### Deprotection

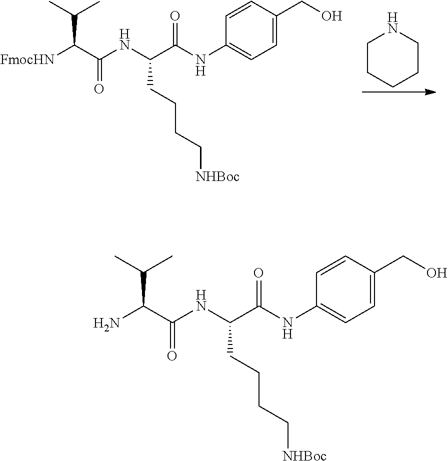

Substrate (716 mg, 1.06 mmol) dissolved in 5 mL of a 20% piperidine in DMF solution. The reaction was stirred for 10 minutes at room temperature. Complete conversion was observed by UPLC-MS. The reaction was concentrated in vacuo, and used in the next step without further purification. Rt=0.94 min General Method UPLC. MS (m/z) [M+H]⁺ calc. for C₂₃H₃₉N₄O₅ 451.29, found 450.72.

### MP-PEG4-OSu Coupling

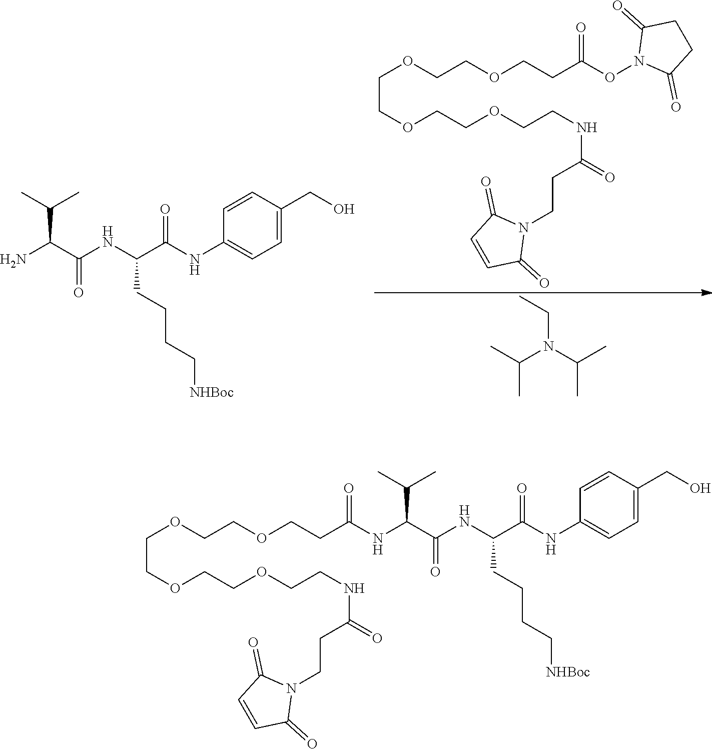

Crude substrate (1.06 mmol) from previous step was dissolved in DMF (1 mL). MP-PEG4-OSu (1.09 mg, 2.13 mmol) was added followed by DIPEA (0.55 mL, 3.19 mmol) and stirred for 30 minutes. Complete conversion was observed by UPLC-MS. Crude reaction mixture was used in the next step. Rt=1.40 min General Method UPLC. MS (m/z) [M+H]⁺ calc. for C₄₁H₆₅N₆O₁₃ 849.46, found 849.06.

### PNP Activation

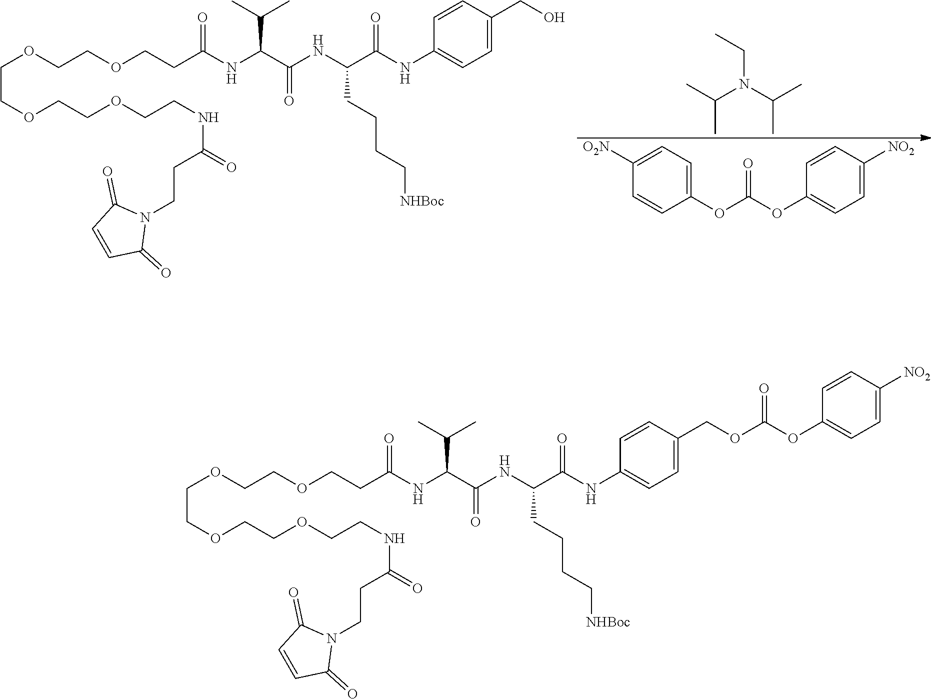

To the crude reaction mixture from the previous was added bis-nitrophenol carbonate (969 mg, 3.19 mmol). The reaction was stirred for 30 minutes. Complete conversion was observed by UPLC-MS. The reaction was quenched with AcOH, and purified by Prep-HPLC 50 mm 10-50-70-95% MeCN in H₂O 0.05% TFA. Fractions containing the desired product were concentrated in vacuo using the HPLC lyo method on the Genevac. Concentrated fractions yielded a white solid (621 mg, 0.612 mmol, 58%). Rt=1.26 min General Method UPLC. MS (m/z) [M+H]⁺ calc. for C₄₈H₆₈N₇O₁₇ 1014.47, found 1014.25.

### Coupling of 7-MAD-MDCPT

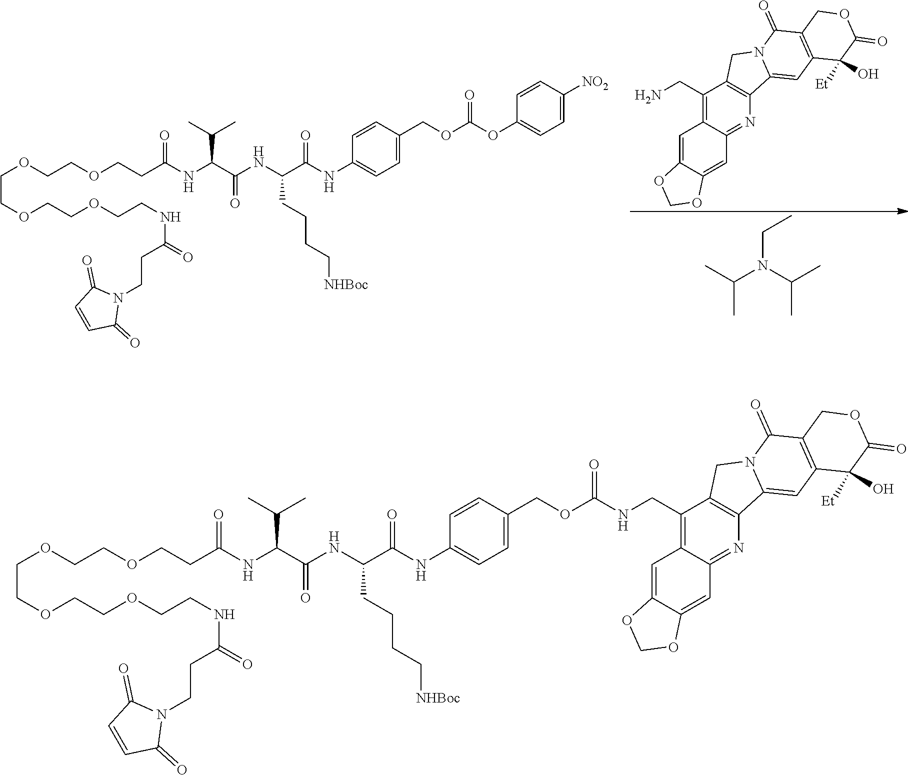

7-MAD-MDCPT (10 mg, 24 mmol) dissolved 50 mg/mL in DMF added directly to activated linker (93 mg, 0.092 mmol). DIPEA (0.047 mL, 36 mmol) was added and the reaction was stirred. The reaction was observed to be slowly progressing to product after 10 minutes. To accelerate the reaction a catalytic amount of DMAP (0.01 mg) was added. Complete conversion was observed by UPLC-MS after 30 minutes. The reaction was quenched with AcOH and purified by prep-HPLC 21 mm 10-36-54-95% MeCN in H₂O 0.05% TFA. Fractions containing the desired product were lyophilized to afford a yellow powder (22.4 mg, 17.3 μmol, 72.5%). Rt=1.66 min General Method UPLC. MS (m/z) [M+H]⁺ calc. for C₆₄H₈₂N₉O₂₀ 1296.57, found 1296.54.

### Boc Deprotection:

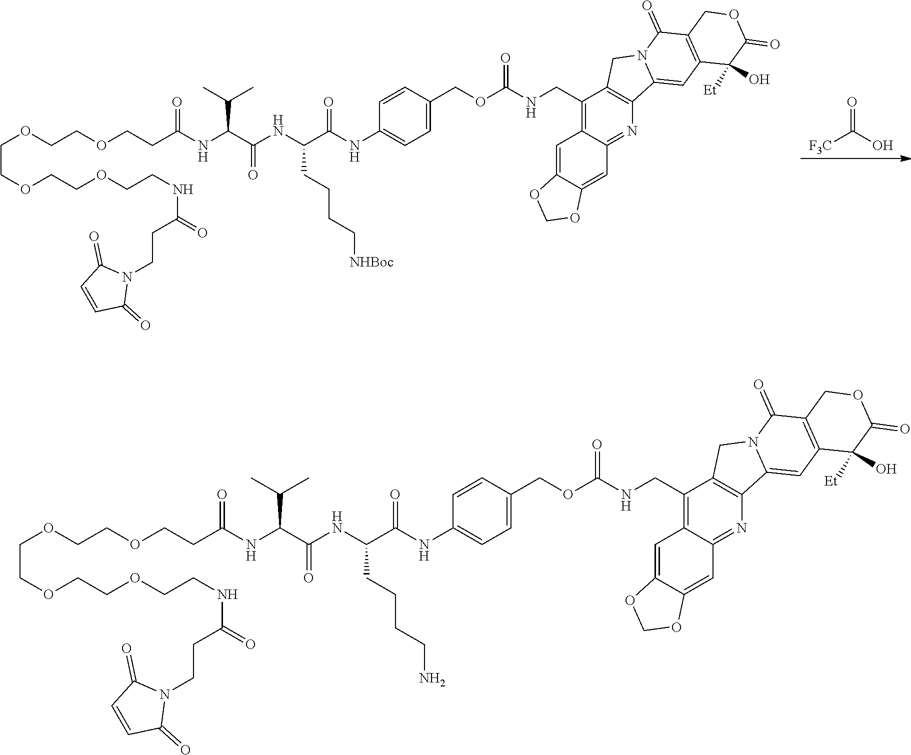 [SMILES: CC[C@@]1(O)C(=O)OCc2c1cc1n(c2=O)Cc2c-1nc1cc3c(cc1c2CNC(=O)OCc1ccc(NC(=O)[C@H](CCCCNC(=O)OC(C)(C)C)NC(=O)[C@@H](NC(=O)CCOCCOCCOCCOCCNC(=O)CCN2C(=O)C=CC2=O)C(C)C)cc1)OCO3 ; O=C(O)C(F)(F)F ; CC[C@@]1(O)C(=O)OCc2c1cc1n(c2=O)Cc2c-1nc1cc3c(cc1c2CNC(=O)OCc1ccc(NC(=O)[C@H](CCCCN)NC(=O)[C@@H](NC(=O)CCOCCOCCOCCOCCNC(=O)CCN2C(=O)C=CC2=O)C(C)C)cc1)OCO3]

Substrate (3.5 mg, 2.7 μmol) was dissolved in 10% TFA in DCM (2 mL). Allowed to stir for 10 minutes at which point nearly complete conversion was observed by UPLC-MS. Reaction was diluted with MeOH (2 mL) and concentrated in vacuo. Reconstituted in 0.3 mL DMSO. No degradation of product was observed after concentration. The reaction was purified by Prep-HPLC 10 mm 5-25-41-95% MeCN in H₂O with 0.05% TFA. Fractions containing the desired product were lyophilized to afford a yellow powder (1.9 mg, 1.6 μmol, 59%). Rt=1.22 min General Method UPLC. MS (m/z) [M+H]⁺ calc. for C₅₉H₇₄N₉O₁₈ 1196.52, found 1196.23.

### Example 8-1

In the Biological Examples and Tables that follow, comparison compounds in this example were prepared and used for evaluation. The structure for those comparison compounds are provided as:

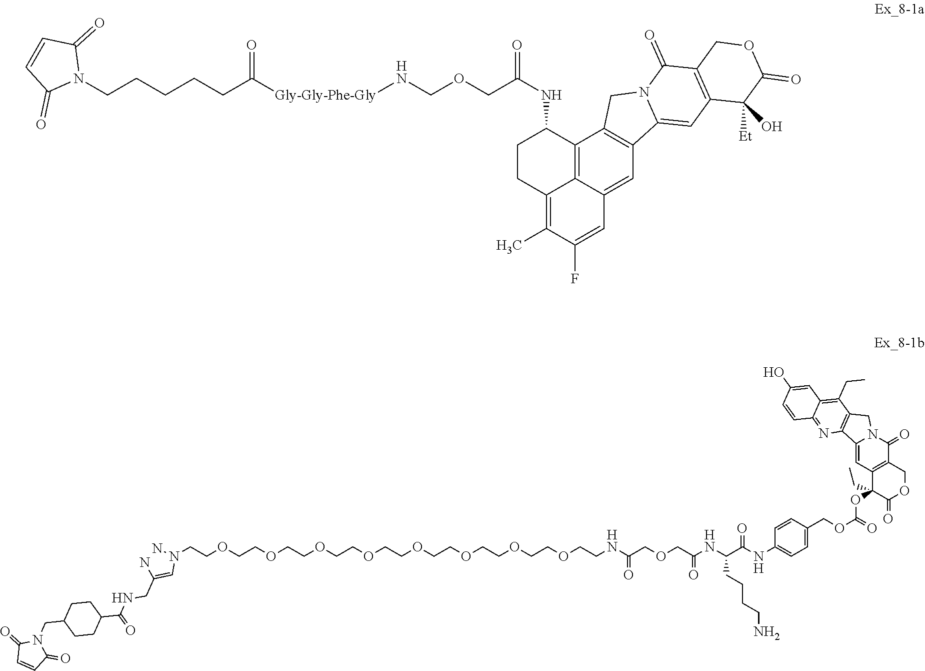

### Example 9-1

Preparation of MP-PEG4-Val-Lys-7-NH(CH₂CH₂O)₂CH₂CH₂NHCH₂-MDCPT

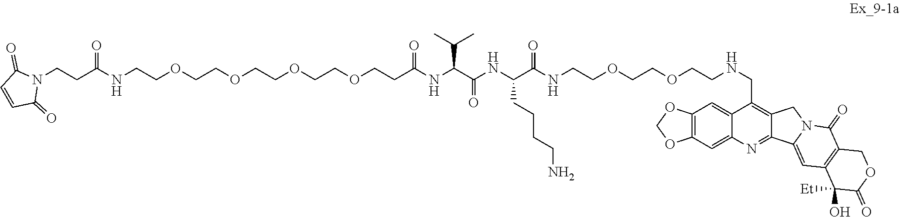 [SMILES: CC[C@@]1(O)C(=O)OCc2c1cc1n(c2=O)Cc2c-1nc1cc3c(cc1c2CNCCOCCOCCNC(=O)[C@H](CCCCN)NC(=O)[C@@H](NC(=O)CCOCCOCCOCCOCCNC(=O)CCN1C(=O)C=CC1=O)C(C)C)OCO3]

MP-PEG4-VK(Boc)-OH peptide (10.0 mg, 0.0181 mmol) was dissolved in anhydrous DMF (0.2 mL). DIPEA (6.3 μL, 0.036 mmol) was added followed by TSTU (5.99 mg, 0.0199 mmol). The acid was allowed to activate to the NHS ester for 20 minutes. The drug (compound 5y) in 0.1 mL DMF was added to the reaction. Complete conversion was observed by UPLC-MS after 5 minutes. The reaction was quenched with AcOH (10 μL), and purified by prep-HPLC 21×250 mm 5-60-95% MeCN in H₂O 0.05% TFA. Fractions containing the desired product were lyophilized to afford a yellow powder (11.62 mg, 9.09 μmol, 50%).

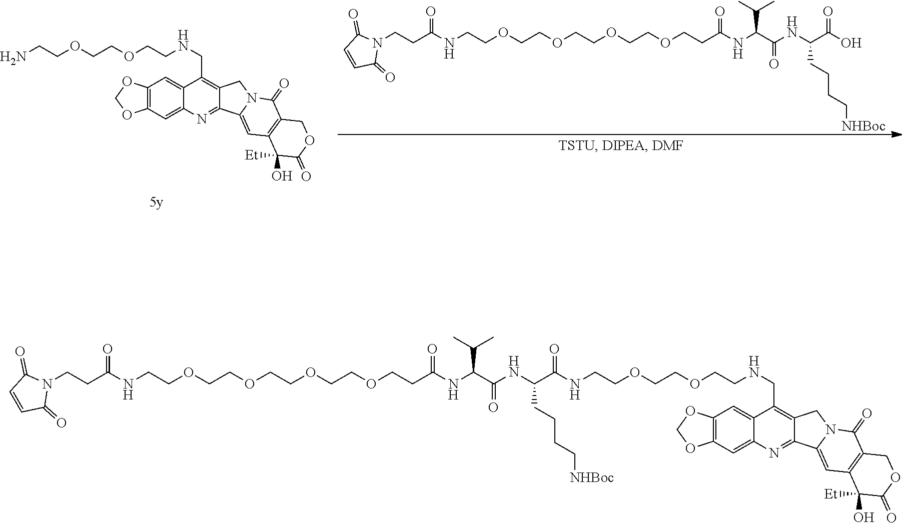 [SMILES: CC[C@@]1(O)C(=O)OCc2c1cc1n(c2=O)Cc2c-1nc1cc3c(cc1c2CNCCOCCOCCN)OCO3 ; CC(C)[C@H](NC(=O)CCOCCOCCOCCOCCNC(=O)CCN1C(=O)C=CC1=O)C(=O)N[C@@H](CCCCNC(=O)OC(C)(C)C)C(=O)O ; CC[C@@]1(O)C(=O)OCc2c1cc1n(c2=O)Cc2c-1nc1cc3c(cc1c2CNCCOCCOCCNC(=O)[C@H](CCCCNC(=O)OC(C)(C)C)NC(=O)[C@@H](NC(=O)CCOCCOCCOCCOCCNC(=O)CCN1C(=O)C=CC1=O)C(C)C)OCO3]

The substrate (11.62 mg, 9.09 μmol) was dissolved in 20% TFA in DCM (2 mL). Complete conversion to the deprotected product was observed by UPLC-MS after 10 minutes. The reaction was concentrated in vacuo and purified by prep-HPLC 10×250 mm MaxRP 5-60-95% MeCN in H2O 0.05% TFA. Fractions containing the desired product were lyophilized to afford a yellow powder (2.96 mg, 2.51 μmol, 28%).

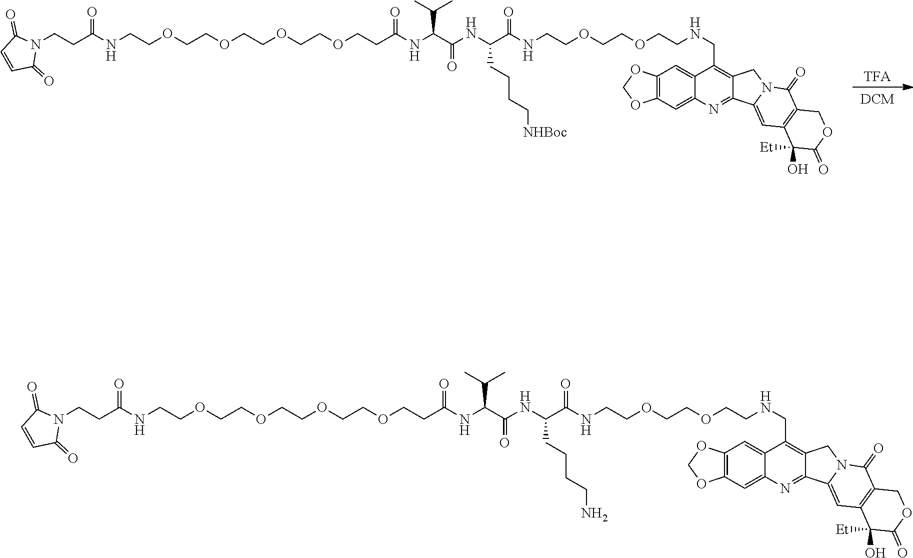 [SMILES: CC[C@@]1(O)C(=O)OCc2c1cc1n(c2=O)Cc2c-1nc1cc3c(cc1c2CNCCOCCOCCNC(=O)[C@H](CCCCNC(=O)OC(C)(C)C)NC(=O)[C@@H](NC(=O)CCOCCOCCOCCOCCNC(=O)CCN1C(=O)C=CC1=O)C(C)C)OCO3 ; CC[C@@]1(O)C(=O)OCc2c1cc1n(c2=O)Cc2c-1nc1cc3c(cc1c2CNCCOCCOCCNC(=O)[C@H](CCCCN)NC(=O)[C@@H](NC(=O)CCOCCOCCOCCOCCNC(=O)CCN1C(=O)C=CC1=O)C(C)C)OCO3]

Preparation of MP-PEG4-Val-Lys-Gly-7-NH(CH₂CH₂O)₂CH₂CH₂NHCH₂-MDCPT

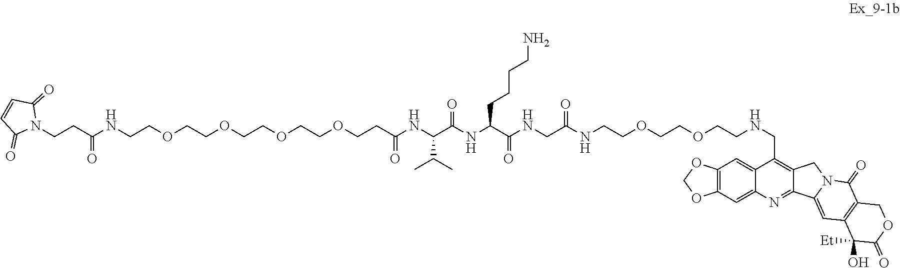 [SMILES: CC[C@@]1(O)C(=O)OCc2c1cc1n(c2=O)Cc2c-1nc1cc3c(cc1c2CNCCOCCOCCNC(=O)CNC(=O)[C@H](CCCCN)NC(=O)[C@@H](NC(=O)CCOCCOCCOCCOCCNC(=O)CCN1C(=O)C=CC1=O)C(C)C)OCO3]

Compound Ex_9-1b was synthesized using the general procedure described above for the preparation of Compound Ex_9-1a.

The following table summarizes the characterization data for Compounds Ex_9-1a and Ex_9-1b.

TABLE VII

| Compound | Parent Exact | Calc'd MS | Observed | |
|---|---|---|---|---|
| No. | Mass | (m/z) [M + H]⁺ | MS (m/z) | RT |
| Ex_9-1a | 1177.554307 | 1178.56 | 1178.68 | 1.06 |
| Ex_9-1b | 1234.57577 | 1235.58 | 1235.52 | 0.99 |

### Example 10-1

Preparation of mDPR-PEG8-Val-Lys-Gly-7-MAD-MDCPT

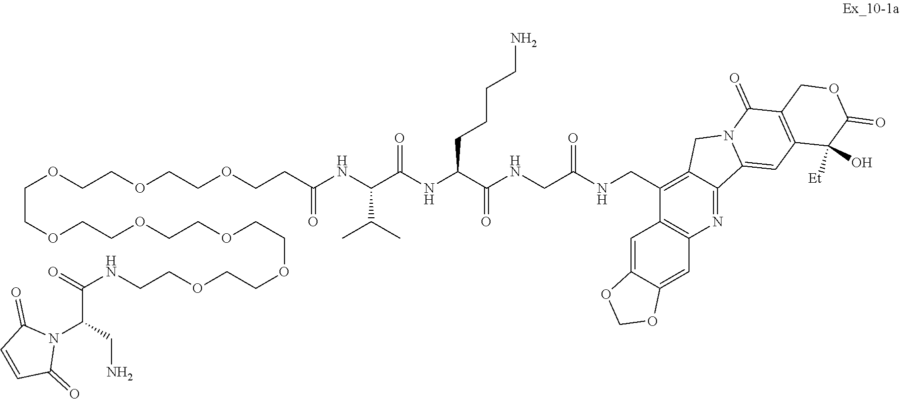 [SMILES: CC[C@@]1(O)C(=O)OCc2c1cc1n(c2=O)Cc2c-1nc1cc3c(cc1c2CNC(=O)CNC(=O)[C@H](CCCCN)NC(=O)[C@@H](NC(=O)CCOCCOCCOCCOCCOCCOCCOCCOCCNC(=O)[C@H](CN)N1C(=O)C=CC1=O)C(C)C)OCO3]

### Solid Phase Peptide Synthesis of Fmoc-VK(Boc)G-OH:

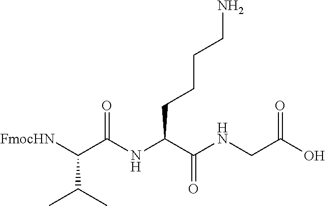

Unprotected glycine pre-loaded 0.87 mmol/g on 2-chlorotryityl resin was purchased from Iris Biotech. Resin (2 gram) was added to reaction vessel. Resin was swelled with DCM for 30 minutes, washed with DMF 3 times and drained completely. Using the general coupling procedure Fmoc-Lys(Boc)-OH was coupled to the resin. The Fmoc was deprotected using the general deprotection procedure. Using the general coupling procedure Fmoc-Val-OH was coupling to the resin. The resin was then washed with DCM 3 times, followed by Et₂O 3 times, and dried under vacuum. The peptide was cleaved off resin by stirring the resin in a solution of 4 mL Acetic Acid, 8 mL trifluoroethanol, and 28 mL DCM for 1 hour. Resin was then filtered and rinsed with DCM 3 times, and then the solution was concentrated in vacuo. The crude residue was dissolved with 2 mL MeCN, and precipitated with 100 mL Et₂O. The precipitate was collected by filtration to afford a white powder (738.6 mg, 1.180 mmol, 68%). Rt=2.06 min General Method UPLC. MS (m/z) [M+H]⁺ calc. for C₃₃H₄₅N₄O₈ 625.32, found 625.30.

### General Fmoc Deprotection Procedure

A solution of 20% piperidine in DMF (20 mL) was added to the resin, shaken for 1 minute, and drained. Another 20 mL of 20% piperidine in DMF was added to the resin, shaken for 30 minutes, and drained. The resin washed with DMF 4 times and drained completely.

### General Coupling Procedure

A solution was prepared in DMF (20 mL) of Fmoc Amino Acid (5 mmol), HATU (5 mmol), DIPEA (5 mmol). The solution was added to the resin and shaken for 60 minutes. The reaction vessel was drained and washed with DMF 4 times.

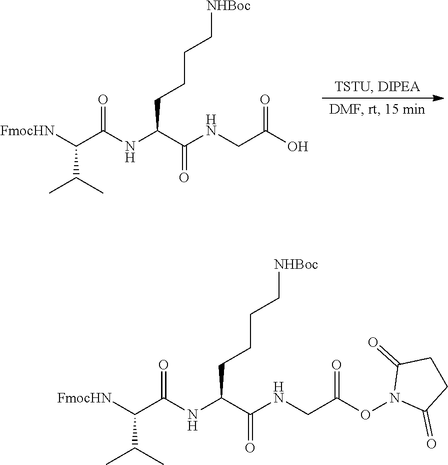

Fmoc-Val-Lys(Boc)-Gly-OH peptide (738.6 mg, 1.180 mmol) was dissolved in anhydrous DMF (4 mL). TSTU (373.7 mg, 1.24 mmol) was added, followed by the DIPEA (0.31 mL, 1.77 mmol). The reaction was stirred at room temperature for 15 minutes at which point complete conversion was observed by UPLC-MS. The reaction was quenched with AcOH (0.20 mL). The reaction was diluted with EtOAc (100 mL), washed with H₂O (3×100 mL), dried MgSO4, filtered and concentrated in vacuo. The residue was resuspended in a minimal amount of DCM (5 mL) and precipitated with Hexanes (100 mL). The precipitate was collected by filtration and dried under vacuum to afford the desired product Fmoc-Val-Lys(Boc)-Gly-OSu as a white powder (759.7 mg, 1.05 mmol, 89%). Rt=2.12 min General Method UPLC. MS (m/z) [M+H]⁺ calc. for C₃₇H₄₈N₅O₁₀ 722.34, found 722.39.

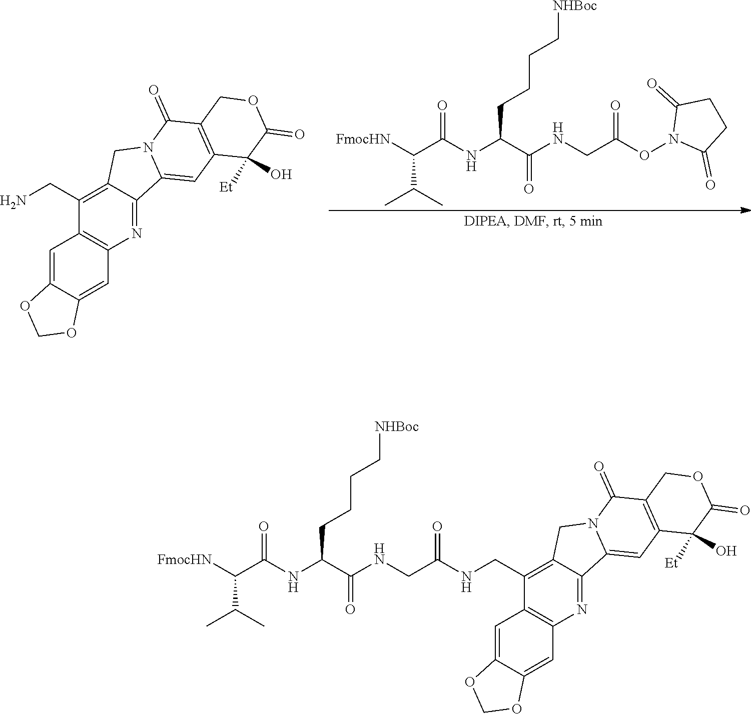

7-MAD-MDCPT (50.0 mg, 0.118 mmoL) was dissolved in anhydrous DMF (1 mL). Fmoc-Val-Lys(Boc)-Gly-OSu (129 mg, 0.178 mmol) was added, followed by DIPEA (0.041 mL, 0.24 mmol). The reaction was stirred at room temperature for 5 minutes, at which point complete conversion to desired product was observed by UPLC-MS. The reaction was concentrated in vacuo and purified by FCC 10G Biotage Ultra 0-6% MeOH in DCM. Fractions containing the desired product were concentrated in vacuo to afford the desired product Fmoc-Val-Lys(Boc)-Gly-7-MAD-MDCPT as a tan solid (97.9 mg, 0.0953 mmol, 80%). Rt=2.07 min General Method UPLC. MS (m/z) [M+H]⁺ calc. for C₅₆H₆₃N₆O₁₃ 1028.44, found 1028.22.

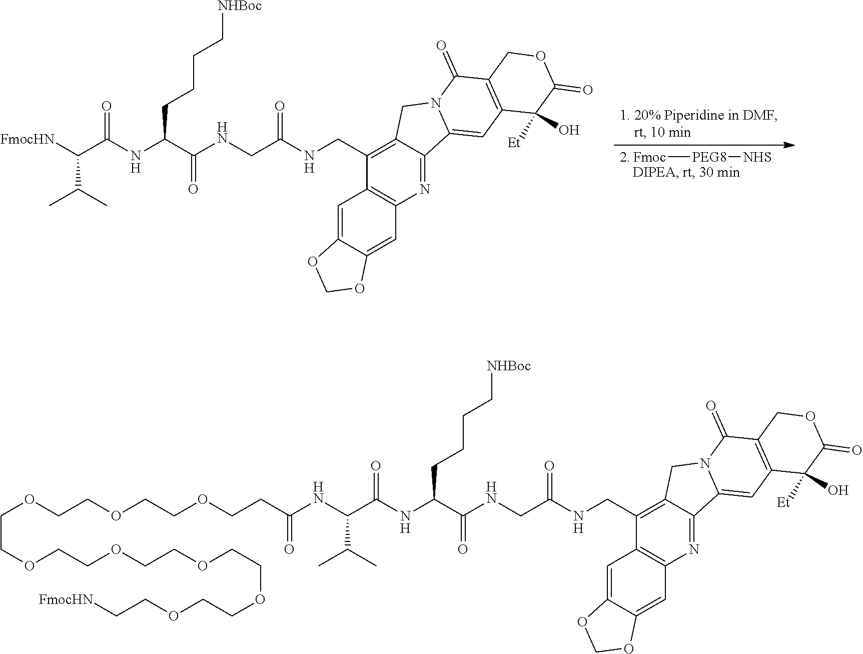

Fmoc-Val-Lys(Boc)-Gly-7-MAD-MDCPT (97.9 mg, 0.0953 mmol) was dissolved in 20% piperidine in DMF. The reaction was stirred at room temperature for 10 minutes. Complete conversion to the Fmoc deprotected product was observed by UPLC-MS. The reaction was concentrated in vacuo to afford the desired H-Val-Lys(Boc)-Gly-7-MAD-MDCPT as a tan solid, which was dissolved in anhydrous DMF (0.5 mL). Fmoc-PEG8-NHS (90.6 mg, 0.119 mmol, Broadpharm: BP-21634, CAS: 1334170-03-4) was added to the reaction, followed by DIPEA (0.025 mL, 0.143 mmol). The reaction was stirred at room temperature for 30 minutes at which point complete conversion was observed by UPLC-MS. The reaction was quenched with AcOH (0.025 mL) and purified by prep-HPLC 21×250 mm Max-RP 5-40-95% MeCN in H2O 0.1% TFA in Formic Acid. Fractions containing the desired product were concentrated to afford the desired product Fmoc-PEG8-Val-Lys(Boc)-Gly-7-MAD-MDCPT as a tan solid (53.2 mg, 0.0367 mmol, 38% over 2 steps). Rt=1.32 min Hydrophobic Method UPLC. MS (m/z) [M+H]⁺ calc. for C₇₄H₉₉N₈O₂₂ 1451.69, found 1452.15.

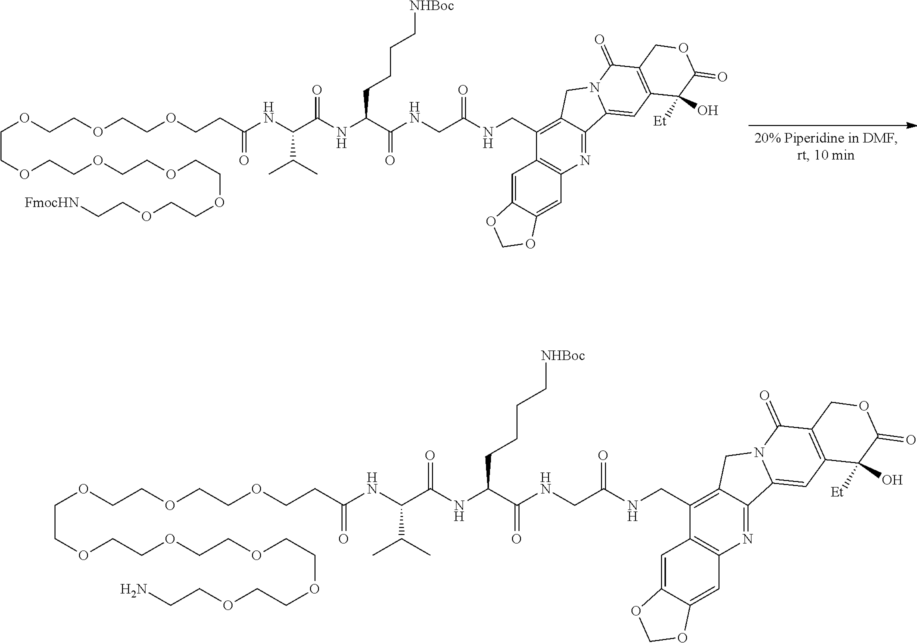

Fmoc-PEG8-Val-Lys(Boc)-Gly-7-MAD-MDCPT (53.2 mg, 0.0367 mmol) was dissolved in 20% piperidine in DMF. The reaction was stirred at room temperature for 10 minutes at which point complete conversion was observed by UPLC-MS. The reaction was concentrated in vacuo to afford H-PEG8-Val-Lys(Boc)-Gly-7-MAD-MDCPT as a tan solid. A 0.0367 M solution in anhydrous DMF of the crude product was prepared and used as a reagent in the next step to form maleimide analogues.

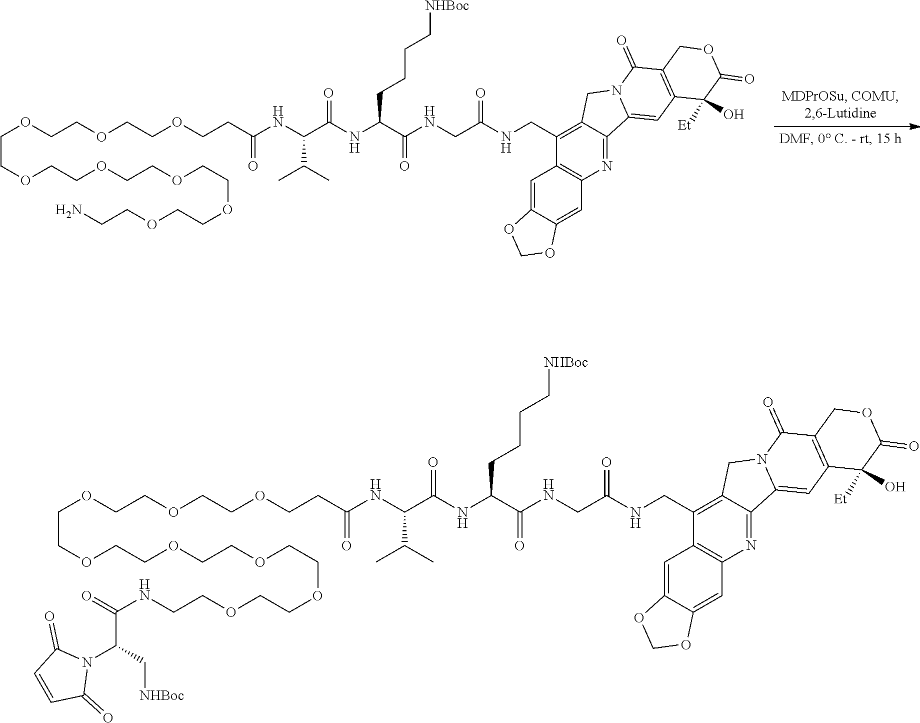 [SMILES: CC[C@@]1(O)C(=O)OCc2c1cc1n(c2=O)Cc2c-1nc1cc3c(cc1c2CNC(=O)CNC(=O)[C@H](CCCCNC(=O)OC(C)(C)C)NC(=O)[C@@H](NC(=O)CCOCCOCCOCCOCCOCCOCCOCCOCCN)C(C)C)OCO3 ; CC[C@@]1(O)C(=O)OCc2c1cc1n(c2=O)Cc2c-1nc1cc3c(cc1c2CNC(=O)CNC(=O)[C@H](CCCCNC(=O)OC(C)(C)C)NC(=O)[C@@H](NC(=O)CCOCCOCCOCCOCCOCCOCCOCCOCCNC(=O)[C@H](CNC(=O)OC(C)(C)C)N1C(=O)C=CC1=O)C(C)C)OCO3]

The crude H-PEG8-Val-Lys(Boc)-Gly-7-MAD-MDCPT 0.0367M in DMF (0.50 mL, 0.018 mmol) was cooled with an ice/water bath. MDPR(Boc)-OH (15.6 mg, 0.0550 mmol, CAS: 1491152-23-8, preparation described in WO 2013173337), and COMU (23.6 mg, 0.0550 mmol) were added to the reaction, followed by 2,6-lutidene (12.8 μL, 0.110 mmol). The reaction was allowed to warm to room temperature over 1 hour and stirred overnight (15 hours). Complete conversion was observed by UPLC-MS. The reaction was quenched by AcOH (0.020 mL) and purified by prep-HPLC 10×250 mm Max-RP 5-60-95% MeCN in H₂O 0.1% Formic Acid. Fractions containing the desired product were concentrated in vacuo to afford mDPR(Boc)-PEG8-Val-Lys(Boc)-Gly-7-MAD-MDCPT as a yellow solid (13.4 mg, 8.97 μmol, 49%). Rt=1.71 min General Method UPLC. MS (m/z) [M+H]⁺ calc. for C₇₁H₁₀₃N₁₀O₂₅ 1495.71, found 1495.04.

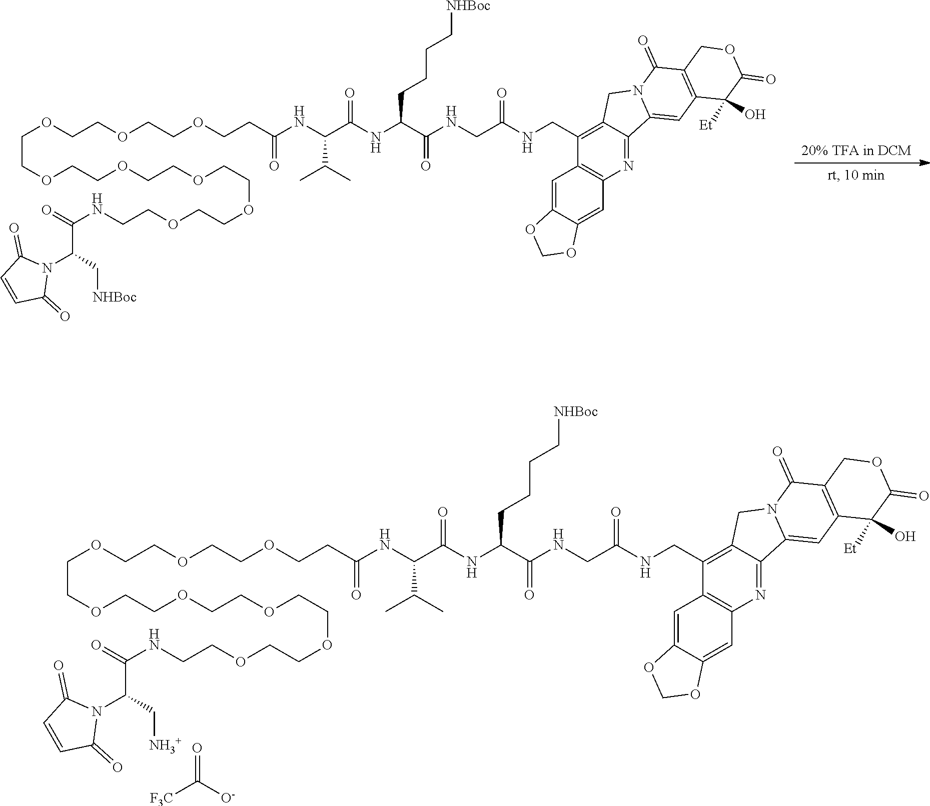 [SMILES: CC[C@@]1(O)C(=O)OCc2c1cc1n(c2=O)Cc2c-1nc1cc3c(cc1c2CNC(=O)CNC(=O)[C@H](CCCCNC(=O)OC(C)(C)C)NC(=O)[C@@H](NC(=O)CCOCCOCCOCCOCCOCCOCCOCCOCCNC(=O)[C@H](CNC(=O)OC(C)(C)C)N1C(=O)C=CC1=O)C(C)C)OCO3 ; CC[C@@]1(O)C(=O)OCc2c1cc1n(c2=O)Cc2c-1nc1cc3c(cc1c2CNC(=O)CNC(=O)[C@H](CCCCNC(=O)OC(C)(C)C)NC(=O)[C@@H](NC(=O)CCOCCOCCOCCOCCOCCOCCOCCOCCNC(=O)[C@H](C[NH3+])N1C(=O)C=CC1=O)C(C)C)OCO3 ; O=C([O-])C(F)(F)F]

MDPR(Boc)-PEG8-Val-Lys(Boc)-Gly-7-MAD-MDCPT (13.4 mg, 8.97 μmol) was dissolved in 20% TFA in DCM and stirred for 10 minutes. Complete conversion was observed by UPLC-MS. The reaction was concentrated in vacuo and purified by prep-HPLC 10×250 mm Max-RP 5-30-95% MeCN in H₂O 0.05% TFA. Fractions containing the desired product were lyophilized to afford mDPR-PEG8-Val-Lys-Gly-7-MAD-MDCPT (Compound Ex_10-1a) as a yellow solid which was presumed to be the double TFA salt (13.4 mg, 8.77 μmol, 98%). Rt=1.06 min General Method UPLC. MS (m/z) [M+Na]⁺ calc. for C₆₁H₈₆N₁₀NaO₂₁ 1317.59, found 1317.50.

### Preparation of MC-PEG8-Val-Lys-Gly-7-MAD-MDCPT

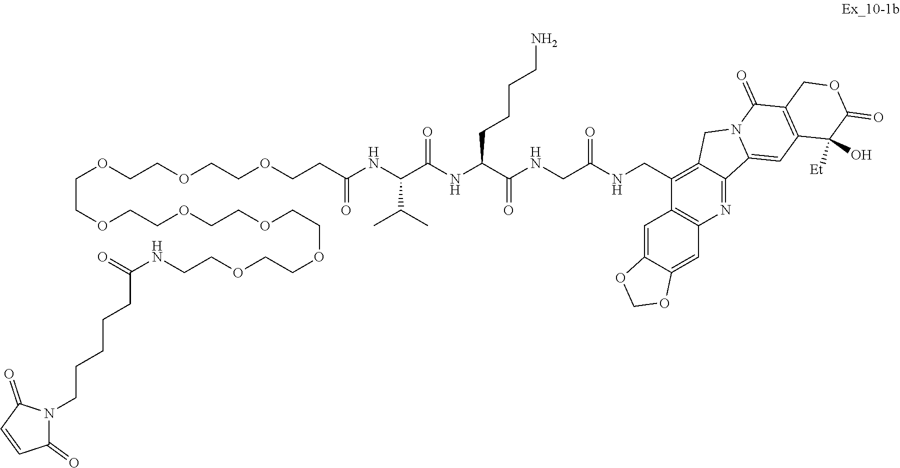 [SMILES: CC[C@@]1(O)C(=O)OCc2c1cc1n(c2=O)Cc2c-1nc1cc3c(cc1c2CNC(=O)CNC(=O)[C@H](CCCCN)NC(=O)[C@@H](NC(=O)CCOCCOCCOCCOCCOCCOCCOCCOCCNC(=O)CCCCCN1C(=O)C=CC1=O)C(C)C)OCO3]

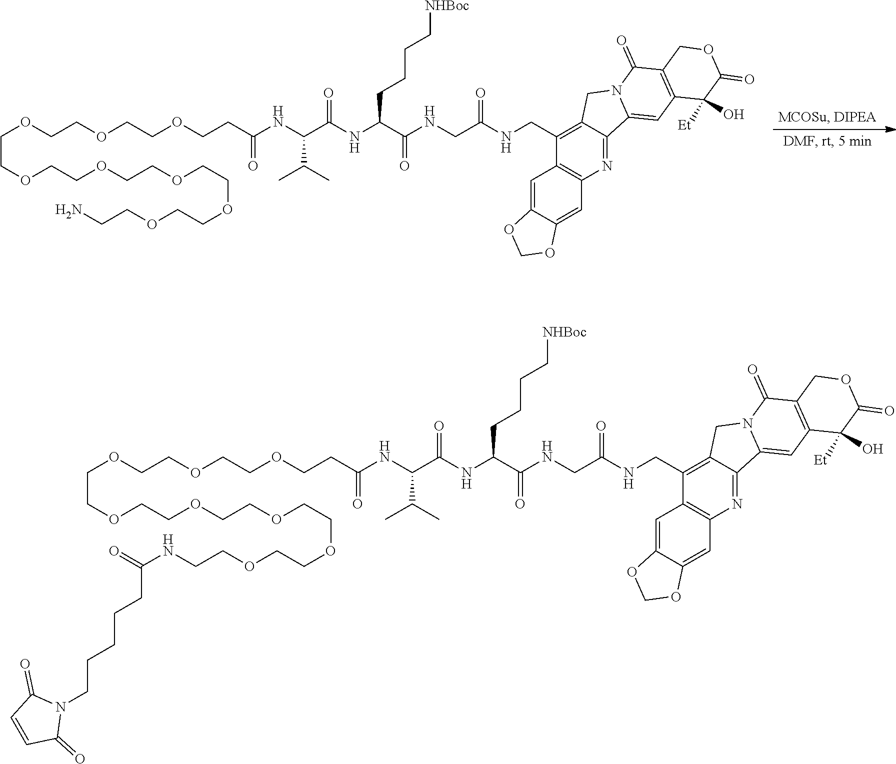 [SMILES: CC[C@@]1(O)C(=O)OCc2c1cc1n(c2=O)Cc2c-1nc1cc3c(cc1c2CNC(=O)CNC(=O)[C@H](CCCCNC(=O)OC(C)(C)C)NC(=O)[C@@H](NC(=O)CCOCCOCCOCCOCCOCCOCCOCCOCCN)C(C)C)OCO3 ; CC[C@@]1(O)C(=O)OCc2c1cc1n(c2=O)Cc2c-1nc1cc3c(cc1c2CNC(=O)CNC(=O)[C@H](CCCCNC(=O)OC(C)(C)C)NC(=O)[C@@H](NC(=O)CCOCCOCCOCCOCCOCCOCCOCCOCCNC(=O)CCCCCN1C(=O)C=CC1=O)C(C)C)OCO3]

To the crude H-PEG8-Val-Lys(Boc)-Gly-7-MAD-MDCPT (from procedure above for the preparation of compound Ex_10-1a) 0.0367M in DMF (0.50 mL, 0.018 mmol) was added MCOSu (17.0 mg, 0.0550 mmol, TCI America: S0428, CAS: 55750-63-5), followed by DIPEA (9.6 μL, 0.055 mmol). The reaction was stirred at room temperature for 5 minutes at which point complete conversion was observed. The reaction was quenched AcOH (0.02 mL), and purified by prep-HPLC 10×250 mm Max-RP 5-60-95% MeCN in H₂O 0.1% Formic Acid. Fractions containing the desired product were concentrated in vacuo to afford MC-PEG8-Val-Lys(Boc)-Gly-7-MAD-MDCPT as a yellow solid (17.4 mg, 18.3 μmol, 67%). Rt=1.63 min General Method UPLC. MS (m/z) [M+H]⁺ calc. for C₆₉H₁₀₀N₉O₂₃ 1422.69, found 1422.27.

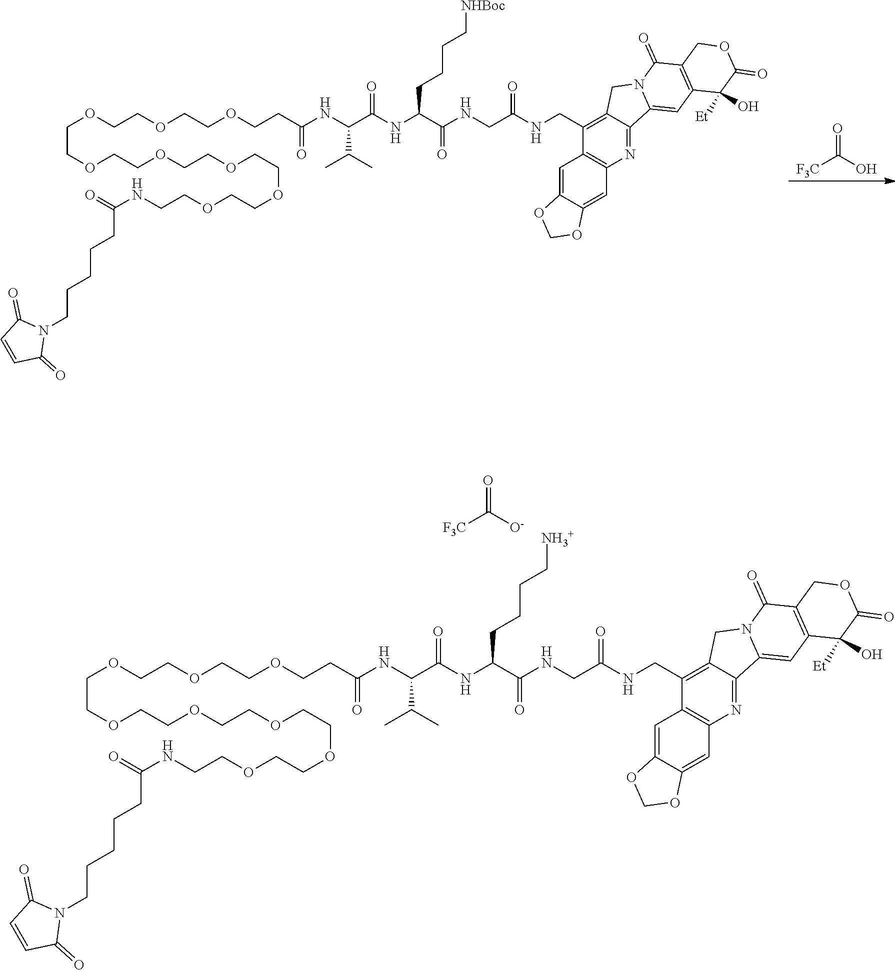 [SMILES: CC[C@@]1(O)C(=O)OCc2c1cc1n(c2=O)Cc2c-1nc1cc3c(cc1c2CNC(=O)CNC(=O)[C@H](CCCCNC(=O)OC(C)(C)C)NC(=O)[C@@H](NC(=O)CCOCCOCCOCCOCCOCCOCCOCCOCCNC(=O)CCCCCN1C(=O)C=CC1=O)C(C)C)OCO3 ; O=C(O)C(F)(F)F ; O=C([O-])C(F)(F)F ; CC[C@@]1(O)C(=O)OCc2c1cc1n(c2=O)Cc2c-1nc1cc3c(cc1c2CNC(=O)CNC(=O)[C@H](CCCC[NH3+])NC(=O)[C@@H](NC(=O)CCOCCOCCOCCOCCOCCOCCOCCOCCNC(=O)CCCCCN1C(=O)C=CC1=O)C(C)C)OCO3]

MC-PEG8-Val-Lys(Boc)-Gly-7-MAD-MDCPT (17.4 mg, 12.2 μmol) was dissolved in 20% TFA in DCM and stirred for 20 minutes. Complete conversion was observed by UPLC-MS. The reaction was concentrated in vacuo and purified by prep-HPLC 10×250 mm Max-RP 5-40-95% MeCN in H₂O 0.05% TFA. Fractions containing the desired product were lyophilized to afford MC-PEG8-Val-Lys-Gly-7-MAD-MDCPT (Compund Ex_10-1b) as a yellow solid which was presumed to be the TFA salt (16.54 mg, 11.52 μmol, 94%). Rt=1.27 min General Method UPLC. MS (m/z) [M+H]⁺ calc. for C₆₄H₉₂N₉O₂₁ 1322.64, found 1322.15.

### Camptothecin Conjugation Method

Fully or partially reduced ADCs were prepared in 50% propylene glycol (PG) 1×PBS mixture. A half portion of the PG was added to reduced mAb, and half PG was added to the 1 mM DMSO camptothecin drug-linker stock. The PG/drug-linker mix was added to reduced mAb in 25% portions. After the addition of drug-linker was complete, excess drug-linker was removed by treating with activated charcoal (1 mg of charcoal to 1 mg of mAb). The charcoal was then removed via filtration, and the resulting ADC was buffer exchanged using a NAP5 or PD10 column, into 5% trehalose in 1×PBS pH 7.4.

### BIOLOGICAL EXAMPLES

### In Vitro Small Molecule and ADC Evaluation

In vitro potency was assessed on multiple cancer cell lines. All cell lines were authenticated by STR profiling at IDEXX Bioresearch and cultured for no more than 2 months after resuscitation. Cells cultured in log-phase growth were seeded for 24 hours in 96-well plates containing 150 μl RPMI 1640 supplemented with 20% FBS. Serial dilutions of antibody-drug conjugates in cell culture media were prepared at 4× working concentrations, and 50 μl of each dilution was added to the 96-well plates. Following addition of test articles, cells were incubated with test articles for 4 days at 37° C. After 96 hours, growth inhibition was assessed by CellTiter-Glo® (Promega, Madison, Wis.) and luminescence was measured on a plate reader. The IC₅₀ value, determined in triplicate, is defined here as the concentration that results in 50% reduction in cell growth relative to untreated controls.

In the following Tables IC₅₀ values for ADCs and CPT free drugs are given in ng/mL and mmol/mL concentrations, respectively, with values in the parenthesis representing percent cells remaining at highest concentration tested (1000 ng/mL for ADCs and 1 μM for CPT free compound, unless otherwise indicated) relative to untreated cells. Cell viability was determined by CellTiter-Glo staining after 96 h exposure to ADC. ND=Not Determined. Ag1 is an antibody targeting a ubiquitous and readily internalizable antigen on cancer cells, Ag2 is cAC10 antibody targeting CD30(+) cancer cells, Ag3 is h1F6 antibody targeting CD70(+) cancer cells, Ag4 is hMEM102 antibody targeting CD48(+) cancer cells, Ag5 is h20F3 antibody targeting NTB-A expressing cancer cells, and h00 is a non-binding control antibody.

Tables 1A-1D. In vitro potency (IC₅₀ values) of camptothecin ADCs (DAR=8). A. anti-Ag1 ADCs targeting renal carcinoma cells (786-O), pancreatic cancer cells (BxPC3), hepatic carcinoma cells (HepG2), acute promyelocytic leukemia cells (HL-60), Hodgkin's lymphoma cells (L540cy), multiple myeloma cells (MM.1R), acute myeloid leukemia cells (MOLM13), Burkitt's lymphoma cells (Ramos), melanoma cells (SK-MEL-5) and B-lymphocyte cancer cells (SU-DHL-4 and U266), B. anti-Ag2 ADCs targeting Hodgkin's lymphoma cells (DEL and L540cy) and non-Hodgkin's lymphoma cells (Karpas 299), which are antigen positive, with testing against renal carcinoma cells (786-O), which are antigen negative, C. anti-Ag3 ADCs targeting renal carcinoma cells (786-O, Caki-1 and UM-RC-3), and Burkitt's lymphoma cells (Raji), and D. anti-Ag4 ADCs and anti-Ag5 ADCs targeting multiple myleoma cells (EJM, KMM-1, MM.1R), and B lymphocyte cancer cells (NCI-H929 and U-266), which are antigen positive, with testing against an antigen negative lymphoblast cell line (TF-1a). Ex_8-1a refers to Ag1-MC-GGFG-NHCH₂-DXd(1) and Ex_4-1 refers to MP-PEG4-VKG-7-MAD-MDCPT.

TABLE 1A
Anti-Ag1 ADCs

| ADC | | | | | | | | | | | | |
|---|---|---|---|---|---|---|---|---|---|---|---|---|
| (antibody-drug) | 786-O | | BxPC3 | | HepG2 | | HL-60 | | L540cy | | MM.1R | |
| Ag1-Ex_4-1 | 9 | (11) | 20 | (41) | 41 | (44) | 81 | (2) | 4 | (1) | 2 | (0) |
| Ag1-Ex_8-1a | 86 | (31) | >1K | (50) | >1K | (ND) | 256 | (16) | 26 | (2) | 13 | (3) |
| Ag1-Ex_9-1a | 625 | (ND) | 329 | (41) | 224 | (35) | 307 | (32) | 53 | (1) | 11 | (1) |
| Ag1-Ex_9-1b | >1K | (50) | 939 | (39) | 490 | (20) | >1K | (51) | 121 | (1) | 19 | (2) |

| ADC | | | | | | | | | | |
|---|---|---|---|---|---|---|---|---|---|---|
| (antibody-drug) | MOLM13 | | Ramos | | SK-MEL-5 | | SU-DHL-4 | | U266 | |
| Ag1-Ex_4-1 | 27 | (0) | 0.1 | (2) | 68 | (35) | 1 | (3) | 15 | (30) |
| Ag1-Ex_8-1a | 89 | (0) | 1 | (1) | 766 | (40) | 12 | (8) | 693 | (45) |
| Ag1-Ex_9-1a | 54 | (0) | 0.5 | (3) | 385 | (47) | 7 | (4) | 192 | (21) |
| Ag1-Ex_9-1b | 80 | (1) | 1 | (4) | >1K | (64) | 11 | (4) | 334 | (37) |

TABLE 1B
Anti-Ag2 ADCs

| ADC | | | | | | | | |
|---|---|---|---|---|---|---|---|---|
| (antibody-drug) | 786-O (ag-) | | DEL | | Karpas 299 | | L540cy | |
| Ag2-Ex_4-1 | >10K | (89) | 2 | (0) | 2 | (9) | 2 | (1) |
| Ag2-Ex_8-1a | >10K | (91) | 5 | (0) | 27 | (25) | 17 | (1) |

TABLE 1C
Anti-Ag3 ADCs

| ADC | | | | | | | | |
|---|---|---|---|---|---|---|---|---|
| (antibody-drug) | 786-O | | Caki-1 | | Raji | | UM-RC-3 | |
| Ag3-Ex_4-1 | 37 | (18) | 36 | (24) | 1130 | (45) | 16 | (30) |
| Ag3-Ex_8-1a | >10K | (54) | >10K | (68) | 5559 | (ND) | 65 | (40) |

TABLE 1D
Anti-Ag4 ADCs and anti-Ag5 ADCs

| ADC* | | | | | | | | |
|---|---|---|---|---|---|---|---|---|
| (antibody/drug) | EJM | | KMM-1 | MM.1R | NCI-H929 | TF-1a (ag-) | U-266 | |
| Ag4-Ex_4-1 | 53 | (27) | 18 (16) | 4 (1) | 6 (2) | >10K (83) | 17 | (38) |
| Ag4-Ex_8-1a | 2785 | (0) | 75 (34) | 9 (0) | 9 (3) | >10K (ND) | >10K | (ND) |
| Ag5-Ex_4-1 | 150 | (31) | 177 (16) | 5 (0) | 80 (2) | >10K (68) | 97 | (28) |
| Ag5-Ex_8-1a | 7192 | (ND) | 5294 (45) | 177 (ND) | 2928 (ND) | >10K (ND) | >10K | (ND) |

### Differential Activity on CD30+ Parental DEL and CD30/MDR+ DEL-BVR Cell Lines

Table 2. Differential activity of camptothecin Ag2-Ex_4-1(DAR=8) on CD30+ parental DEL and CD30/MDR+ DEL-BVR cell lines. The parental DEL lymphoma cell line was cultured in the presence of brentuximab vedotin to induce over-expression of the MDR phenotype, resulting in the DEL brentuximab vedotin resistant line (DEL-BVR). Brentuximab vedotin, which is Ag2-vc-MMAE (DAR=4) was included as a control. Ex_4-1 refers to MP-PEG4-VKG-7-MAD-MDCPT.

| | | |
|---|---|---|
| ADC | | |
| (antibody/drug) | DEL | DEL-BVR |

| | | | | |
|---|---|---|---|---|
| Ag2-Ex_4-1 (8) | 1 | (0) | 4 | (0) |
| Ag2-vcMMAE (4) | 0.5 | (1) | >1000 | (93) |

### Aggregation Levels

Table 3. ADC aggregations levels for peptide-based camptothecin drug-linkers (DAR=4). ADC aggregation was determined by Size Exclusion Chromatography (SEC). Lower levels of aggregation were observed when hydrophilic peptide sequences and/or PEG4 Units were included in peptide-based camptothecin drug-linker constructs.

TABLE 3

| ADC | | |
|---|---|---|
| (antibody- | | % |
| drug) | Drug-linker Description | aggregation |
| Ag1-Ex_5-1 | MP-PEG4-Gly-Gly-7-MAD-MDCPT | 4.37 |
| Ag1-Ex_5-1a | MP-PEG4-Gly-Gly-Gly-7-MAD-MDCPT | 3.22 |
| Ag1-Ex_5-1n | MP-PEG4-Gly-Gly-Gly-Gly-7-MAD-MDCPT | 4.03 |
| Ag1-Ex_5-1b | MP-PEG4-Val-Gly-Gly-7-MAD-MDCPT | 2.88 |
| Ag1-Ex_5-1o | MP-PEG4-Val-Cit-Gly-7-MAD-MDCPT | 6.69 |
| Ag1-Ex_5-1d | MP-PEG4-Val-Gln-Gly-7-MAD-MDCPT | 4.35 |
| Ag1-Ex_5-1c | MP-PEG4-Val-Glu-Gly-7-MAD-MDCPT | 3.07 |
| Ag1-Ex_5-1h | MP-PEG4-Phe-Lys-Gly-7-MAD-MDCPT | 3.1 |
| Ag1-Ex_5-1e | MP-PEG4-Leu-Lys-Gly-7-MAD-MDCPT | 3.14 |
| Ag1-Ex_5-1f | MP-PEG4-Gly-Val-Lys-Gly-7-MAD-MDCPT | 3.32 |
| Ag1-Ex_5-1g | MP-PEG4-Val-Lys-Gly-Gly-7-MAD-MDCPT | 3.3 |
| Ag1-Ex_5-1i | MC-Val-Lys-Gly-7-MAD-MDCPT* | high |
| Ag1-Ex_5-1m | MP-PEG4-Val-Lys-Ala-7-MAD-MDCPT | 3.89 |
| Ag1-Ex_5-1j | MP-PEG4-Leu-Leu-Gly-7-MAD-MDCPT | 7.66 |
| Ag1-Ex_5-1k | MC-Gly-Gly-Phe-Gly-7-MAD-MDCPT | 23.79 |
| Ag1-Ex_5-1l | MC-Gly-Gly-Phe-Gly-Gly-7-MAD-MDCPT | 3.79 |
| Ag1-Ex_5-1p | MP-PEG4-Val-Gly-7-MAD-MDCPT | 5.25 |
| Ag1-Ex_6-1 | MC-GGFG-HAPI-7-BAD-MDCPT | 4.12 |
| Ag1-Ex_5-1q | MP-PEG4-Val-Lys-B-Ala-7-MAD-MDCPT | 3.69 |

DAR less than 4

Table 4. In vitro potency (IC₅₀ values) of peptide-based camptothecin anti-Ag1 DAR4 ADCs against various cancer cell lines demonstrate sequence-dependent potency.

Table 4A. renal cancer cells (786-O), pancreatic cancer cells (BxPC3), hepatic cancer cells (HepG2) and acute promyelocytic leukemia cells (HL-60).

Table 4B. multiple drug resistant acute promyelocytic leukemia cells (HL-60/RV), Hodgkin's lymphoma cells (L540cy), multiple myeloma cells (MM.R1) and acute myeloid leukemia cells (MOLM13).

Table 4C. Burkitt's lymphoma cells (Ramos), melanoma cells (SK-MEL-5) and B-lymphocyte cancer cells (SU-DHL-4 and U266).

TABLE 4A

| ADC | | | | | | | | | |
|---|---|---|---|---|---|---|---|---|---|
| (antibody-drug) | Drug-linker Description | 786-O | | BxPC3 | | HepG2 | | HL-60 | |
| Ag1-Ex_5-1 | MP-PEG4-Gly-Gly-7-MAD-MDCPT | 500 | (45) | 90 | (43) | >1K | (70) | 322 | (30) |
| Ag1-Ex_5-1a | MP-PEG4-Gly-Gly-Gly-7-MAD-MDCPT | >1K | (51) | 136 | (44) | >1K | (61) | 411 | (ND) |
| Ag1-Ex_5-1n | MP-PEG4-Gly-Gly-Gly-Gly-7-MAD-MDCPT | >1K | (51) | >1K | (52) | >1K | (65) | 683 | (ND) |
| Ag1-Ex_5-1b | MP-PEG4-Val-Gly-Gly-7-MAD-MDCPT | >1K | (49) | 142 | (46) | >1K | (65) | 264 | (12) |
| Ag1-Ex_5-1o | MP-PEG4-Val-Cit-Gly-7-MAD-MDCPT | 141 | (41) | 150 | (40) | >1K | (67) | 335 | (1) |
| Ag1-Ex_5-1d | MP-PEG4-Val-Gln-Gly-7-MAD-MDCPT | 194 | (38) | 140 | (48) | >1K | (62) | 252 | (8) |
| Ag1-Ex_5-1c | MP-PEG4-Val-Glu-Gly-7-MAD-MDCPT | 179 | (36) | 269 | (49) | >1K | (57) | 284 | (10) |
| Ag1-Ex_5-1m | MP-PEG4-Val-Lys-Ala-7-MAD-MDCPT | >1K | (63) | 108 | (46) | >1K | (54) | 212 | (5) |
| Ag1-Ex_5-1h | MP-PEG4-Phe-Lys-Gly-7-MAD-MDCPT | 124 | (34) | 87 | (46) | >1K | (62) | 384 | (0) |
| Ag1-Ex_5-1j | MP-PEG4-Leu-Leu-Gly-7-MAD-MDCPT | 91 | (29) | 107 | (48) | >1K | (57) | 164 | (9) |
| Ag1-Ex_5-1k | MC-Gly-Gly-Phe-Gly-7-MAD-MDCPT | 205 | (44) | >1K | (52) | >1K | (57) | 798 | (ND) |
| Ag1-Ex_5-1e | MP-PEG4-Leu-Lys-Gly-7-MAD-MDCPT | 77 | (35) | 192 | (49) | >1K | (57) | 325 | (0) |
| Ag1-Ex_5-1l | MC-Gly-Gly-Phe-Gly-Gly-7-MAD-MDCPT | 89 | (34) | 200 | (48) | >1K | (39) | 238 | (7) |
| Ag1-Ex_5-1f | MP-PEG4-Gly-Val-Lys-Gly-7-MAD-MDCPT | 75 | (30) | >1K | (51) | >1K | (59) | 292 | (9) |
| Ag1-Ex_5-1p | MP-PEG4-Val-Gly-7-MAD-MDCPT | >1K | (85) | >1K | (65) | >1K | (92) | 299 | (22) |
| Ag1-Ex_5-1g | MP-PEG4-Val-Lys-Gly-Gly-7-MAD-MDCPT | 973 | (48) | >1K | (51) | >1K | (57) | 296 | (27) |
| Ag1-Ex_6-1 | MC-GGFG-HAPI-7-BAD-MDCPT | >1K | (86) | >1K | (ND) | >1K | (ND) | >1K | (ND) |
| Ag1-Ex_5-1q | MP-PEG4-Val-Lys-B-Ala-7-MAD-MDCPT | 71 | (29) | 56 | (41) | >1K | (63) | 115 | (3) |

TABLE 4B

| ADC | | | | | | | | | |
|---|---|---|---|---|---|---|---|---|---|
| (antibody-drug) | Drug-linker Description | HL60/RV | | L540cy | | MM.R1 | | MOLM13 | |
| Ag1-Ex_5-1 | MP-PEG4-Gly-Gly-7-MAD-MDCPT | >1K | (96) | 49 | (5) | 13 | (1) | 101 | (1) |
| Ag1-Ex_5-1a | MP-PEG4-Gly-Gly-Gly-7-MAD-MDCPT | >1K | (ND) | 33 | (4) | 11 | (0) | 78 | (2) |
| Ag1-Ex_5-1n | MP-PEG4-Gly-Gly-Gly-Gly-7-MAD-MDCPT | >1K | (85) | 55 | (4) | 14 | (0) | 112 | (1) |
| Ag1-Ex_5-1b | MP-PEG4-Val-Gly-Gly-7-MAD-MDCPT | >1K | (100) | 16 | (3) | 15 | (0) | 85 | (2) |
| Ag1-Ex_5-1o | MP-PEG4-Val-Cit-Gly-7-MAD-MDCPT | >1K | (ND) | 19 | (1) | 16 | (0) | 88 | (0) |
| Ag1-Ex_5-1d | MP-PEG4-Val-Gln-Gly-7-MAD-MDCPT | >1K | (100) | 15 | (2) | 13 | (0) | 69 | (2) |
| Ag1-Ex_5-1c | MP-PEG4-Val-Glu-Gly-7-MAD-MDCPT | >1K | (72) | 13 | (1) | 18 | (2) | 85 | (1) |
| Ag1-Ex_5-1m | MP-PEG4-Val-Lys-Ala-7-MAD-MDCPT | >1K | (ND) | 20 | (2) | 17 | (0) | 78 | (0) |
| Ag1-Ex_5-1h | MP-PEG4-Phe-Lys-Gly-7-MAD-MDCPT | >1K | (100) | 22 | (2) | 25 | (1) | 100 | (1) |
| Ag1-Ex_5-1j | MP-PEG4-Leu-Leu-Gly-7-MAD-MDCPT | >1K | (74) | 13 | (2) | 9 | (0) | 52 | (0) |
| Ag1-Ex_5-1k | MC-Gly-Gly-Phe-Gly-7-MAD-MDCPT | >1K | (41) | 21 | (2) | 15 | (0) | 103 | (0) |
| Ag1-Ex_5-1e | MP-PEG4-Leu-Lys-Gly-7-MAD-MDCPT | >1K | (82) | 15 | (2) | 20 | (1) | 82 | (1) |
| Ag1-Ex_5-1l | MC-Gly-Gly-Phe-Gly-Gly-7-MAD-MDCPT | >1K | (ND) | 16 | (2) | 22 | (1) | 90 | (0) |
| Ag1-Ex_5-1f | MP-PEG4-Gly-Val-Lys-Gly-7-MAD-MDCPT | >1K | (97) | 18 | (2) | 22 | (1) | 93( | 1) |
| Ag1-Ex_5-1p | MP-PEG4-Val-Gly-7-MAD-MDCPT | >1K | (95) | >1K | (88) | 189 | (30) | 154 | (1) |
| Ag1-Ex_5-1g | MP-PEG4-Val-Lys-Gly-Gly-7-MAD-MDCPT | >1K | (ND) | 28 | (3) | 28 | (3) | 108 | (1) |
| Ag1-Ex_6-1 | MC-GGFG-HAPI-7-BAD-MDCPT | >1K | (ND) | 77 | (9) | >1K | (38) | 255 | (16) |
| Ag1-Ex_5-1q | MP-PEG4-Val-Lys-B-Ala-7-MAD-MDCPT | >1K | (89) | 8 | (2) | 4 | (0) | 38 | (0) |

TABLE 4C

| ADC | | | | | | | | | |
|---|---|---|---|---|---|---|---|---|---|
| (antibody-drug) | Drug-linker Description | Ramos | | SK-MEL-5 | | SU-DHL-4 | | U266 | |
| Ag1-Ex_5-1 | MP-PEG4-Gly-Gly-7-MAD-MDCPT | 2 | (4) | >1K | (51) | 11 | (6) | 98 | (27) |
| Ag1-Ex_5-1a | MP-PEG4-Gly-Gly-Gly-7-MAD-MDCPT | 1 | (4) | >1K | (ND) | 11 | (4) | 99 | (33) |
| Ag1-Ex_5-1n | MP-PEG4-Gly-Gly-Gly-Gly-7-MAD-MDCPT | 4 | (4) | >1K | (72) | 13 | (4) | 95 | (38) |
| Ag1-Ex_5-1b | MP-PEG4-Val-Gly-Gly-7-MAD-MDCPT | 1 | (4) | >1K | (ND) | 7 | (2) | 62 | (20) |
| Ag1-Ex_5-1o | MP-PEG4-Val-Cit-Gly-7-MAD-MDCPT | 1 | (3) | >1K | (57) | 8 | (2) | 67 | (23) |
| Ag1-Ex_5-1d | MP-PEG4-Val-Gln-Gly-7-MAD-MDCPT | 1 | (4) | >1K | (ND) | 7 | (3) | 61 | (23) |
| Ag1-Ex_5-1c | MP-PEG4-Val-Glu-Gly-7-MAD-MDCPT | 2 | (5) | >1K | (ND) | 7 | (3) | 63 | (25) |
| Ag1-Ex_5-1m | MP-PEG4-Val-Lys-Ala-7-MAD-MDCPT | 2 | (4) | >1K | (ND) | 6 | (3) | 77 | (28) |
| Ag1-Ex_5-1h | MP-PEG4-Phe-Lys-Gly-7-MAD-MDCPT | 1 | (4) | >1K | (57) | 10 | (3) | 104 | (28) |
| Ag1-Ex_5-1j | MP-PEG4-Leu-Leu-Gly-7-MAD-MDCPT | 1 | (4) | >1K | (56) | 4 | (3) | 36 | (26) |
| Ag1-Ex_5-1k | MC-Gly-Gly-Phe-Gly-7-MAD-MDCPT | 2 | (4) | >1K | (ND) | 15 | (3) | 51 | (26) |
| Ag1-Ex_5-1e | MP-PEG4-Leu-Lys-Gly-7-MAD-MDCPT | 1 | (3) | >1K | (58) | 7 | (2) | 69 | (23) |
| Ag1-Ex_5-1l | MC-Gly-Gly-Phe-Gly-Gly-7-MAD-MDCPT | 2 | (5) | 996 | (ND) | 9 | (3) | 84 | (29) |
| Ag1-Ex_5-1f | MP-PEG4-Gly-Val-Lys-Gly-7-MAD-MDCPT | 1 | (3) | >1K | (59) | 9 | (3) | 67 | (26) |
| Ag1-Ex_5-1p | MP-PEG4-Val-Gly-7-MAD-MDCPT | 11 | (11) | >1K | (ND) | >1K | (80) | >1K | (ND) |
| Ag1-Ex_5-1g | MP-PEG4-Val-Lys-Gly-Gly-7-MAD-MDCPT | 1 | (4) | >1K | (ND) | 7 | (3) | 70 | (25) |
| Ag1-Ex_6-1 | MC-GGFG-HAPI-7-BAD-MDCPT | 14 | (6) | >1K | (71) | 421 | (10) | >1K | (ND) |
| Ag1-Ex_5-1q | MP-PEG4-Val-Lys-B-Ala-7-MAD-MDCPT | 0.2 | (4) | >1K | (ND) | 2 | (3) | 25 | (25) |

Table 5. Evaluation of select peptide-based camptothecin anti-Ag1 (DAR=8) ADCs varying in hydrophobicity against various cancer cell lines

Table 5A. renal cancer cells (786-O), pancreatic cancer cells (BxPC3), hepatic cancer cells (HepG2), MDR(−) and MDR(+) acute promyelocytic leukemia cells (HL-60 and HL60/RV, respectively), and Hodgkin's lymphoma cells (L54cy).

Table 5B. multiple myeloma cells (MM.R1), acute myeloid leukemia cells (MOLM13), Burkitt's lymphoma cells (Ramos), melanoma cells (SK-MEL-5) and B-lymphocyte cancer cells (SU-DHL-4 and U266).

TABLE 5A

| ADC | Drug-linker | | | | HepG2 | | | |
|---|---|---|---|---|---|---|---|---|
| (antibody-drug) | Description | Aggregation | 786-O | BxPC3 | (800) | HL-60 | HL60/RV | L540cy |
| Ag1-Ex_5-1d | MP-PEG4-VQG- | 25% | 7 | 21 | 136 | 145 | 898 | 5 |
| | 7-MAD-MDCPT | | (6) | (35) | (42) | (3) | (ND) | (2) |
| Ag1-Ex_5-1g | MP-PEG4- | | 13 | 13 | >1K | 96 | >1K | 5 |
| | VKBetaA-7- | | (11) | (34) | (53) | (2) | (79) | (1) |
| | MAD-MDCPT | | | | | | | |
| Ag1-Ex_5-1c | MP-PEG4-VEG- | | 13 | 15 | >1K | 139 | >1K | 5 |
| | 7-MAD-MDCPT | | (10) | (34) | (50) | (3) | (ND) | (0) |
| Ag1-Ex_5-1j | MP-PEG4-LLG-7- | 86% | 6 | 21 | 138 | 165 | 294 | 5 |
| | MAD-MDCPT | | (6) | (35) | (38) | (3) | (27) | (1) |
| Ag1-Ex_5-1o | MP-PEG4-VCG- | 55% | 10 | 17 | 136 | 122 | >1K | 5 |
| | 7-MAD-MDCPT | | (9) | (35) | (47) | (3) | (72) | (1) |
| Ag1-Ex_6-1 | MC-GGFG-HAPI- | | >1K | 80 | >1K | >1K | 98 | 21 |
| | 7-BAD-MDCPT | | (55) | (44) | (56) | (ND) | (47) | (1) |
| Ag1-Ex_5-1b | MP-PEG4-VGG- | | 20 | 20 | 344 | 156 | >1K | 6 |
| | 7-MAD-MDCPT | | (16) | (39) | (46) | (5) | (ND) | (2) |
| Ag1-Ex_5-1m | MP-PEG4-VKA- | | 34 | 30 | 88 | 129 | >1K | 4 |
| | 7-MAD-MDCPT | | (21) | (40) | (49) | (4) | (72) | (1) |
| Ag1-Ex_4-1 | MP-PEG4-VKG- | 1.7% | 14 | 25 | 291 | 165 | >1K | 5 |
| | 7-MAD-MDCPT | | (12) | (37) | (50) | (5) | (ND) | (1) |
| Ag1-Ex_4-2 | MP-PEG2-VKG- | | 9 | 26 | 17 | 96 | >1K | 4 |
| | 7-MAD-MDCPT | | (7) | (33) | (28) | (3) | (64) | (2) |
| Ag1-Ex_4-3 | MP-PEG8-VKG- | 1.6% | 11 | 18 | 19 | 117 | >1K | 5 |
| | 7-MAD-MDCPT | | (11) | (35) | (34) | (3) | (82) | (2) |
| Ag1-Ex_4-4 | MP-PEG12-VKG- | 1.9% | 9 | 18 | 8 | 98 | >1K | 4 |
| | 7-MAD-MDCPT | | (10) | (34) | (39) | (53 | (76) | (3) |
| Ag1-Ex_4-5 | MP-Lys(PEG12)- | 2.3% | 11 | 27 | 21 | 122 | >1K | 5 |
| | VKG-7-MAD- | | (10) | (40) | (31) | (4) | (83) | (3) |
| | MDCPT | | | | | | | |
| Ag1-Ex_5-1r | MP-PEG4-VKE- | | 33 | 51 | — | 173 | >1K | 15 |
| | 7-MAD-MDCPT | | (12) | (34) | | (3) | (67) | (2) |
| Ag1-Ex_5-1s | MP-PEG4-VEE-7- | | 13 | 31 | — | 52 | 13 | 3 |
| | MAD-MDCPT | | (19) | (48) | | (8) | (3) | (3) |
| Ag1-Ex_8-1a | mc-gly-gly-phe- | | >1K | 162 | >1K | 314 | >1K | 19 |
| | gly-NHCH2- | | (51) | (46) | (59) | (40) | (96) | (2) |
| | DXd(1) | | | | | | | |

TABLE 5B

| ADC | Drug-linker | | | | | | | |
|---|---|---|---|---|---|---|---|---|
| (antibody-drug) | Description | Aggregation | MM.1R | MOLM13 | Ramos | SK-MEL-5 | SU-DHL-4 | U266 |
| Ag1-Ex_5-1d | MP-PEG4-VQG- | 25% | 3 | 61 | 0.03 | 180 | 1 | 22 |
| | 7-MAD-MDCPT | | (0) | (0) | (2) | (40) | (1) | (32) |
| Ag1-Ex_5-1g | MP-PEG4- | | 2 | 36 | 0.1 | >1K | 1 | 16 |
| | VKBetaA-7- | | (0) | (0) | (0) | (ND) | (1) | (29) |
| | MAD-MDCPT | | | | | | | |
| Ag1-Ex_5-1c | MP-PEG4-VEG- | | 3 | 62 | 0.2 | 387 | 1 | 13 |
| | 7-MAD-MDCPT | | (0) | (0) | (2) | (45) | (2) | (25) |
| Ag1-Ex_5-1j | MP-PEG4-LLG-7- | 86% | 3 | 82 | 0.2 | 118 | 2 | 9 |
| | MAD-MDCPT | | (0) | (0) | (1) | (39) | (1) | (27) |
| Ag1-Ex_5-1o | MP-PEG4-VCG- | 55% | 3 | 55 | 0.1 | 321 | 1 | 11 |
| | 7-MAD-MDCPT | | (0) | (0) | (1) | (42) | (2) | (25) |
| Ag1-Ex_6-1 | MC-GGFG-HAPI- | | 42 | 223 | 4 | >1K | 23 | 777 |
| | 7-BAD-MDCPT | | (18) | (5) | (1) | (76) | (6) | (48) |
| Ag1-Ex_5-1b | MP-PEG4-VGG- | | 4 | 57 | 0.2 | >1K | 2 | 13 |
| | 7-MAD-MDCPT | | (0) | (0) | (2) | (54) | (2) | (25) |
| Ag1-Ex_5-1m | MP-PEG4-VKA- | | 3 | 61 | 0.1 | >1K | 1 | 13 |
| | 7-MAD-MDCPT | | (0) | (1) | (2) | (50) | (3) | (29) |
| Ag1-Ex_4-1 | MP-PEG4-VKG- | 1.7% | 5 | 64 | 0.1 | >1K | 2 | 24 |
| | 7-MAD-MDCPT | | (0) | (0) | (3) | (ND) | (2) | (32) |
| Ag1-Ex_4-2 | MP-PEG2-VKG- | | 2 | 30 | 0.2 | 31 | 1 | 20 |
| | 7-MAD-MDCPT | | (2) | (0) | (3) | (34) | (4) | (28) |
| Ag1-Ex_4-3 | MP-PEG8-VKG- | 1.6% | 3 | 33 | 0.3 | 84 | 2 | 20 |
| | 7-MAD-MDCPT | | (2) | (0) | (3) | (27) | (4) | (28) |
| Ag1-Ex_4-4 | MP-PEG12-VKG- | 1.9% | 2 | 36 | 0.3 | 33 | 2 | 20 |
| | 7-MAD-MDCPT | | (2) | (1) | (3) | (33) | (3) | (27) |
| Ag1-Ex_4-5 | MP-Lys(PEG12)- | 2.3% | 3 | 42 | 0.4 | 86 | 2 | 19 |
| | VKG-7-MAD- | | (2) | (0) | (4) | (36) | (3) | (32) |
| | MDCPT | | | | | | | |
| Ag1-Ex_5-1r | MP-PEG4-VKE- | | — | >1K | 1 | — | >1K | — |
| | 7-MAD-MDCPT | | | (ND) | (7) | | (ND) | |
| Ag1-Ex_5-1s | MP-PEG4-VEE-7- | | — | 14 | 0.04 | — | 0.4 | — |
| | MAD-MDCPT | | | (2) | (2) | | (2) | |
| Ag1-Ex_8-1a | mc-gly-gly-phe- | | 15 | 89 | 0.5 | >1K | 10 | >1K |
| | gly-NHCH2- | | (4) | (1) | (2) | (68) | (5) | (57) |
| | DXd(1) | | | | | | | |

Table 6. In vitro evaluation of peptide-based camptothecin (DAR=8) targeting various cancer cells expressing Ag1 in comparison to non-binding control (h00) ADCs

Table 6A. renal cancer cells (786-O), pancreatic cancer cells (BxPC3), hepatic cancer cells (HepG2), MDR(−) and MDR(+) acute promyelocytic leukemia cells (HL-60 and HL60/RV, respectively) and Hodgkin's lymphoma cells (L540cy).

Table 6B. multiply myeloma cells (MM.R1), acute myeloid leukemia cells (MOLM13), Burkitt's lymphoma cells (Ramos), melanoma cells (SK-MEL-5) and B-lymphocyte cancer cells (SU-DHL-4 and U266).

TABLE 6A

| ADC | | | | | | |
|---|---|---|---|---|---|---|
| (antibody-drug) | 786-O | BxPC3 | HepG2 | HL-60 | HL60/RV | L540cy |
| Ag1-Ex_1-1 | 27 | 47 | 36 | 216 | 104 | 7 |
| | (23) | (33) | (36) | (3) | (33) | (2) |
| h00-Ex_1-1 | >1K | >1K | >1K | >1K | >1K | >1K |
| | (100) | (98) | (ND) | (ND) | (83) | (ND) |
| Ag1-Ex_2-1 | >1K | 855 | 68 | 987 | >1K | 15 |
| | (50) | (50) | (41) | (ND) | (ND) | (1) |
| h00-Ex_2-1 | >1K | >1K | >1K | >1K | >1K | >1K |
| | (97) | (ND) | (ND) | (ND) | (84) | (100) |
| Ag1-Ex_3-1 | >1K | 190 | 199 | >1K | >1K | 160 |
| | (44) | (43) | (15) | (ND) | (62) | (36) |
| h00-Ex_3-1 | >1K | >1K | >1K | >1K | >1K | >1K |
| | (92) | (100) | (95) | (94) | (ND) | (100) |
| Ag1-Ex_7-1 | 197 | 46 | 122 | 843 | >1K | 23 |
| | (21) | (36) | (43) | (ND) | (ND) | (1) |
| Ag1-Ex_6-1 | >1K | >1K | >1K | >1K | >1K | 77 |
| | (86) | (ND) | (ND) | (ND) | (ND) | (9) |
| Ag1-Ex_9-1a | 625 | 328 | 224 | 307 | >1K | 53 |
| | (ND) | (41) | (35) | (32) | (ND) | (1) |
| h00-Ex_9-1a | >1K | 635 | 646 | >1K | >1K | 930 |
| | (ND) | (49) | (35) | (ND) | (ND) | (ND) |
| Ag1-Ex_9-1b | >1K | 939 | 490 | >1K | >1K | 121 |
| | (50) | (39) | (20) | (51) | (94) | (1) |
| h00-Ex_9-1b | >1K | >1K | 923 | >1K | >1K | >1K |
| | (82) | (ND) | (ND) | (ND) | (ND) | (ND) |
| Ag1-Ex_10-1a | 7 | 31 | — | 113 | >1K | 4 |
| | (9) | (31) | | (4) | (97) | (3) |
| h00-Ex_10-1a | >1K | >1K | — | >1K | >1K | >1K |
| | (99) | (100) | | (ND) | (ND) | (86) |
| Ag1-Ex_10-1b | 10 | 37 | — | 119 | >1K | 5 |
| | (10) | (34) | | (4) | (93) | (3) |
| h00-Ex_10-1b | >1K | >1K | — | >1K | >1K | >1K |
| | (98) | (100) | | (ND) | (94) | (89) |

TABLE 6B

| ADC | | | | | | |
|---|---|---|---|---|---|---|
| (antibody— | | | | SK- | SU- | |
| drug) | MM.1R | MOLM13 | Ramos | MEL-5 | DHL-4 | U266 |
| Ag1-Ex_1-1 | 4 | 58 | ND | 164 | 2 | 17 |
| | (0) | (0) | (ND) | (31) | (4) | (27) |
| h00-Ex_1-1 | >1K | >1K | ND | >1K | >1K | >1K |
| | (ND) | (92) | (ND) | (ND) | (ND) | (ND) |
| Ag1-Ex_2-1 | 18 | 429 | 4 | >1K | 27 | 254 |
| | (0) | (ND) | (2) | (57) | (3) | (30) |
| h00-Ex_2-1 | >1K | >1K | >1K | >1K | >1K | ~1K |
| | (94) | (ND) | (77) | (88) | (86) | (ND) |
| Ag1-Ex_3-1 | >1K | 183 | ND | >1K | >1K | 127 |
| | (85) | (4) | (ND) | (55) | (ND) | (37) |
| h00-Ex_3-1 | >1K | >1K | ND | >1K | >1K | >1K |
| | (87) | (100) | (ND) | (ND) | (ND) | (ND) |
| Ag1-Ex_7-1 | 38 | 170 | 3 | 367 | 14 | 85 |
| | (6) | (2) | (2) | (14) | (2) | (24) |
| Ag1-Ex_6-1 | >1K | 255 | 14 | >1K | 421 | >1K |
| | (38) | (16) | (6) | (71) | (10) | (ND) |
| Ag1-Ex_9-1a | 11 | 54 | 0.5 | 385 | 7 | 192 |
| | (1) | (0) | (3) | (47) | (4) | (21) |
| h00-Ex_9-1a | 811 | >1K | 329 | >1K | 572 | >1K |
| | (ND) | (ND) | (ND) | (ND) | (ND) | (ND) |
| Ag1-Ex_9-1b | 19 | 80 | 1 | >1K | 11 | 334 |
| | (2) | (1) | (4) | (64) | (4) | (37) |
| h00-Ex_9-1b | 981 | >1K | 377 | >1K | 569 | >1K |
| | (ND) | (ND) | (ND) | (ND) | (ND) | (ND) |
| Ag1-Ex_10-1a | 3 | 59 | 0.2 | 128 | 2 | 37 |
| | (2) | (2) | (3) | (44) | (4) | (40) |
| h00-Ex_10-1a | >1K | >1K | >1K | >1K | >1K | >1K |
| | (65) | (66) | (86) | (93) | (89) | (69) |
| Ag1-Ex_10-1b | 2 | 65 | 1 | 273 | 1 | 30 |
| | (0) | (0) | (3) | (38) | (3) | (38) |
| h00-Ex_10-1b | >1K | >1K | >1K | >1K | >1K | >1K |
| | (73) | (68) | (100) | (ND) | (ND) | (65) |

Table 7. Cytotoxic potency of camptothecin compounds as free drugs.

Table 7A. renal cancer cells (786-O), pancreatic cancer cells (BxPC3), hepatic cancer cells (HepG2), MDR(−) and MDR(+) acute promyelocytic leukemia cells (HL-60 and HL60/RV, respectively), Hodgkin's lymphoma cells (L540cy) and multiple myeloma cells (MM.1R)

Table 7B. acute myeloid leukemia cells (MOLM-13), Burkitt's lymphoma cells (Ramos), melanoma cells (SK-MEL-5) and B-lymphocyte cancer cells (SU-DHL-4 and U266).

++++ IC₅₀ between 0.1 to <1 nM, +++ IC₅₀ between 1 to <10 nM, ++ IC₅₀ between >10 nM to ≤100 nM, + IC₅₀ between >100 nM to ≤1000 nMm, [symbol] IC₅₀>1000 nM.

TABLE 7A

| Compound | | | | | | |
|---|---|---|---|---|---|---|
| No. | 786-O | BxPC3 | HL-60 | HL60/RV | L540cy | MM.1R |
| 6 | +++ | +++ | ND | ++++ | ++++ | +++ |
| 6b | +++ | +++ | ND | +++ | +++ | +++ |
| 6c | +++ | +++ | ND | +++ | +++ | +++ |
| 6d | +++ | +++ | +++ | +++ | +++ | +++ |
| 6j | +++ | +++ | ++ | +++ | +++ | +++ |
| 6e | ++++ | +++ | +++ | +++ | ++++ | ++++ |
| 6k | +++ | +++ | + | ++ | +++ | +++ |
| 6f | +++ | +++ | ++ | +++ | +++ | +++ |
| 6l | +++ | +++ | +++ | +++ | +++ | +++ |
| 6g | +++ | +++ | ++ | +++ | +++ | +++ |
| 6p | ++++ | ++++ | ++ | +++ | ++++ | ++++ |
| 6h | ++++ | +++ | ++ | ++ | +++ | +++ |
| 6m | ++++ | +++ | ++ | +++ | ++++ | ++++ |
| 6i | +++ | +++ | ++ | +++ | +++ | +++ |
| 6n | +++ | +++ | ++ | +++ | +++ | +++ |
| 6o | +++ | +++ | + | ++ | +++ | +++ |
| 5e | ++ | ++ | ++ | + | +++ | +++ |
| 5 | +++ | ++ | ++ | ++ | +++ | +++ |
| 5f | +++ | ++ | ++ | + | +++ | +++ |
| 5a | +++ | +++ | ++ | +++ | +++ | +++ |
| 5g | +++ | +++ | +++ | +++ | +++ | +++ |
| 5b | +++ | +++ | ++ | +++ | ++++ | +++ |
| 5h | +++ | ++ | ++ | ++ | +++ | +++ |
| 5c | +++ | +++ | +++ | +++ | +++ | +++ |
| 5i | +++ | +++ | ++ | +++ | +++ | +++ |
| 5d | +++ | +++ | ++ | +++ | +++ | +++ |
| 5j | +++ | +++ | ++ | +++ | +++ | +++ |
| 5k | +++ | +++ | ++ | +++ | +++ | +++ |
| 5q | ++ | + | + | + | ++ | ++ |
| 5l | ++ | ++ | ++ | ++ | ++ | +++ |
| 5r | +++ | +++ | ++ | ++ | +++ | +++ |
| 5m | ++ | + | + | [symbol] | ++ | ++ |
| 5s | ++ | ++ | + | ++ | ++ | ++ |
| 5n | +++ | ++ | + | + | +++ | +++ |
| 5t | +++ | ++ | + | ++ | +++ | +++ |
| 5o | ++ | ++ | + | ++ | +++ | ++ |
| 5u | ++ | ++ | + | + | ++ | ++ |
| 5p | ++ | ++ | + | + | ++ | +++ |
| 5w | ++ | ++ | + | ++ | ++ | ++ |
| 5x | ++ | + | + | + | ++ | ++ |
| 5y | +++ | +++ | ++ | ++ | +++ | +++ |
| 4c | ++ | + | + | + | ++ | ++ |
| 4d | + | + | + | [symbol] | + | +++ |
| 4e | [symbol] | + | [symbol] | [symbol] | + | ND |
| 5z | +++ | ++ | ++ | ++ | +++ | +++ |
| 8b | +++ | +++ | ++ | ++ | +++ | +++ |
| 5aa | ++ | + | + | + | ++ | ++ |
| 8a | +++ | ++ | + | ++ | +++ | +++ |
| 6q | ++ | ++ | + | + | ++ | ++ |
| 9b | ++++ | +++ | ++ | ++ | ++++ | ++++ |
| 4 | +++ | +++ | ++ | ++ | +++ | +++ |
| 4b | + | + | + | [symbol] | ++ | ++ |
| 6r | +++ | +++ | ++ | +++ | +++ | +++ |
| 8c | +++ | +++ | ++ | +++ | ++++ | +++ |
| 9a | +++ | ++ | + | + | +++ | +++ |
| 4a | ++ | ++ | ++ | ++ | ++ | +++ |
| 8d | +++ | +++ | +++ | ++ | ++ | +++ |
| 6a | +++ | +++ | +++ | +++ | ++++ | +++ |

TABLE 7B

| Compound | | | | | |
|---|---|---|---|---|---|
| No. | MOLM13 | Ramos | SK-MEL-5 | SU-DHL-4 | U266 |
| 6 | ++++ | ++++ | +++ | ++++ | +++ |
| 6b | +++ | +++ | +++ | +++ | +++ |
| 6c | +++ | ++++ | +++ | ++++ | +++ |
| 6d | +++ | ++++ | +++ | ++++ | +++ |
| 6j | +++ | ++++ | +++ | +++ | +++ |
| 6e | +++ | ++++ | +++ | ++++ | +++ |
| 6k | ++ | ++++ | +++ | +++ | +++ |
| 6f | +++ | +++ | +++ | +++ | +++ |
| 6l | +++ | ++++ | +++ | +++ | +++ |
| 6g | +++ | _++++ | +++ | +++ | +++ |
| 6p | +++ | ++++ | ++++ | ++++ | ++++ |
| 6h | ++ | ++++ | +++ | ++++ | +++ |
| 6m | +++ | ++++ | +++ | ++++ | +++ |
| 6i | +++ | ++++ | +++ | ++++ | +++ |
| 6n | +++ | +++ | +++ | +++ | +++ |
| 6o | ++ | ++++ | +++ | +++ | +++ |
| 5e | +++ | +++ | ++ | +++ | ++ |
| 5 | +++ | +++ | +++ | +++ | +++ |
| 5f | +++ | +++ | +++ | +++ | +++ |
| 5a | +++ | +++ | +++ | +++ | +++ |
| 5g | +++ | ++++ | +++ | +++ | +++ |
| 5b | ++ | ++++ | +++ | ++++ | +++ |
| 5h | +++ | +++ | ++ | +++ | ++ |
| 5c | +++ | ++++ | +++ | +++ | +++ |
| 5i | +++ | +++ | +++ | +++ | +++ |
| 5d | +++ | +++ | +++ | +++ | +++ |
| 5j | +++ | +++ | +++ | +++ | +++ |
| 5k | +++ | +++ | +++ | +++ | +++ |
| 5q | ++ | ++ | + | ++ | ++ |
| 5l | +++ | +++ | ++ | +++ | ++ |
| 5r | ++ | +++ | +++ | +++ | +++ |
| 5m | ++ | +++ | + | ++ | ++ |
| 5s | ++ | ++ | ++ | ++ | ++ |
| 5n | + | +++ | ++ | +++ | ++ |
| 5t | ++ | +++ | ++ | +++ | ++ |
| 5o | ++ | +++ | ++ | ++ | ++ |
| 5u | ++ | +++ | ++ | ++ | ++ |
| 5p | ++ | +++ | ++ | ++ | ++ |
| 5w | ++ | +++ | ++ | ++ | ++ |
| 5x | ++ | ++ | ++ | ++ | ++ |
| 5y | +++ | ++++ | +++ | +++ | +++ |
| 4c | +++ | +++ | + | ++ | ++ |
| 4d | ++ | ++ | ++ | ++ | ++ |
| 4e | + | + | ND | † | ND |
| 5z | ++ | +++ | ++ | +++ | ++ |
| 8b | ++ | ++++ | +++ | +++ | +++ |
| 5aa | ++ | +++ | ++ | ++ | ++ |
| 8a | ++ | +++ | ++ | +++ | ++ |
| 6q | ++ | ++ | + | ++ | ++ |
| 9b | ++ | ++++ | +++ | ++++ | +++ |
| 4 | +++ | +++ | +++ | ++++ | +++ |
| 4b | ++ | ++ | + | ++ | ++ |
| 6r | +++ | ++++ | +++ | +++ | +++ |
| 8c | +++ | ++++ | +++ | ++++ | +++ |
| 9a | ++ | ++++ | +++ | +++ | +++ |
| 4a | +++ | ++ | +++ | ++ | +++ |
| 8d | +++ | ++ | +++ | +++ | +++ |
| 6a | +++ | ++++ | +++ | ++++ | +++ |

### In Vivo Model Methods

All experiments were conducted in concordance with the Animal Care and Use Committee in a facility fully accredited by the Association for Assessment and Accreditation of Laboratory Animal Care. Efficacy experiments were conducted in the 786-O, L540cy and Karpas/Karpas-BVR, DelBVR, Karpas 299, L428, DEL-15, and L82 xenografts models. Tumor cells, as a cell suspension, were implanted sub-cutaneous in immune-compromised SCID or nude mice. Upon tumor engraftment, mice were randomized to study groups (5 mice per group) when the average tumor volume reached about 100 mm3. The ADC or controls were dosed once via intraperitoneal injection. The average number of drug-linker attached to an antibody is indicated in the parenthesis next to the ADC (also referred to herein as Drug-Antibody Ratio (DAR) number, e.g., DAR4, DAR8, etc.). Tumor volume as a function of time was determined using the formula (L×W2)/2. Animals were euthanized when tumor volumes reached 750 mm³. Mice showing durable regressions were terminated after 10-12 weeks post implant.

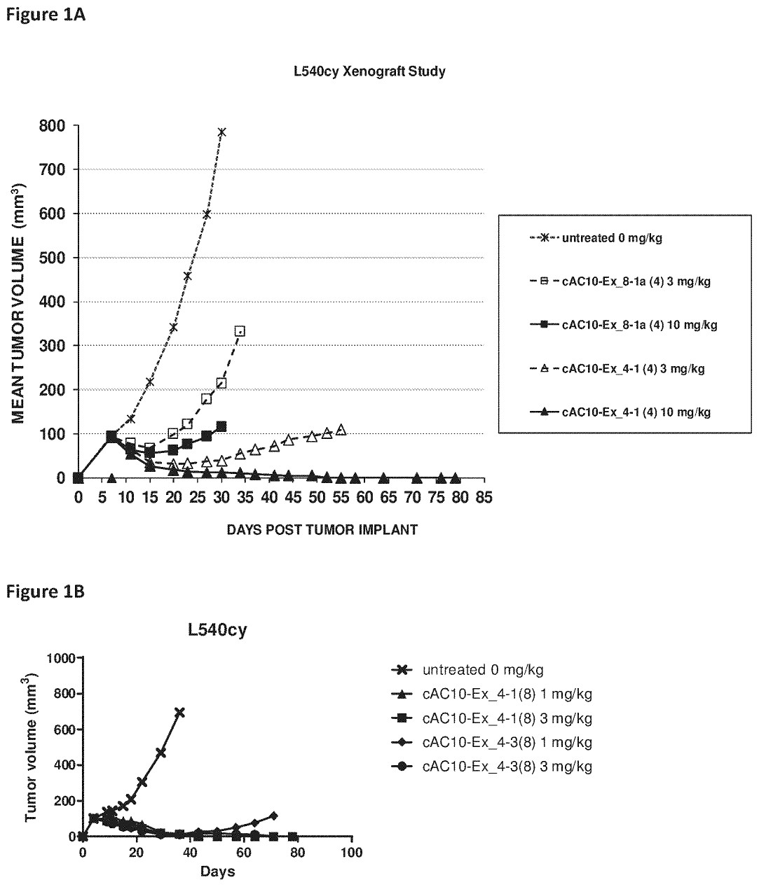

Animals were implanted with L540cy cells. After 7 days, the animals were sorted into groups with an average tumor size of 100 mm³, and then treated with a single dose of camptothecin ADC cAC10-Ex_8-1a (4) or cAC10-Ex_4-1 (4), at 3 or 10 mg/kg. In another experiment, treated with a single dose of camptothecin ADC cAC10-Ex_4-1 (8) or cAC10-Ex_4-3 (8), at 1 or 3 mg/kg. Animals were evaluated for tumor size and in-life signs during the course of the study. The results are shown in FIGS. 1A and 1B.

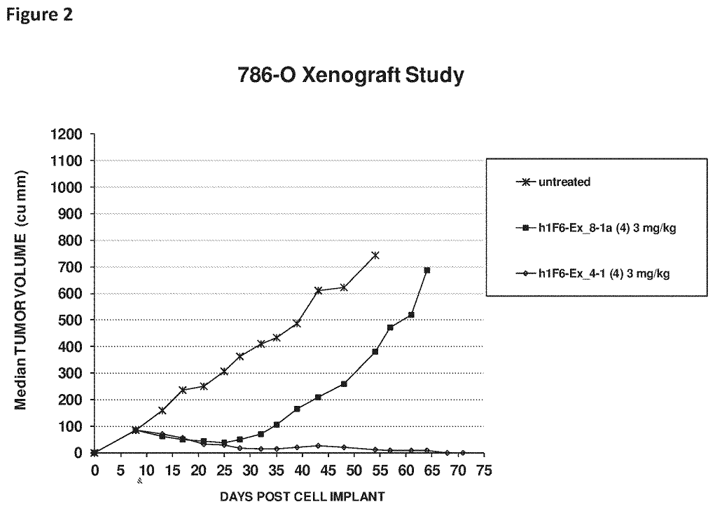

Animals were implanted with 786-O cells. On day 10, the animals were sorted into groups with an average tumor size of 100 mm³, and then treated with a single dose of camptothecin ADC cAC10-Ex_8-1a (4) or cAC10-Ex_4-1 (4), at 10 mg/kg. Animals were evaluated for tumor size and in-life signs during the course of the study. The results are shown in FIG. 2.

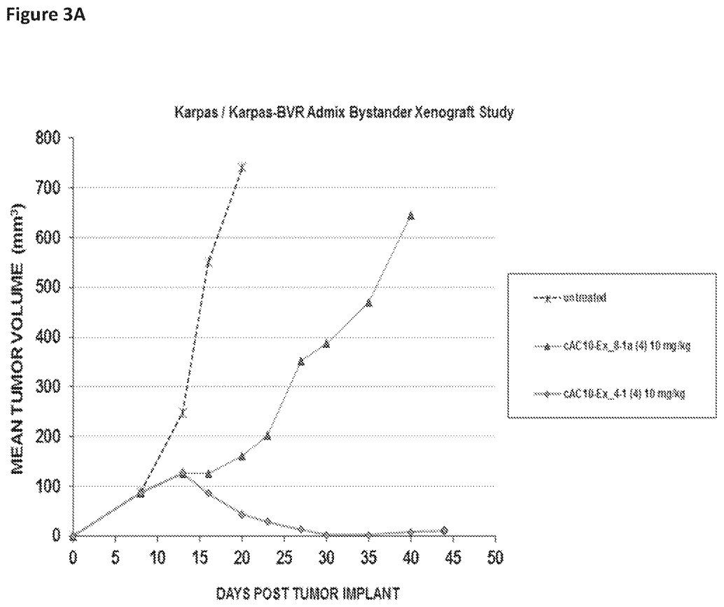

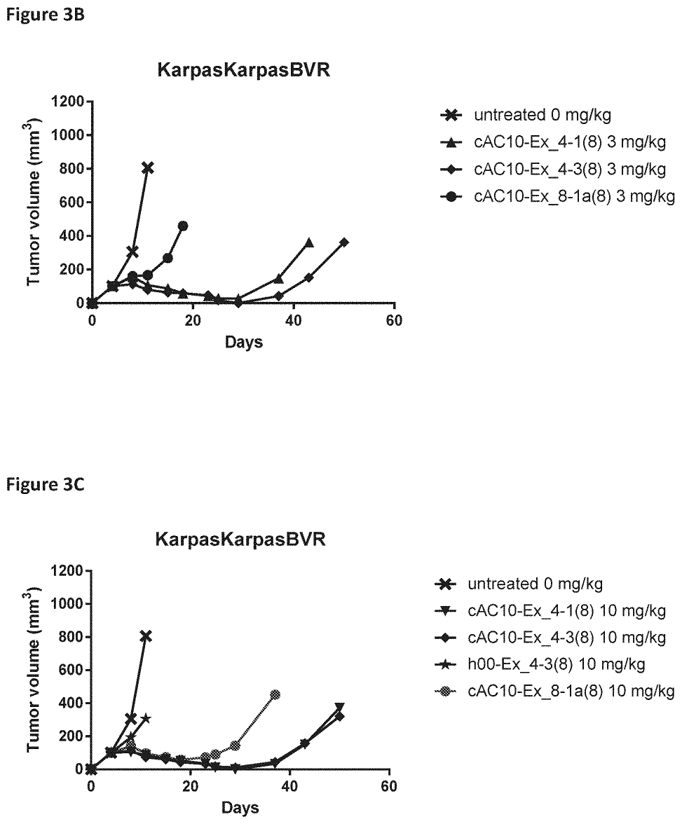

Animals were implanted with a 1:1 mixture of CD30+ Karpas299 and CD30-Karpas299-brentuximab vedotin resistant (Karpas299-BVR) cells. After 8 days, the animals were sorted into groups with an average tumor size of 100 mm³, and then treated with a single dose of camptothecin ADC cAC10-Ex_8-1a (4) or cAC10-Ex_4-1 (4), at 10 mg/kg. In another experiment, animals were treated with a single dose of camptothecin ADC cAC10-Ex_8-1a (8), cAC10-Ex_4-1 (8), or cAC10-Ex_4-3 (8), at 3 or 10 mg/kg. Animals were evaluated for tumor size and in-life signs during the course of the study. The results are shown in FIG. 3A-3C.

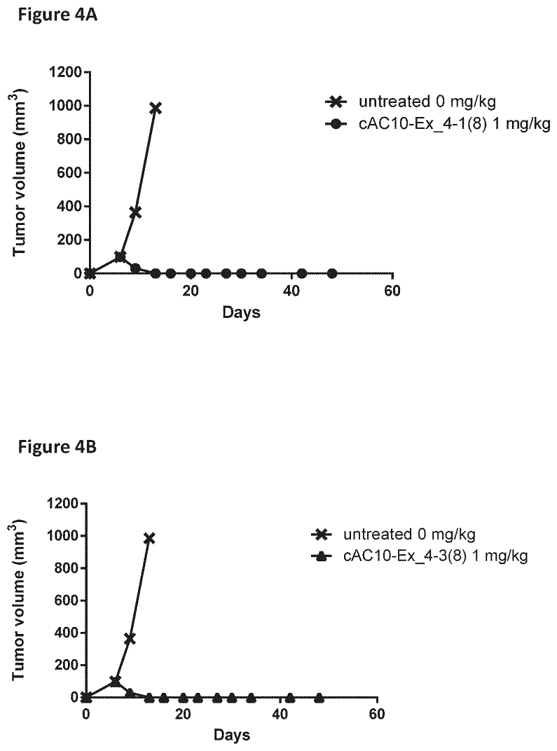

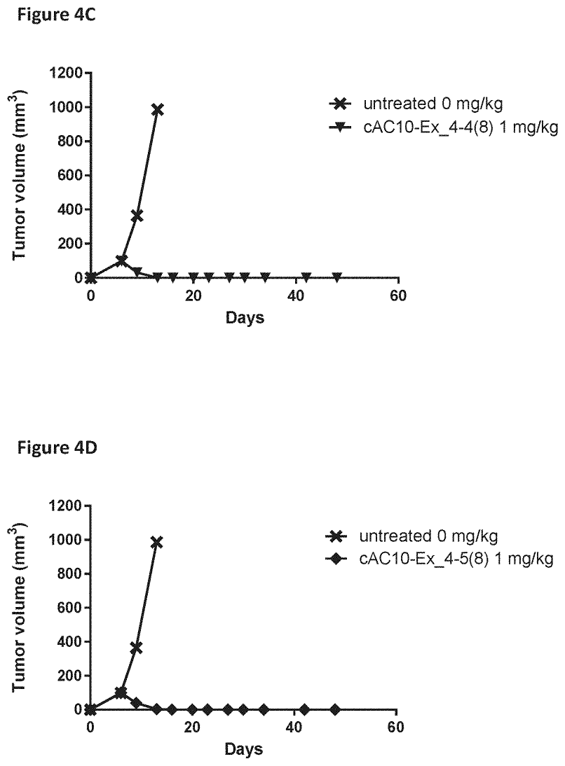

Animals were implanted with DelBVR cells. On day 7, the animals were sorted into groups with an average tumor size of 100 mm³, and then treated with a single dose of camptothecin ADC cAC10-Ex_4-1(8), cAC10-Ex_4-3(8), cAC10-Ex_4-4(8), or cAC10-Ex_4-5(8), at 0.3 or 1 mg/kg. Animals were evaluated for tumor size and in-life signs during the course of the study. The results are shown in FIG. 4.

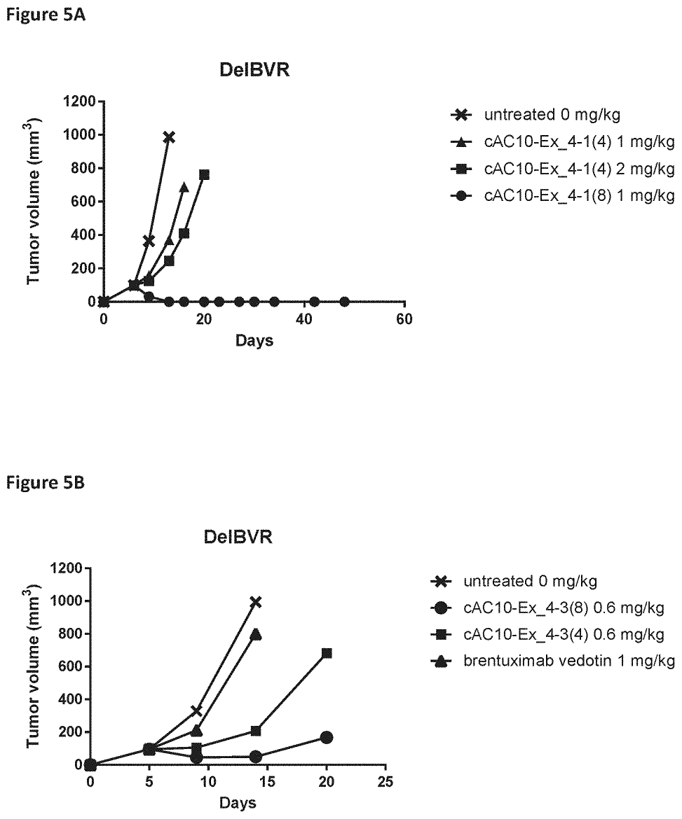

Animals were implanted with DelBVR cells. On day 7, the animals were sorted into groups with an average tumor size of 100 mm³, and then treated with a single dose of camptothecin ADC cAC10-Ex_4-1(4) or cAC10-Ex_4-1(8), at 1 or 2 mg/kg, or with a single dose of camptothecin ADC cAC10-Ex_4-3(4) or cAC10-Ex_4-3(8), at 0.6 or 1 mg/kg. Animals were evaluated for tumor size and in-life signs during the course of the study. The results are shown in FIG. 5.

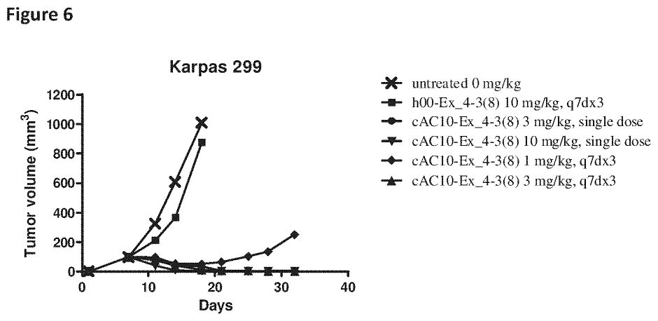

Animals were implanted with Karpas299 cells. After 7 days, the animals were sorted into groups with an average tumor size of 100 mm³, and then treated with a single dose of non-binding control h00-Ex_4-3(8), or camptothecin ADC cAC10-Ex_4-3 (8), at 1, 3 or 10 mg/kg with either single or multi-dose. Animals were evaluated for tumor size and in-life signs during the course of the study. The results are shown in FIG. 6.

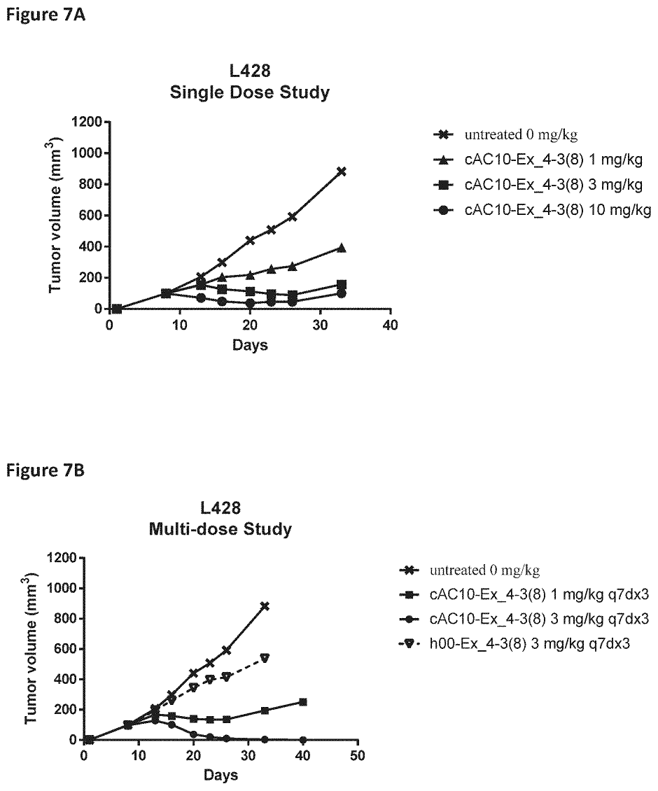

Animals were implanted with L428 cells. After 7 days, the animals were sorted into groups with an average tumor size of 100 mm³, and then treated with camptothecin ADC cAC10-Ex_4-3(8), at 1, 3 or 10 mg/kg with either single or multi-dose. Animals were evaluated for tumor size and in-life signs during the course of the study. The results are shown in FIG. 7.

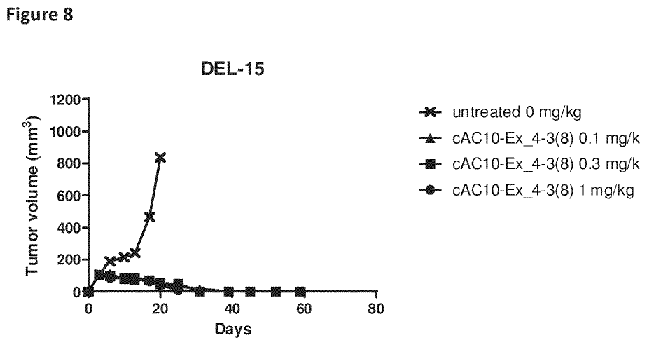

Animals were implanted with DEL-15 cells. After 7 days, the animals were sorted into groups with an average tumor size of 100 mm³, and then treated with a single dose of camptothecin ADC cAC10-Ex_4-3(8), at 0.1, 0.3 or 1 mg/kg. Animals were evaluated for tumor size and in-life signs during the course of the study. The results are shown in FIG. 8.

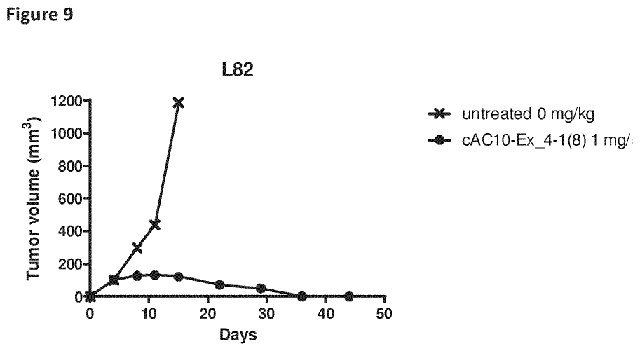

Animals were implanted with L82 cells. After 7 days, the animals were sorted into groups with an average tumor size of 100 mm³, and then treated with a single dose of camptothecin ADC cAC10-Ex_4-1(8), at 1 mg/kg. Animals were evaluated for tumor size and in-life signs during the course of the study. The results are shown in FIG. 9.

Data in FIGS. 1-9 showed cAC10-Ex_4-1, cAC10-Ex_4-3, cAC10-Ex_4-4 and cAC10-Ex_4-5 ADCs all displayed in vivo anti-tumor activities on models tested. Data in FIGS. 1-9 also showed that cAC10-Ex_4-1 and cAC10-Ex_4-3 ADCs displayed improved in vivo potency compared to cAC10-Ex_8-1a ADC, including improved activity in Karpas/Karpas BVR bystander model (as shown in FIG. 3A-3C).

### ADC Plasma Stability Determination

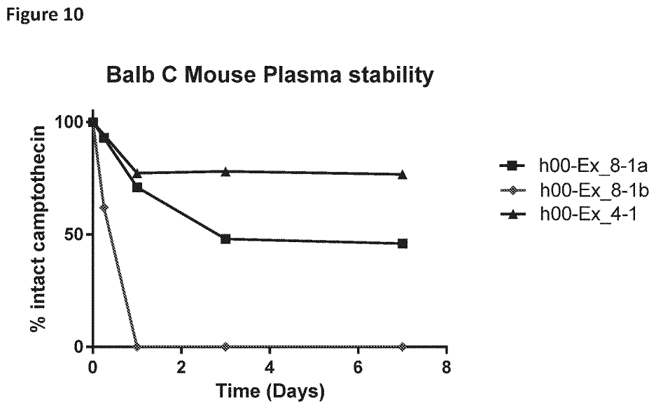

All ADC stocks were normalized to 2.5 mg/mL. The 2.5 mL single use aliquots of citrated mouse (Balb C) were stored at −80 C prior to use. A stock solution in ADC in mouse plasma was made as follows. ADC (50 μg) in 200 μL of plasma (per time point, 0.25 mg/mL) with final PBS concentration at 13.85. Plasma samples were incubated at 37 degrees Celsius for 6 h, 1-day, 3-day, and 7-day time points, and were sampled in duplicate. After each time point, the samples were stored at −80 degrees Celsius until they were processes for analysis. A 50% slurry of IgSelect in 1×PBS was prepared. For each time point sample, 50 μL of the IgSelect slurry was added to a 3 uM filter plate, and vacuum was applied to remove supernatant. The resin was washed (2×1 mL 1×PBS), with vacuum applied after each wash. Sample (180 uL) was applied, and the filter plate was shaken (1200 rpm for 1 h at 4 degrees Celsius. Vacuum was then applied to remove plasma. The resin was washed with 1 mL PBS+50 mM NaCl, 1 mL PBS, and with 1 mL water, with vacuum being applied after each wash. The sample plate was then centrifuged at 500×g for 2 mins over a Waters 350 μL collection plate. The ADC was eluted from the resin by treatment with 50 μL Gly pH3 (2×50 uL), mixing at 500 rpm for 2 min at 4 C, centrifuged at 500×g for 3 min into a 350 μL 96 well plate, each well containing 10 μL of 1M Tris pH7.4 buffer. ADC concentration was determined using a UV-Vis plate reader. The samples were deglycosylated using 1 μL of PNGase per sample and incubation for 1 h at 37 degrees Celsius. Each ADC was reduced by adding 12 μL of 100 mM DTT and incubation for 15 min at 37 degrees Celsius. Finally, the samples (10 or 50 μL injection) were analyzed using a 15 min PLRP-MS method to assess light and heavy chain composition to quantify drug-loading for at each timepoint. As shown in FIG. 10, Ex_4-1 based ADC demonstrated improved ex vivo drug-linker stability in mouse plasma, relative to Ex_8-1a and Ex_8-1b based ADCs, contributing to improved in vivo activity.

### ADC PK Analysis Experimental Method

This procedure describes a method for the quantification of the total human IgG in rodent K₂EDTA plasma.

The method uses a biotin-conjugated murine anti-human light chain kappa mAb (SDIX) as the capture reagent, and the same antibody conjugated to Alexafluor-647 as the detection reagent, for quantification of human antibody and/or antibody-drug conjugate test article as Total Antibody (TAb) in K₂EDTA rodent plasma. The assay was carried out using the GyroLab xPlore platform, which utilizes a disc containing microfluidic structures with nanoliter scale streptavidin-coated bead columns on which the ligand-binding assay takes place. Briefly, study samples were diluted with naïve pooled rodent K₂EDTA plasma as needed, and then, along with calibrators, controls, and a plasma blank, were diluted with Rexxip-HX buffer at a Minimal Required Dilution (MRD) of 1:10 prior to being loaded into a 96-well sample plate. Biotin-anti-human kappa capture reagent at 1 ug/mL in Phosphate Buffered Saline pH 7.4 with Tween-20 (PBS-T), AF647-anti-human kappa detection reagent at 25 nM in Rexxip F buffer, and PBS-T wash buffer was added to a 96-well reagent plate, and both plates were sealed and added to the instrument. A run file was established in the GyroLab Control software, and a sample template was exported to Excel to allow input of sample designations and dilution factors. This template was then imported back into GyroLab Control prior to starting the run. The assay was sequential: the biotinylated capture reagent was applied to the BioAffy1000 CD first, the disc was rinsed with PBS-T, and then the diluted plasma blank, standards, controls, and samples were added. After a subsequent PBS-T rinse, the AF647-conjugated detection reagent was applied. After a final PBS-T rinse, each column of the disc was read with laser-induced fluorescence detection (excitation wavelength: 635 nm). The detected response at 1% PMT was subjected to a 5-parameter logistic regression (5-PL) using Gyrolab Evaluator software for conversion of the fluorescence response to ng/mL Total Antibody present in the samples.

The range of the assay for quantitation of total human IgG in rodent K₂EDTA plasma was 22.9 ng/mL (LLOQ) to 50,000 ng/mL (ULOQ) for unconjugated antibody test articles and 22.9 ng/mL (LLOQ) to 100,000 ng/mL (ULOQ) for ADCs. The quality control levels were established at 80.0 ng/mL (LQC), 800 ng/mL (MQC), and 8,000 ng/mL (HQC2) and 40,000 ng/mL (HQC1).

Camptothecin (DAR8) ADCs were incubated at 37 degrees Celsius in mouse plasma (Balb C). The plasma was sampled at 6 h, 24 h, 72 h, and 7 days. The ADCs were isolated from plasma with IgSelect, deglycosylated with PNGase and reduced with dithiothreitol. Both ADC heavy and light chain were assessed by PLRP-MS to quantify drug-loading for at each timepoint.

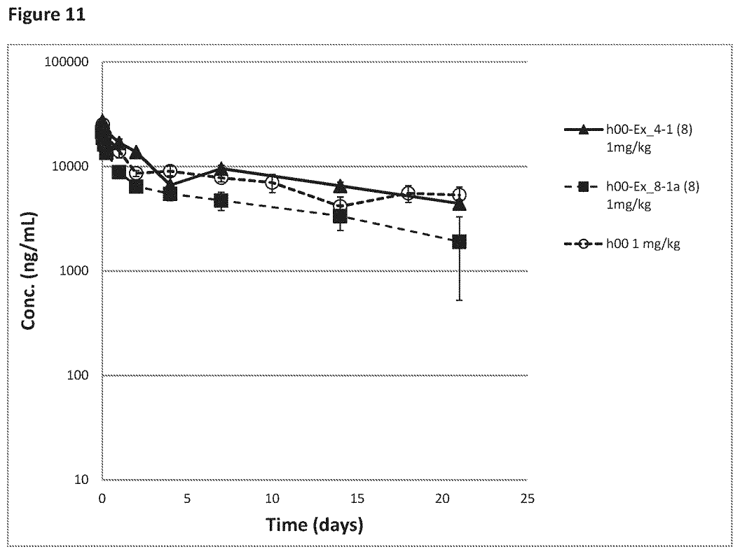

Rats were injected with 1 mg/kg of parental IgG, or IgG-camptothecin Ex_4-1 and Ex_8a ADCs. Samples from scheduled blood draws were processes and human IgG antibody and ADCs were captured from plasma via a biotin-conjugated murine anti-human light chain kappa mAb and a streptavidin-coated beads. Human IgG antibody and ADCs were quantified via ELISA using a AF647-anti-human kappa detection reagent. As shown in FIG. 11, ADC based on Ex_4-1 showed low uptake by Kupffer cells (liver macrophage), relative to ADC based on Ex_8-1a. Assay is a proxy for in vivo ADC clearance by the liver and suggests a low clearance rate for ADCs based on Ex_4-1.

### Kupffer Cell In Vitro Assay

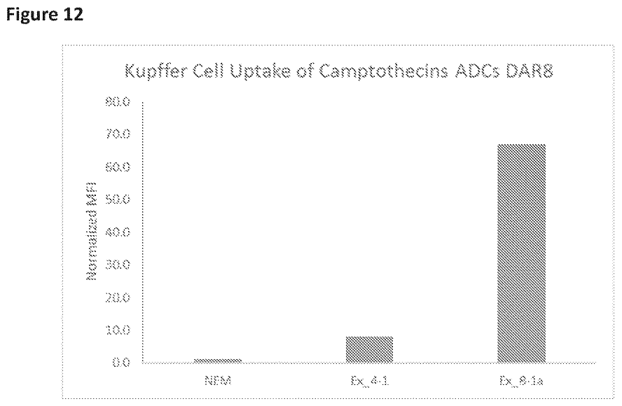

ADCs tested in the Kupffer cell assay were dually labeled with fluorescent dye and cytotoxic maleimide drug-linkers. Antibodies were first conjugated with fluorescent dye (AlexaFluor 647 NHS ester, ThermoFisher, Part #A20006,) to an average DAR=4. Dye labeled antibodies were then reduced using TCEP and conjugated with maleimide drug-linkers to an average DAR=8. Purified rat Kupffer cells (Life Technologies Corp. Part #RTKCCS) were plated on collagen I coated 96 well plates (ThermoFisher, Part #A1142803) at a density of 50,000 cells/well and allowed to adhere to the plate for 24-48 hr prior to adding ADCs. Kupffer Cells were incubated with ADCs at a concentration of 0.1 mg/mL in cell culture media for 24 hrs. After 24 hr incubation, media was removed, cells were dissociated with Versene, transferred to a conical bottom plate and washed one time by pelleting cells in a centrifuge at 400×g for 5 min, then resuspended in PBS+2% BSA. An Intellicyte iQue Screener equipped with ForeCyt software was used to count and measure ADC uptake into cells by mean fluorescent intensity (MFI) for each treatment condition. As shown in FIG. 12, ADC based on Ex_4-1 (DAR8) showed low uptake by Kupffer cells (liver macrophage), relative to ADC based on Ex_8-1a (DAR8). Assay is a proxy for in vivo ADC clearance by the liver and suggests a low clearance rate for ADCs based on Ex_4-1.

### Hydrophobicity Study Using Hydrophobic Interaction Chromatography (HIC)

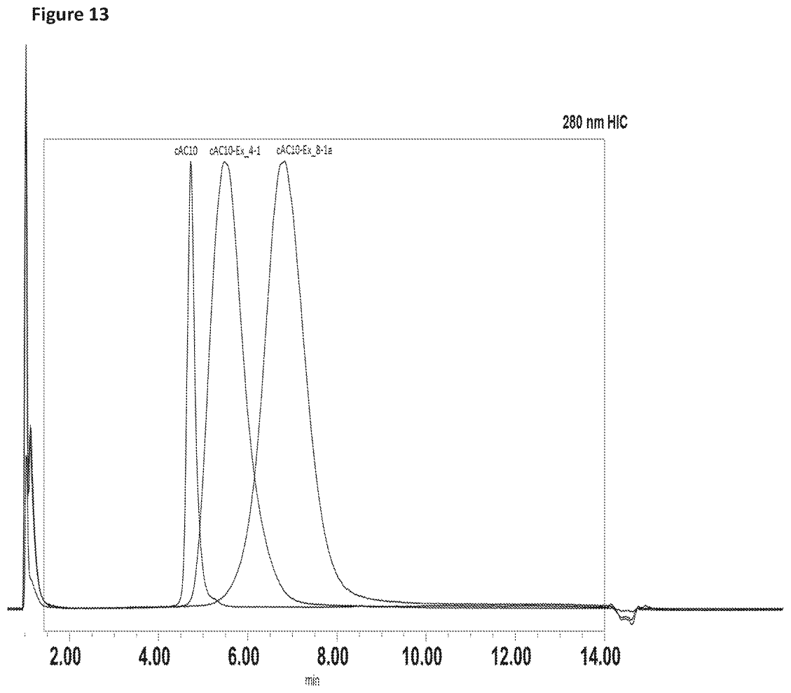

Naked cAC10, cAC10-Ex_4-1(8) and cAC10-Ex_8-1a(8) (approx. 75 μg,) were injected onto a Butyl HIC NPR column (2.5 μm, 4.6 mm×3.5 mm, Tosoh Bioscience, PN 14947) at 25° C. and eluted with a 12 minute linear gradient from 0-100% B at a flow rate of 0.8 mL/min (Mobile Phase A, 1.5 M ammonium sulfate in 25 mM potassium phosphate, pH 7; Mobile Phase B, 25 mM potassium phosphate, pH 7, 25% isopropanol). A Waters Alliance HPLC system equipped with a multi-wavelength detector and Empower3 software was used to resolve and quantify antibody species with different ratios of drugs per antibody. As shown in FIG. 13, cAC10-Ex_4-1 ADC displayed reduced hydrophobicity compared to cAC10-Ex_8-1a ADC or naked cAC10 antibody. ADC hydrophobicity is a contributor to ADC clearance and non-specific ADC uptake.

### Drug Release Study

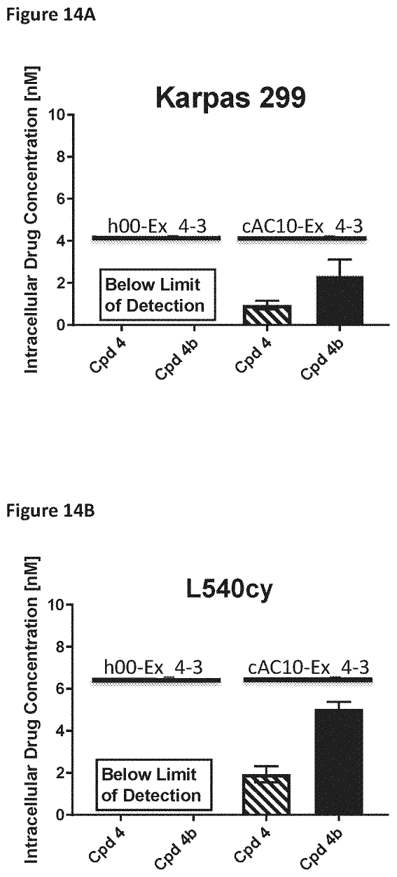

In vitro drug release from cAC10-Ex_4-3 ADC (DAR 8) was studied in ALCL cell line Karpas 299 and HL cell line L540cy. A non-binding h00-Ex_4-3 ADC (DAR 8) was used as the control. Karpas 299 (CD30 positive, T-cell lymphoma) and L540cy (CD30 positive, Hodgkin's lymphoma) cells were plated at 5E6 cells/mL (total of 5E6 cells) in fresh media (RPMI+10% FBS, RPMI+20% FBS, respectively). Upon plating, cells were dosed with cAC10-Ex_4-3 ADC (DAR 8) and h00-Ex_4-3 (DAR 8) at 10 ng/mL of culture. Treated cells were incubated at 37° C. and harvested 24 hours post-dose. Upon harvesting, cells were pelleted, washed with PBS and frozen down in a small volume of PBS. For analytical mass spec (LC-MS/MS) sample preparation, cells were extracted in cold methanol containing an internal standard and incubated on ice. After incubation, samples were centrifuged and supernatant (containing extracted small molecule) was removed and dried under nitrogen. Dried samples were reconstituted in 95% water containing 0.1% formic acid, and injected onto Waters Acquity BEH C18 (1.7 μm, 2.1×50 mm) column connected to Sciex 6500+ Triple Quadrupole Mass Spectrometer. As shown in FIGS. 14A and 14B, free drugs Compound 4 and Compound 4b are present in cells treated with cAC10-Ex_4-3 ADC (DAR 8), but not detectable in cells treated with h00-Ex_4-3 ADC (DAR 8).

Table of Sequences

| SEQ | | |
|---|---|---|
| ID | | |
| NO | Description | Sequence |
| 1 | cAC 10 | DYYIT |
| | CDR-H1 | |
| 2 | cAC 10 | WIYPGSGNTKYNEKFKG |
| | CDR-H2 | |
| 3 | cAC 10 | YGNYWFAY |
| | CDR-H3 | |
| 4 | cAC 10 | KASQSVDFDGDSYMN |
| | CDR-L1 | |
| 5 | cAC 10 | AASNLES |
| | CDR-L2 | |
| 6 | cAC 10 | QQSNEDPWT |
| | CDR-L3 | |
| 7 | cAC 10 VH | QIQLQQSGPEVVKPGASVKISCKASGYTFTDYYITWVKQKPGQGLEWIGWIYPGSGNTKY |
| | | NEKFKGKATLTVDTSSSTAFMQLSSLTSEDTAVYFCANYGNYWFAYWGQGTQVTVSA |
| 8 | cAC10 VL | DIVLTQSPASLAVSLGQRATISCKASQSVDFDGDSYMNWYQQKPGQPPKVLIYAASNLES |
| | | GIPARFSGSGSGTDFTLNIHPVEEEDAATYYCQQSNEDPWTFGGGTKLEIK |
| 9 | cAC10 HC | QIQLQQSGPEVVKPGASVKISCKASGYTFTDYYITWVKQKPGQGLEWIGWIYPGSGNTKY |
| | | NEKFKGKATLTVDTSSSTAFMQLSSLTSEDTAVYFCANYGNYWFAYWGQGTQVTVSAAST |
| | | KGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 10 | cAC 10 HC | QIQLQQSGPEVVKPGASVKISCKASGYTFTDYYITWVKQKPGQGLEWIGWIYPGSGNTKY |
| | V2 | NEKFKGKATLTVDTSSSTAFMQLSSLTSEDTAVYFCANYGNYWFAYWGQGTQVTVSAAST |
| | | KGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPG |
| 11 | cAC 10 LC | DIVLTQSPASLAVSLGQRATISCKASQSVDFDGDSYMNWYQQKPGQPPKVLIYAASNLES |
| | | GIPARFSGSGSGTDFTLNIHPVEEEDAATYYCQQSNEDPWTFGGGTKLEIKR |
| | | TVAAPSVFIFPPSDEQLKSGTASVVCLLNNFYPREAKVQWKVDNALQSGNSQESVTEQDS |
| | | KDSTYSLSSTLTLSKADYEKHKVYACEVTHQGLSSPVTKSFNRGEC |

1. A Camptothecin Conjugate having a formula:
L-(Q-D)ₚ (I)
or a pharmaceutically acceptable salt thereof, wherein
L is a Ligand Unit;
Q is a Linker Unit having a formula selected from:
Z-A-S*—RL-; —Z-A-L^{P}(S*)—RL-; —Z-A-S*—RL-Y—; or —Z-A-L^{P}(S*)—RL-Y—,
wherein Z is a Stretcher Unit, A is a bond or a Connector Unit; L^{P} is a Parallel Connector Unit; S* is a bond or a Partitioning Agent;
RL is a peptide comprising from 2 to 8 amino acids; and
Y is a Spacer Unit,
D is a Drug Unit selected from: 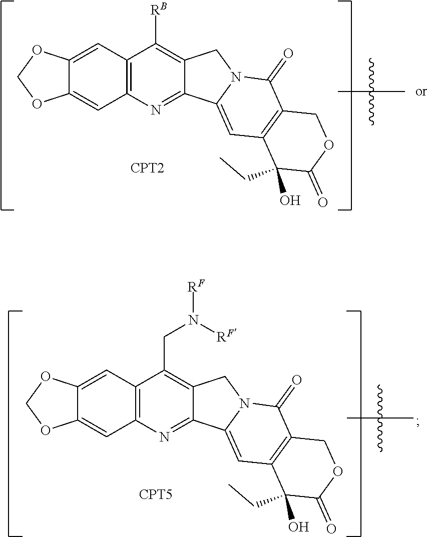
wherein
R^{B} is a member selected from the group consisting of H, —(C₁-C₄)alkyl-OH, —(C₁-C₄)alkyl-O—(C₁-C₄)alkyl-NH₂, —C₁-C₈ alkyl, C₁-C₈ haloalkyl, C₃-C₈ cycloalkyl, C₃-C₈cycloalkylC₁-C₄ alkyl, phenyl and phenylC₁-C₄ alkyl;
each R^{F} and R^{F′} is a member independently selected from the group consisting of H, C₁-C₈ alkyl, C₁-C₈ hydroxyalkyl, C₁-C₈ aminoalkyl, C₁-C₄ alkylaminoC₁-C₈ alkyl, (C₁-C₄ hydroxyalkyl)(C₁-C₄ alkyl)aminoC₁-C₈ alkyl, di(C₁-C₄ alkyl)aminoC₁-C₈ alkyl, C₁-C₄ hydroxyalkylC₁-C₈ aminoalkyl, C₂-C₆ heteroalkyl, C₁-C₈ alkylC(O)—, C₁-C₈ hydroxyalkylC(O)—, C₁-C₈ aminoalkylC(O)—, C₃-C₁₀ cycloalkyl, C₃-C₁₀cycloalkylC₁-C₄ alkyl, C₃-C₁₀ heterocycloalkyl, C₃-C₁₀ heterocycloalkylC₁-C₄ alkyl, phenyl, phenylC₁-C₄ alkyl, diphenylC₁-C₄ alkyl, heteroaryl and heteroarylC₁-C₄ alkyl; or R^{F} and R^{F′} are combined with the nitrogen atom to which each is attached to form a 5-, 6- or 7-membered ring having 0 to 3 substituents selected from halogen, C₁-C₄ alkyl, OH, OC₁-C₄ alkyl, NH₂, NHC₁-C₄ alkyl and N(C₁-C₄ alkyl)₂;
and wherein cycloalkyl, heterocycloalkyl, phenyl and heteroaryl portions of R^{B}, R^{F} and R^{F′} are substituted with from 0 to 3 substituents selected from halogen, C₁-C₄ alkyl, OH, OC₁-C₄ alkyl, NH₂, NHC₁-C₄ alkyl and N(C₁-C₄ alkyl)₂; and
p is from about 1 to about 16;
wherein Q is attached through any one of the hydroxyl or amine groups present on CPT2 or CPT5.

2. The Camptothecin Conjugate of claim 1, wherein D has formula CPT2.

3-6. (canceled)

7. The Camptothecin Conjugate of claim 1, wherein D has formula CPT5.

8. The Camptothecin Conjugate of claim 7, wherein the -Q-D component of the Conjugate has a formula selected from (CPT5iN), (CPT5iiN), (CPT5iiiN), (CPT5ivN), (CPT5vN), (CPT5viN), (CPT5iO), (CPT5iiO), (CPT5iiiO), (CPT5ivO), (CPT5vO), and (CPT5viO):

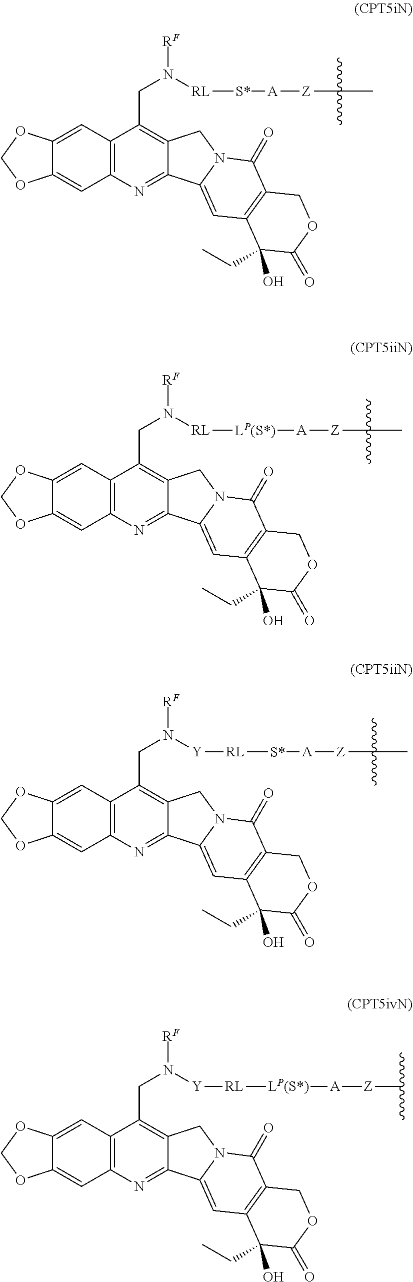

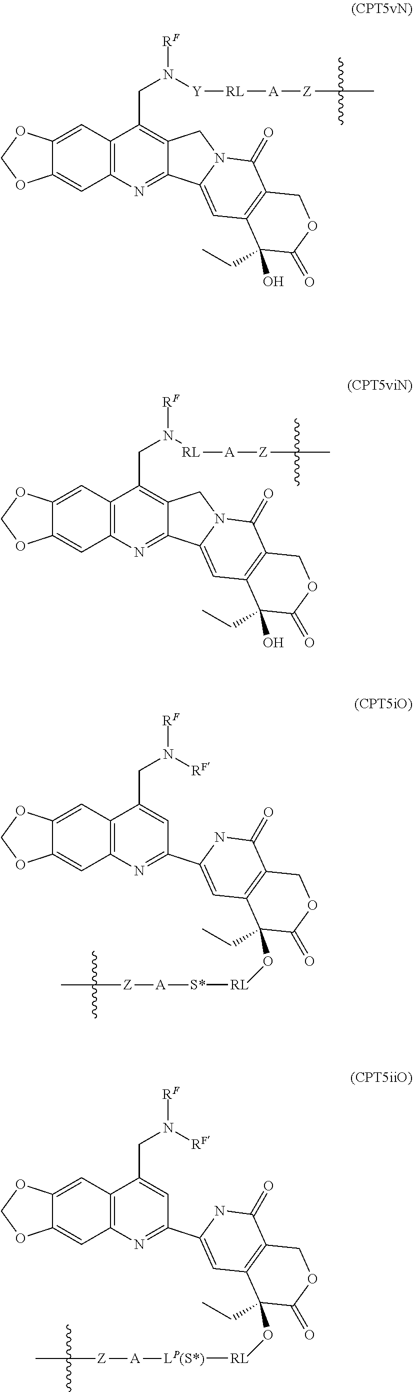

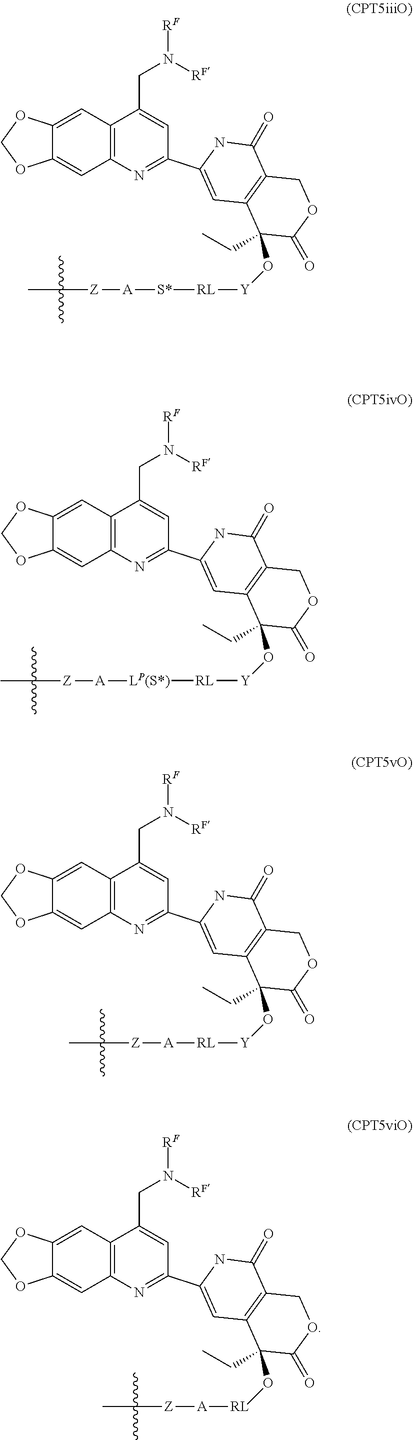

9-11. (canceled)

12. The Camptothecin Conjugate of claim 8, wherein the -Q-D component of the Camptothecin Conjugate has a formula selected from (CPT5iN), (CPT5iiN), (CPT5iiiN), (CPT5ivN), (CPT5vN), and (CPT5viN).

13. (canceled)

14. The Camptothecin Conjugate of claim 12, wherein R^{F} is selected from the group consisting of —H, C₁-C₈ alkyl, C₁-C₈ hydroxyalkyl, C₁-C₈ aminoalkyl, C₁-C₄ alkylaminoC₁-C₈ alkyl, (C₁-C₄ hydroxyalkyl)(C₁-C₄ alkyl)aminoC₁-C₈ alkyl, di(C₁-C₄ alkyl)aminoC₁-C₈ alkyl, C₁-C₄ hydroxyalkylC₁-C₈ aminoalkyl, C₁-C₈ alkylC(O)—, C₁-C₈ hydroxyalkylC(O)—, and C₁-C₈ aminoalkylC(O)—;
and wherein cycloalkyl, heterocycloalkyl, phenyl and heteroaryl portions of R^{F} are substituted with from 0 to 3 substituents selected from halogen, C₁-C₄ alkyl, OH, OC₁-C₄ alkyl, NH₂, NHC₁-C₄ alkyl and N(C₁-C₄ alkyl)₂.

15. (canceled)

16. The Camptothecin Conjugate of claim 1, wherein S* is a bond and Q is —Z-A-RL- or —Z-A-RL-Y—.

17. The Camptothecin Conjugate of claim 1, wherein S* is a Partitioning Agent, and Q is
—Z-A-S*—RL-; —Z-A-L^{P}(S*)—RL-; —Z-A-S*—RL-Y—; or —Z-A-L^{P}(S*)—RL-Y—.

18. (canceled)

19. The Camptothecin Conjugate of claim 18, the PEG Unit has the formula: 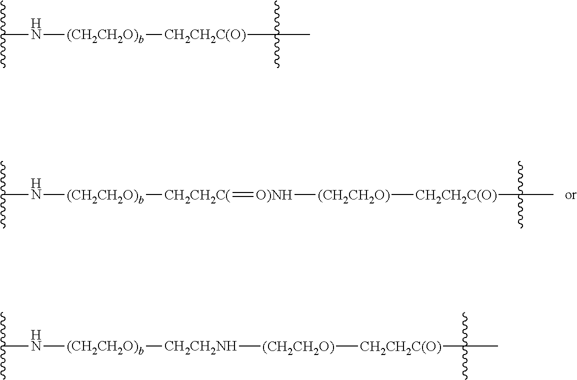
wherein the wavy line on the left indicates the site of attachment to A, the wavy line on the right indicates the site of attachment to RL, and b is an integer from 2 to 20, or is 2, 4, 8, or 12.

20. The Camptothecin Conjugate of claim 19, wherein the PEG Unit has the formula: 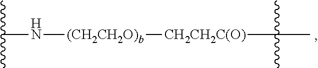
wherein the wavy line on the left indicates the site of attachment to A, the wavy line on the right indicates the site of attachment to RL, and b is an integer from 2 to 20, or is 2, 4, 8, or 12.

21. The Camptothecin Conjugate of 17, wherein Q is of formula —Z-A-L^{P}(S*)—RL- or —Z-A-L^{P}(S*)—RL-Y— and S* is a PEG Unit which comprises 2, 4, 8, or 12 —CH₂CH₂O— subunits and a PEG Unit terminal cap group that is C₁₋₄alkyl or C₁₋₄alkyl-CO₂H.

22. The Camptothecin Conjugate of claim 21, wherein S* is of formula: 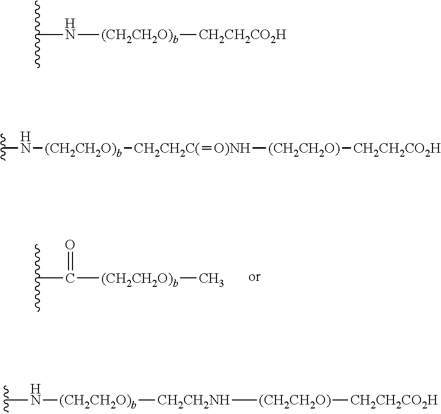
wherein the wavy line indicates the site of attachment to the Parallel Connector Unit (L^{P}), and b is an integer from 2 to 20, or is 2, 4, 8, or 12.

23. The Camptothecin Conjugate of claim 22, wherein S* is of formula: 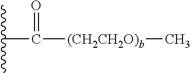
wherein the wavy line indicates the site of attachment to the Parallel Connector Unit (L^{P}), and b is an integer from 2 to 20, or is 2, 4, 8, or 12.

24. (canceled)

25. The Camptothecin Conjugate of claim 24, wherein L^{P} is of formula: 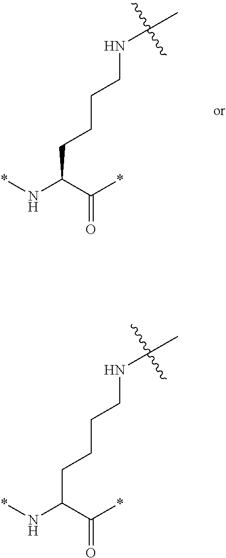 [SMILES: *NCCCC[C@H](N*)C(*)=O ; *NCCCCC(N*)C(*)=O]
wherein the wavy line indicates the position of attachment to the Partitioning Agent and asterisks indicate positions of attachment to A and RL.

26. The Camptothecin Conjugate of claim 1, wherein Z has Formula Za: 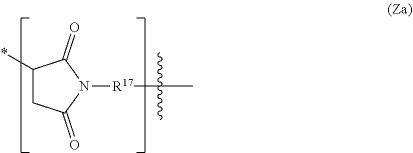
wherein the asterisk indicates the position of attachment to the Ligand Unit (L);
the wavy line indicates the position of attachment to the Connector Unit (A); and
R¹⁷ is —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₅ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₅ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₅ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₅ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—;
wherein R¹⁷ is optionally substituted with a Basic Unit (BU) that is —(CH₂)ₓNH₂, —(CH₂)ₓNHR^{a}, or —(CH₂)ₓNR^{a}₂;
wherein x is an integer of from 1-4; and
each R^{a} is independently selected from the group consisting of C₁₋₆ alkyl and C₁₋₆ haloalkyl, or two R^{a} groups are combined with the nitrogen to which they are attached to form a 4- to 6-membered heterocycloalkyl ring, or an azetidinyl, pyrrolidinyl or piperidinyl group.

27. (canceled)

28. The Camptothecin Conjugate of claim 26, wherein Z is:

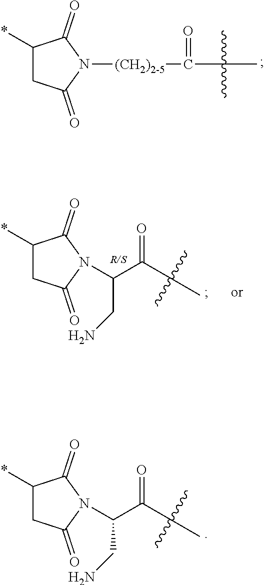 [SMILES: *C(=O)CN1C(=O)CC(*)C1=O ; *C(=O)C(CN)N1C(=O)CC(*)C1=O ; *C(=O)[C@H](CN)N1C(=O)CC(*)C1=O]

29. The Camptothecin Conjugate of claim 28, wherein Z is:

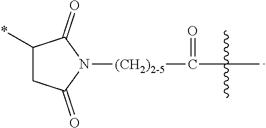 [SMILES: *C(=O)CN1C(=O)CC(*)C1=O]

30. (canceled)

31. The Camptothecin Conjugate of claim 1, wherein A is a bond.

32. The Camptothecin Conjugate of claim 1, wherein RL is a dipeptide, tripeptide, or tetrapeptide.

33. (canceled)

34. (canceled)

35. The Camptothecin Conjugate of claim 32, wherein RL is gly-gly, gly-gly-gly, gly-gly-gly-gly, val-gly-gly, val-cit-gly, val-gln-gly, val-glu-gly, phe-lys-gly, leu-lys-gly, gly-val-lys-gly, val-lys-gly-gly, val-lys-gly, val-lys-ala, val-lys-leu, leu-leu-gly, gly-gly-phe-gly, gly-gly-phe-gly-gly, val-gly, or val-lys-β-ala.

36. The Camptothecin Conjugate of claim 32, wherein RL is a tripeptide having the formula: AA₁-AA₂-AA₃, wherein AA₁, AA₂ and AA₃ are each independently an amino acid, wherein AA₁ attaches to —NH— and AA₃ attaches to S*.

37. The Camptothecin Conjugate of claim 36, wherein AA₃ is gly or β-ala.

38. The Camptothecin Conjugate of claim 37, wherein RL is val-lys-gly, wherein val attaches to —NH— and gly attaches to S*.

39. The Camptothecin Conjugate claim 1, wherein Y is of the formula:

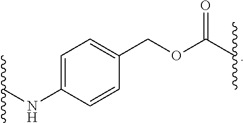 [SMILES: *Nc1ccc(COC(*)=O)cc1]

40. The Camptothecin Conjugate of claim 1, wherein p is 1 to 16.

41-46. (canceled)

47. The Camptothecin Conjugate of claim 1, having the Formula (IC): 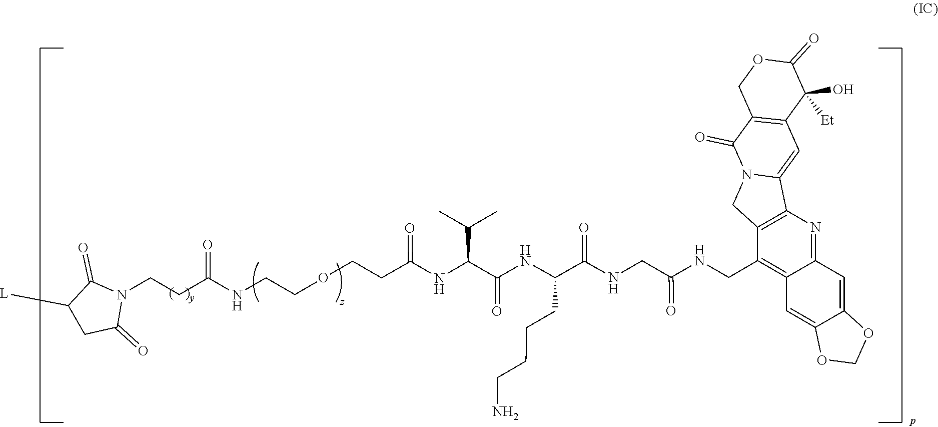
or a pharmaceutically acceptable salt thereof;
wherein
y is 1, 2, 3, or 4; and
z is 2, 4, 8, or 12;
and p is 1-16.

48. The Camptothecin Conjugate of claim 47, wherein p is 4 or 8.

49-52. (canceled)

53. The Camptothecin Conjugate of claim 1, wherein the Ligand Unit is an antibody or an antigen-binding fragment thereof.

54. (canceled)

55. (canceled)

56. A Camptothecin-Linker Compound of the formula:
Q′-D,
or a pharmaceutically acceptable salt thereof, wherein
Q′ is a Linker Unit Precursor having a formula selected from the group consisting of:
Z′-A-S*—RL-; Z′-A-L^{P}(S*)—RL-; Z′-A-S*—RL-Y—; Z′-A-L^{P}(S*)—RL-Y—;
wherein
Z′ is a Stretcher Unit Precursor;
A is a bond or a Connector Unit;
S* is a bond or a Partitioning Agent;
L^{P} is a Parallel Connector Unit;
RL is a Peptide Releasable Linker comprising a peptide comprising 2 to 8 amino acids; and
Y is a Spacer Unit,
D is a Drug Unit selected from: 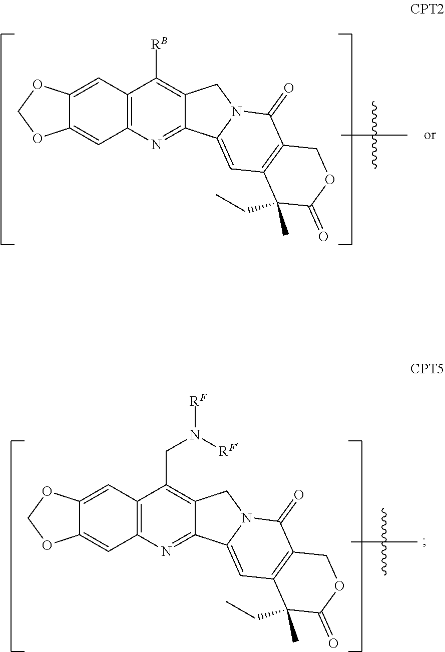
wherein
R^{B} is a member selected from the group consisting of H, —(C₁-C₄)alkyl-OH, —(C₁-C₄)alkyl-O—(C₁-C₄)alkyl-NH₂, —C₁-C₈ alkyl, C₁-C₈ haloalkyl, C₃-C₈ cycloalkyl, C₃-C₈cycloalkylC₁-C₄ alkyl, phenyl and phenylC₁-C₄ alkyl;
each R^{F} and R^{F′} is a member independently selected from the group consisting of H, C₁-C₈ alkyl, C₁-C₈ hydroxyalkyl, C₁-C₈ aminoalkyl, C₁-C₄ alkylaminoC₁-C₈ alkyl, (C₁-C₄ hydroxyalkyl)(C₁-C₄ alkyl)aminoC₁-C₈ alkyl, di(C₁-C₄ alkyl)aminoC₁-C₈ alkyl, C₁-C₄ hydroxyalkylC₁-C₈ aminoalkyl, C₁-C₈ alkylC(O)—, C₁-C₈ hydroxyalkylC(O)—, C₁-C₈ aminoalkylC(O)—, C₃-C₁₀ cycloalkyl, C₃-C₁₀cycloalkylC₁-C₄ alkyl, C₃-C₁₀ heterocycloalkyl, C₃-C₁₀ heterocycloalkylC₁-C₄ alkyl, phenyl, phenylC₁-C₄ alkyl, diphenylC₁-C₄ alkyl, heteroaryl and heteroarylC₁-C₄ alkyl; or R^{F} and R^{F′} are combined with the nitrogen atom to which each is attached to form a 5-, 6- or 7-membered ring having 0 to 3 substituents selected from halogen, C₁-C₄ alkyl, OH, OC₁-C₄ alkyl, NH₂, NHC₁-C₄ alkyl and N(C₁-C₄ alkyl)₂;
and wherein cycloalkyl, heterocycloalkyl, phenyl and heteroaryl portions of R^{B}, R^{F} and R^{F′} are substituted with from 0 to 3 substituents selected from halogen, C₁-C₄ alkyl, OH, OC₁-C₄ alkyl, NH₂, NHC₁-C₄ alkyl and N(C₁-C₄ alkyl)₂,
wherein Q is attached through any one of the hydroxyl or amine groups present on CPT2 or CPT5.

57. The Camptothecin-Linker Compound of claim 56, wherein D has formula CPT2.

58-61. (canceled)

62. The Camptothecin-Linker Compound of claim 56, wherein D has formula CPT5.

63-110. (canceled)

111. A Camptothecin Compound of formula: 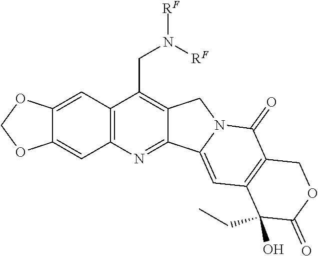
wherein each R^{F} and R^{F′} is independently H, glycyl, hydroxyacetyl, ethyl, or 2-(2-(2-aminoethoxy)ethoxy)ethyl, or wherein R^{F} and R^{F′} are combined with the nitrogen atom to which each is attached to form a 5-, 6-, or 7-membered heterocycloalkyl ring.

112. The Camptothecin Compound of claim 111, wherein R^{F} and R^{F′} are combined with the nitrogen atom to which each is attached to form a 6-membered ring.

113. (canceled)

114. The Camptothecin Compound of claim 111, wherein R^{F′} is H and R^{F} is glycyl, hydroxyacetyl, ethyl, or 2-(2-(2-aminoethoxy)ethoxy)ethyl, an aliphatic group, an aryl group, an amide group, or an ethylene oxide group.

115-118. (canceled)

119. The Camptothecin Compound of claim 111, wherein the compound is selected from the group selected from Compound 4, Compound 5, and compounds in Tables I and II.

120. A Camptothecin Compound of formula: 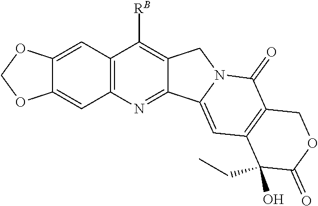
or a pharmaceutically acceptable salt thereof,
wherein R^{B} is —(C₁-C₄)alkyl-OH, —(C₁-C₄)alkyl-O—(C₁-C₄)alkyl-NH₂, —C₁-C₈ alkyl, C₁-C₈ haloalkyl, C₃-C₈ cycloalkyl, C₃-C₈cycloalkylC₁-C₄ alkyl, phenyl or phenylC₁-C₄ alkyl.

121. (canceled)

122. (canceled)

123. The Camptothecin Compound of claim 120, wherein the compound is selected from the group consisting of Compound 6 and compounds in Table III.

124. The Camptothecin Conjugate of claim 53, wherein the antibody or antigen-binding fragment thereof comprises CDR-H1, CDR-H2, CDR-H3, CDR-L1, CDR-L2, and CDR-L3 comprising the amino acid sequences of SEQ ID NOs: 1, 2, 3, 4, 5, and 6, respectively.

125. The Camptothecin Conjugate of claim 124, wherein the antibody or antigen-binding fragment thereof comprises a heavy chain variable region comprising an amino acid sequence that is at least 95% identical to the amino acid sequence of SEQ ID NO: 7 and a light chain variable region comprising an amino acid sequence that is at least 95% identical to the amino acid sequence of SEQ ID NO: 8.

126. The Camptothecin Conjugate of claim 124, wherein the antibody or antigen-binding fragment thereof comprises a heavy chain variable region comprising the amino acid sequence of SEQ ID NO: 7 and a light chain variable region comprising the amino acid sequence of SEQ ID NO: 8.

127. The Camptothecin Conjugate of claim 124, wherein the antibody or comprises a heavy chain comprising the amino acid sequence of SEQ ID NO: 9 or SEQ ID NO: 10 and a light chain comprising the amino acid sequence of SEQ ID NO: 11.

128. The Camptothecin Conjugate of claim 124, having Formula (IC): 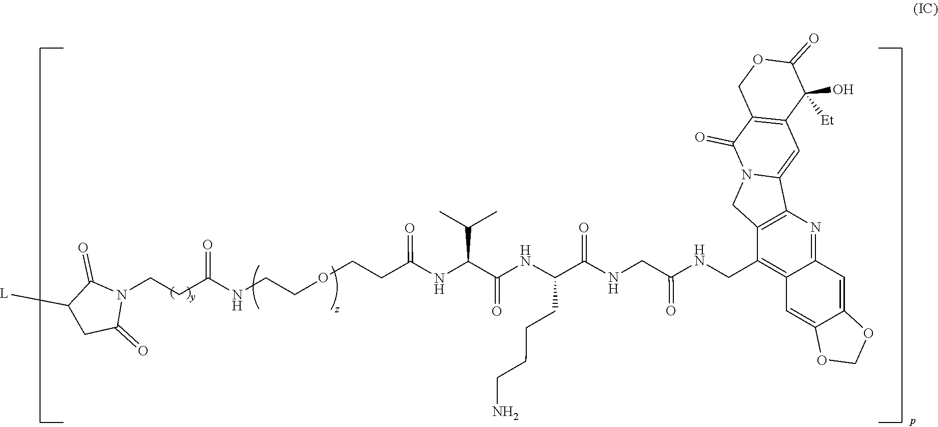
or a pharmaceutically acceptable salt thereof;
wherein
y is 1, 2, 3, or 4, or is 1 or 4; and
z is an integer from 2 to 12, or is 2, 4, 8, or 12;
and p is 1-16.

129. The Camptothecin Conjugate of claim 128, wherein p is 2, 4 or 8.

130. The Camptothecin Conjugate of claim 53, having formula: 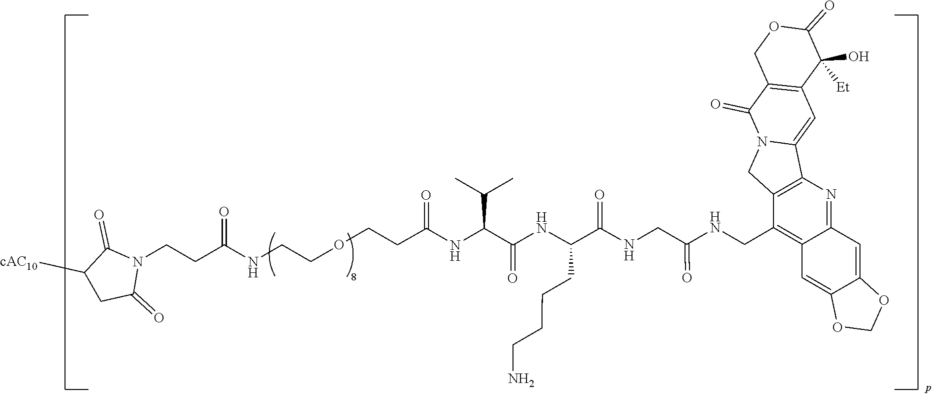
or a pharmaceutically acceptable salt thereof;
wherein
p is 2, 4, or 8.

131. (canceled)

132. A method of treating cancer or an autoimmune disease in a subject in need thereof, comprising administering to the subject an effective amount of a Camptothecin Conjugate of claim 1.

133-136. (canceled)

137. A method of treating cancer in a subject in need thereof, comprising contacting the cancer cells with the Camptothecin Compound of claim 111.

138. (canceled)

139. A method of preparing a Camptothecin Conjugate of claim 1, comprising reacting an antibody or antigen-binding fragment thereof with a Camptothecin-Linker Compound of claim 56.

140. A pharmaceutical composition comprising the Camptothecin Conjugate of claim 1 and a pharmaceutically acceptable carrier.

141-143. (canceled)
